(19) United States
(12) Patent Application Publication
(10) Pub. No.: US 2022/0359832 A1
(43) Pub. Date: Nov. 10, 2022

(54) ORGANIC ELECTROLUMINESCENT DEVICE

(71) Applicant: BEIJING SUMMER SPROUT TECHNOLOGY CO., LTD., Beijing (CN)

(72) Inventors: Jing Wang, Beijing (CN); Zhihao Cui, Beijing (CN); Chi Yuen Raymond Kwong, Beijing (CN); Huiqing Pang, Beijing (CN); Menglan Xie, Beijing (CN)

(73) Assignee: BEIJING SUMMER SPROUT TECHNOLOGY CO., LTD., Beijing (CN)

(21) Appl. No.: 17/649,874

(22) Filed: Feb. 3, 2022

(30) Foreign Application Priority Data
Feb. 6, 2021 (CN) 202110165072.1
Dec. 8, 2021 (CN) 202111488167.3

(51) Int. Cl. H01L 51/00; C07D 498/04; C09K 11/06; C07C 211/61

(52) U.S. Cl. CPC H01L 51/0071; C07D 498/04; C09K 11/06; C07C 211/61; H01L 51/006; H01L 51/5088

(57) ABSTRACT

Disclosed is an organic electroluminescent device. The organic electroluminescent device comprises an anode, a cathode and a first organic layer disposed between the anode and the cathode, wherein the first organic layer contains a first compound having a structure of Formula 1 and a second compound having a structure of Formula 2. Such a novel material combination composed of the first compound and the second compound may be used in a hole injection layer in the organic electroluminescent device and can endow the organic electroluminescent device with excellent characteristics of low voltage, high efficiency and a long lifetime and provide better device performance. Further disclosed are a display assembly and a compound combination.

### CROSS-REFERENCE TO RELATED APPLICATION(S)

This application claims priority to Chinese Patent Application No. CN 202110165072.1 filed on Feb. 6, 2021 and Chinese Patent Application No. CN 202111488167.3 filed on Dec. 8, 2021, the disclosure of which are incorporated herein by reference in their entireties.

### TECHNICAL FIELD

The present disclosure relates to organic electronic devices and, in particular, to an organic electroluminescent device. More particularly, the present disclosure relates to an organic electroluminescent device containing a first compound having a structure of Formula 1 and a second compound having a structure of Formula 2 in a first organic layer, and a display assembly including the organic electroluminescent device.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

Organic electroluminescent devices convert electrical energy into light by applying voltages across the devices. Generally, an organic electroluminescent device includes an anode, a cathode and organic layers disposed between the anode and the cathode. The organic layers of the electroluminescent device include hole injection layer, hole transporting layer, electron blocking layer, light-emitting layer (containing a host material and a doping material), electron buffer layer, hole blocking layer, electron transporting layer, electron injection layer and the like. According to different functions of materials, the materials that constitute the organic layer may be divided into hole injection material, hole transporting material, electron blocking material, host material, light-emitting material, electron buffer material, hole blocking material, electron transporting material, electron injection material and the like. When a bias voltage is applied to the device, holes are injected into the light-emitting layer from the anode, and electrons are injected into the light-emitting layer from the cathode. The holes and the electrons meet each other to form excitons, and the excitons are recombined to emit light. The hole injection layer is one of important function layers that affect the performance of the organic electroluminescent device. The selection and matching of materials of the hole injection layer can have an important effect on the performance of the organic electroluminescent device, such as driving voltage, efficiency and lifetime. It is expected commercially to obtain the organic electroluminescent device with low driving voltage, high efficiency, a long service lifetime and other characteristics. Therefore, the development of a novel hole injection layer is a very critical research field.

Most of early OLED devices include only one layer of organic material between the anode and the light-emitting layer, to implement the functions of hole injection, hole transporting and even electron blocking. Such a device structure, limited by a single hole transporting material, cannot achieve ideal matching of energy levels. Thus, it is difficult to obtain very ideal performance. As the industry has increasing requirements on device performance, increasing requirements are imposed on the performance of a hole transporting region between the anode and the light-emitting layer and the hole transporting material is refined into two layers: the hole injection layer and the hole transporting layer. In this case, a single triarylamine material is generally used as the hole injection layer. Common triarylamine materials are as follows:

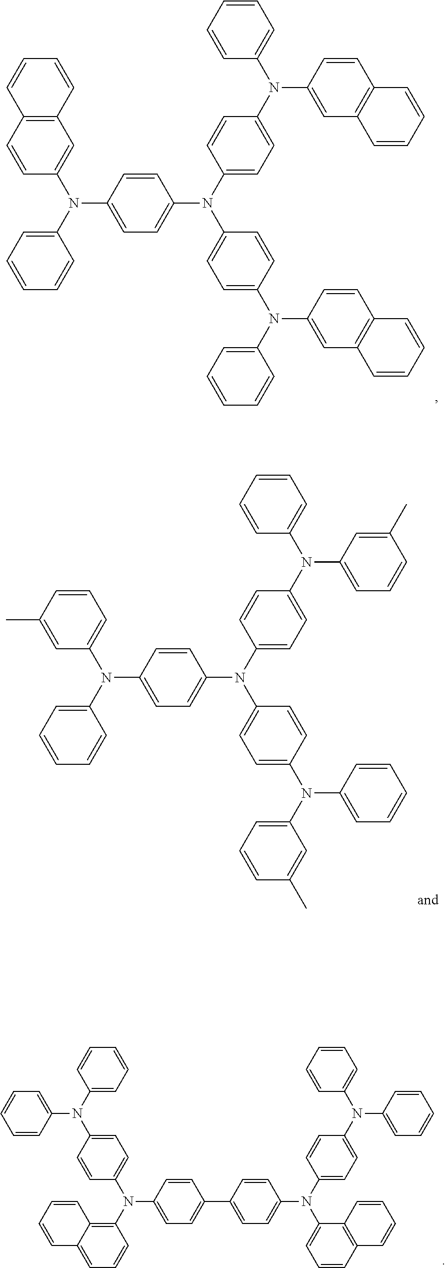 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; c1ccc(N(c2ccccc2)c2ccc(N(c3ccc(-c4ccc(N(c5ccc(N(c6ccccc6)c6ccccc6)cc5)c5cccc6ccccc56)cc4)cc3)c3cccc4ccccc34)cc2)cc1]

As another example, US20160190447A1 discloses an organic compound having a spirobifluorene-triarylamine structure:

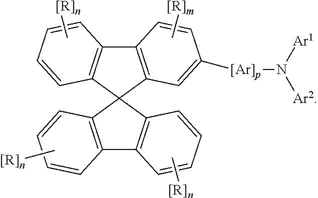

The compound may be used as a hole transporting material in a hole transporting layer or a hole injection layer or an exciton blocking layer or used as a fluorescent emitter or a matrix material of a phosphorescent emitter. However, this reference pays no attention to an effect of the matching and selection of the compound and a p-type doping material on device performance.

At present, in the most advanced device structure in the industry, multiple organic layers are generally arranged between the anode and the light-emitting layer to implement a hole injection function, a hole transporting function and an electron blocking function, respectively. To obtain a better hole injection effect, the hole transporting material (such as arylamine compounds) of the hole injection layer is often doped with a certain proportion of p-type doping materials. Common p-type doping materials are as follows:

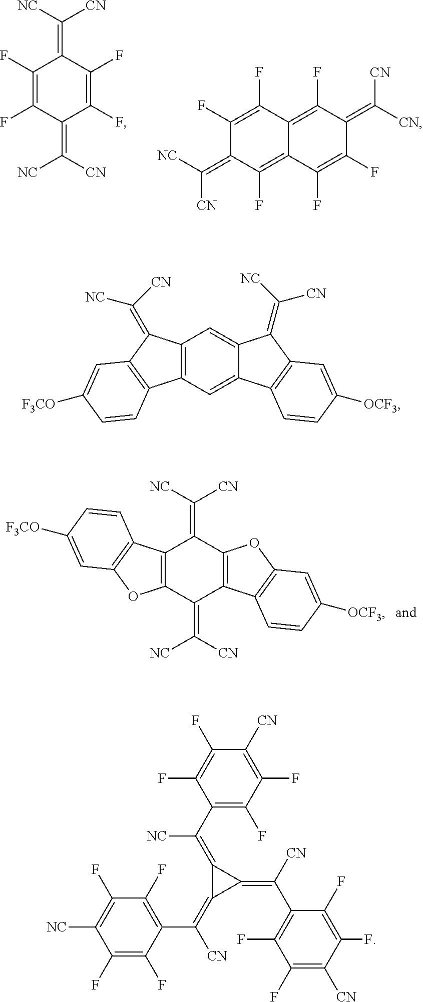 [SMILES: N#CC(C#N)=c1c(F)c(F)c(=C(C#N)C#N)c(F)c1F ; N#CC(C#N)=c1c(F)c(F)c2c(F)c(=C(C#N)C#N)c(F)c(F)c2c1F ; N#CC(C#N)=C1c2cc(OC(F)(F)F)ccc2-c2cc3c(cc21)C(=C(C#N)C#N)c1cc(OC(F)(F)F)ccc1-3 ; N#CC(C#N)=c1c2oc3cc(OC(F)(F)F)ccc3c2c(=C(C#N)C#N)c2oc3cc(OC(F)(F)F)ccc3c12 ; "and" ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

As another example, US20200087311A1 discloses an organic compound having dehydrobenzodioxazole, dehydrobenzodithiazole or dehydrobenzodiselenazole and similar structures

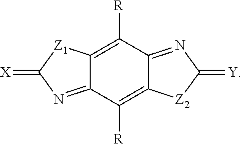

The compound may be used as a p-type doping material or a hole injection material with deep LUMO. This application only focuses on such p-type doping materials themselves and pays no attention to an effect of the matching and selection of such p-type doping materials and a hole transport material on device performance.

Such doped hole injection layers achieve a p-type doping effect through a strong electron trapping ability of the p-type doping materials and can obtain effectively improved hole injection performance and conductivity. In such doped hole injection layers, on the one hand, it is very important to research and develop better p-type doping materials and/or better hole transporting materials; on the other hand, the matching of the p-type doping materials and the hole transporting materials is more important, and mismatching often results in greatly reduced device performance. Therefore, the reasonable matching and selection of p-type doping materials and hole transport materials is very critical.

### SUMMARY

The present disclosure aims to provide a series of novel organic electroluminescent devices to solve at least part of the above-mentioned problems. The novel organic electroluminescent device comprises an anode, a cathode and a first organic layer disposed between the anode and the cathode, wherein the first organic layer at least contains a first compound having a structure of Formula 1 and a second compound having a structure of Formula 2. Such a novel material combination composed of the first compound and the second compound may be used in a hole injection layer in the organic electroluminescent device and can endow the organic electroluminescent device with excellent characteristics of low voltage, high efficiency and a long lifetime and provide better device performance.

According to an embodiment of the present disclosure, an organic electroluminescent device is disclosed, which comprises:

an anode,

a cathode, and

a first organic layer disposed between the anode and the cathode, wherein the first organic layer at least contains a first compound and a second compound, wherein the first compound has a structure represented by Formula 1:

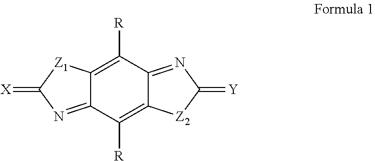

wherein in Formula 1,

X and Y are, at each occurrence identically or differently, selected from NR′, CR″R′″, O, S or Se;

Z₁ and Z₂ are, at each occurrence identically or differently, selected from O, S or Se;

R, R′, R″ and R′″ are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,

each R may be the same or different, and at least one of R, R′, R″ and R′″ is a group having at least one electron withdrawing group;

in Formula 1, adjacent substituents can be optionally joined to form a ring;

wherein the second compound has a structure represented by Formula 2:

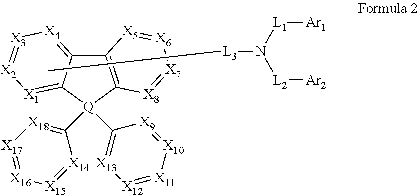

wherein in Formula 2,

X₁ to X₈ are, at each occurrence identically or differently, selected from C, CR₁ or N; and X₉ to X₁₈ are, at each occurrence identically or differently, selected from CR₁ or N;

Q is selected from C, Si or Ge;

L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof,

Ar₁ and Ar₂ are, at each occurrence identically or differently, selected from substituted or unsubstituted aryl having 6 to 30 carbon atoms or substituted or unsubstituted heteroaryl having 3 to 30 carbon atoms;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and

adjacent substituents L₁, L₂, L₃, R₁, Ar₁ and Ar₂ can be optionally joined to form a ring.

According to another embodiment of the present disclosure, a display assembly is further disclosed, which includes the organic electroluminescent device in the preceding embodiment.

According to another embodiment of the present disclosure, a compound combination is further disclosed, which contains a first compound and a second compound, wherein the first compound has a structure represented by Formula 1:

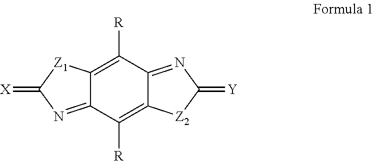

wherein in Formula 1,

X and Y are, at each occurrence identically or differently, selected from NR′, CR″R′″, O, S or Se;

Z₁ and Z₂ are, at each occurrence identically or differently, selected from O, S or Se;

R, R′, R″ and R′″ are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,

each R may be the same or different, and at least one of R, R′, R″ and R′″ is a group having at least one electron withdrawing group;

in Formula 1, adjacent substituents can be optionally joined to form a ring;

wherein the second compound has a structure represented by Formula 2:

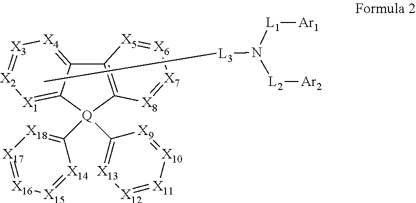

wherein in Formula 2,

X₁ to X₈ are, at each occurrence identically or differently, selected from C, CR₁ or N; and X₉ to X₁₈ are, at each occurrence identically or differently, selected from CR₁ or N;

Q is selected from C, Si or Ge;

L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof;

Ar₁ and Ar₂ are, at each occurrence identically or differently, selected from substituted or unsubstituted aryl having 6 to 30 carbon atoms or substituted or unsubstituted heteroaryl having 3 to 30 carbon atoms;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and

adjacent substituents L₁, L₂, L₃, R₁, Ar₁ and Ar₂ can be optionally joined to form a ring.

The novel organic electroluminescent device disclosed in the present disclosure comprises the anode, the cathode and the first organic layer disposed between the anode and the cathode, wherein the first organic layer contains at least the first compound having the structure of Formula 1 and the second compound having the structure of Formula 2. Such a novel material combination composed of the first compound and the second compound may be used in the hole injection layer in the organic electroluminescent device and can endow the organic electroluminescent device with the excellent characteristics of low voltage, high efficiency and a long lifetime and provide the better device performance.

### BRIEF DESCRIPTION OF DRAWINGS

FIG. 1 is a schematic diagram of an organic light-emitting apparatus that may include an organic electroluminescent device disclosed herein.

FIG. 2 is a schematic diagram of another organic light-emitting apparatus that may include an organic electroluminescent device disclosed herein.

### DETAILED DESCRIPTION

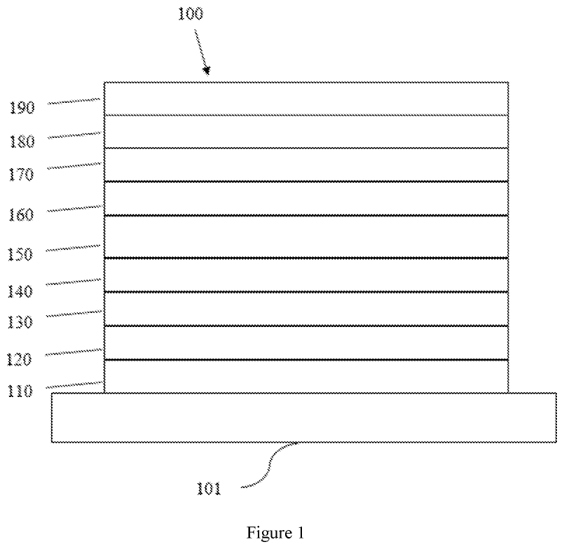

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

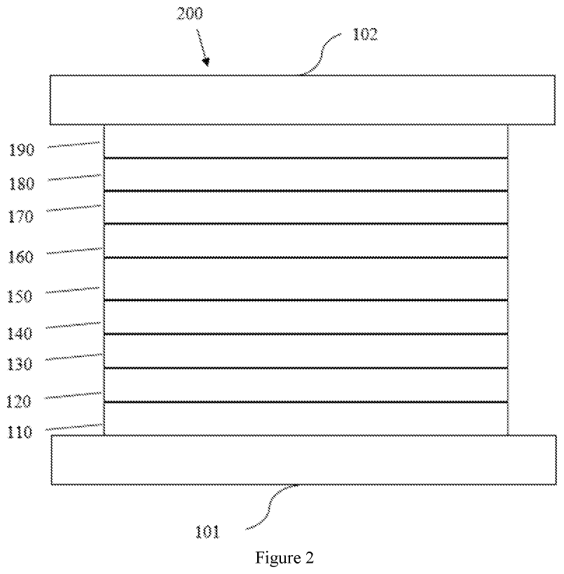

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitutions (including di-, tri-, and tetra-substitutions etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fused cyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

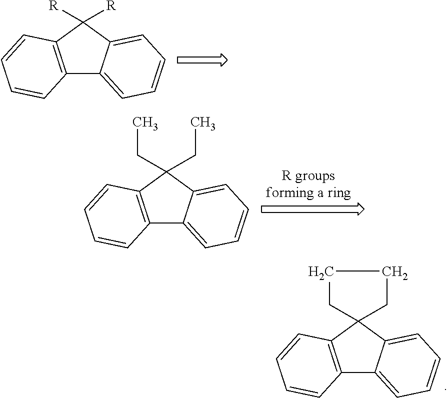

The expression that adjacent substituents can be optionally joined to form a ring is also intended to mean that two substituents bonded to carbon atoms which are directly bonded to each other are joined to each other via a chemical bond to form a ring, which can be exemplified by the following formula:

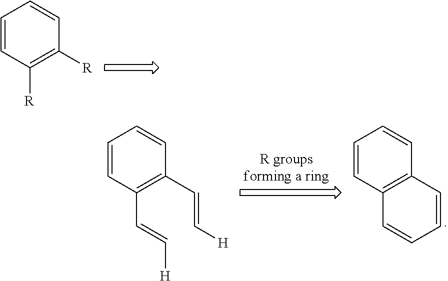

The expression that adjacent substituents can be optionally joined to form a ring is also intended to mean that two substituents bonded to further distant carbon atoms are joined to each other via a chemical bond to form a ring, which can be exemplified by the following formula:

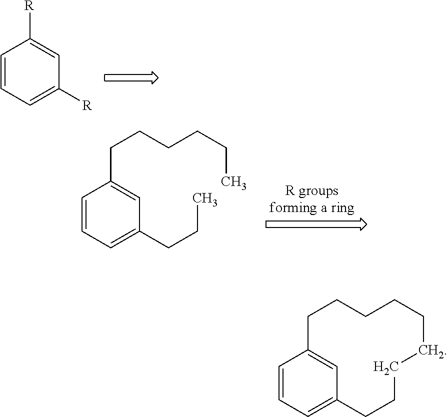

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

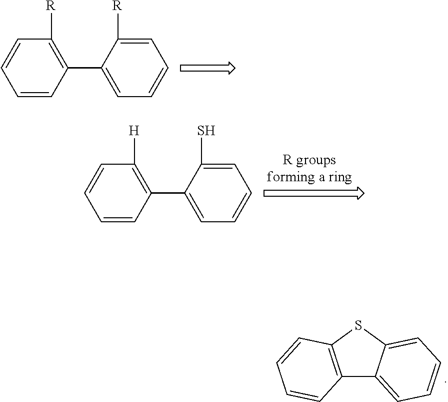

According to an embodiment of the present disclosure, an organic electroluminescent device is disclosed, which comprises:

an anode,

a cathode, and

a first organic layer disposed between the anode and the cathode, wherein the first organic layer at least contains a first compound and a second compound, wherein the first compound has a structure represented by Formula 1:

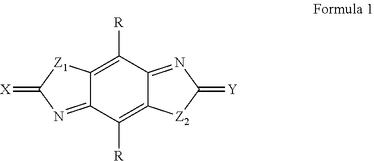

wherein in Formula 1,

X and Y are, at each occurrence identically or differently, selected from NR′, CR″R′″, O, S or Se;

Z₁ and Z₂ are, at each occurrence identically or differently, selected from O, S or Se;

R, R′, R″ and R′″ are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,

each R may be the same or different, and at least one of R, R′, R″ and R′″ is a group having at least one electron withdrawing group;

in Formula 1, adjacent substituents can be optionally joined to form a ring;

wherein the second compound has a structure represented by Formula 2:

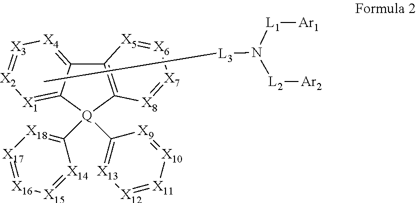

wherein in Formula 2,

X₁ to X₈ are, at each occurrence identically or differently, selected from C, CR₁ or N; and X₉ to X₁₈ are, at each occurrence identically or differently, selected from CR₁ or N;

Q is selected from C, Si or Ge;

L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof,

Ar₁ and Ar₂ are, at each occurrence identically or differently, selected from substituted or unsubstituted aryl having 6 to 30 carbon atoms or substituted or unsubstituted heteroaryl having 3 to 30 carbon atoms;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and

adjacent substituents L₁, L₂, L₃, R₁, Ar₁ and Ar₂ can be optionally joined to form a ring.

In the present embodiment, the expression that “in Formula 1, adjacent substituents can be optionally joined to form a ring” is intended to mean that in Formula 1, any one or more of groups of adjacent substituents, such as adjacent substituents R″ and R′″, adjacent substituents R and R″, adjacent substituents R and R′, adjacent substituents R and R′″, and two adjacent substituents R, can be joined to form a ring. Obviously, it is possible that none of these groups of adjacent substituents are joined to form a ring.

In the present disclosure, the expression that “L₁, L₂, L₃, R₁, Ar₁ and Ar₂ can be optionally joined to form a ring” is intended to mean that in Formula 2, any one or more of groups of adjacent substituents, such as adjacent substituents R₁, adjacent substituents R₁ and L₃, adjacent substituents L₁ and L₂, adjacent substituents L₁ and L₃, adjacent substituents L₂ and L₃, adjacent substituents Ar₁ and Ar₂, adjacent substituents Ar₁ and L₃, adjacent substituents Ar₂ and L₃, adjacent substituents Ar₁ and R₁, and adjacent substituents Ar₂ and R₁, can be joined to form a ring. Obviously, it is possible that none of these groups of adjacent substituents are joined to form a ring.

According to an embodiment of the present disclosure, wherein, in Formula 1, X and Y are, at each occurrence identically or differently, selected from CR″R′″ or NR′, and R′, R″ and R′″ each are a group having at least one electron withdrawing group.

According to an embodiment of the present disclosure, wherein, in Formula 1, R, R′, R″ and R′″ each are a group having at least one electron withdrawing group.

According to an embodiment of the present disclosure, wherein, in Formula 1, X and Y are, at each occurrence identically or differently, selected from O, S or Se, and at least one of R is a group having at least one electron withdrawing group.

According to an embodiment of the present disclosure, wherein, in Formula 1, each R is a group having at least one electron withdrawing group.

According to an embodiment of the present disclosure, wherein, a Hammett constant of the electron withdrawing group is greater than or equal to 0.05, preferably greater than or equal to 0.3, more preferably greater than or equal to 0.5.

In the present disclosure, the electron withdrawing group has a Hammett substituent constant greater than or equal to 0.05, and thus has a relatively strong electron withdrawing ability, which can significantly reduce the LUMO energy level of the compound and improve charge mobility.

It is to be noted that the Hammett substituent constant includes a para Hammett substituent constant and/or a meta Hammett substituent constant, and as long as one of the para constant and the meta constant is greater than or equal to 0.05, the substituent can be used as the group preferably selected in the present disclosure.

According to an embodiment of the present disclosure, wherein, the electron withdrawing group is selected from the group consisting of: halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, an aza-aromatic ring group and any one of the following groups substituted by one or more of halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group and an aza-aromatic ring group: alkyl having 1 to 20 carbon atoms, cycloalkyl having 3 to 20 ring carbon atoms, heteroalkyl having 1 to 20 carbon atoms, arylalkyl having 7 to 30 carbon atoms, alkoxy having 1 to 20 carbon atoms, aryloxy having 6 to 30 carbon atoms, alkenyl having 2 to 20 carbon atoms, alkynyl having 2 to 20 carbon atoms, aryl having 6 to 30 carbon atoms, heteroaryl having 3 to 30 carbon atoms, alkylsilyl having 3 to 20 carbon atoms, arylsilyl having 6 to 20 carbon atoms and combinations thereof.

According to an embodiment of the present disclosure, wherein, the electron withdrawing group is selected from the group consisting of: F, CF₃, OCF₃, SF₅, SO₂CF₃, a cyano group, an isocyano group, SCN, OCN, a pyrimidyl group, a triazinyl group and combinations thereof.

According to an embodiment of the present disclosure, wherein, X and Y are, at each occurrence identically or differently, selected from the group consisting of the following structures:

### O, S, Se,

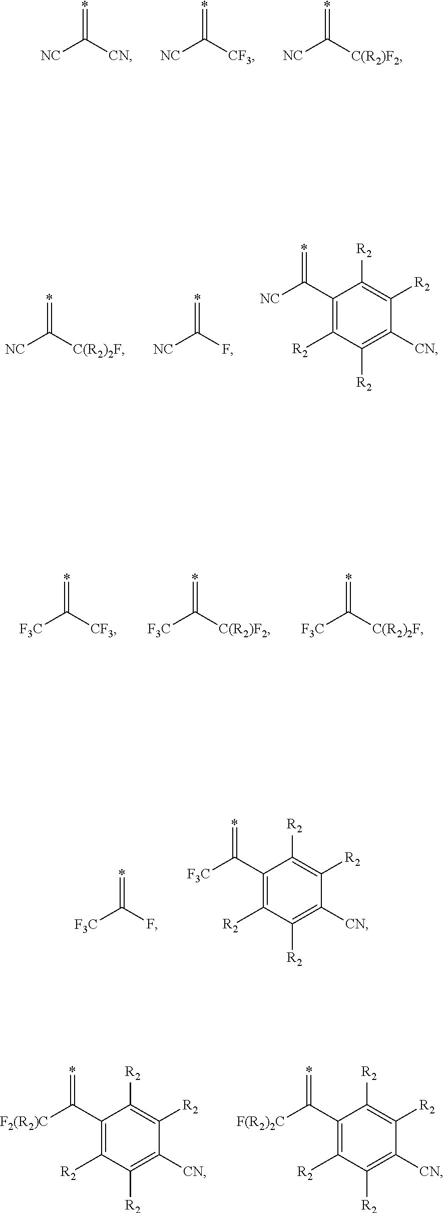

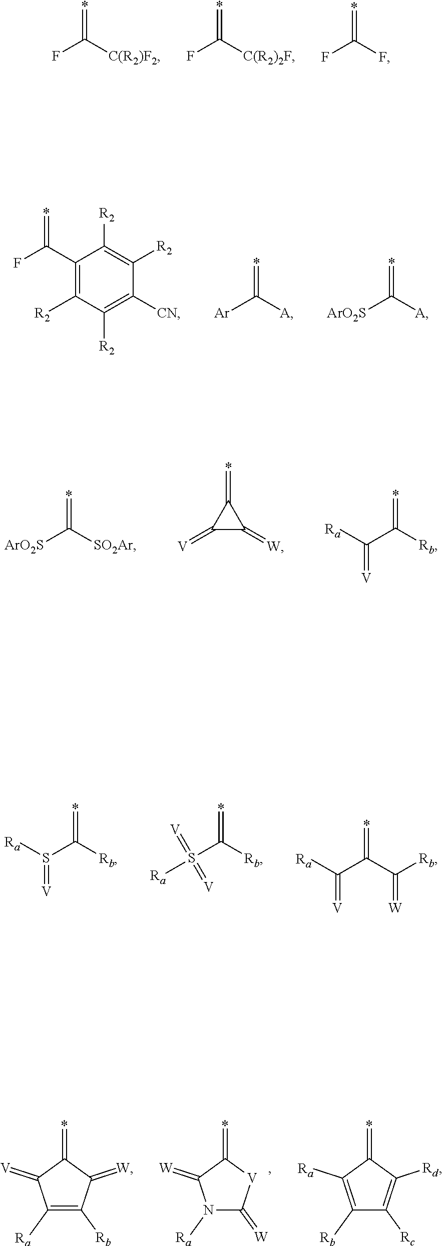

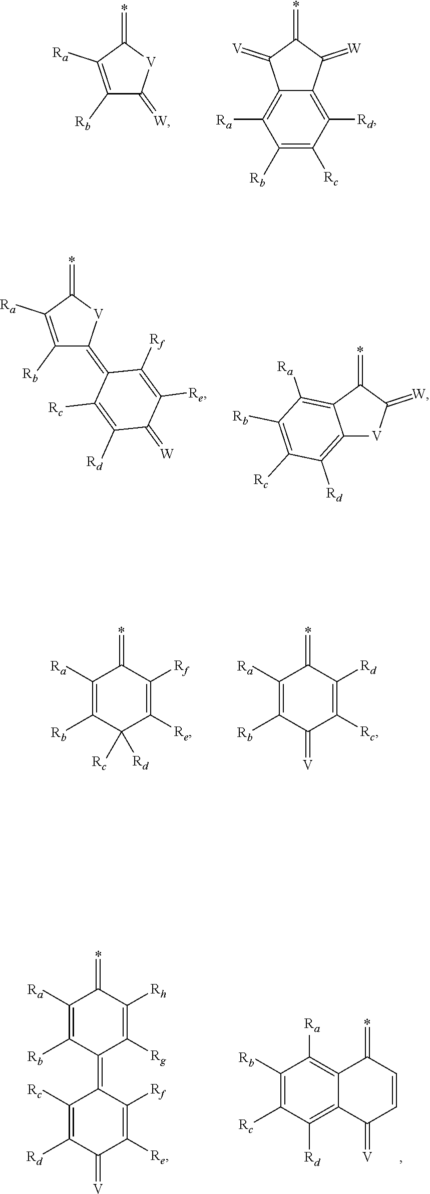

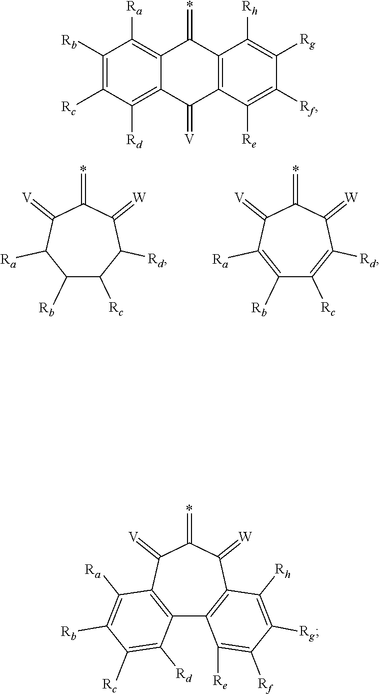

R₂ is, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,

preferably, R₂ is, at each occurrence identically or differently, selected from the group consisting of: F, CF₃, OCF₃, SF₅, SO₂CF₃, a cyano group, an isocyano group, SCN, OCN, pentafluorophenyl, 4-cyanotetrafluorophenyl, tetrafluoropyridyl, pyrimidyl, triazinyl and combinations thereof;

V and W are, at each occurrence identically or differently, selected from CRᵥR_{w}, NRᵥ, O, S or Se;

Ar is, at each occurrence identically or differently, selected from substituted or unsubstituted aryl having 6 to 30 carbon atoms or substituted or unsubstituted heteroaryl having 3 to 30 carbon atoms;

A, Rₐ, R_{b}, R_{c}, R_{d}, Rₑ, R_{f}, R_{g}, Rₕ, Rᵥ and R_{w} are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,

A is a group having at least one electron withdrawing group, and for any one of the structures, when one or more of Rₐ, R_{b}, R_{c}, R_{d}, Rₑ, R_{f}, R_{g}, Rₕ, Rᵥ and R_{w} are present, at least one of Rₐ, R_{b}, R_{c}, R_{d}, Rₑ, R_{f}, R_{g}, Rₕ, Rᵥ and R_{w} is a group having at least one electron withdrawing group; preferably, the group having at least one electron withdrawing group is selected from the group consisting of: F, CF₃, OCF₃, SF₅, SO₂CF₃, a cyano group, an isocyano group, SCN, OCN, pentafluorophenyl, 4-cyanotetrafluorophenyl, tetrafluoropyridyl, pyrimidyl, triazinyl and combinations thereof.

In this embodiment, * represents a position where X or Y is joined to a dehydrobenzodioxazole ring, a dehydrobenzodithiazole ring or a dehydrobenzodiselenazole ring in Formula 1.

According to an embodiment of the present disclosure, wherein, X and Y are, at each occurrence identically or differently, selected from the group consisting of the following structures:

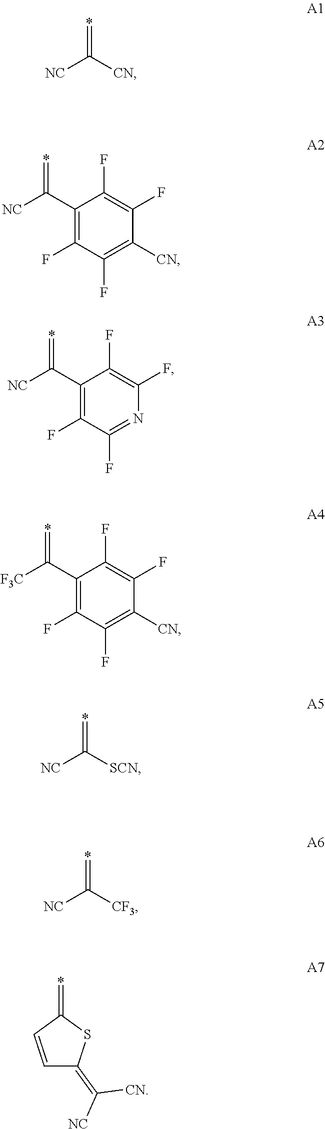

In this embodiment, “*” represents a position where X or Y is joined to a dehydrobenzodioxazole ring, a dehydrobenzodithiazole ring or a dehydrobenzodiselenazole ring in Formula 1.

According to an embodiment of the present disclosure, wherein, R is, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, an unsubstituted alkyl group having 1 to 20 carbon atoms, an unsubstituted cycloalkyl group having 3 to 20 ring carbon atoms, an unsubstituted alkoxy group having 1 to 20 carbon atoms, an unsubstituted alkenyl group having 2 to 20 carbon atoms, an unsubstituted aryl group having 6 to 30 carbon atoms, an unsubstituted heteroaryl group having 3 to 30 carbon atoms and any one of the following groups substituted by one or more of halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group and a phosphoroso group: an alkyl group having 1 to 20 carbon atoms, a cycloalkyl group having 3 to 20 ring carbon atoms, an alkoxy group having 1 to 20 carbon atoms, an alkenyl group having 2 to 20 carbon atoms, an aryl group having 6 to 30 carbon atoms, a heteroaryl group having 3 to 30 carbon atoms and combinations thereof.

According to an embodiment of the present disclosure, wherein, R is, at each occurrence identically or differently, selected from the group consisting of: hydrogen, deuterium, methyl, isopropyl, NO₂, SO₂CH₃, SCF₃, C₂F₅, OC₂F₅, OCH₃, diphenylmethylsilyl, phenyl, methoxyphenyl, p-methylphenyl, 2,6-diisopropylphenyl, biphenyl, polyfluorophenyl, difluoropyridyl, nitrophenyl, dimethylthiazolyl, vinyl substituted by one or more of CN or CF₃, acetenyl substituted by one of CN or CF₃, dimethylphosphoroso, diphenylphosphoroso, F, CF₃, OCF₃, SF₅, SO₂CF₃, cyano, isocyano, SCN, OCN, trifluoromethylphenyl, trifluoromethoxyphenyl, bis(trifluoromethyl)phenyl, bis(trifluoromethoxy)phenyl, 4-cyanotetrafluorophenyl, phenyl or biphenyl substituted by one or more of F, CN or CF₃, tetrafluoropyridyl, pyrimidyl, triazinyl, diphenylboryl, oxaboraanthryl and combinations thereof.

According to an embodiment of the present disclosure, wherein, X and Y each are

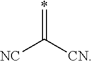 [SMILES: *=C(C#N)C#N]

According to an embodiment of the present disclosure, wherein, R is, at each occurrence identically or differently, selected from the group consisting of the following structures:

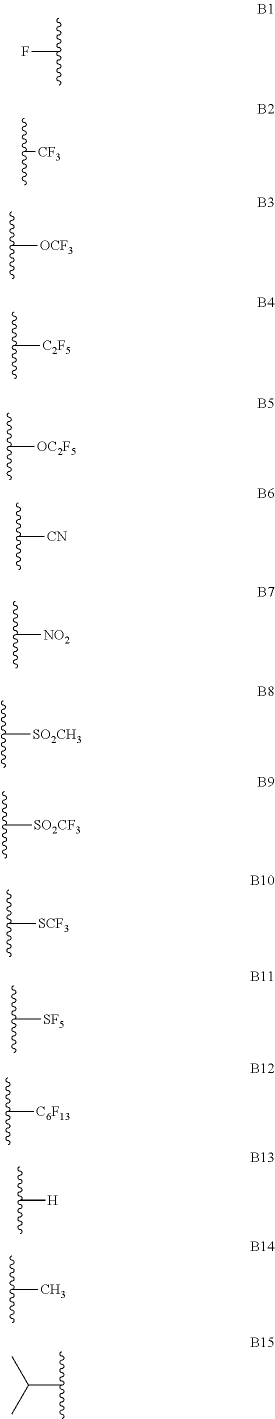

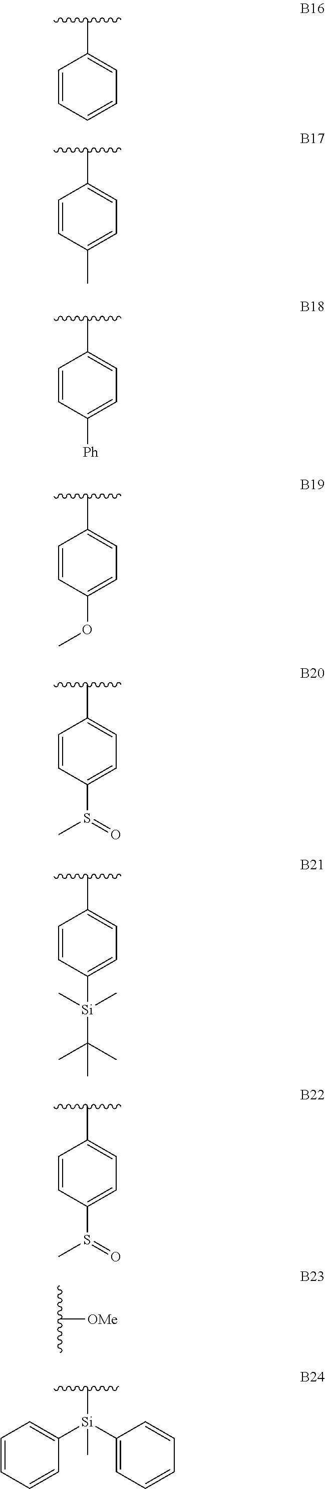

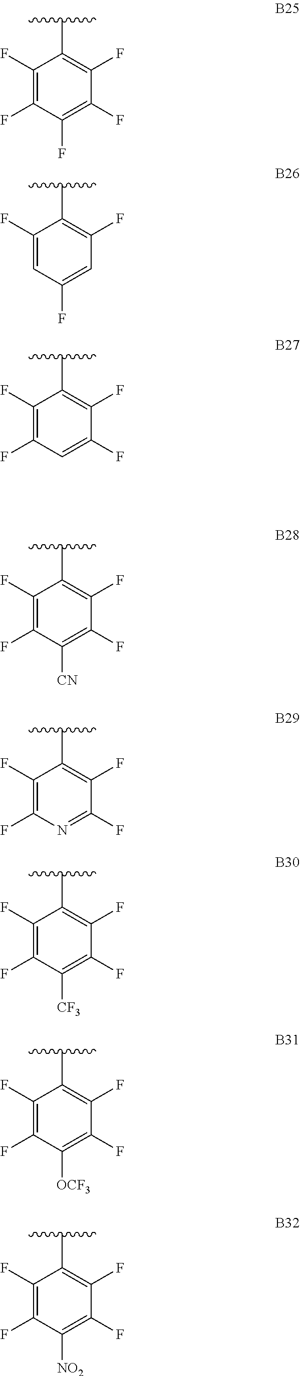

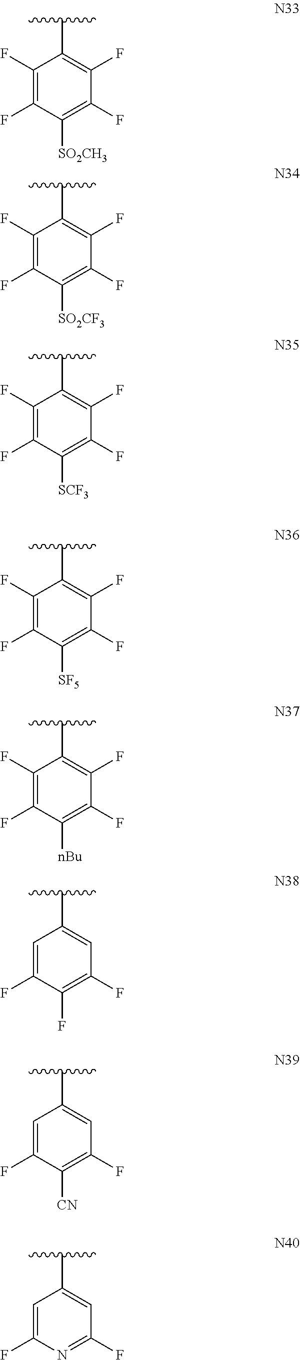

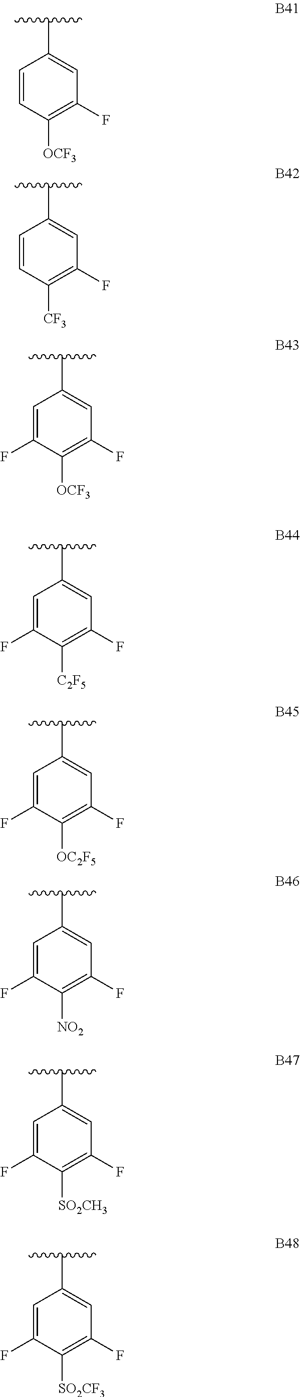

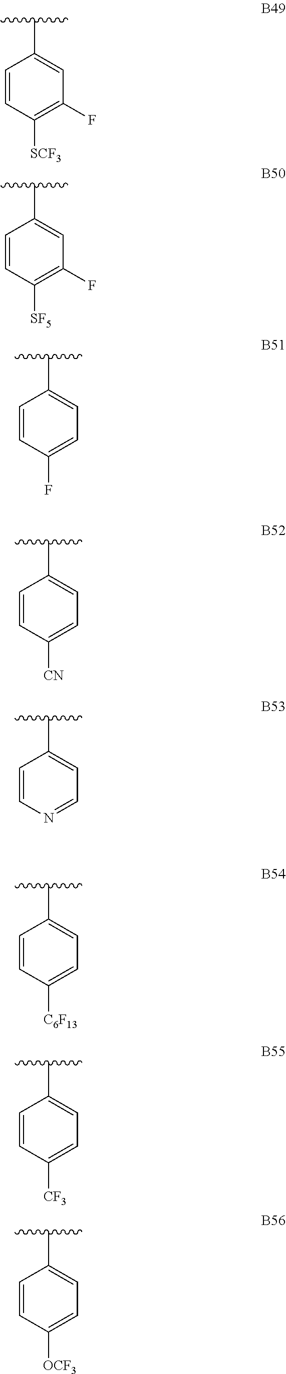

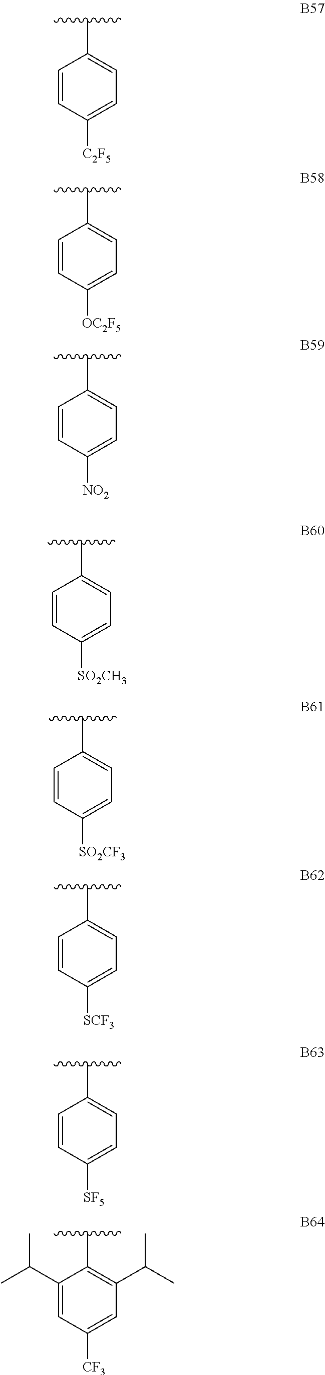

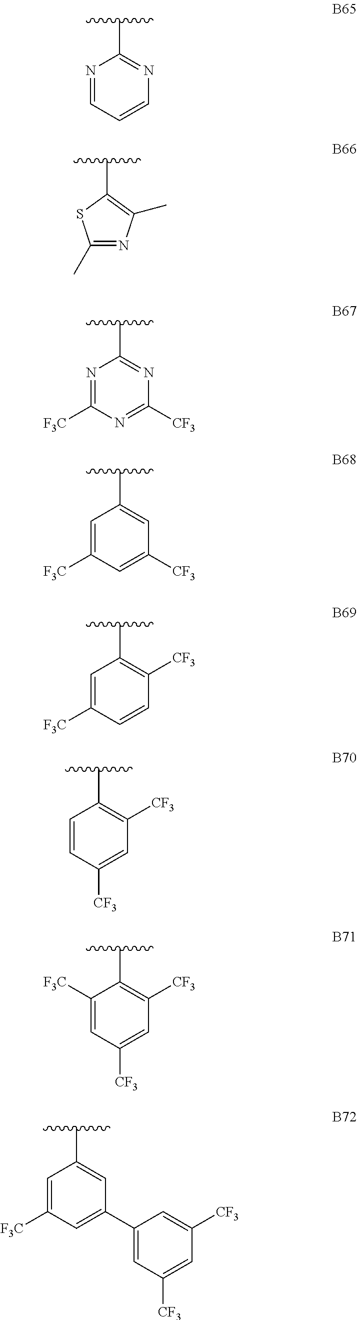

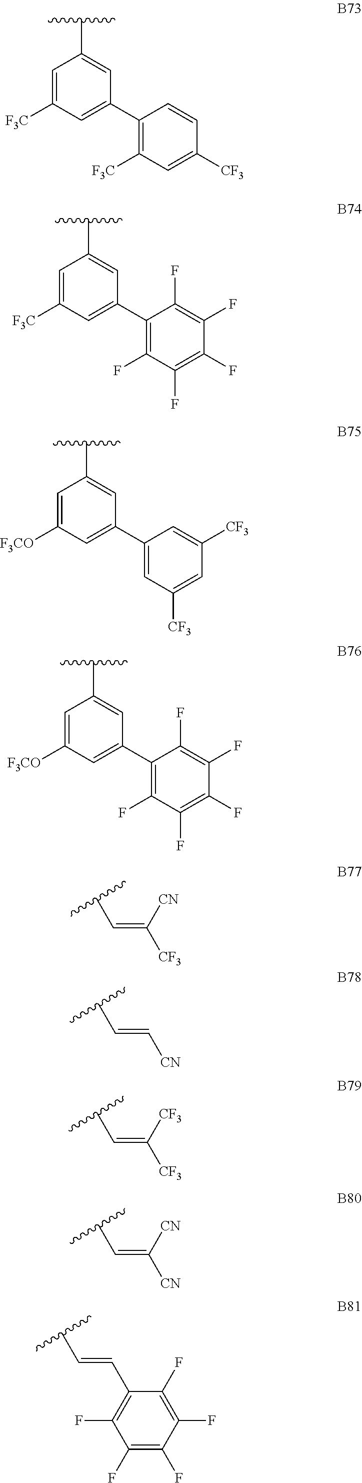

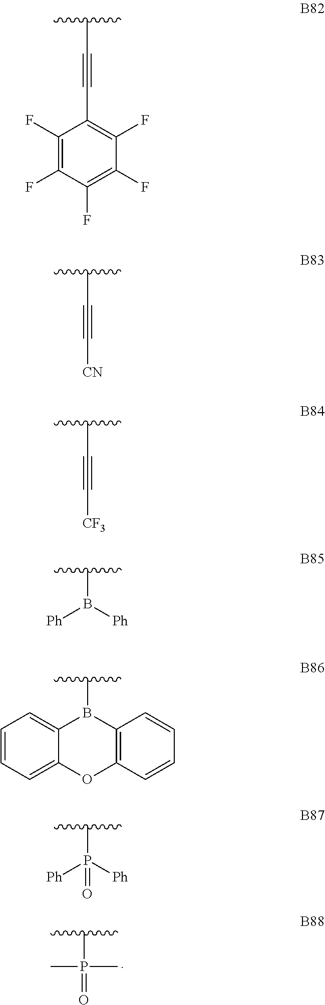

In this embodiment, “ [symbol] ” represents a position where the group R is joined to a dehydrobenzodioxazole ring, a dehydrobenzodithiazole ring or a dehydrobenzodiselenazole ring in Formula 1.

According to an embodiment of the present disclosure, wherein, two R in one first compound represented by Formula 1 are the same.

According to an embodiment of the present disclosure, wherein, the first compound is selected from the group consisting of Compound 1 to Compound 1356; wherein the specific structures of Compound 1 to Compound 1356 are referred to claim 10.

According to an embodiment of the present disclosure, wherein, the second compound has a structure represented by any one of Formula 2-1 to Formula 2-12:

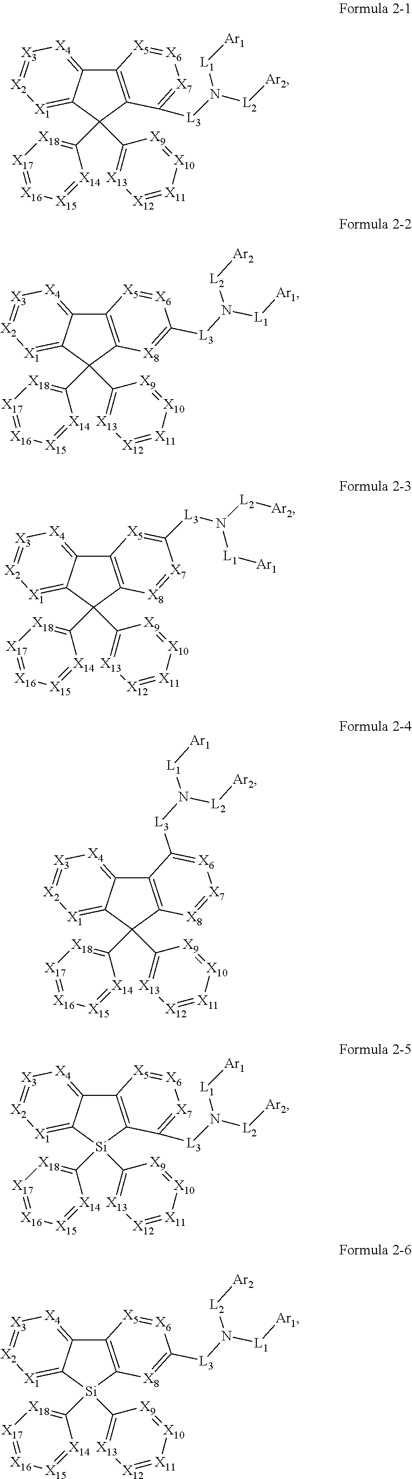

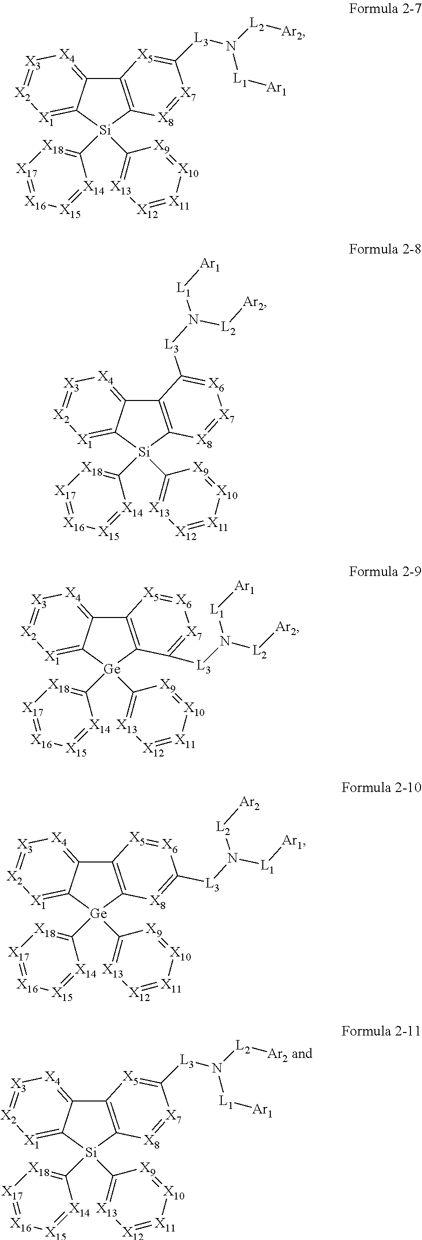

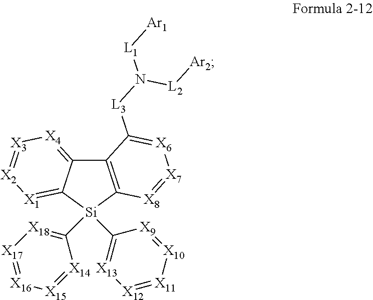

X₁ to X₁₈ are, at each occurrence identically or differently, selected from CR₁;

L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof;

Ar₁ and Ar₂ are, at each occurrence identically or differently, selected from substituted or unsubstituted aryl having 6 to 30 carbon atoms or substituted or unsubstituted heteroaryl having 3 to 30 carbon atoms;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and

adjacent substituents L₁, L₂, L₃, R₁, Ar₁ and Ar₂ can be optionally joined to form a ring.

According to an embodiment of the present disclosure, wherein, the second compound has a structure represented by Formula 2-1, Formula 2-2, Formula 2-3, Formula 2-4, Formula 2-6 or Formula 2-10.

According to an embodiment of the present disclosure, wherein, in Formula 2, at least one of X₁ to X₁₈ is N.

According to an embodiment of the present disclosure, wherein, the second compound has a structure represented by any one of Formula 2-13 to Formula 2-24:

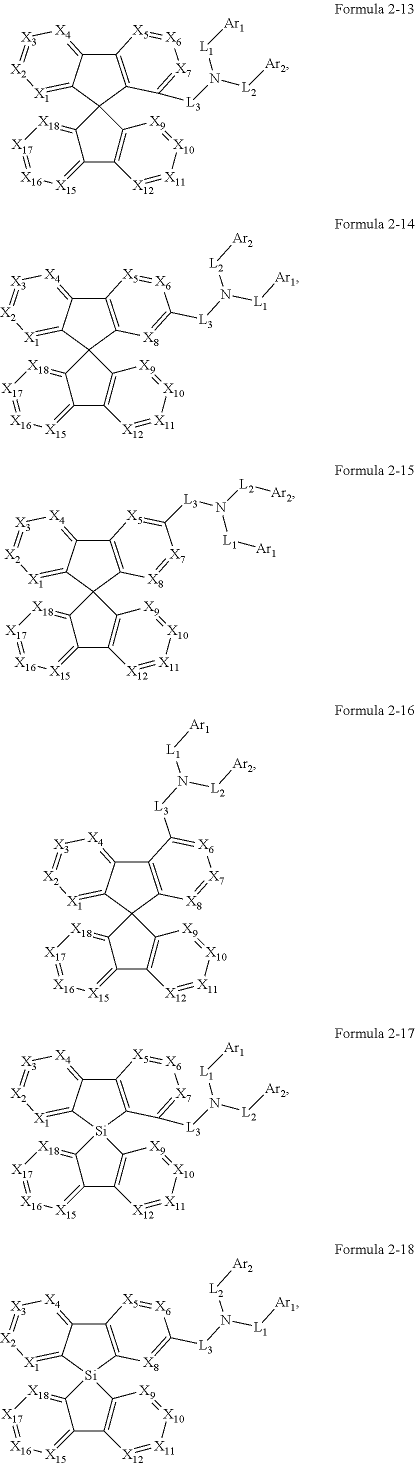

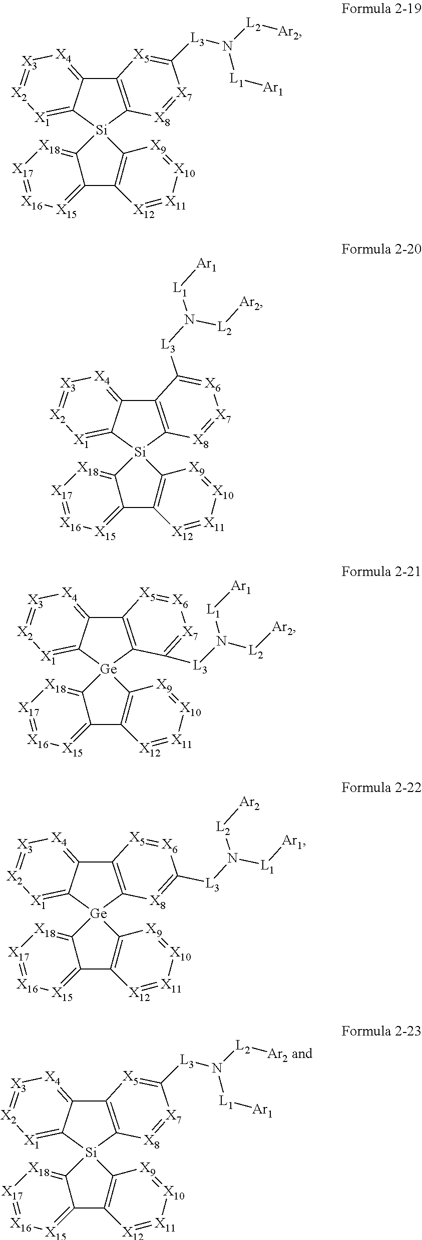

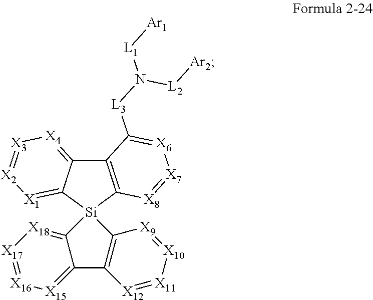

X₁ to X₁₈ are, at each occurrence identically or differently, selected from CR₁ or N;

L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof;

Ar₁ and Ar₂ are, at each occurrence identically or differently, selected from substituted or unsubstituted aryl having 6 to 30 carbon atoms or substituted or unsubstituted heteroaryl having 3 to 30 carbon atoms;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and

adjacent substituents L₁, L₂, L₃, R₁, Ar₁ and Ar₂ can be optionally joined to form a ring.

According to an embodiment of the present disclosure, wherein, the second compound has a structure represented by Formula 2-13, Formula 2-14, Formula 2-15, Formula 2-16, Formula 2-18 or Formula 2-22.

According to an embodiment of the present disclosure, wherein, in Formula 2-13 to Formula 2-24, X₁ to X₁₈ are, at each occurrence identically or differently, selected from CR₁.

According to an embodiment of the present disclosure, wherein, in Formula 2-13 to Formula 2-24, at least one of X₁ to X₁₈ is selected from N.

In this embodiment, the expression that “in Formula 2-13 to Formula 2-24, at least one of X₁ to X₁₈ is selected from N” is intended to mean that in Formula 2-13, Formula 2-17 and Formula 2-21, at least one of X₁ to X₇, X₉ to X₁₂ and X₁₅ to X₁₈ is N; in Formula 2-14, Formula 2-18 and Formula 2-22, at least one of X₁ to X₆, X₈ to X₁₂ and X₁₅ to X₁₈ is N; in Formula 2-15, Formula 2-19 and Formula 2-23, at least one of X₁ to X₅, X₇ to X₁₂ and X₁₅ to X₁₈ is N; and in Formula 2-16, Formula 2-20 and Formula 2-24, at least one of X₁ to X₄, X₆ to X₁₂ and X₁₅ to X₁₈ is N.

According to an embodiment of the present disclosure, wherein, the L₁ to L₃ are, at each occurrence identically or differently, selected from a single bond, a substituted or unsubstituted arylene group having 6 to 24 carbon atoms, a substituted or unsubstituted heteroarylene group having 3 to 24 carbon atoms or a combination thereof.

According to an embodiment of the present disclosure, wherein, L₁ to L₃ are, at each occurrence identically or differently, selected from a single bond, a substituted or unsubstituted phenylene group, a substituted or unsubstituted biphenylene group, a substituted or unsubstituted terphenylene group, a substituted or unsubstituted naphthylene group, a substituted or unsubstituted fluorenylidene group, a substituted or unsubstituted silafluorenylidene group, a substituted or unsubstituted carbazolylene group, a substituted or unsubstituted dibenzofuranylene group, a substituted or unsubstituted dibenzothienylene group, a substituted or unsubstituted dibenzoselenophenylene group, a substituted or unsubstituted phenanthrylene group, a substituted or unsubstituted triphenylenylene group, a substituted or unsubstituted pyridylene group, a substituted or unsubstituted spirobifluorenylidene group, a substituted or unsubstituted anthrylene group, a substituted or unsubstituted pyrenylene group or a combination thereof.

According to an embodiment of the present disclosure, wherein, L₁ to L₃ are, at each occurrence identically or differently, selected from the group consisting of the following:

a single bond

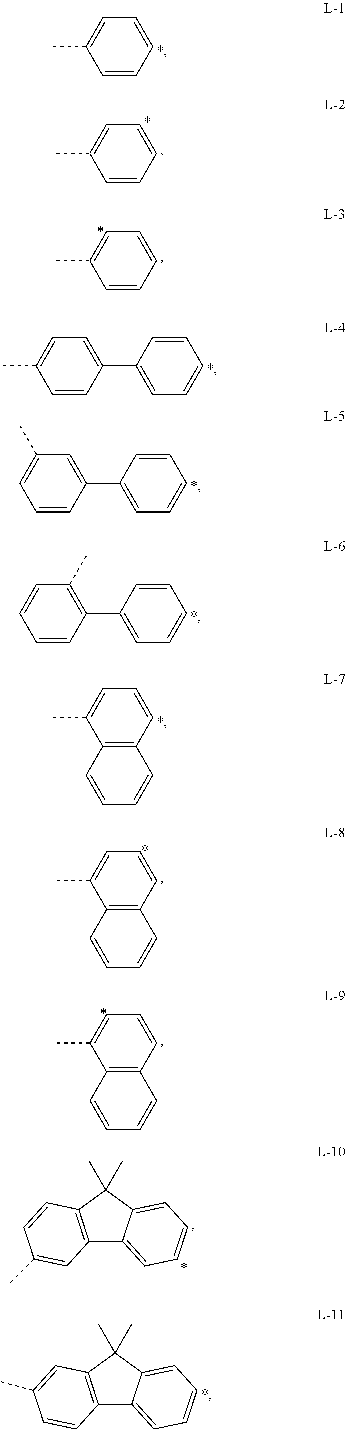

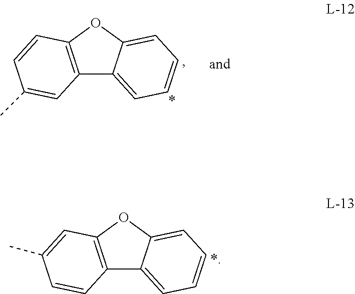

In this embodiment, “*” represents a position where the nitrogen in Formula 2 is bonded in L-1 to L-13, and the dashed line represents a position where Ar₁, Ar₂ or any one of X₁ to X₈ in Formula 2 is bonded in L-1 to L-13.

According to an embodiment of the present disclosure, wherein, Ar₁ and Ar₂ have, at each occurrence identically or differently, a structure represented by any one of Formula 3-1 to Formula 3-4:

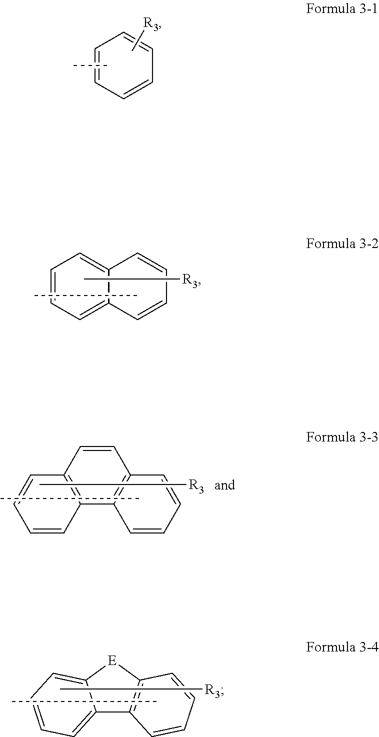

E is, at each occurrence identically or differently, selected from O, S, Se, C(R₄)₂, Si(R₄)₂ or Ge(R₄)₂; when two R₄ are present at the same time, the two R₄ may be the same or different;

R₃ represents, at each occurrence identically or differently, mono-substitution, multiple substitutions or non-substitution;

R₃ and R₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and

adjacent substituents R₃, R₄ can be optionally joined to form a ring.

In this embodiment, the expression that “adjacent substituents R₃, R₄ can be optionally joined to form a ring” is intended to mean that any one or more of groups of adjacent substituents, such as adjacent substituents R₃ and R₃, adjacent substituents R₃ and R₄, and adjacent substituents R₄ and R₄, can be joined to form a ring. Obviously, it is possible that none of these groups of adjacent substituents are joined to form a ring.

In this embodiment, the dashed line represents a position where L₁ is joined in the structure of Ar₁; and the dashed line also represents a position where L₂ is joined in the structure of Ar₂.

According to an embodiment of the present disclosure, wherein, R₃ and R₄ are, at each occurrence identically or differently, selected from hydrogen, deuterium, halogen, substituted or unsubstituted alkyl having 1 to 20 carbon atoms, substituted or unsubstituted aryl having 6 to 30 carbon atoms, substituted or unsubstituted heteroaryl having 3 to 30 carbon atoms or a combination thereof.

According to an embodiment of the present disclosure, wherein, Ar₁ and Ar₂ are, at each occurrence identically or differently, selected from the group consisting of G1 to G37:

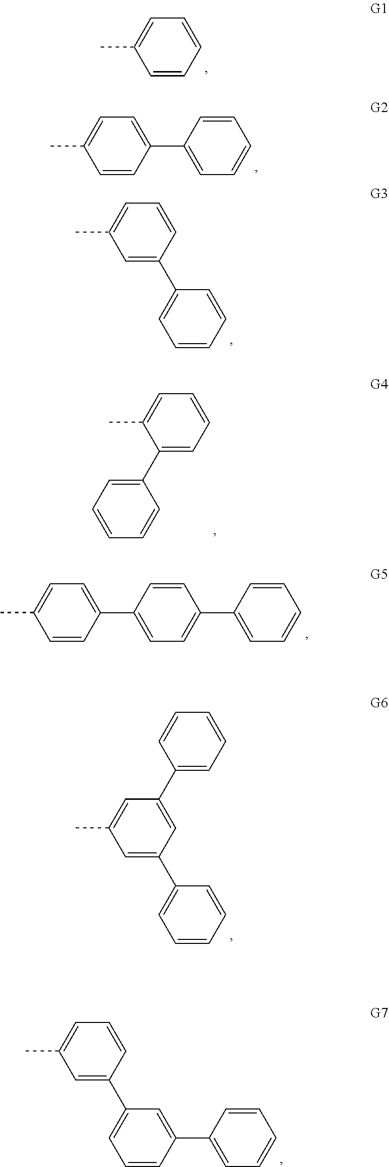

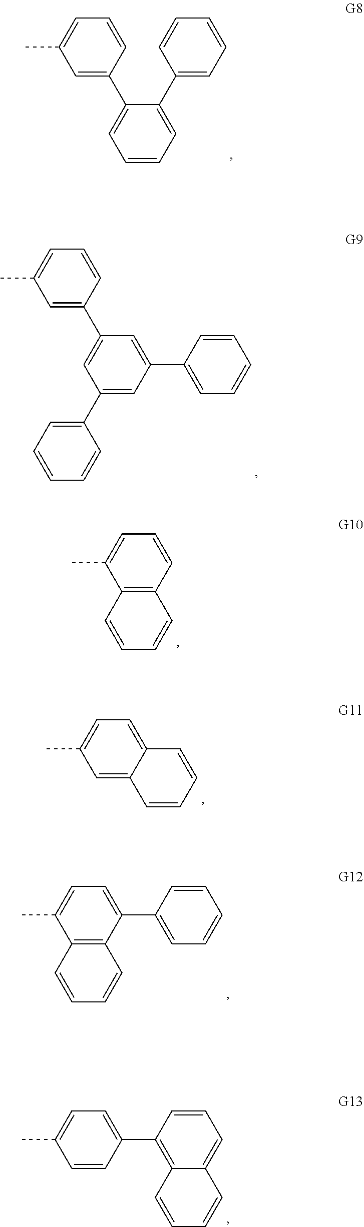

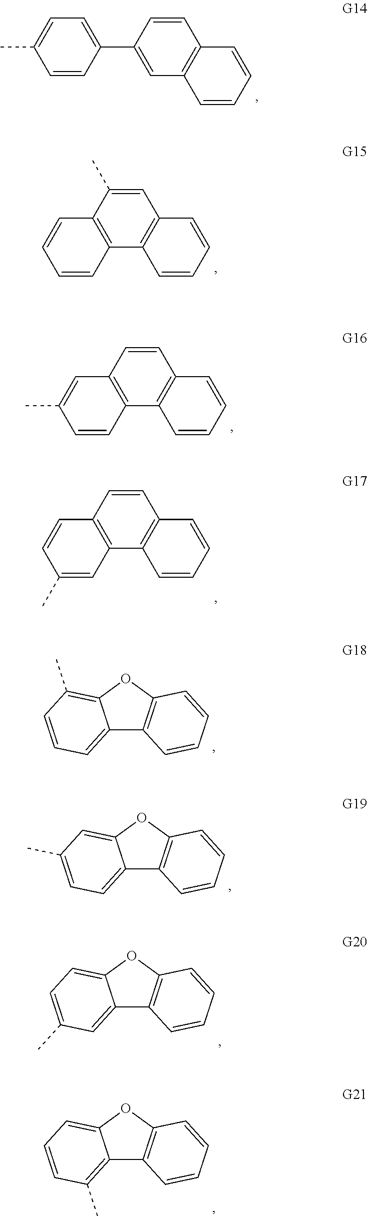

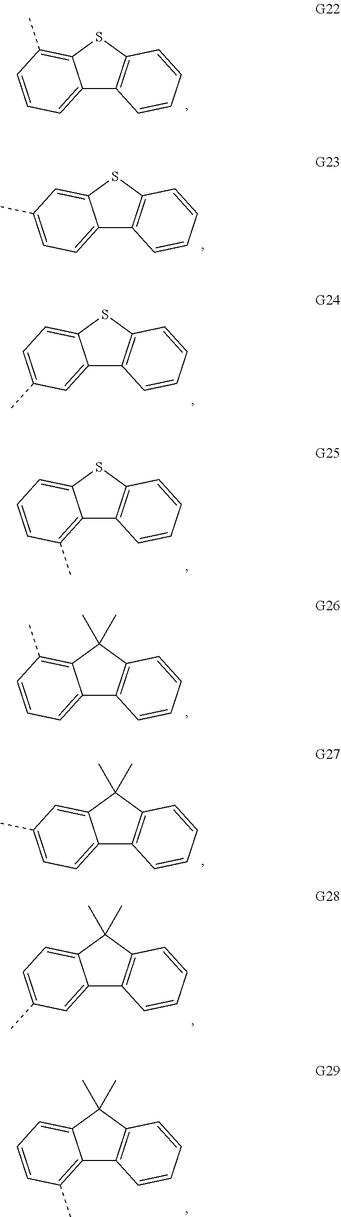

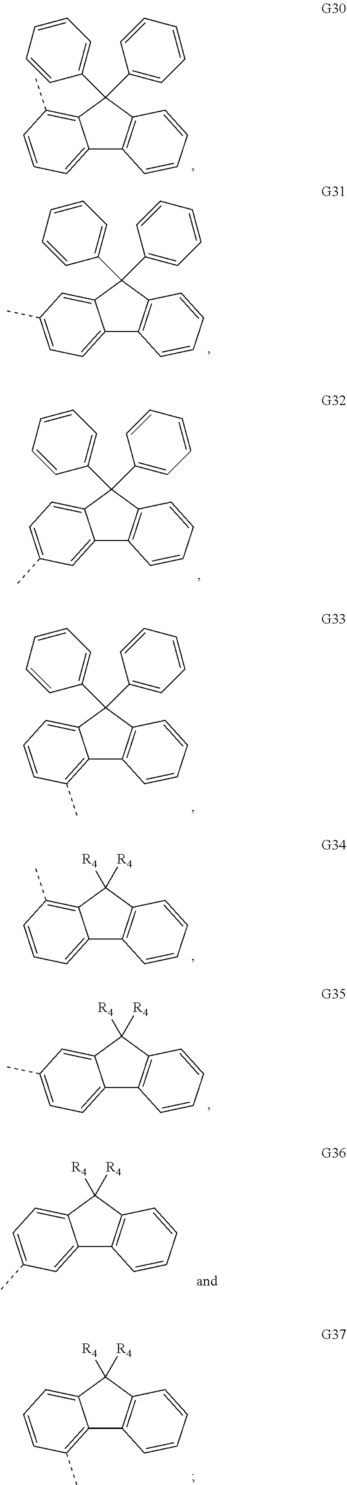

R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and

adjacent substituents R₄ can be optionally joined to form a ring.

In this embodiment, the expression that “adjacent substituents R₄ can be optionally joined to form a ring” is intended to mean that any two adjacent substituents R₄ can be joined to form a ring. Obviously, it is possible that none of any two adjacent substituents R₄ can be joined to form a ring.

According to an embodiment of the present disclosure, wherein, R₄ is, at each occurrence identically or differently, selected from hydrogen, deuterium, substituted or unsubstituted alkyl having 1 to 20 carbon atoms, substituted or unsubstituted aryl having 6 to 30 carbon atoms, substituted or unsubstituted heteroaryl having 3 to 30 carbon atoms or a combination thereof.

According to an embodiment of the present disclosure, wherein, R₄ is, at each occurrence identically or differently, selected from hydrogen, deuterium, methyl, ethyl, isopropyl, fluorenyl, phenyl, biphenyl, naphthyl or a combination thereof.

According to an embodiment of the present disclosure, wherein, R₁ is, at each occurrence identically or differently, selected from hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms or a combination thereof.

According to an embodiment of the present disclosure, wherein, at least one or two of X₁ to X₁₈ are, at each occurrence identically or differently, selected from CR₁, and the R₁ is, at each occurrence identically or differently, selected from deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms or a combination thereof.

According to an embodiment of the present disclosure, wherein, in Formula 2-1 to Formula 2-24, at least one or two of X₉ to X₁₈ are, at each occurrence identically or differently, selected from CR₁, and the R₁ is, at each occurrence identically or differently, selected from deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms or a combination thereof.

According to an embodiment of the present disclosure, wherein, R₁ is, at each occurrence identically or differently, selected from hydrogen, deuterium, fluorine, methyl, n-propyl, isopropyl, n-butyl, isobutyl, sec-butyl, t-butyl, 2-methylbutyl, n-pentyl, sec-pentyl, neopentyl, cyclopentyl, n-hexyl, neohexyl, cyclohexyl, n-heptyl, phenyl, biphenyl, terphenyl, naphthyl, fluorenyl or a combination thereof.

According to an embodiment of the present disclosure, wherein, the Ar₁ and Ar₂ in the second compound are joined to form a ring.

According to an embodiment of the present disclosure, wherein, L₁ and L₂ each are a single bond.

According to an embodiment of the present disclosure, wherein, the second compound has a structure represented by Formula 2-25:

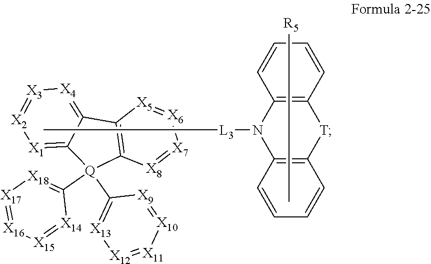

X₁ to X₈ are, at each occurrence identically or differently, selected from C, CR₁ or N;

X₉ to X₁₈ are, at each occurrence identically or differently, selected from CR₁ or N;

Q is, at each occurrence, selected from C, Si or Ge;

T is, at each occurrence identically or differently, selected from CR₅′R₅′, O, S or NR₅′;

R₅ represents, at each occurrence identically or differently, mono-substitution, multiple substitutions or non-substitution;

R₅ and R₅′ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof,

L₃ is, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof, and

adjacent substituents R₁, R₅ and R₅′ can be optionally joined to form a ring.

In this embodiment, the expression that “adjacent substituents R₁, R₅ and R₅′ can be optionally joined to form a ring” is intended to mean that any one or more of groups of adjacent substituents, such as adjacent substituents R₁ and R₁, adjacent substituents R₅ and R₅, adjacent substituents R₅ and R₅′, and adjacent substituents R₅′ and R₅′, can be joined to form a ring. Obviously, it is possible that none of these groups of adjacent substituents are joined to form a ring.

According to an embodiment of the present disclosure, wherein, the second compound is selected from Compound I-1 to Compound I-7, Compound I-12 to Compound I-182, Compound I-185 to Compound I-229, Compound I-232 to Compound I-273, Compound II-1 to Compound II-7, Compound II-9 to Compound II-30, Compound II-32 to Compound II-35, Compound II-39 to Compound II-79, Compound II-81 to Compound II-95, Compound II-97 to Compound II-110, Compound II-112 to Compound II-208, Compound II-210 to Compound II-221, Compound II-223, Compound II-225 to Compound II-243, Compound II-245 to Compound II-273, Compound II-275 to Compound II-286, Compound II-288, Compound II-290 to Compound II-308, Compound II-310 to Compound II-332, Compound III-1 to Compound III-7, Compound III-12 to Compound III-182, Compound III-185 to Compound III-229, Compound III-232 to Compound III-273, Compound IV-1 to Compound IV-7, Compound IV-12 to Compound IV-182, Compound IV-185 to Compound IV-229, Compound IV-232 to Compound IV-273, Compound V-1 to Compound V-7, Compound V-12 to Compound V-182, Compound V-185 to Compound V-229, Compound V-232 to Compound V-273, Compound VI-1 to Compound VI-7, Compound VI-12 to Compound VI-182, Compound VI-185 to Compound VI-229, Compound VI-232 to Compound VI-273, Compound VII-1 to Compound VII-7, Compound VII-12 to Compound VII-182, Compound VII-185 to Compound VII-229, Compound VII-232 to Compound VII-273, Compound VIII-1 to Compound VIII-7, Compound VIII-12 to Compound VIII-182, Compound VIII-185 to Compound VIII-229, Compound VIII-232 to Compound VIII-273, Compound IX-1 to Compound IX-7, Compound IX-12 to Compound IX-182, Compound IX-185 to Compound IX-229, Compound IX-232 to Compound IX-273, Compound X-1 to Compound X-7, Compound X-12 to Compound X-182, Compound X-185 to Compound X-229 or Compound X-232 to Compound X-273. The specific structures of Compound I-1 to Compound I-7, Compound I-12 to Compound I-182, Compound I-185 to Compound I-229, Compound I-232 to Compound I-273, Compound II-1 to Compound II-7, Compound II-9 to Compound II-30, Compound II-32 to Compound II-35, Compound II-39 to Compound II-79, Compound II-81 to Compound II-95, Compound II-97 to Compound II-110, Compound II-112 to Compound II-208, Compound II-210 to Compound II-221, Compound II-223, Compound II-225 to Compound II-243, Compound II-245 to Compound II-273, Compound II-275 to Compound II-286, Compound II-288, Compound II-290 to Compound II-308, Compound II-310 to Compound II-332, Compound III-1 to Compound III-7, Compound III-12 to Compound III-182, Compound III-185 to Compound III-229, Compound III-232 to Compound III-273, Compound IV-1 to Compound IV-7, Compound IV-12 to Compound IV-182, Compound IV-185 to Compound IV-229, Compound IV-232 to Compound IV-273, Compound V-1 to Compound V-7, Compound V-12 to Compound V-182, Compound V-185 to Compound V-229, Compound V-232 to Compound V-273, Compound VI-1 to Compound VI-7, Compound VI-12 to Compound VI-182, Compound VI-185 to Compound VI-229, Compound VI-232 to Compound VI-273, Compound VII-1 to Compound VII-7, Compound VII-12 to Compound VII-182, Compound VII-185 to Compound VII-229, Compound VII-232 to Compound VII-273, Compound VIII-1 to Compound VIII-7, Compound VIII-12 to Compound VIII-182, Compound VIII-185 to Compound VIII-229, Compound VIII-232 to Compound VIII-273, Compound IX-1 to Compound IX-7, Compound IX-12 to Compound IX-182, Compound IX-185 to Compound IX-229, Compound IX-232 to Compound IX-273, Compound X-1 to Compound X-7, Compound X-12 to Compound X-182, Compound X-185 to Compound X-229 and Compound X-232 to Compound X-273 are referred to claim 19.

According to an embodiment of the present disclosure, wherein, the first organic layer is a hole injection layer, and the hole injection layer is in contact with the anode.

According to an embodiment of the present disclosure, wherein, in the first organic layer, the weight ratio of the first compound to the second compound is from 10000:1 to 1:10000; preferably, the weight ratio of the first compound to the second compound is from 100:1 to 1:10000; more preferably, the weight ratio of the first compound to the second compound is from 10:1 to 1:10000.

According to an embodiment of the present disclosure, wherein, in the first organic layer, the first compound accounts for 0.01% to 10% of a total weight of the first organic layer; or the first compound accounts for 0.01% to 5% of the total weight of the first organic layer; or the first compound accounts for 0.01% to 3% of the total weight of the first organic layer; or the first compound accounts for 0.01% to 2% of the total weight of the first organic layer; or the first compound accounts for 0.01% to 1.5% of the total weight of the first organic layer; or the first compound accounts for 0.01% to 1% of the total weight of the first organic layer.

According to an embodiment of the present disclosure, wherein, the organic electroluminescent device further comprises at least one light-emitting layer.

According to an embodiment of the present disclosure, wherein, the at least one light-emitting layer contains at least one host material and at least one doping material.

According to an embodiment of the present disclosure, wherein, a maximum emission wavelength of the organic electroluminescent device is from 300 nm to 1200 nm.

According to an embodiment of the present disclosure, wherein, the organic electroluminescent device further comprises a second organic layer disposed between the first organic layer and the at least one light-emitting layer.

According to an embodiment of the present disclosure, wherein, the second organic layer contains one compound containing any one or more chemical structural units selected from the following group: triarylamine, carbazole, fluorene, spirobifluorene, thiophene, furan, phenyl, oligophenylene ethylene, oligofluorene and combinations thereof.

According to an embodiment of the present disclosure, wherein, the one compound in the second organic layer is the second compound.

According to an embodiment of the present disclosure, wherein, the organic electroluminescent device further comprises a third organic layer disposed between the second organic layer and the light-emitting layer.

According to an embodiment of the present disclosure, wherein, the third organic layer contains another compound containing any one or more chemical structural units selected from the following group: triarylamine, carbazole, fluorene, spirobifluorene, thiophene, furan, phenyl, oligophenylene ethylene, oligofluorene and combinations thereof.

According to an embodiment of the present disclosure, wherein, the another compound in the third organic layer is the second compound.

According to an embodiment of the present disclosure, wherein, in the device, only the first organic layer is p-type doped among all the organic layers disposed between the anode and the light-emitting layer.

According to an embodiment of the present disclosure, wherein, a thickness of the first organic layer is from 0.1 nm to 40 nm, and a thickness of the second organic layer is from 0.1 nm to 300 nm.

According to another embodiment of the present disclosure, a display assembly is further disclosed. The display assembly comprises an organic electroluminescent device, wherein the specific structure of the organic electroluminescent device is shown in any one of the preceding embodiments.

According to another embodiment of the present disclosure, a compound combination is further disclosed, which contains a first compound and a second compound, wherein the first compound has a structure represented by Formula 1:

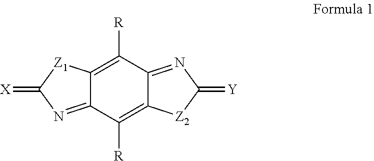

wherein in Formula 1,

X and Y are, at each occurrence identically or differently, selected from NR′, CR″R′″, O, S or Se;

Z₁ and Z₂ are, at each occurrence identically or differently, selected from O, S or Se;

R, R′, R″ and R′″ are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,

each R may be the same or different, and at least one of R, R′, R″ and R′″ is a group having at least one electron withdrawing group;

in Formula 1, adjacent substituents can be optionally joined to form a ring;

wherein the second compound has a structure represented by Formula 2:

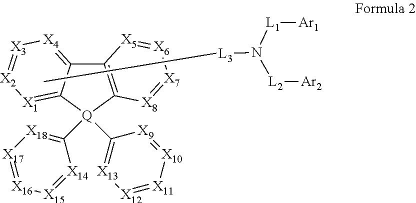

wherein in Formula 2,

X₁ to X₈ are, at each occurrence identically or differently, selected from C, CR₁ or N; and X₉ to X₁₈ are, at each occurrence identically or differently, selected from CR₁ or N;

Q is selected from C, Si or Ge;

L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof,

Ar₁ and Ar₂ are, at each occurrence identically or differently, selected from substituted or unsubstituted aryl having 6 to 30 carbon atoms or substituted or unsubstituted heteroaryl having 3 to 30 carbon atoms;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and adjacent substituents L₁, L₂, L₃, R₁, Ar₁ and Ar₂ can be optionally joined to form a ring.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, compound composition disclosed herein may be used in combination with a wide variety of emissive dopants, hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The device of the present disclosure may include charge injection and transporting layers such as a hole transporting layer, an electron transporting layer and an electron injection layer. The device may further include a light-emitting layer which contains at least a light-emitting dopant and at least one host compound. The light-emitting dopant may be a fluorescent light-emitting dopant and/or a phosphorescent light-emitting dopant. The device may further include a blocking layer such as a hole blocking layer and an electron blocking layer.

Conventional hole transporting materials in the related art may be used in the hole transporting layer. For example, the hole transporting layer may typically contain the following hole transporting materials without limitation:

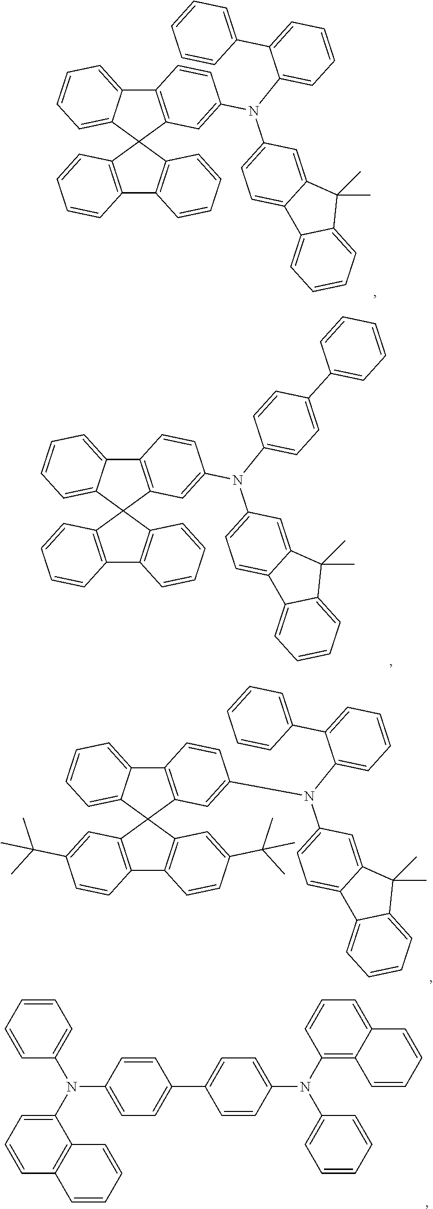

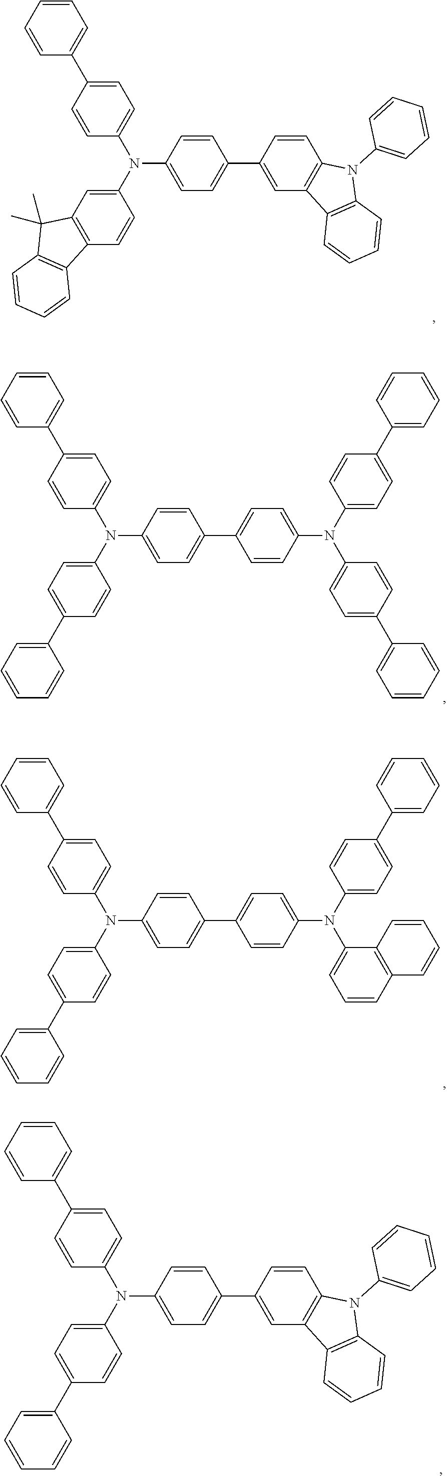

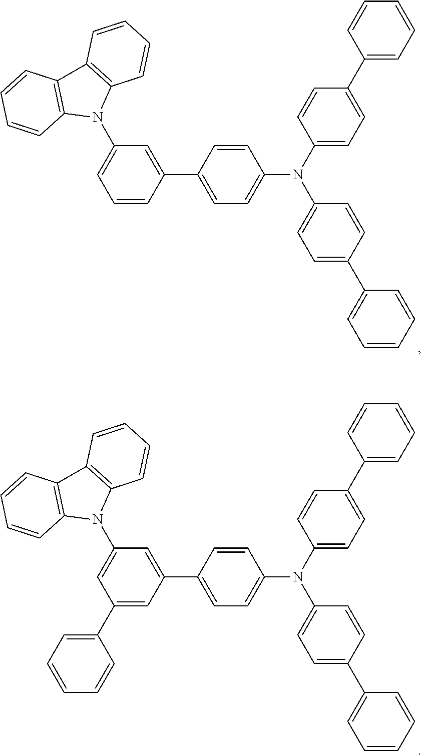 [SMILES: c1ccc(-c2ccc(N(c3ccc(-c4ccccc4)cc3)c3ccc(-c4cccc(-n5c6ccccc6c6ccccc65)c4)cc3)cc2)cc1 ; "," ; c1ccc(-c2ccc(N(c3ccc(-c4ccccc4)cc3)c3ccc(-c4cc(-c5ccccc5)cc(-n5c6ccccc6c6ccccc65)c4)cc3)cc2)cc1 ; "."]

Conventional electron transporting materials in the related art may be used in the electron transporting layer. For example, the electron transporting layer may typically contain the following electron transporting materials without limitation:

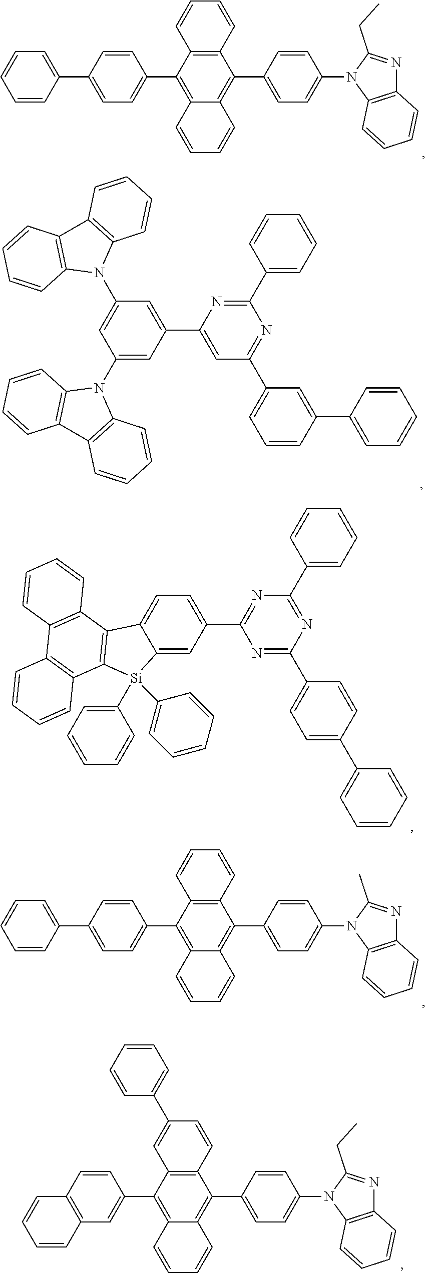

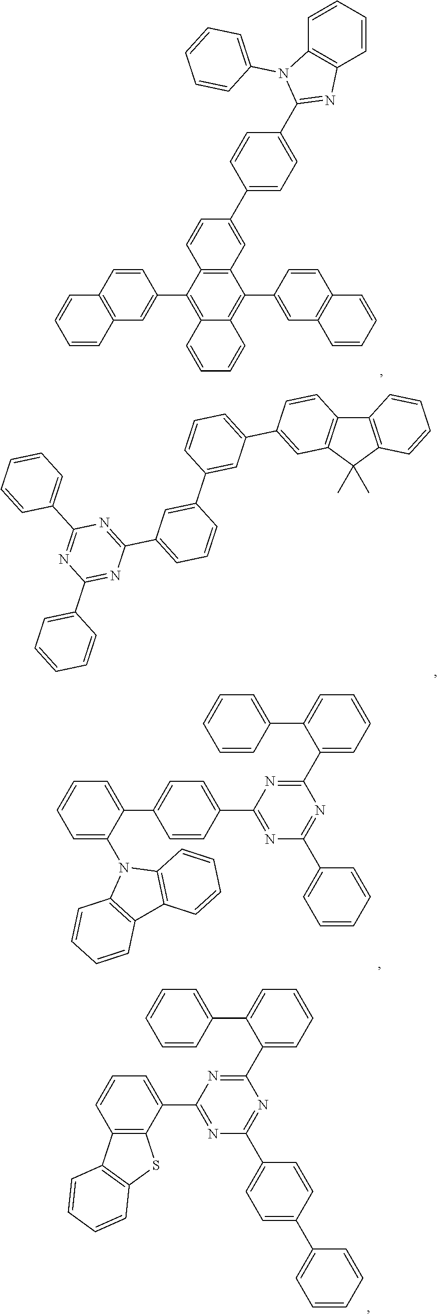

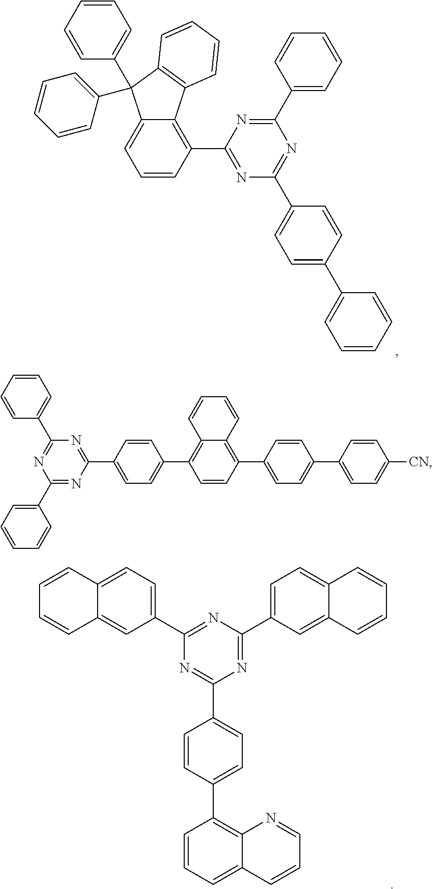

Conventional light-emitting materials and host materials in the related art may be used in the light-emitting layer. For example, the light-emitting layer may typically contain the following fluorescent light-emitting materials and fluorescent host materials without limitation:

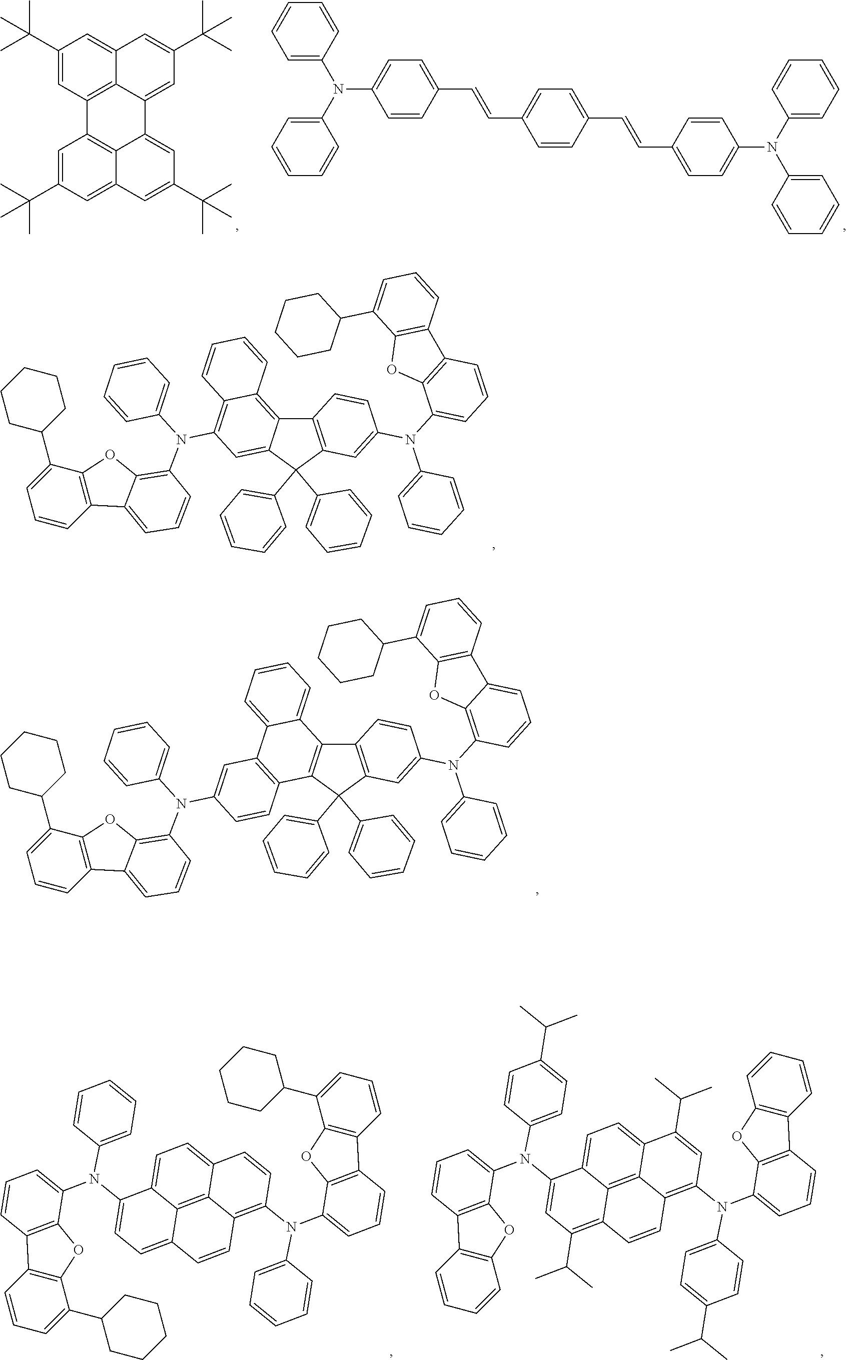

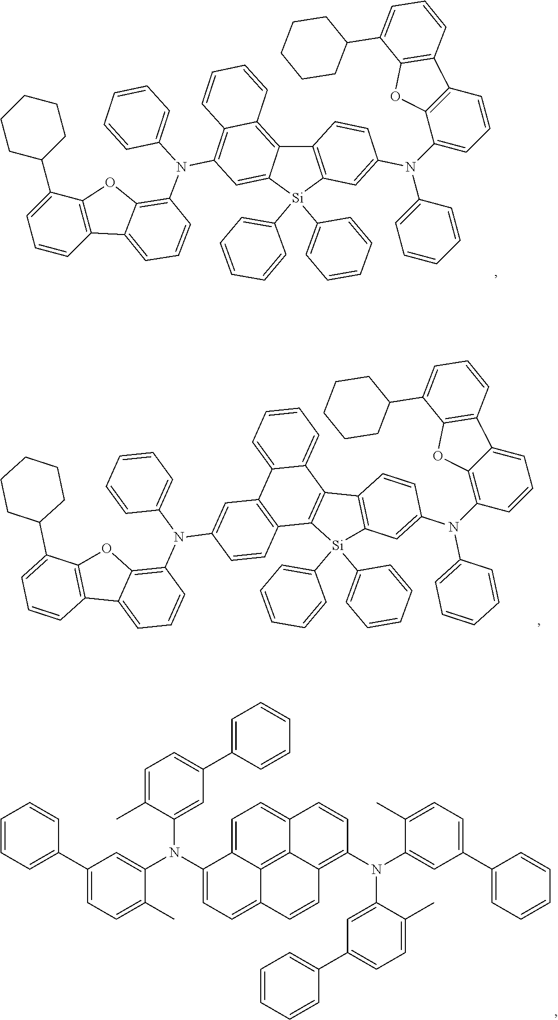 [SMILES: c1ccc(N(c2ccc3c(c2)[Si](c2ccccc2)(c2ccccc2)c2cc(N(c4ccccc4)c4cccc5c4oc4c(C6CCCCC6)cccc45)c4ccccc4c2-3)c2cccc3c2oc2c(C4CCCCC4)cccc23)cc1 ; c1ccc(N(c2ccc3c(c2)[Si](c2ccccc2)(c2ccccc2)c2c-3c3ccccc3c3cc(N(c4ccccc4)c4cccc5c4oc4c(C6CCCCC6)cccc45)ccc23)c2cccc3c2oc2c(C4CCCCC4)cccc23)cc1 ; Cc1ccc(-c2ccccc2)cc1N(c1cc(-c2ccccc2)ccc1C)c1ccc2ccc3c(N(c4cc(-c5ccccc5)ccc4C)c4cc(-c5ccccc5)ccc4C)ccc4ccc1c2c43]

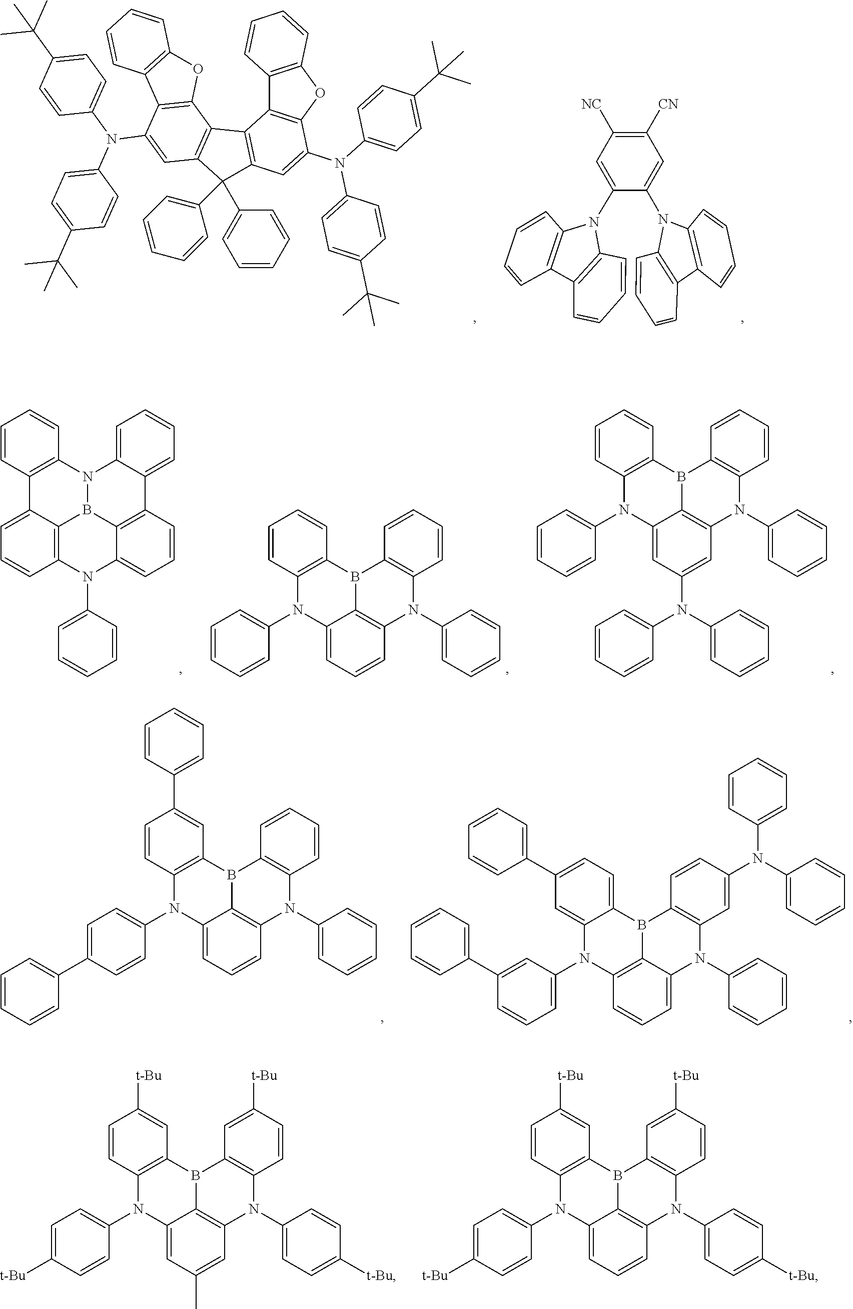

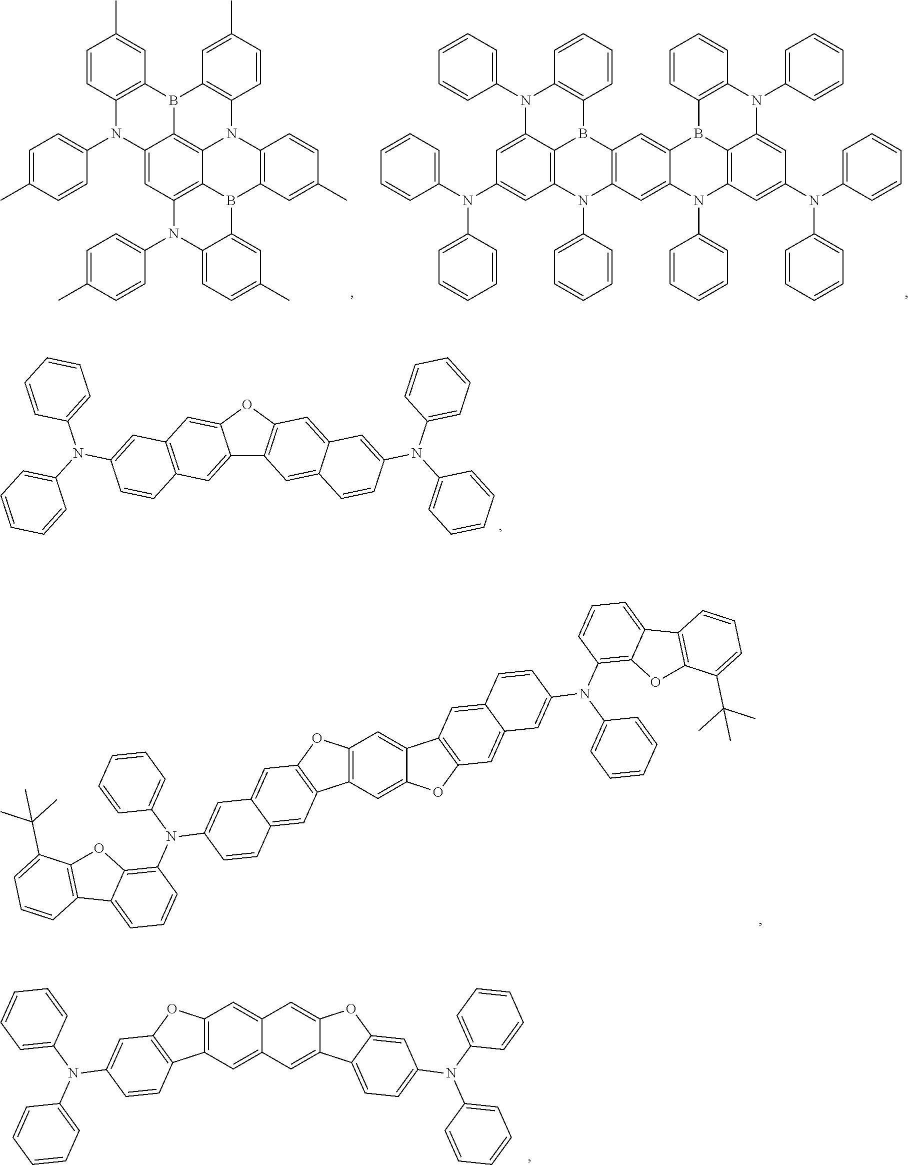

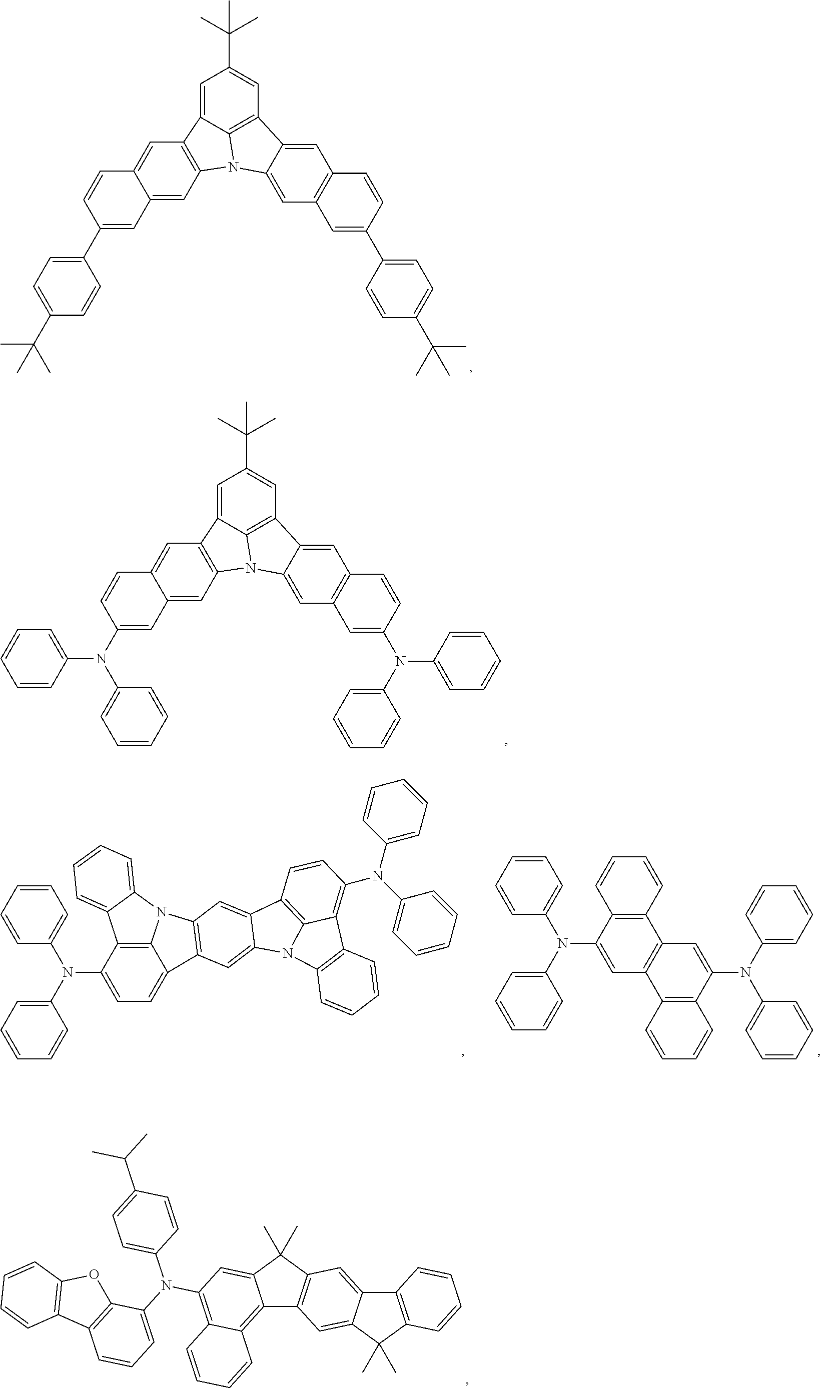

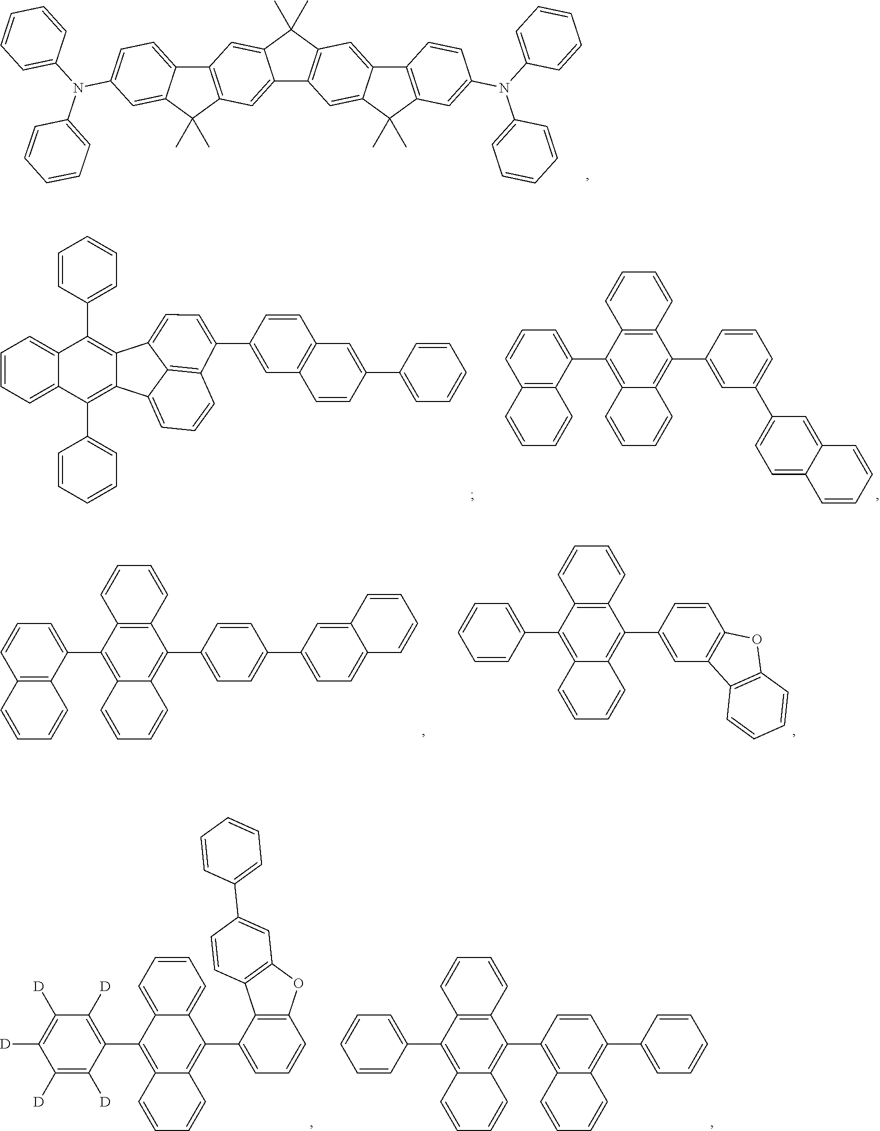

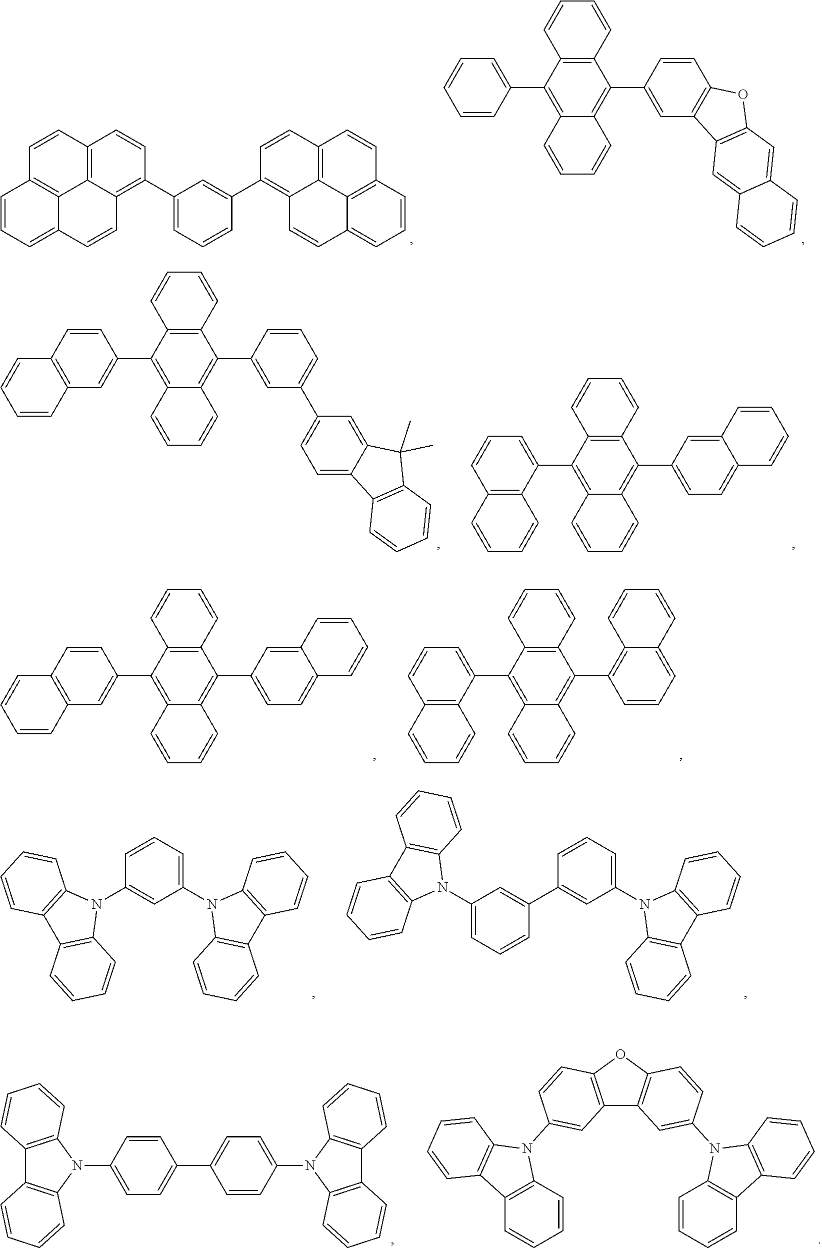

The light-emitting layer may also typically contain the following phosphorescent light-emitting materials and phosphorescent host materials without limitation:

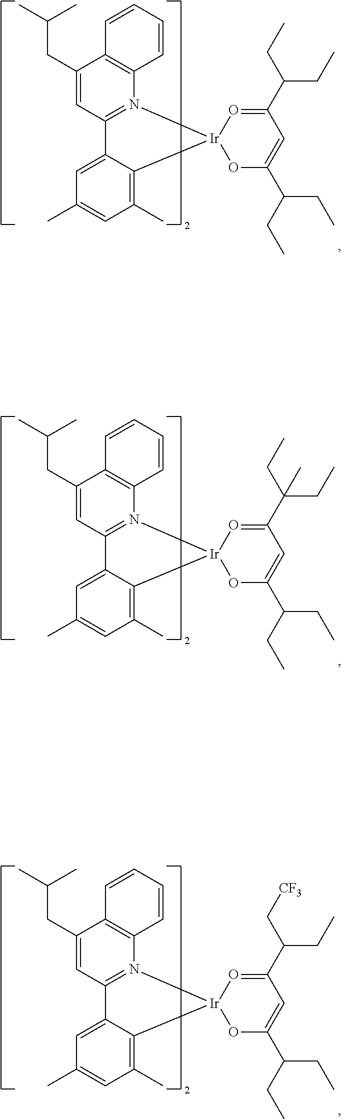

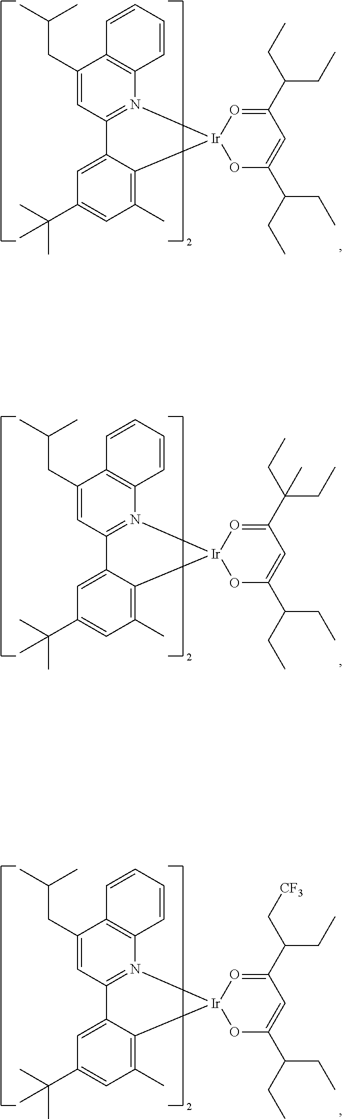

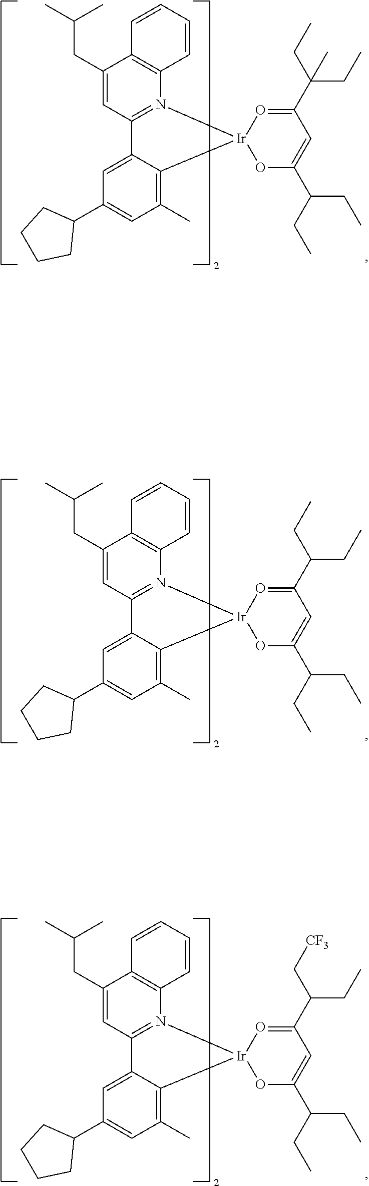

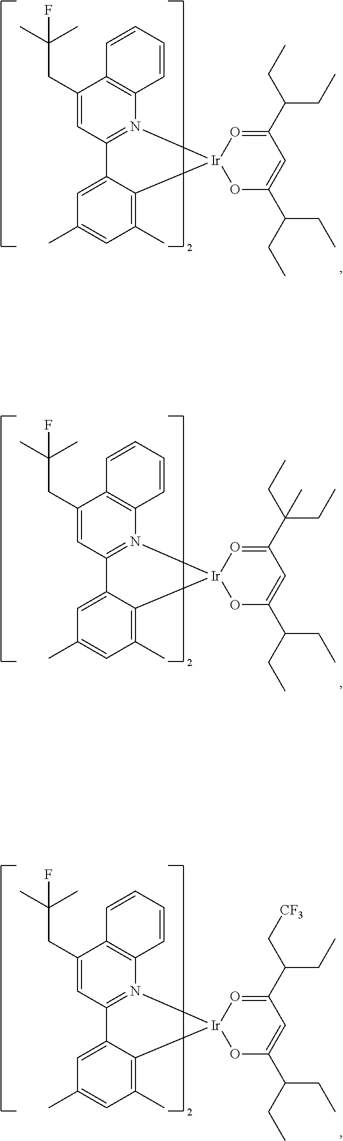

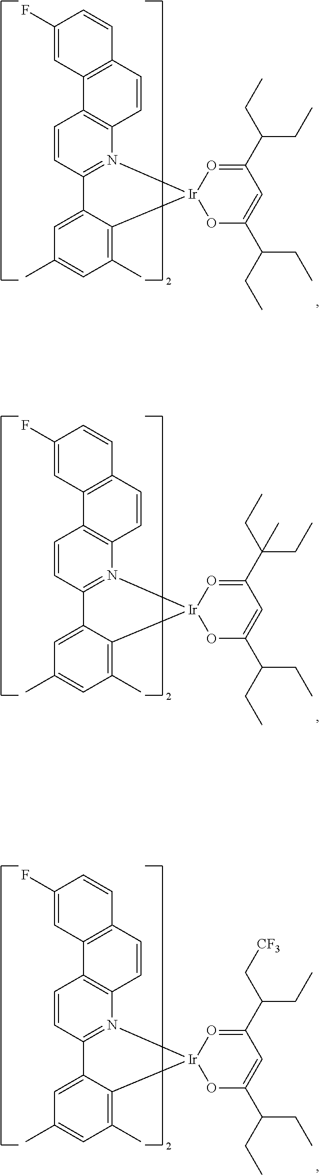

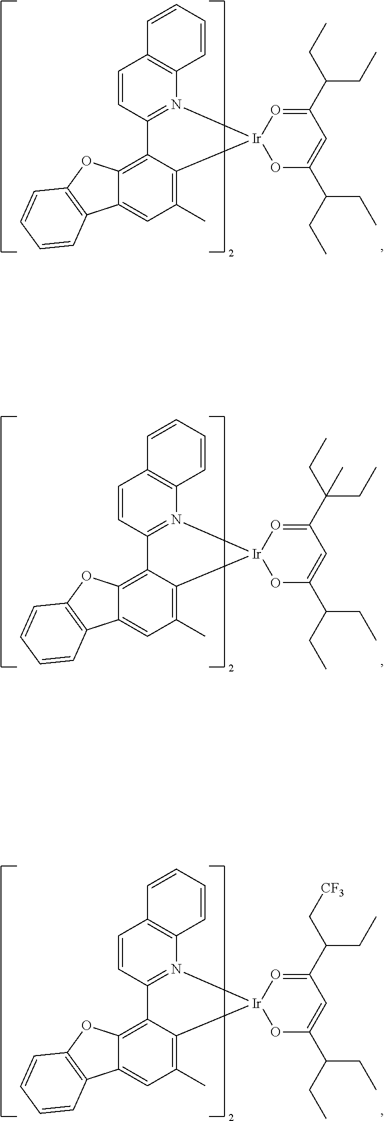

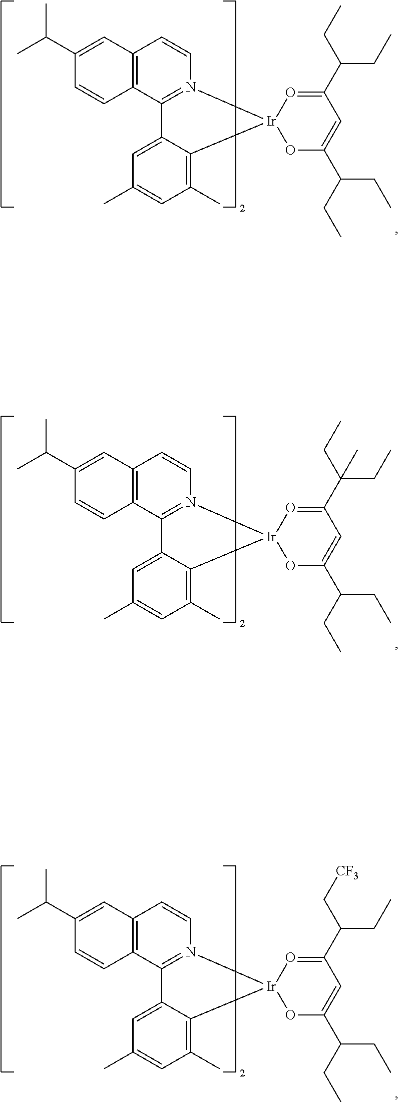

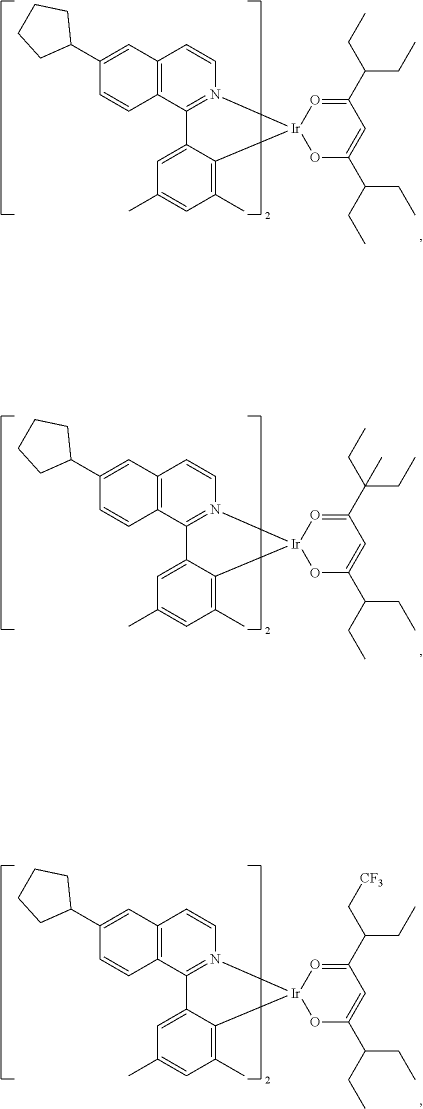

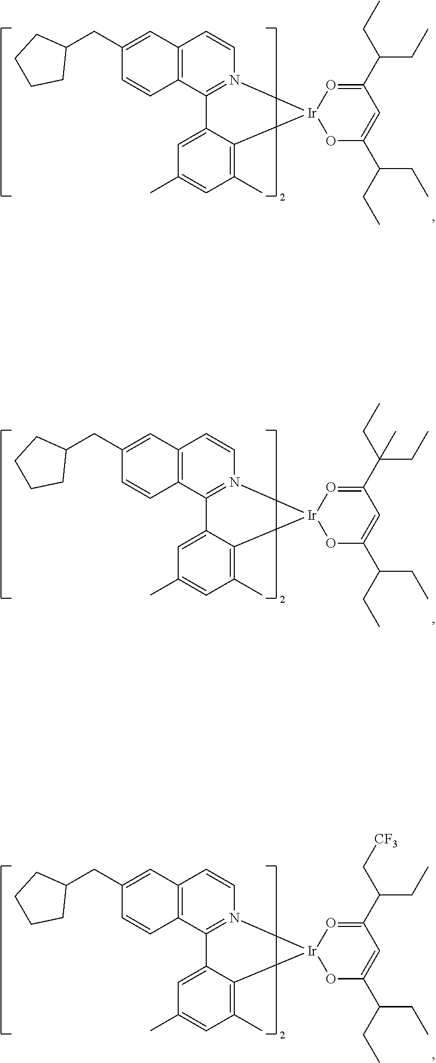

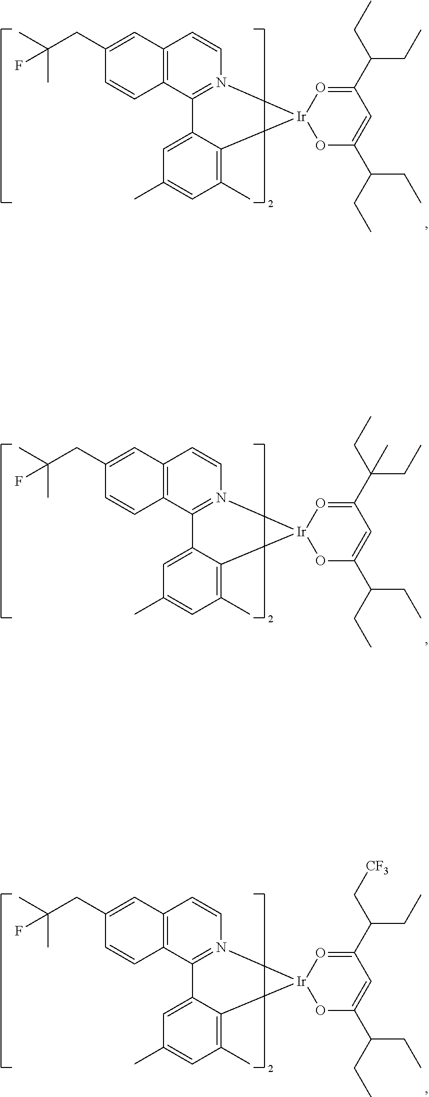

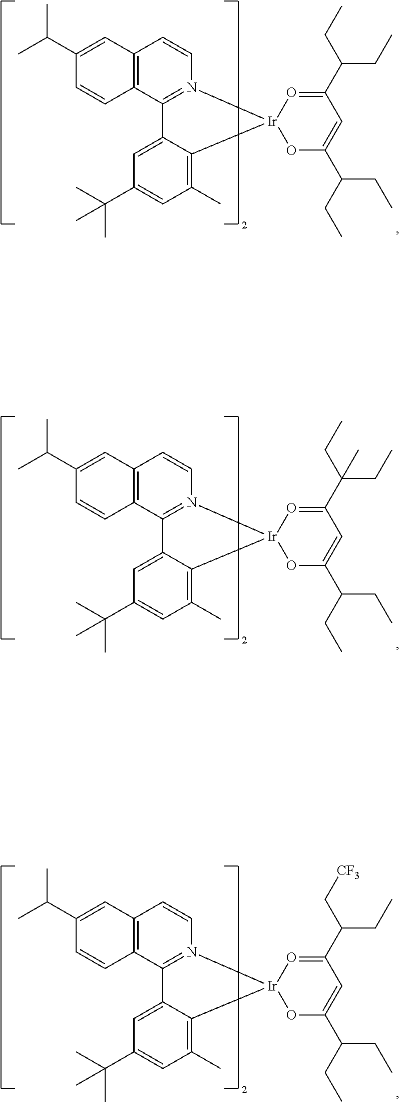

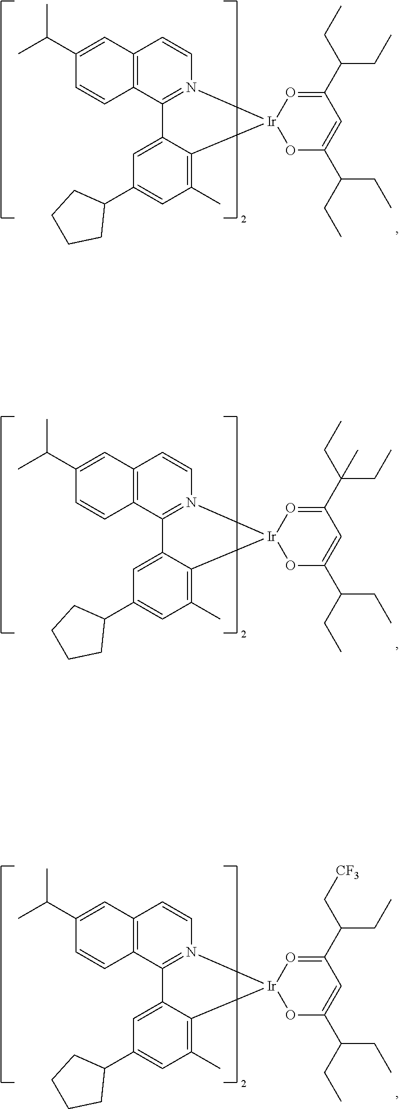

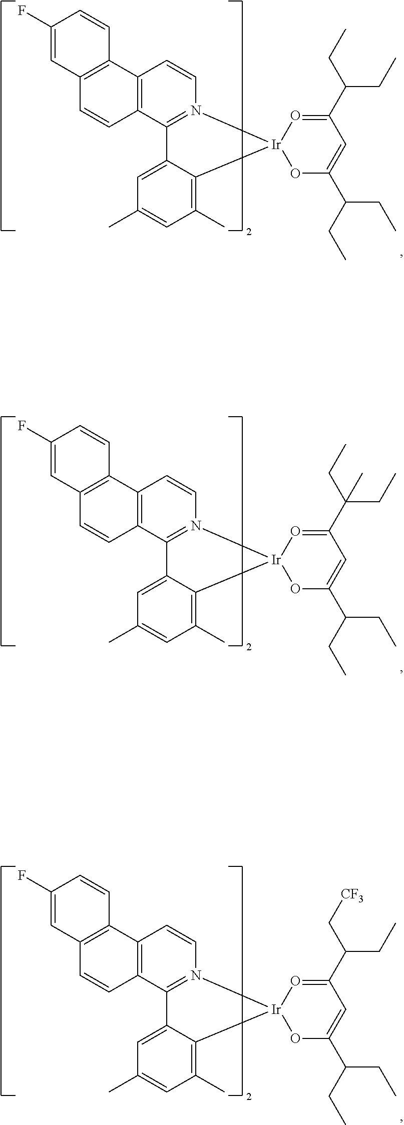

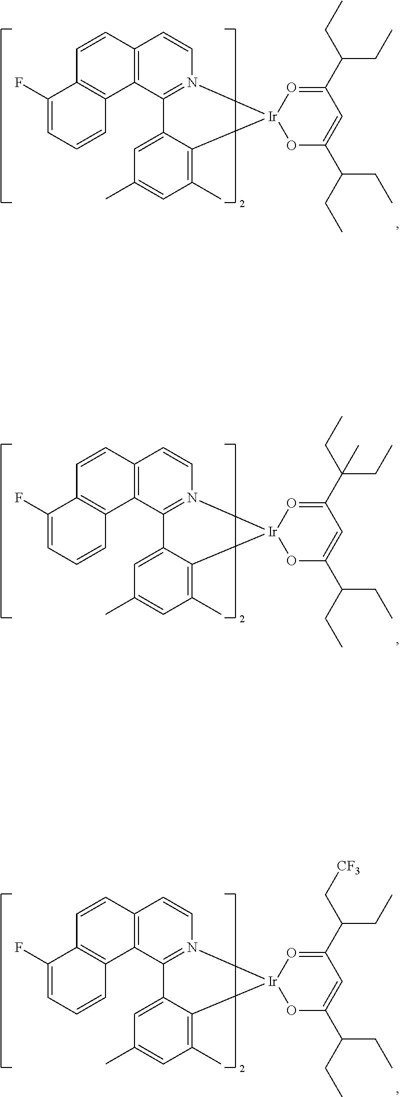

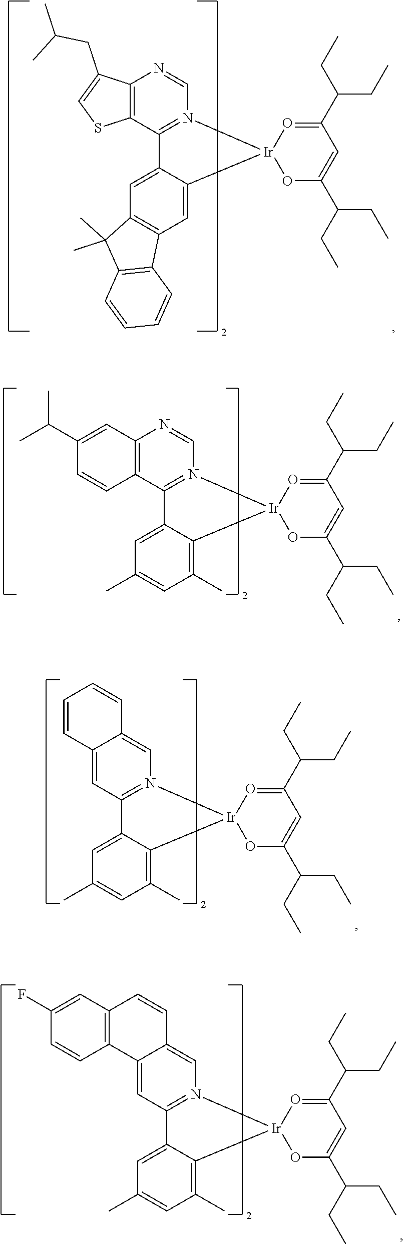

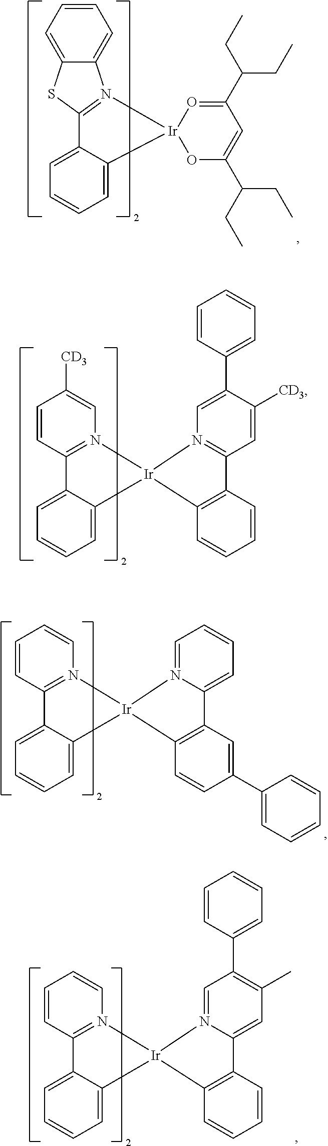

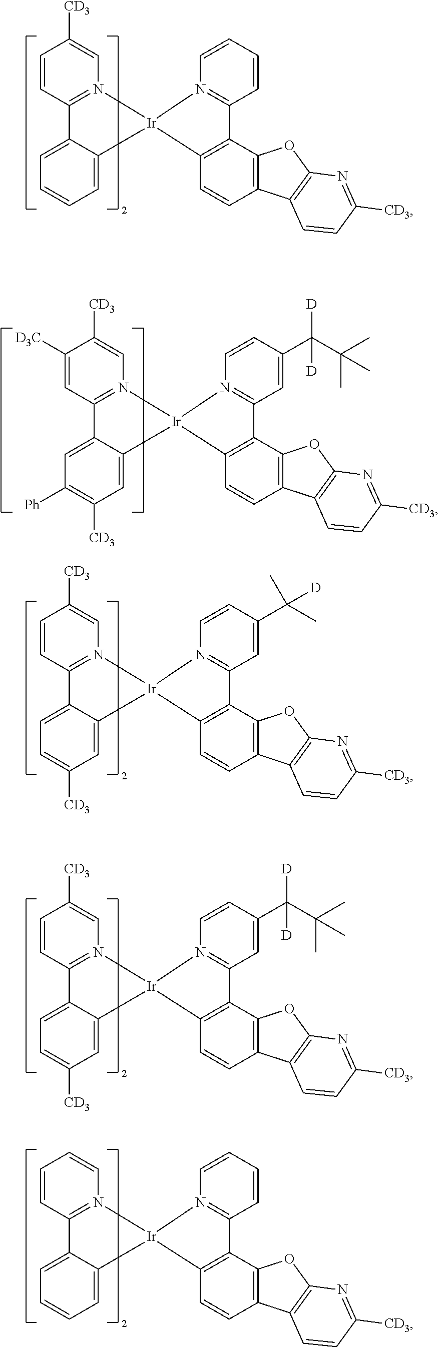

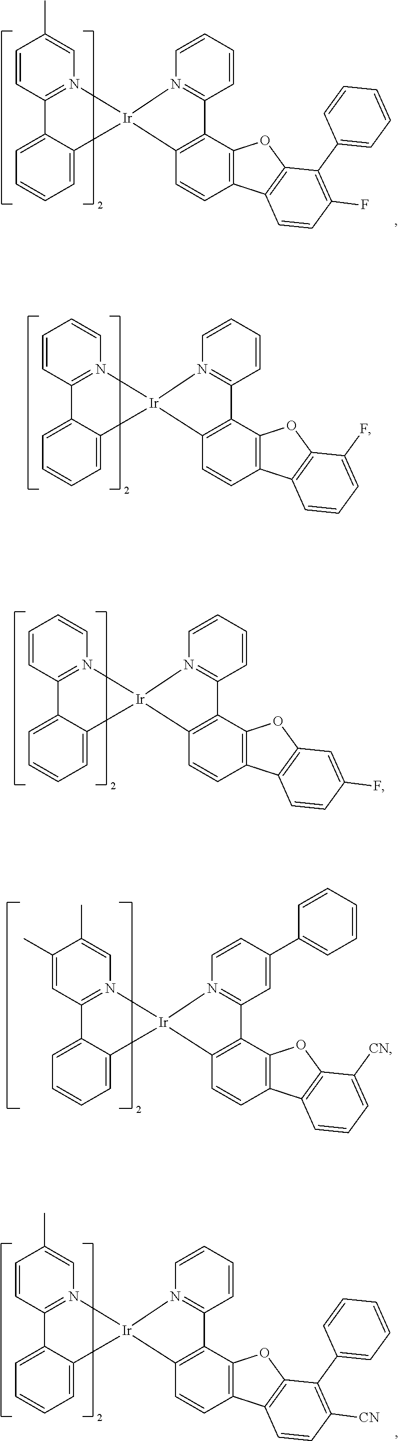

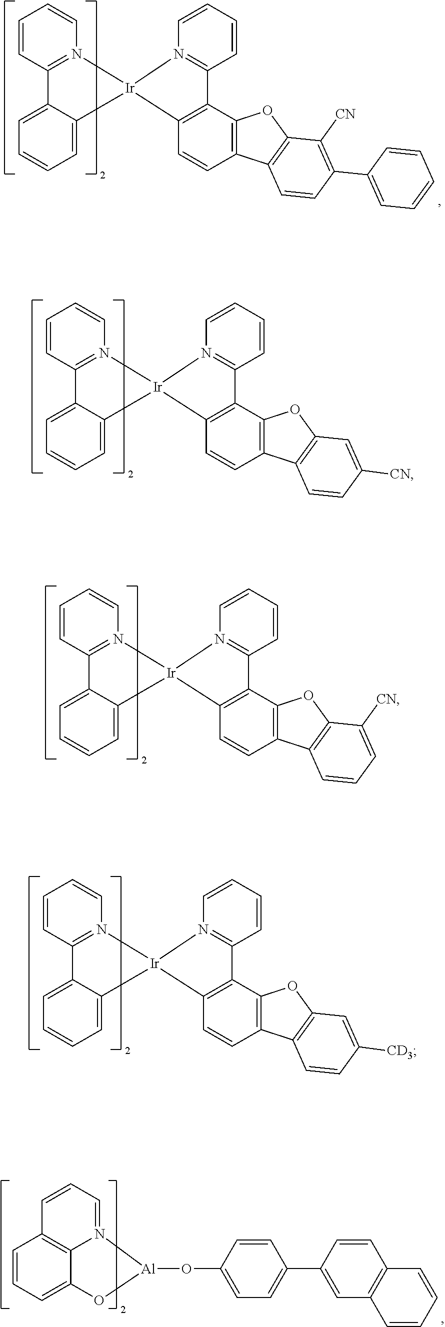

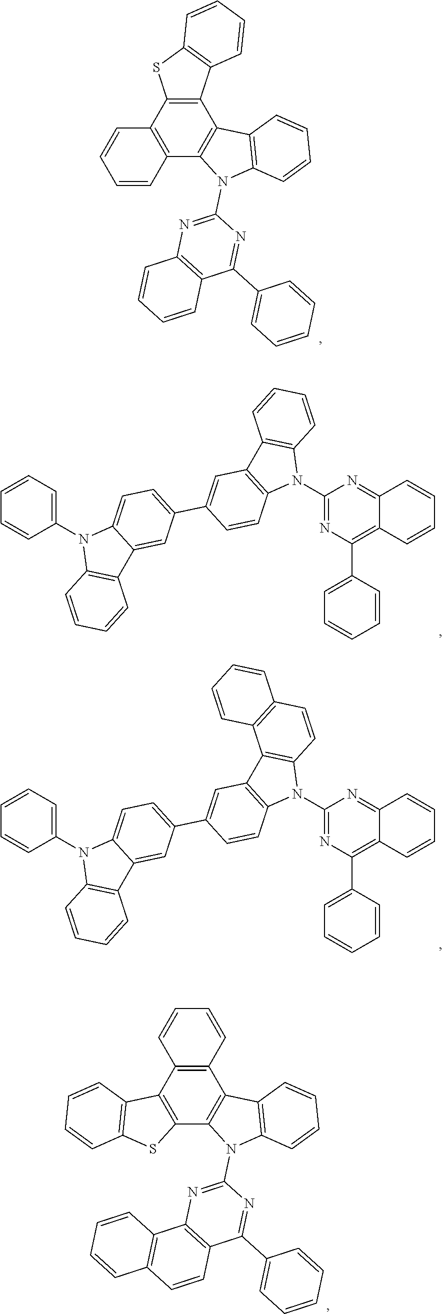

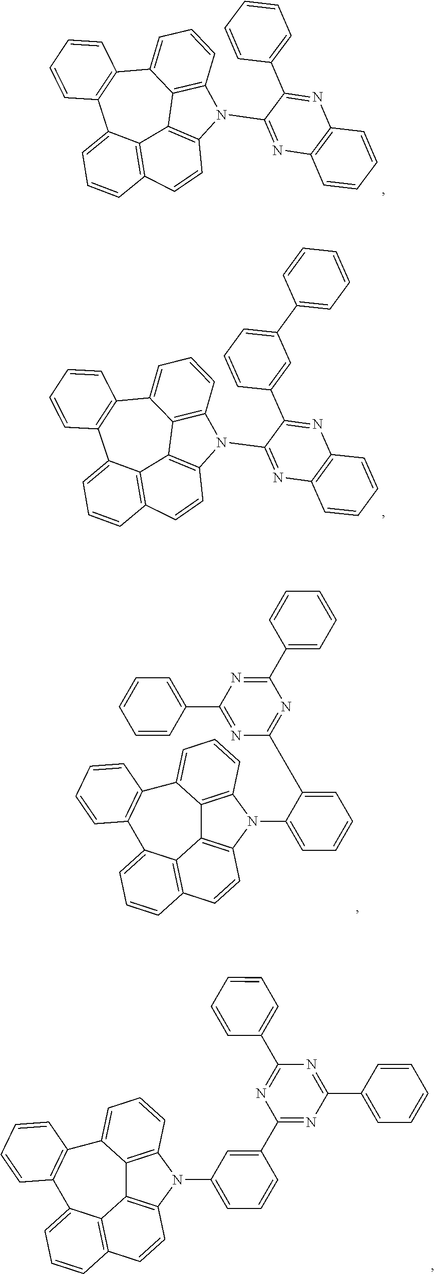 [SMILES: c1ccc(-c2nc3ccccc3nc2-n2c3cccc4c3c3c5c(cccc5ccc32)-c2ccccc2-4)cc1 ; c1ccc(-c2cccc(-c3nc4ccccc4nc3-n3c4cccc5c4c4c6c(cccc6ccc43)-c3ccccc3-5)c2)cc1 ; c1ccc(-c2nc(-c3ccccc3)nc(-c3ccccc3-n3c4cccc5c4c4c6c(cccc6ccc43)-c3ccccc3-5)n2)cc1 ; c1ccc(-c2nc(-c3ccccc3)nc(-c3cccc(-n4c5cccc6c5c5c7c(cccc7ccc54)-c4ccccc4-6)c3)n2)cc1]

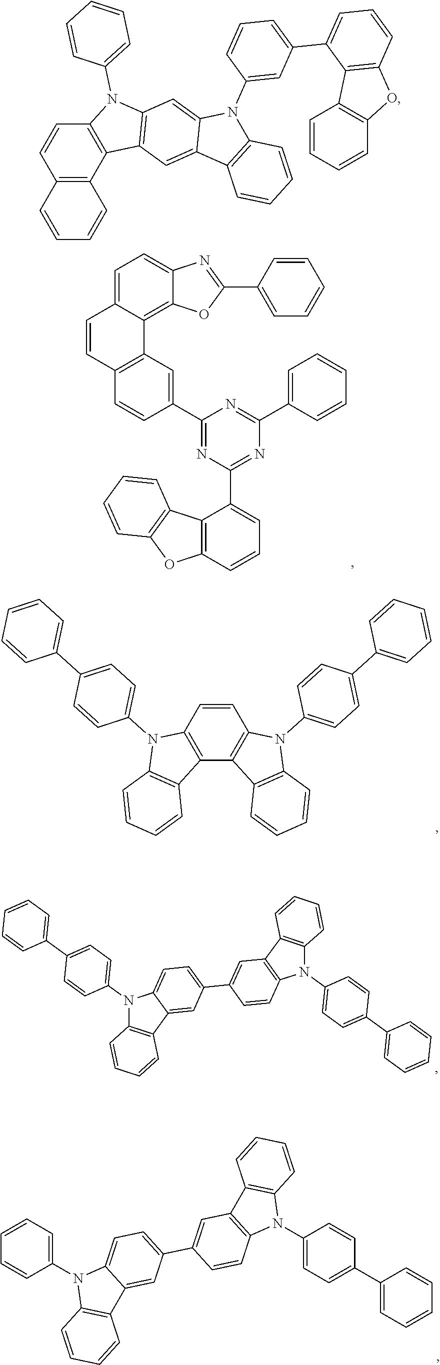

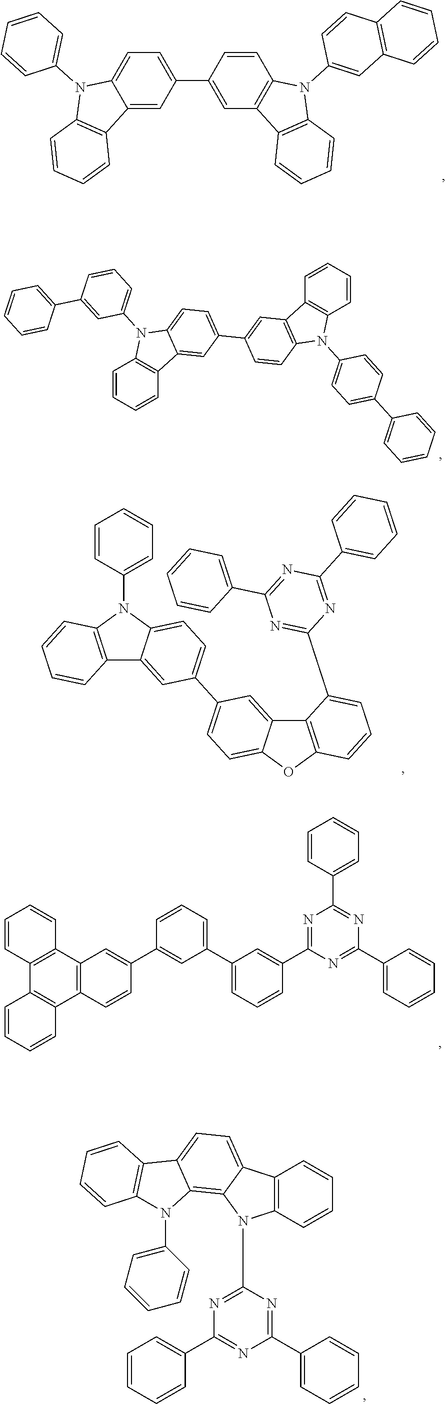

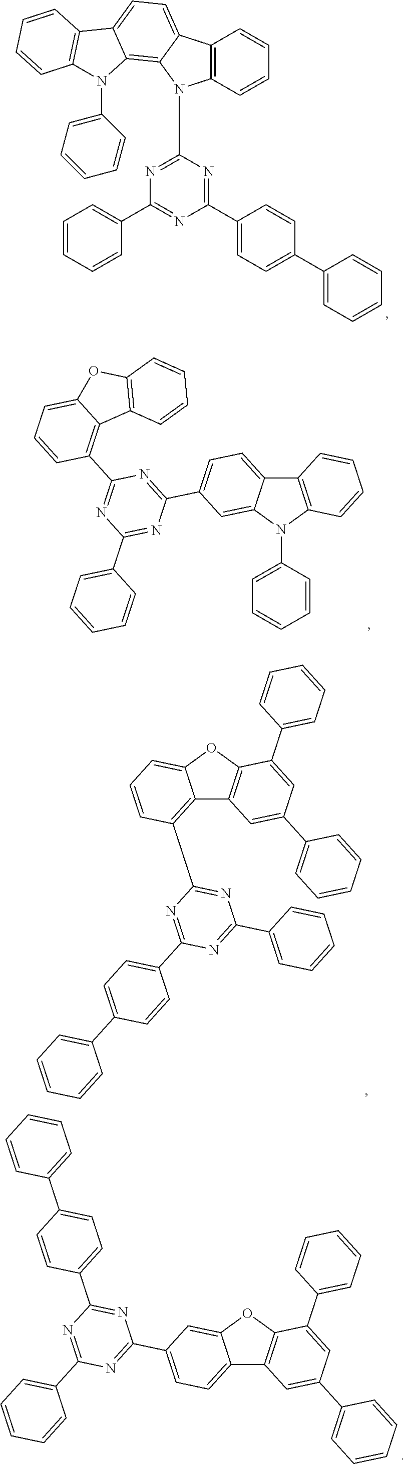

Conventional electron blocking materials in the related art may be used in the electron blocking layer. For example, the electron blocking layer may typically contain the following electron blocking materials without limitation:

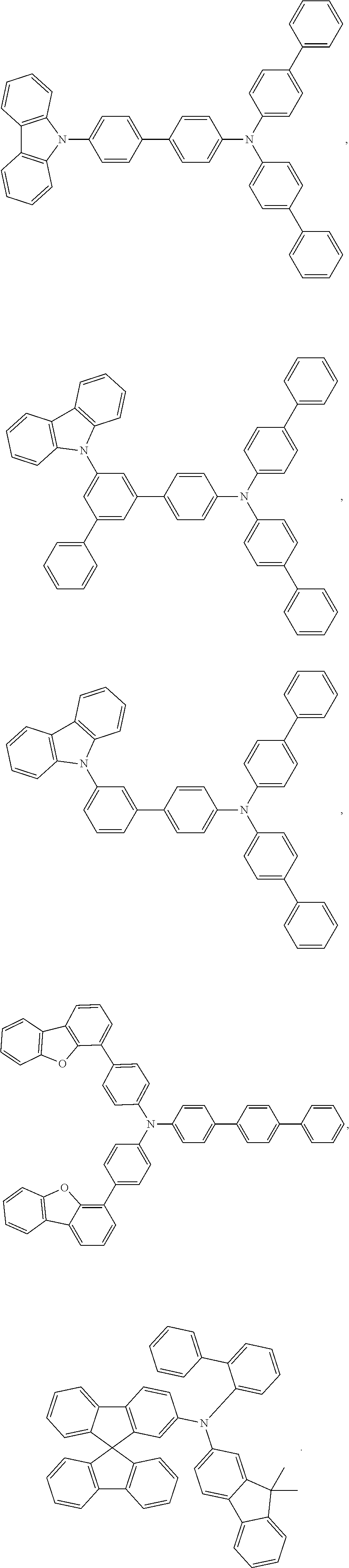

The first compound and the second compound used in the present disclosure may be obtained with reference to preparation methods in the related art or may also be easily prepared with reference to patent applications with Publication Nos. US20200087311A1 and US20160190447A1 and so on, which is not repeated here. The method for preparing an electroluminescent device is not limited. The preparation methods in the following example are merely examples and not to be construed as limitations. Those skilled in the art can make reasonable improvements on the preparation methods in the following examples based on the related art. Exemplarily, a ratio of various materials in each organic layer is not particularly limited, and those skilled in the art can make a reasonable selection within a certain range based on the related art. In device examples, the characteristics of the devices are also tested using conventional equipment in the art (including, but not limited to, an evaporator produced by ANGSTROM ENGINEERING, an optical testing system produced by SUZHOU FATAR, a life testing system produced by SUZHOU FATAR, and an ellipsometer produced by BEIJING ELLITOP, etc.) by methods well-known to those skilled in the art. As the persons skilled in the art are aware of the above-mentioned equipment use, test methods and other related contents, the inherent data of the sample can be obtained with certainty and without influence, so the above related contents are not further described in this patent.

### DEVICE EXAMPLE

### Example 1-1: Preparation of a Fluorescent Organic Electroluminescent Device

Firstly, a glass substrate having a thickness of 0.7 mm, on which an indium tin oxide (ITO) anode with a thickness of 800 Å had been patterned, was washed with deionized water and a detergent, and then the ITO surface was treated with oxygen plasma and UV ozone. Then, the substrate was dried in a glovebox to remove moisture, mounted on a support and transferred into a vacuum chamber. The organic layers specified below were sequentially deposited on the anode layer through vacuum thermal evaporation at a rate of 0.01-10/s and at a vacuum degree of about 10-6 Torr. Compound II-130 and Compound 70 were co-deposited as a hole injection layer (HIL, 99:1, 100 Å). Compound II-130 was deposited as a hole transporting layer (HTL, 1200 Å). Compound EB1 was deposited as an electron blocking layer (EBL, 50 Å). Compound BH and Compound BD were co-deposited as an emissive layer (EML, 96:4, 250 Å). Compound HB1 was deposited as a hole blocking layer (HBL, 50 Å). Compound ET and Liq were co-deposited as an electron transporting layer (ETL, 40:60, 300 Å). Liq was deposited as an electron injection layer (EIL) with a thickness of 10 Å. Finally, metal aluminum was deposited as a cathode (1200 Å). The device was transferred back to the glove box and encapsulated with a glass lid to complete the device.

Example 1-2: This example was prepared by the same method as Example 1-1 except that Compound II-7 and Compound 70 were deposited as a hole injection layer (HIL, 99:1, 100 Å) and Compound II-7 was deposited as a hole transporting layer (HTL, 1200 Å).

Comparative Example 1-1: This comparative example was prepared by the same method as Example 1-1 except that Compound HT and Compound 70 were deposited as a hole injection layer (HIL, 99:1, 100 Å) and Compound HT was deposited as a hole transporting layer (HTL, 1200 Å).

Comparative Example 1-2: This comparative example was prepared by the same method as Example 1-1 except that Compound II-130 and Compound PD-1 were deposited as a hole injection layer (HIL, 99:1, 100 Å).

Comparative Example 1-3: This comparative example was prepared by the same method as Example 1-2 except that Compound II-7 and Compound PD-1 were deposited as a hole injection layer (HIL, 99:1, 100 Å).

Comparative Example 1-4: This comparative example was prepared by the same method as Comparative Example 1-1 except that Compound HT and Compound PD-1 were deposited as a hole injection layer (HIL, 99:1, 100 Å).

Detailed structures and thicknesses of layers of the devices are shown in the following table. Layers using more than one material were obtained by doping different compounds at their weight ratio as recorded.

TABLE 1
Device structures of organic layers in Examples 1-1 and 1-2 and Comparative Examples 1-1 to 1-4

| Device No. | HIL | HTL | EBL | EML | HBL | ETL | EIL |
|---|---|---|---|---|---|---|---|
| Example 1-1 | Compound | Compound | EB1 (50 Å) | BH:BD | HB1 (50 Å) | ET:Liq | Liq (10 Å) |
| | II-130:Compound 70 | II-130 (1200 Å) | | (96:4) (250 Å) | | (40:60) (300 Å) | |
| | (99:1) (100 Å) | | | | | | |
| Example 1-2 | Compound | Compound | EB1 (50 Å) | BH:BD | HB1 (50 Å) | ET:Liq | Liq (10 Å) |
| | II-7:Compound 70 | II-7 (1200 Å) | | (96:4) (250 Å) | | (40:60) (300 Å) | |
| | (99:1) (100 Å) | | | | | | |
| Comparative | HT:Compound 70 | HT (1200 Å) | EB1 (50 Å) | BH:BD | HB1 (50 Å) | ET:Liq | Liq (10 Å) |
| Example 1-1 | (99:1) (100 Å) | | | (96:4) (250 Å) | | (40:60) (300 Å) | |
| Comparative | Compound | Compound | EB1 (50 Å) | BH:BD | HB1 (50 Å) | ET:Liq | Liq (10 Å) |
| Example 1-2 | II-130:PD-1 | II-130 (1200 Å) | | (96:4) (250 Å) | | (40:60) (300 Å) | |
| | (99:1) (100 Å) | | | | | | |
| Comparative | Compound | Compound | EB1 (50 Å) | BH:BD | HB1 (50 Å) | ET:Liq | Liq (10 Å) |
| Example 1-3 | II-7:PD-1 | II-7 (1200 Å) | | (96:4) (250 Å) | | (40:60) (300 Å) | |
| | (99:1) (100 Å) | | | | | | |
| Comparative | HT:PD-1 | HT (1200 Å) | EB1 (50 Å) | BH:BD | HB1 (50 Å) | ET:Liq | Liq (10 Å) |
| Example 1-4 | (99:1) (100 Å) | | | (96:4) (250 Å) | | (40:60) (300 Å) | |

The structures of the materials used in the devices are shown as follows:

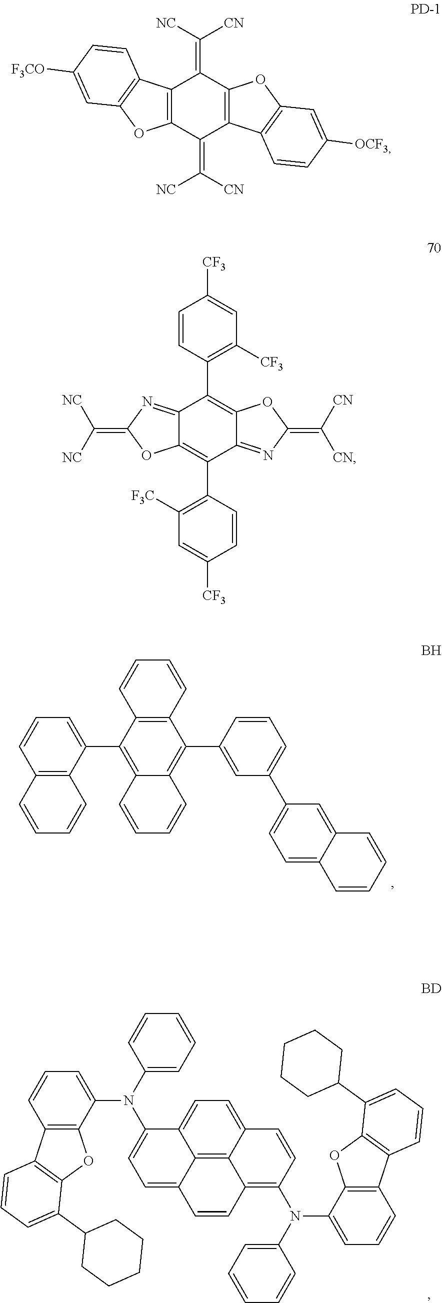

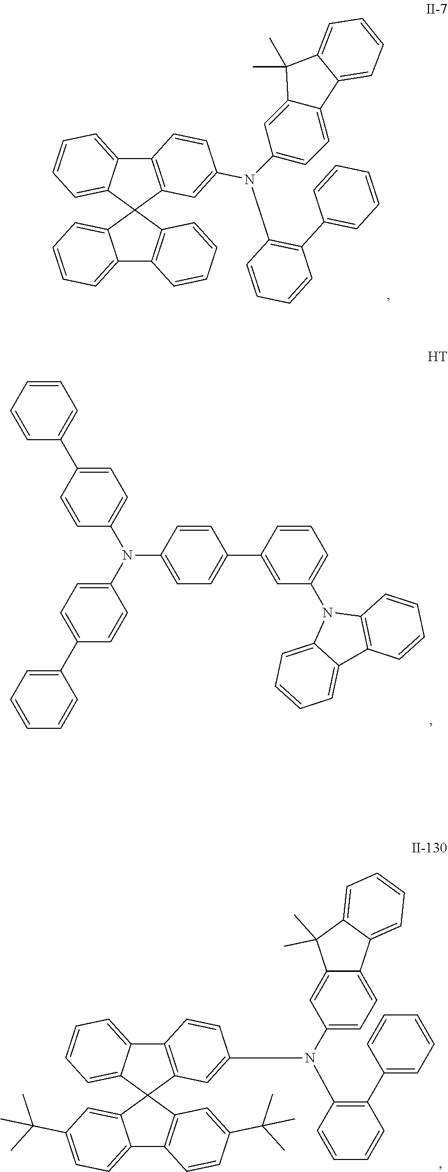 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc(-c4cccc(-n5c6ccccc6c6ccccc65)c4)cc3)cc2)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

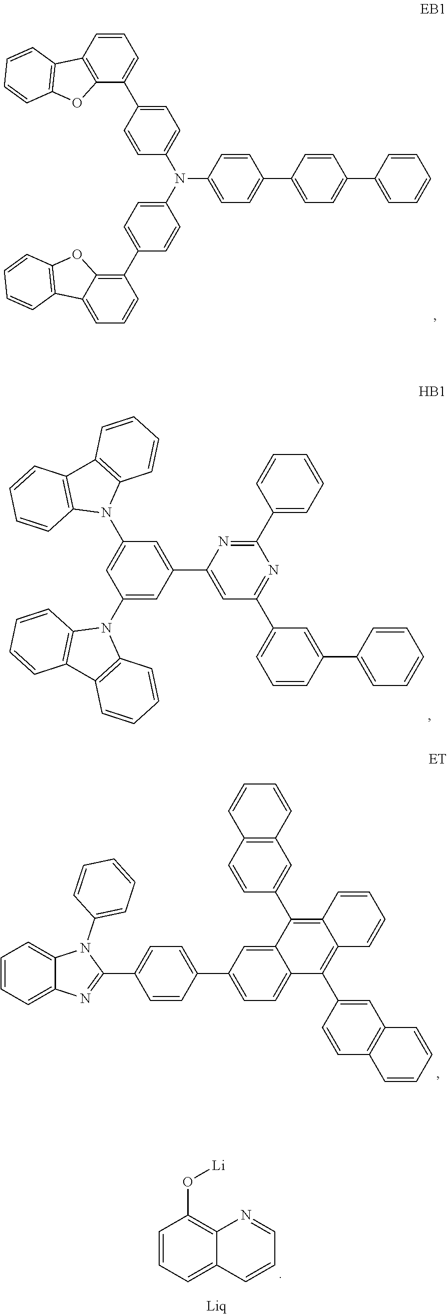

Device performance of Examples 1-1 and 1-2 and Comparative Examples 1-1 to 1-4 is shown in Table 2. Chromaticity coordinates (CIE), voltage and power efficiency (PE) were measured at a current density of 10 mA/cm², and a device lifetime (LT95) was a lifetime taken for the device to decay to 95% of initial brightness and measured at a constant current of 80 mA/cm².

TABLE 2
Device performance of Examples 1-1 and
1-2 and Comparative Examples 1-1 to 1-4

| | | | Voltage | PE | LT95 |
|---|---|---|---|---|---|
| Device No. | CIEx | CIEy | (V) | (lm/W) | (h) |
| Example 1-1 | 0.141 | 0.104 | 4.1 | 4.3 | 112 |
| Example 1-2 | 0.140 | 0.105 | 4.0 | 4.8 | 85 |
| Comparative Example 1-1 | 0.140 | 0.094 | 4.9 | 3.6 | 102 |
| Comparative Example 1-2 | 0.141 | 0.110 | 10.5 | 2.2 | 3 |
| Comparative Example 1-3 | 0.141 | 0.105 | 8.4 | 3.0 | 1 |
| Comparative Example 1-4 | 0.140 | 0.094 | 12.8 | 1.7 | 19 |

As can be seen from the data in Table 2, examples have substantially the same chromaticity coordinates as comparative examples.

Compared with Comparative Example 1-1, Example 1-1 has a voltage reduced by 0.8 V, power efficiency improved by 0.7 lm/W and a lifetime further significantly improved by nearly 10% on the basis of a very high level of Comparative Example 1-1, which is very rare. Compared with Comparative Example 1-2, Example 1-1 has a voltage greatly reduced by 6.4 V, power efficiency improved by 2.1 lm/W and a lifetime greatly improved 36 times. Compared with Comparative Example 1-4, Example 1-1 has a voltage greatly reduced by 8.7 V, power efficiency improved by 2.6 lm/W and a lifetime greatly improved 5 times.

Compared with Comparative Example 1-1, Example 1-2 has a voltage reduced by 0.9 V, power efficiency improved by 1.2 lm/W and a lifetime slightly reduced but still at a very high level in the industry. Compared with Comparative Example 1-3, Example 1-2 has a voltage greatly reduced by 4.4 V, power efficiency improved by 1.8 lm/W and a lifetime greatly improved 84 times. Compared with Comparative Example 1-4, Example 1-2 has a voltage greatly reduced by 8.8 V, power efficiency improved by 3.1 lm/W and a lifetime greatly improved 3.5 times.

As can be seen from the preceding comparison, a combination of the first compound and the second compound selected in the present disclosure, when used in the fluorescent organic electroluminescent device, enables the organic electroluminescent device to obtain lower voltage, higher efficiency and a longer lifetime, which proves the excellent performance and broad application prospect of the combination of the first compound and the second compound selected in the present disclosure.

### Example 2-1: Preparation of a Phosphorescent Organic Electroluminescent Device

Firstly, a glass substrate having a thickness of 0.7 mm, on which an indium tin oxide (ITO) anode with a thickness of 1200 Å had been patterned, was washed with deionized water and a detergent, and then the ITO surface was treated with oxygen plasma and UV ozone. Then, the substrate was dried in a glovebox to remove moisture, mounted on a support and transferred into a vacuum chamber. The organic layers specified below were sequentially deposited on the anode layer through vacuum thermal evaporation at a rate of 0.01-10/s and at a vacuum degree of about 10-6 Torr. Compound II-130 and Compound 70 were co-deposited as a hole injection layer (HIL, 99:1, 100 Å). Compound II-130 was deposited as a hole transporting layer (HTL, 2000 Å). Compound EB2 was deposited as an electron blocking layer (EBL, 50 Å). Compound RH and Compound RD were co-deposited as an emissive layer (EML, 98:2, 400 Å). Compound HB2 was deposited as a hole blocking layer (HBL, 50 Å). Compound ET and Liq were co-deposited as an electron transporting layer (ETL, 40:60, 350 Å). Liq was deposited as an electron injection layer (EIL) with a thickness of 10 Å. Finally, a metal aluminum was deposited for used as a cathode (1200 Å). The device was transferred back to the glovebox and encapsulated with a glass lid to complete the device.

Example 2-2: This example was prepared by the same method as Example 2-1 except that Compound II-7 and Compound 70 were deposited as a hole injection layer (HIL, 99:1, 100 Å) and Compound II-7 was deposited as a hole transporting layer (HTL, 2000 Å).

Comparative Example 2-1: This comparative example was prepared by the same method as Example 2-1 except that Compound HT and Compound 70 were deposited as a hole injection layer (HIL, 99:1, 100 Å) and Compound HT was deposited as a hole transporting layer (HTL, 2000 Å).

Comparative Example 2-2: This comparative example was prepared by the same method as Example 2-1 except that Compound II-130 and Compound PD-1 were deposited as a hole injection layer (HIL, 99:1, 100 Å).

Comparative Example 2-3: This comparative example was prepared by the same method as Example 2-2 except that Compound II-7 and Compound PD-1 were deposited as a hole injection layer (HIL, 99:1, 100 Å).

Comparative Example 2-4: This comparative example was prepared by the same method as Comparative Example 2-1 except that Compound HT and Compound PD-1 were deposited as a hole injection layer (HIL, 99:1, 100 Å).

Detailed structures and thicknesses of layers of the devices are shown in the following table. Layers using more than one material were obtained by doping different compounds at their weight ratio as recorded.

TABLE 3
Device structures of organic layers in Examples 2-1 and 2-2 and Comparative Examples 2-1 to 2-4

| Device No. | HIL | HTL | EBL | EML | HBL | ETL | EIL |
|---|---|---|---|---|---|---|---|
| Example 2-1 | Compound | Compound | EB2 (50 Å) | RH:RD | HB2 (50 Å) | ET:Liq | Liq (10 Å) |
| | II-130:Compound 70 | II-130 (2000 Å) | | (98:2) (400 Å) | | (40:60) (350 Å) | |
| | (99:1) (100 Å) | | | | | | |
| Example 2-2 | Compound | Compound | EB2 (50 Å) | RH:RD | HB2 (50 Å) | ET:Liq | Liq (10 Å) |
| | II-7:Compound 70 | II-7 (2000 Å) | | (98:2) (400 Å) | | (40:60) (350 Å) | |
| | (99:1) (100 Å) | | | | | | |
| Comparative | HT:Compound 70 | HT (2000 Å) | EB2 (50 Å) | RH:RD | HB2 (50 Å) | ET:Liq | Liq (10 Å) |
| Example 2-1 | (99:1) (100 Å) | | | (98:2) (400 Å) | | (40:60) (350 Å) | |
| Comparative | Compound | Compound | EB2 (50 Å) | RH:RD | HB2 (50 Å) | ET:Liq | Liq (10 Å) |
| Example 2-2 | II-130:PD-1 | II-130 (2000 Å) | | (98:2) (400 Å) | | (40:60) (350 Å) | |
| | (99:1) (100 Å) | | | | | | |
| Comparative | Compound | Compound | EB2 (50 Å) | RH:RD | HB2 (50 Å) | ET:Liq | Liq (10 Å) |
| Example 2-3 | II-7:PD-1 | II-7 (2000 Å) | | (98:2) (400 Å) | | (40:60) (350 Å) | |
| | (99:1) (100 Å) | | | | | | |
| Comparative | HT:PD-1 | HT (2000 Å) | EB2 (50 Å) | RH:RD | HB2 (50 Å) | ET:Liq | Liq (10 Å) |
| Example 2-4 | (99:1) (100 Å) | | | (98:2) (400 Å) | | (40:60) (350 Å) | |

The structures of the materials newly used in the devices are shown as follows:

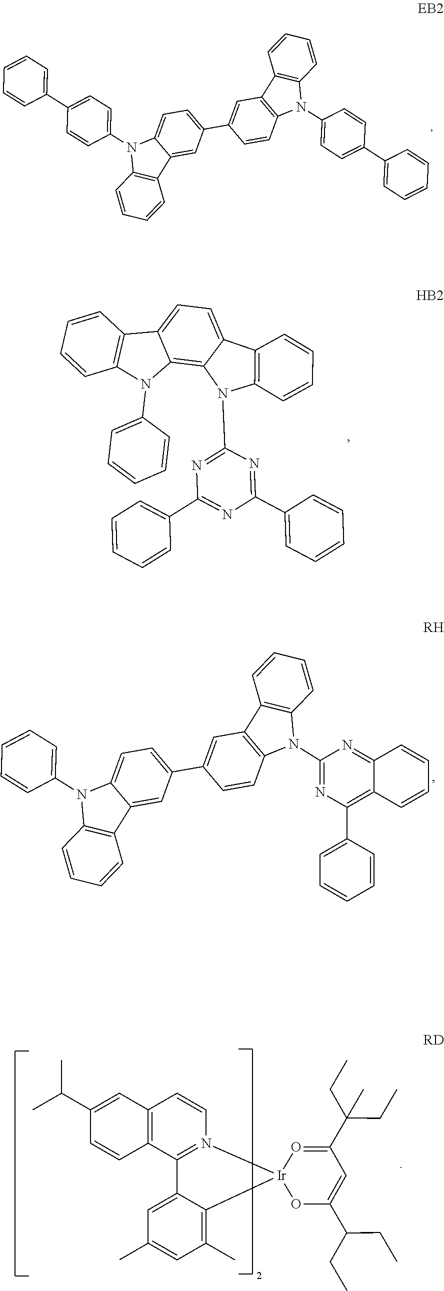

Device performance of Examples 2-1 and 2-2 and Comparative Examples 2-1 to 2-4 is shown in Table 4. Chromaticity coordinates (CIE), voltage and power efficiency (PE) were measured at a current density of 10 mA/cm², and a device lifetime (LT95) was a lifetime taken for the device to decay to 95% of initial brightness and measured at a constant current of 80 mA/cm².

TABLE 4
Device performance of Examples 2-1 and
2-2 and Comparative Examples 2-1 to 2-4

| | | | Voltage | PE | LT95 |
|---|---|---|---|---|---|
| Device No. | CIEx | CIEy | (V) | (lm/W) | (h) |
| Example 2-1 | 0.681 | 0.318 | 4.5 | 14.7 | 132 |
| Example 2-2 | 0.680 | 0.318 | 4.3 | 15.2 | 131 |
| Comparative Example 2-1 | 0.681 | 0.318 | 6.4 | 11.4 | 81 |
| Comparative Example 2-2 | 0.679 | 0.319 | 14.6 | 6.8 | 20 |
| Comparative Example 2-3 | 0.678 | 0.320 | 12.8 | 7.4 | 65 |
| Comparative Example 2-4 | 0.678 | 0.320 | 25.9 | 3.1 | 1 |

Compared with Comparative Example 2-1, Example 2-1 has a voltage reduced by 1.9 V, power efficiency improved by 3.3 lm/W and a lifetime improved 0.6 times. Compared with Comparative Example 2-2, Example 2-1 has a voltage greatly reduced by 10.1 V, power efficiency improved by 7.9 lm/W and a lifetime greatly improved 5.6 times. Compared with Comparative Example 2-4, Example 2-1 has a voltage greatly reduced by 21.4 V, power efficiency improved by 11.6 lm/W and a lifetime greatly improved 131 times.

Compared with Comparative Example 2-1, Example 2-2 has a voltage reduced by 2.1 V, power efficiency improved by 3.8 lm/W and a lifetime improved 0.6 times. Compared with Comparative Example 2-3, Example 2-2 has a voltage greatly reduced by 8.5 V, power efficiency improved by 7.8 lm/W and a lifetime improved 1 times. Compared with Comparative Example 2-4, Example 2-2 has a voltage greatly reduced by 21.6 V, power efficiency improved by 12.1 lm/W and a lifetime greatly improved 130 times.

As can be seen from the preceding comparison, the combination of the first compound and the second compound selected in the present disclosure, when used in the phosphorescent organic electroluminescent device, enables the organic electroluminescent device to obtain the lower voltage, the higher efficiency and the longer lifetime, which proves the excellent performance and broad application prospect of the combination of the first compound and the second compound selected in the present disclosure.

To conclude, no matter whether it is used in the fluorescent organic electroluminescent device or the phosphorescent organic electroluminescent device, the material combination of the first compound and the second compound selected in the present disclosure can achieve the excellent effects of reducing voltage, improving efficiency and greatly improving the device lifetime or maintaining a high level of device lifetime, which suggests the broad application prospect of the material combination in industrial applications.

It is to be understood that various embodiments described herein are merely examples and not intended to limit the scope of the present disclosure. Therefore, it is apparent to those skilled in the art that the present disclosure as claimed may include variations of specific embodiments and preferred embodiments described herein. Many of the materials and structures described herein may be replaced with other materials and structures without departing from the spirit of the present disclosure. It is to be understood that various theories as to why the present disclosure works are not intended to be limitative.

What is claimed is:

1. An organic electroluminescent device, comprising:
an anode,
a cathode, and
a first organic layer disposed between the anode and the cathode, wherein the first organic layer at least contains a first compound and a second compound, wherein the first compound has a structure represented by Formula 1: 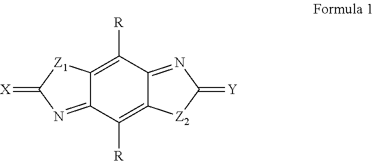
wherein in Formula 1,
X and Y are, at each occurrence identically or differently, selected from NR′, CR″R′″, O, S or Se;
Z₁ and Z₂ are, at each occurrence identically or differently, selected from O, S or Se;
R, R′, R″ and R′″ are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,
each R may be the same or different, and at least one of R, R′, R″ and R′″ is a group having at least one electron withdrawing group;
in Formula 1, adjacent substituents can be optionally joined to form a ring;
wherein the second compound has a structure represented by Formula 2: 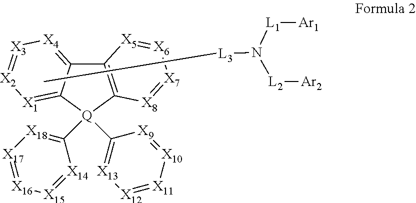
wherein in Formula 2,
X₁ to X₈ are, at each occurrence identically or differently, selected from C, CR₁ or N; and X₉ to X₁₈ are, at each occurrence identically or differently, selected from CR₁ or N;
Q is selected from C, Si or Ge;
L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof,
Ar₁ and Ar₂ are, at each occurrence identically or differently, selected from substituted or unsubstituted aryl having 6 to 30 carbon atoms or substituted or unsubstituted heteroaryl having 3 to 30 carbon atoms;
R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents L₁, L₂, L₃, R₁, Ar₁ and Ar₂ can be optionally joined to form a ring.

2. The organic electroluminescent device of claim 1, wherein in Formula 1, X and Y are, at each occurrence identically or differently, selected from CR″R′″ or NR′, and R′, R″ and R′″ each are a group having at least one electron withdrawing group; preferably, R, R′, R″ and R′″ each are a group having at least one electron withdrawing group.

3. The organic electroluminescent device of claim 1, wherein in Formula 1, X and Y are, at each occurrence identically or differently, selected from O, S or Se, and at least one R is a group having at least one electron withdrawing group; preferably, each R is a group having at least one electron withdrawing group.

4. The organic electroluminescent device of claim 1, wherein a Hammett constant of the electron withdrawing group is greater than or equal to 0.05, preferably greater than or equal to 0.3, more preferably greater than or equal to 0.5.

5. The organic electroluminescent device of claim 1, wherein the electron withdrawing group is selected from the group consisting of: halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, an aza-aromatic ring group and any one of the following groups substituted by one or more of halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group and an aza-aromatic ring group: alkyl having 1 to 20 carbon atoms, cycloalkyl having 3 to 20 ring carbon atoms, a heteroalkyl having 1 to 20 carbon atoms, arylalkyl having 7 to 30 carbon atoms, alkoxy having 1 to 20 carbon atoms, aryloxy having 6 to 30 carbon atoms, alkenyl having 2 to 20 carbon atoms, alkynyl having 2 to 20 carbon atoms, aryl having 6 to 30 carbon atoms, heteroaryl having 3 to 30 carbon atoms, alkylsilyl having 3 to 20 carbon atoms, arylsilyl having 6 to 20 carbon atoms and combinations thereof;
preferably, the electron withdrawing group is selected from the group consisting of: F, CF₃, OCF₃, SF₅, SO₂CF₃, a cyano group, an isocyano group, SCN, OCN, a pyrimidyl group, a triazinyl group and combinations thereof.

6. The organic electroluminescent device of claim 1, wherein X and Y are, at each occurrence identically or differently, selected from the group consisting of the following structures:
O, S, Se, 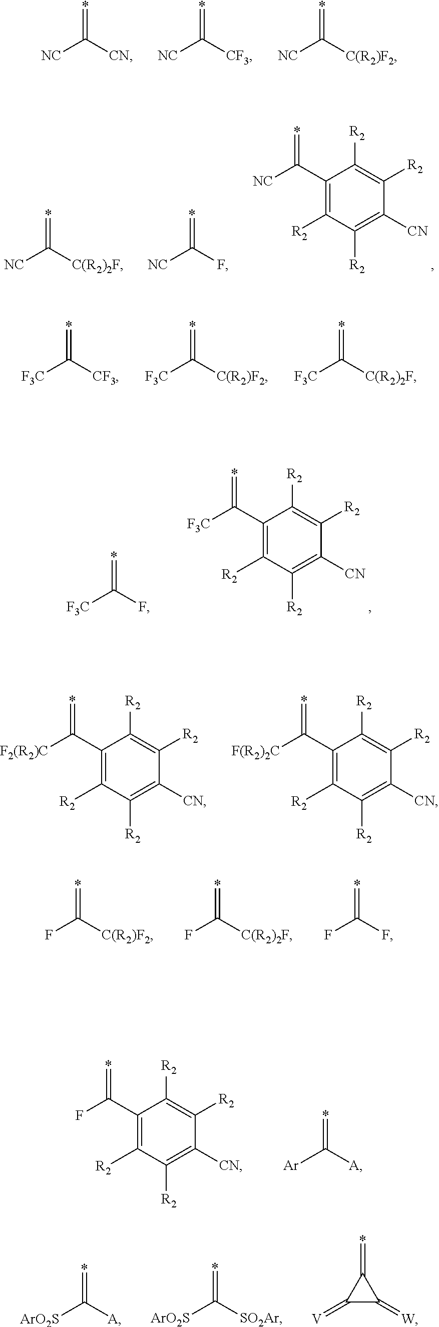 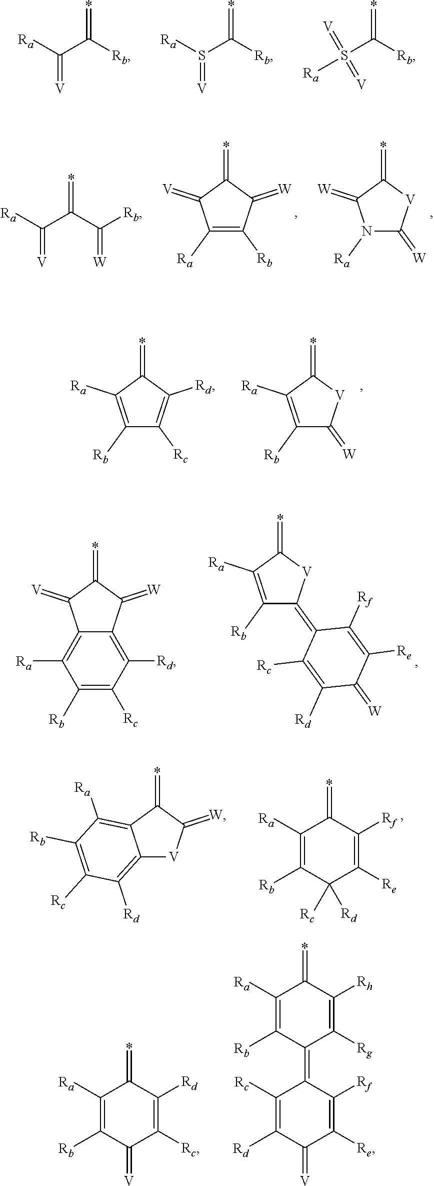 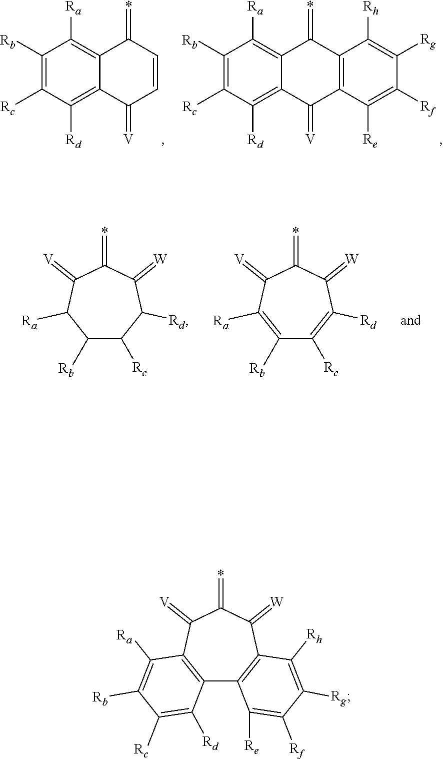
wherein
R₂ is, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,
preferably, R₂ is, at each occurrence identically or differently, selected from the group consisting of: F, CF₃, OCF₃, SF₅, SO₂CF₃, a cyano group, an isocyano group, SCN, OCN, pentafluorophenyl, 4-cyanotetrafluorophenyl, tetrafluoropyridyl, pyrimidyl, triazinyl and combinations thereof,
V and W are, at each occurrence identically or differently, selected from CRᵥR_{w}, NR_{y}, O, S or Se;
Ar is, at each occurrence identically or differently, selected from substituted or unsubstituted aryl having 6 to 30 carbon atoms or substituted or unsubstituted heteroaryl having 3 to 30 carbon atoms;
A, Rₐ, R_{b}, R_{c}, R_{d}, Rₑ, R_{f}, R_{g}, Rₕ, Rᵥ and R_{w} are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,
A is a group having at least one electron withdrawing group, and for any one of the structures, when one or more of Rₐ, R_{b}, R_{c}, R_{d}, Rₑ, R_{f}, R_{g}, Rₕ, Rᵥ and R_{w} are present, at least one of Rₐ, R_{b}, R_{c}, R_{d}, Rₑ, R_{f}, R_{g}, Rₕ, Rᵥ and R_{w} is a group having at least one electron withdrawing group; preferably, the group having at least one electron withdrawing group is selected from the group consisting of: F, CF₃, OCF₃, SF₅, SO₂CF₃, a cyano group, an isocyano group, SCN, OCN, pentafluorophenyl, 4-cyanotetrafluorophenyl, tetrafluoropyridyl, pyrimidyl, triazinyl and combinations thereof;
preferably, X and Y are, at each occurrence identically or differently, selected from the group consisting of the following structures:
O, S, Se, 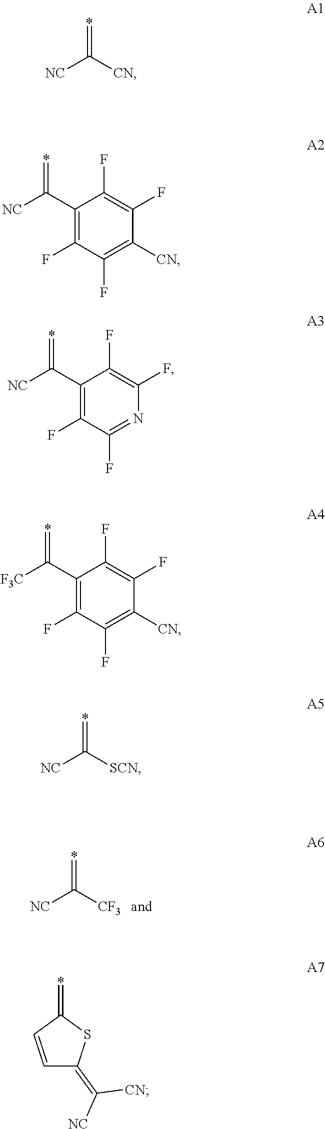
wherein “*” represents a position where X or Y is joined to a dehydrobenzodioxazole ring, a dehydrobenzodithiazole ring or a dehydrobenzodiselenazole ring in Formula 1.

7. The organic electroluminescent device of claim 1, wherein R is, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, unsubstituted alkyl having 1 to 20 carbon atoms, unsubstituted cycloalkyl having 3 to 20 ring carbon atoms, unsubstituted alkoxy having 1 to 20 carbon atoms, unsubstituted alkenyl having 2 to 20 carbon atoms, unsubstituted aryl having 6 to 30 carbon atoms, unsubstituted heteroaryl having 3 to 30 carbon atoms and any one of the following groups substituted by one or more of halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group and a phosphoroso group: alkyl having 1 to 20 carbon atoms, cycloalkyl having 3 to 20 ring carbon atoms, alkoxy having 1 to 20 carbon atoms, alkenyl having 2 to 20 carbon atoms, aryl having 6 to 30 carbon atoms, heteroaryl having 3 to 30 carbon atoms and combinations thereof;
preferably, R is, at each occurrence identically or differently, selected from the group consisting of: hydrogen, deuterium, methyl, isopropyl, NO₂, SO₂CH₃, SCF₃, C₂F₅, OC₂F₅, OCH₃, diphenylmethylsilyl, phenyl, methoxyphenyl, p-methylphenyl, 2,6-diisopropylphenyl, biphenyl, polyfluorophenyl, difluoropyridyl, nitrophenyl, dimethylthiazolyl, vinyl substituted by one or more of CN or CF₃, acetenyl substituted by one of CN or CF₃, dimethylphosphoroso, diphenylphosphoroso, F, CF₃, OCF₃, SF₅, SO₂CF₃, cyano, isocyano, SCN, OCN, trifluoromethylphenyl, trifluoromethoxyphenyl, bis(trifluoromethyl)phenyl, bis(trifluoromethoxy)phenyl, 4-cyanotetrafluorophenyl, phenyl or biphenyl substituted by one or more of F, CN or CF₃, tetrafluoropyridyl, pyrimidyl, triazinyl, diphenylboryl, oxaboraanthryl and combinations thereof.

8. The organic electroluminescent device of claim 7, wherein X and Y each are

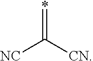 [SMILES: *=C(C#N)C#N]

9. The organic electroluminescent device of claim 1, wherein R is, at each occurrence identically or differently, selected from the group consisting of the following structures: 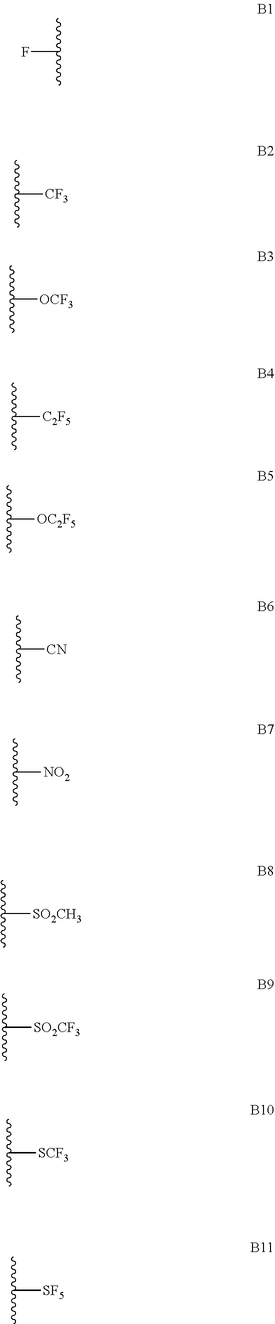 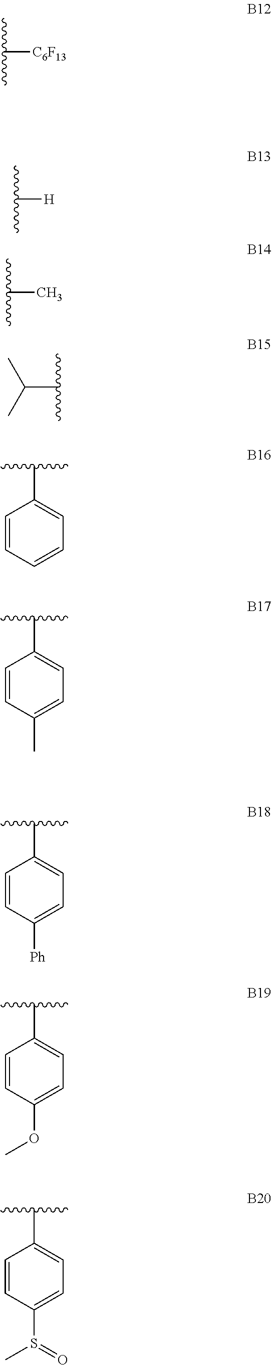 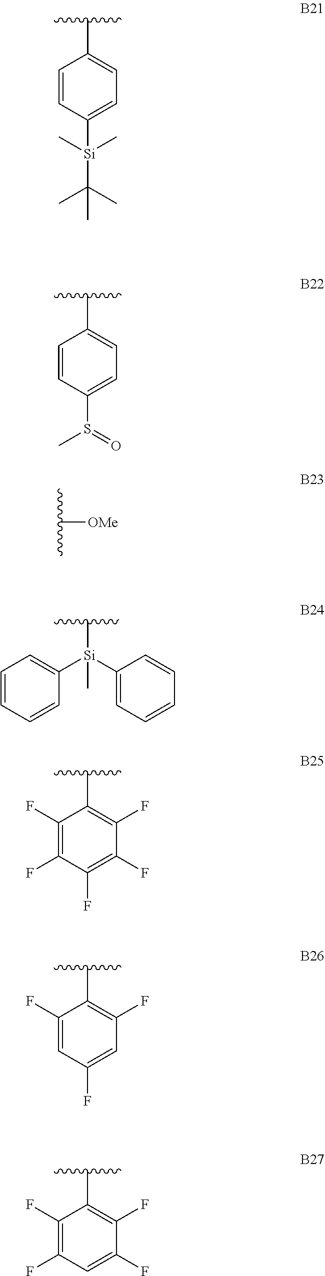 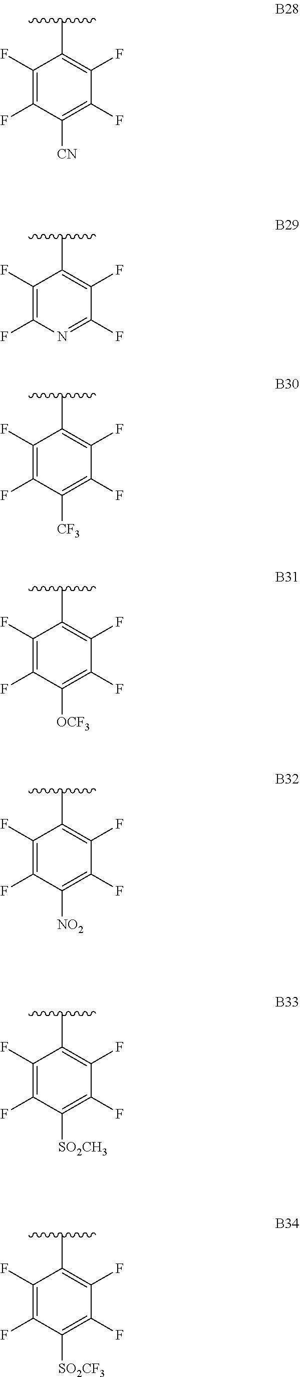 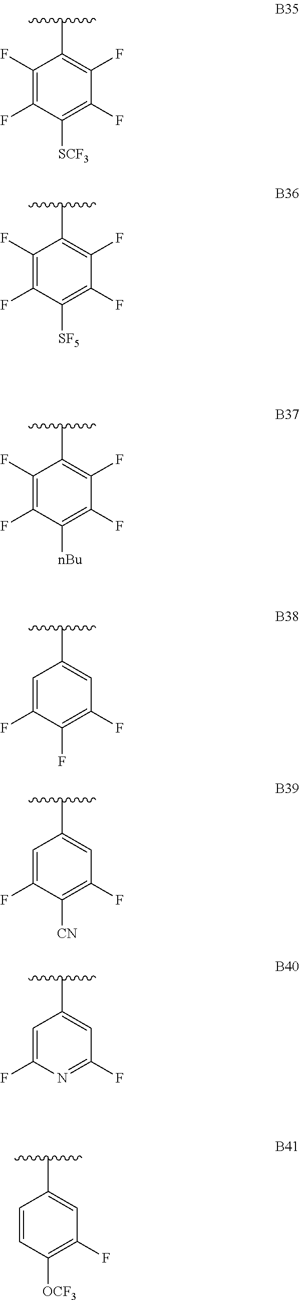 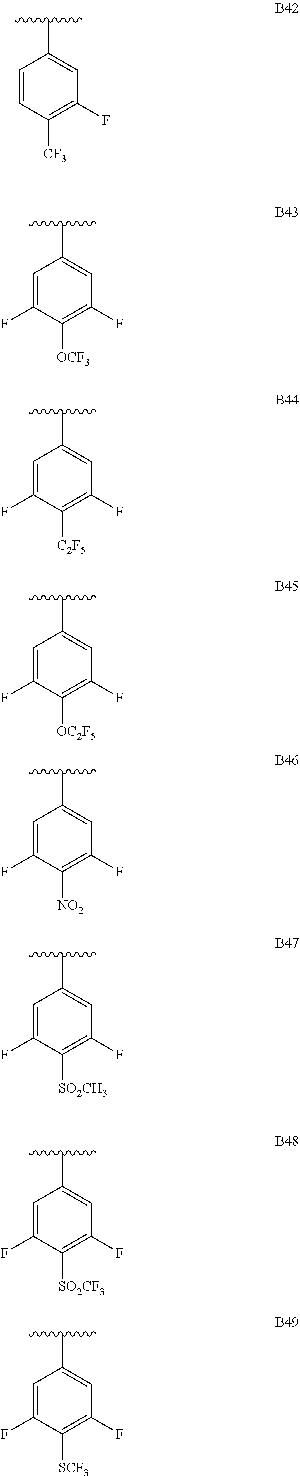 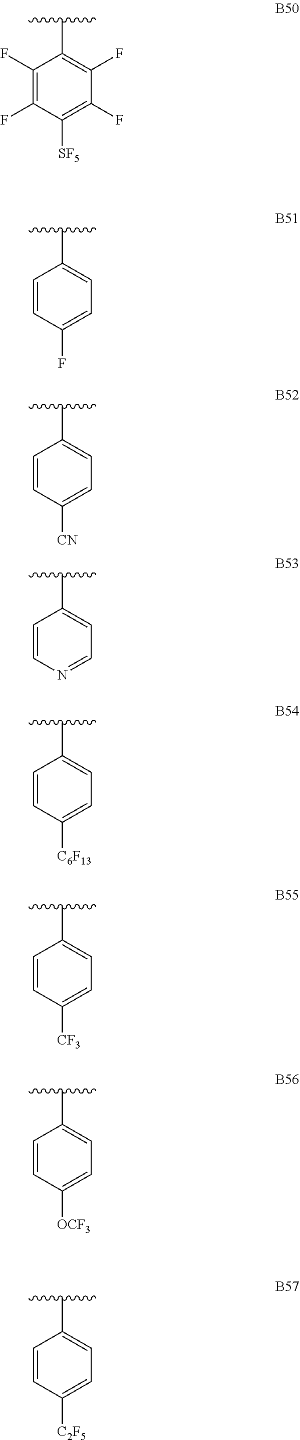 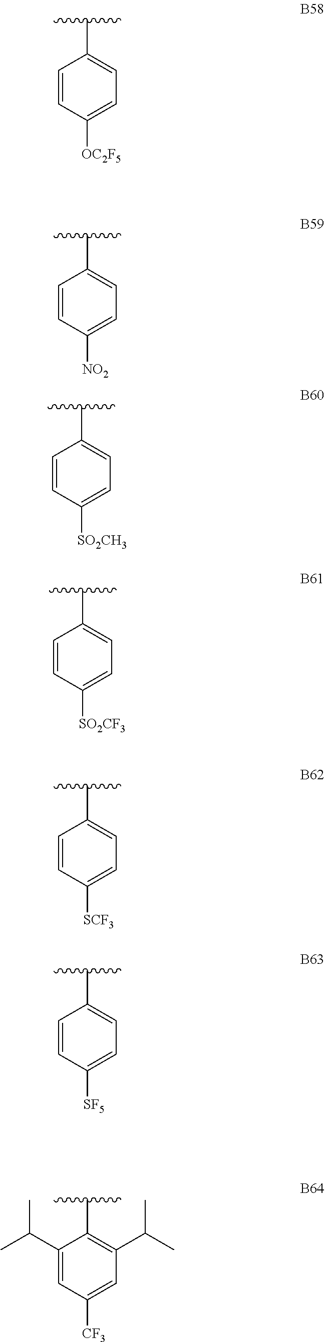 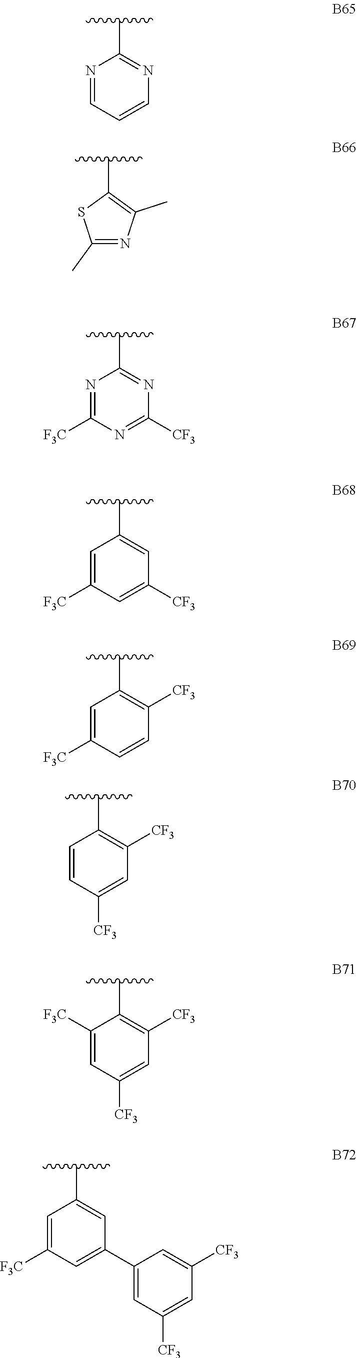 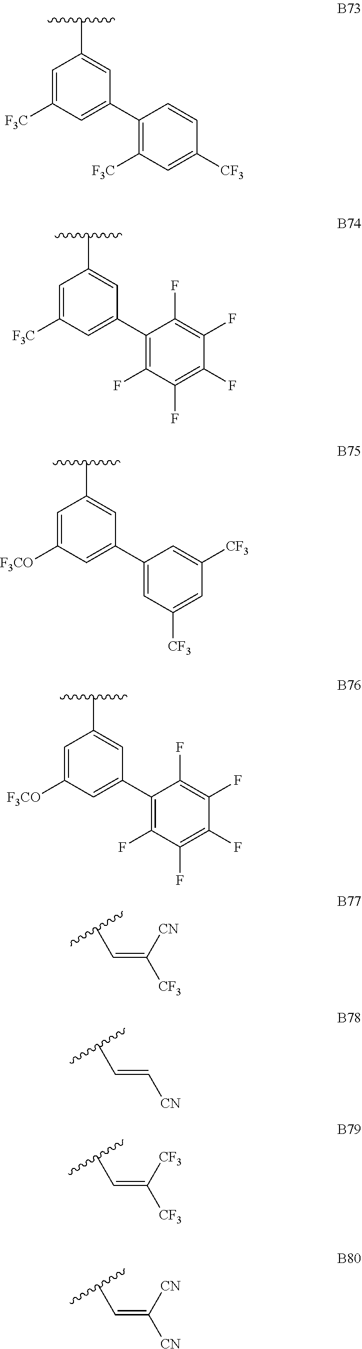 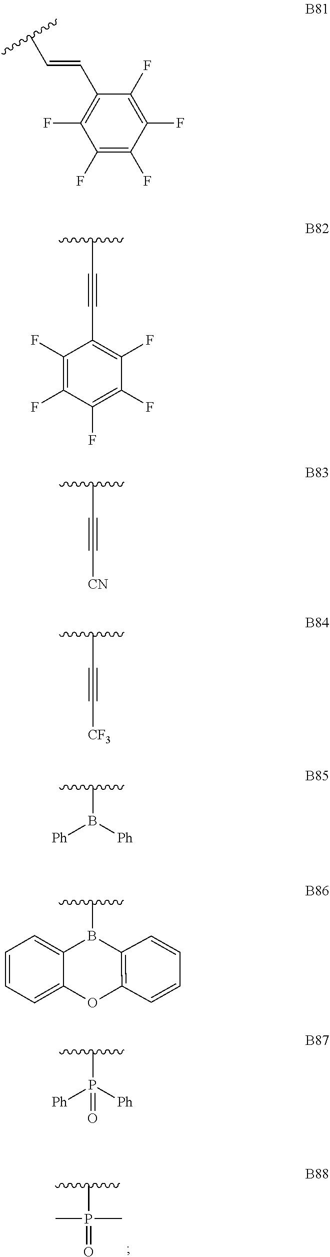
preferably, two R in a first compound represented by Formula 1 are the same;
“ [symbol] ” represents a position where the group R is joined to a dehydrobenzodioxazole ring, a dehydrobenzodithiazole ring or a dehydrobenzodiselenazole ring in Formula 1.

10. The organic electroluminescent device of claim 9, wherein the first compound is selected from the group consisting of Compound 1 to Compound 1356; wherein Compound 1 to Compound 1356 have a structure represented by Formula 1-1: 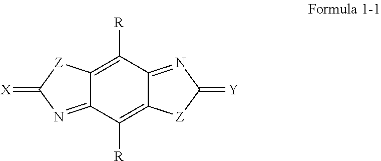
wherein two Z are the same, and Z, X, Y and R correspond to atoms or groups listed in the following table, respectively:

| Compound No. | Z | X | Y | R | R | Compound No. | Z | X | Y | R | R |
|---|---|---|---|---|---|---|---|---|---|---|---|
| 1 | O | A1 | A1 | B1 | B1 | 2 | O | A1 | A1 | B2 | B2 |
| 3 | O | A1 | A1 | B3 | B3 | 4 | O | A1 | A1 | B4 | B4 |
| 5 | O | A1 | A1 | B5 | B5 | 6 | O | A1 | A1 | B6 | B6 |
| 7 | O | A1 | A1 | B7 | B7 | 8 | O | A1 | A1 | B8 | B8 |
| 9 | O | A1 | A1 | B9 | B9 | 10 | O | A1 | A1 | B10 | B10 |
| 11 | O | A1 | A1 | B11 | B11 | 12 | O | A1 | A1 | B12 | B12 |
| 13 | O | A1 | A1 | B13 | B13 | 14 | O | A1 | A1 | B14 | B14 |
| 15 | O | A1 | A1 | B15 | B15 | 16 | O | A1 | A1 | B16 | B16 |
| 17 | O | A1 | A1 | B17 | B17 | 18 | O | A1 | A1 | B18 | B18 |
| 19 | O | A1 | A1 | B19 | B19 | 20 | O | A1 | A1 | B20 | B20 |
| 21 | O | A1 | A1 | B21 | B21 | 22 | O | A1 | A1 | B22 | B22 |
| 23 | O | A1 | A1 | B23 | B23 | 24 | O | A1 | A1 | B24 | B24 |
| 25 | O | A1 | A1 | B25 | B25 | 26 | O | A1 | A1 | B26 | B26 |
| 27 | O | A1 | A1 | B27 | B27 | 28 | O | A1 | A1 | B28 | B28 |
| 29 | O | A1 | A1 | B29 | B29 | 30 | O | A1 | A1 | B30 | B30 |
| 31 | O | A1 | A1 | B31 | B31 | 32 | O | A1 | A1 | B32 | B32 |
| 33 | O | A1 | A1 | B33 | B33 | 34 | O | A1 | A1 | B34 | B34 |
| 35 | O | A1 | A1 | B35 | B35 | 36 | O | A1 | A1 | B36 | B36 |
| 37 | O | A1 | A1 | B37 | B37 | 38 | O | A1 | A1 | B38 | B38 |
| 39 | O | A1 | A1 | B39 | B39 | 40 | O | A1 | A1 | B40 | B40 |
| 41 | O | A1 | A1 | B41 | B41 | 42 | O | A1 | A1 | B42 | B42 |
| 43 | O | A1 | A1 | B43 | B43 | 44 | O | A1 | A1 | B44 | B44 |
| 45 | O | A1 | A1 | B45 | B45 | 46 | O | A1 | A1 | B46 | B46 |
| 47 | O | A1 | A1 | B47 | B47 | 48 | O | A1 | A1 | B48 | B48 |
| 49 | O | A1 | A1 | B49 | B49 | 50 | O | A1 | A1 | B50 | B50 |
| 51 | O | A1 | A1 | B51 | B51 | 52 | O | A1 | A1 | B52 | B52 |
| 53 | O | A1 | A1 | B53 | B53 | 54 | O | A1 | A1 | B54 | B54 |
| 55 | O | A1 | A1 | B55 | B55 | 56 | O | A1 | A1 | B56 | B56 |
| 57 | O | A1 | A1 | B57 | B57 | 58 | O | A1 | A1 | B58 | B58 |
| 59 | O | A1 | A1 | B59 | B59 | 60 | O | A1 | A1 | B60 | B60 |
| 61 | O | A1 | A1 | B61 | B61 | 62 | O | A1 | A1 | B62 | B62 |
| 63 | O | A1 | A1 | B63 | B63 | 64 | O | A1 | A1 | B64 | B64 |
| 65 | O | A1 | A1 | B65 | B65 | 66 | O | A1 | A1 | B66 | B66 |
| 67 | O | A1 | A1 | B67 | B67 | 68 | O | A1 | A1 | B68 | B68 |
| 69 | O | A1 | A1 | B69 | B69 | 70 | O | A1 | A1 | B70 | B70 |
| 71 | O | A1 | A1 | B71 | B71 | 72 | O | A1 | A1 | B72 | B72 |
| 73 | O | A1 | A1 | B73 | B73 | 74 | O | A1 | A1 | B74 | B74 |
| 75 | O | A1 | A1 | B75 | B75 | 76 | O | A1 | A1 | B76 | B76 |
| 77 | O | A1 | A1 | B77 | B77 | 78 | O | A1 | A1 | B78 | B78 |
| 79 | O | A1 | A1 | B79 | B79 | 80 | O | A1 | A1 | B80 | B80 |
| 81 | O | A1 | A1 | B81 | B81 | 82 | O | A1 | A1 | B82 | B82 |
| 83 | O | A1 | A1 | B83 | B83 | 84 | O | A1 | A1 | B84 | B84 |
| 85 | O | A1 | A1 | B85 | B85 | 86 | O | A1 | A1 | B86 | B86 |
| 87 | O | A1 | A1 | B87 | B87 | 88 | O | A1 | A1 | B88 | B88 |
| 89 | S | A1 | A1 | B1 | B1 | 90 | S | A1 | A1 | B2 | B2 |
| 91 | S | A1 | A1 | B3 | B3 | 92 | S | A1 | A1 | B4 | B4 |
| 93 | S | A1 | A1 | B5 | B5 | 94 | S | A1 | A1 | B6 | B6 |
| 95 | S | A1 | A1 | B7 | B7 | 96 | S | A1 | A1 | B8 | B8 |
| 97 | S | A1 | A1 | B9 | B9 | 98 | S | A1 | A1 | B10 | B10 |
| 99 | S | A1 | A1 | B11 | B11 | 100 | S | A1 | A1 | B12 | B12 |
| 101 | S | A1 | A1 | B13 | B13 | 102 | S | A1 | A1 | B14 | B14 |
| 103 | S | A1 | A1 | B15 | B15 | 104 | S | A1 | A1 | B16 | B16 |
| 105 | S | A1 | A1 | B17 | B17 | 106 | S | A1 | A1 | B18 | B18 |
| 107 | S | A1 | A1 | B19 | B19 | 108 | S | A1 | A1 | B20 | B20 |
| 109 | S | A1 | A1 | B21 | B21 | 110 | S | A1 | A1 | B22 | B22 |
| 111 | S | A1 | A1 | B23 | B23 | 112 | S | A1 | A1 | B24 | B24 |
| 113 | S | A1 | A1 | B25 | B25 | 114 | S | A1 | A1 | B26 | B26 |
| 115 | S | A1 | A1 | B27 | B27 | 116 | S | A1 | A1 | B28 | B28 |
| 117 | S | A1 | A1 | B29 | B29 | 118 | S | A1 | A1 | B30 | B30 |
| 119 | S | A1 | A1 | B31 | B31 | 120 | S | A1 | A1 | B32 | B32 |
| 121 | S | A1 | A1 | B33 | B33 | 122 | S | A1 | A1 | B34 | B34 |
| 123 | S | A1 | A1 | B35 | B35 | 124 | S | A1 | A1 | B36 | B36 |
| 125 | S | A1 | A1 | B37 | B37 | 126 | S | A1 | A1 | B38 | B38 |
| 127 | S | A1 | A1 | B39 | B39 | 128 | S | A1 | A1 | B40 | B40 |
| 129 | S | A1 | A1 | B41 | B41 | 130 | S | A1 | A1 | B42 | B42 |
| 131 | S | A1 | A1 | B43 | B43 | 132 | S | A1 | A1 | B44 | B44 |
| 133 | S | A1 | A1 | B45 | B45 | 134 | S | A1 | A1 | B46 | B46 |
| 135 | S | A1 | A1 | B47 | B47 | 136 | S | A1 | A1 | B48 | B48 |
| 137 | S | A1 | A1 | B49 | B49 | 138 | S | A1 | A1 | B50 | B50 |
| 139 | S | A1 | A1 | B51 | B51 | 140 | S | A1 | A1 | B52 | B52 |
| 141 | S | A1 | A1 | B53 | B53 | 142 | S | A1 | A1 | B54 | B54 |
| 143 | S | A1 | A1 | B55 | B55 | 144 | S | A1 | A1 | B56 | B56 |
| 145 | S | A1 | A1 | B57 | B57 | 146 | S | A1 | A1 | B58 | B58 |
| 147 | S | A1 | A1 | B59 | B59 | 148 | S | A1 | A1 | B60 | B60 |
| 149 | S | A1 | A1 | B61 | B61 | 150 | S | A1 | A1 | B62 | B62 |
| 151 | S | A1 | A1 | B63 | B63 | 152 | S | A1 | A1 | B64 | B64 |
| 153 | S | A1 | A1 | B65 | B65 | 154 | S | A1 | A1 | B66 | B66 |
| 155 | S | A1 | A1 | B67 | B67 | 156 | S | A1 | A1 | B68 | B68 |
| 157 | S | A1 | A1 | B69 | B69 | 158 | S | A1 | A1 | B70 | B70 |
| 159 | S | A1 | A1 | B71 | B71 | 160 | S | A1 | A1 | B72 | B72 |
| 161 | S | A1 | A1 | B73 | B73 | 162 | S | A1 | A1 | B74 | B74 |
| 163 | S | A1 | A1 | B75 | B75 | 164 | S | A1 | A1 | B76 | B76 |
| 165 | S | A1 | A1 | B77 | B77 | 166 | S | A1 | A1 | B78 | B78 |
| 167 | S | A1 | A1 | B79 | B79 | 168 | S | A1 | A1 | B80 | B80 |
| 169 | S | A1 | A1 | B81 | B81 | 170 | S | A1 | A1 | B82 | B82 |
| 171 | S | A1 | A1 | B83 | B83 | 172 | S | A1 | A1 | B84 | B84 |
| 173 | S | A1 | A1 | B85 | B85 | 174 | S | A1 | A1 | B86 | B86 |
| 175 | S | A1 | A1 | B87 | B87 | 176 | S | A1 | A1 | B88 | B88 |
| 177 | Se | A1 | A1 | B1 | B1 | 178 | Se | A1 | A1 | B2 | B2 |
| 179 | Se | A1 | A1 | B3 | B3 | 180 | Se | A1 | A1 | B4 | B4 |
| 181 | Se | A1 | A1 | B5 | B5 | 182 | Se | A1 | A1 | B6 | B6 |
| 183 | Se | A1 | A1 | B7 | B7 | 184 | Se | A1 | A1 | B8 | B8 |
| 185 | Se | A1 | A1 | B9 | B9 | 186 | Se | A1 | A1 | B10 | B10 |
| 187 | Se | A1 | A1 | B11 | B11 | 188 | Se | A1 | A1 | B12 | B12 |
| 189 | Se | A1 | A1 | B13 | B13 | 190 | Se | A1 | A1 | B14 | B14 |
| 191 | Se | A1 | A1 | B15 | B15 | 192 | Se | A1 | A1 | B16 | B16 |
| 193 | Se | A1 | A1 | B17 | B17 | 194 | Se | A1 | A1 | B18 | B18 |
| 195 | Se | A1 | A1 | B19 | B19 | 196 | Se | A1 | A1 | B20 | B20 |
| 197 | Se | A1 | A1 | B21 | B21 | 198 | Se | A1 | A1 | B22 | B22 |
| 199 | Se | A1 | A1 | B23 | B23 | 200 | Se | A1 | A1 | B24 | B24 |
| 201 | Se | A1 | A1 | B25 | B25 | 202 | Se | A1 | A1 | B26 | B26 |
| 203 | Se | A1 | A1 | B27 | B27 | 204 | Se | A1 | A1 | B28 | B28 |
| 205 | Se | A1 | A1 | B29 | B29 | 206 | Se | A1 | A1 | B30 | B30 |
| 207 | Se | A1 | A1 | B31 | B31 | 208 | Se | A1 | A1 | B32 | B32 |
| 209 | Se | A1 | A1 | B33 | B33 | 210 | Se | A1 | A1 | B34 | B34 |
| 211 | Se | A1 | A1 | B35 | B35 | 212 | Se | A1 | A1 | B36 | B36 |
| 213 | Se | A1 | A1 | B37 | B37 | 214 | Se | A1 | A1 | B38 | B38 |
| 215 | Se | A1 | A1 | B39 | B39 | 216 | Se | A1 | A1 | B40 | B40 |
| 217 | Se | A1 | A1 | B41 | B41 | 218 | Se | A1 | A1 | B42 | B42 |
| 219 | Se | A1 | A1 | B43 | B43 | 220 | Se | A1 | A1 | B44 | B44 |
| 221 | Se | A1 | A1 | B45 | B45 | 222 | Se | A1 | A1 | B46 | B46 |
| 223 | Se | A1 | A1 | B47 | B47 | 224 | Se | A1 | A1 | B48 | B48 |
| 225 | Se | A1 | A1 | B49 | B49 | 226 | Se | A1 | A1 | B50 | B50 |
| 227 | Se | A1 | A1 | B51 | B51 | 228 | Se | A1 | A1 | B52 | B52 |
| 229 | Se | A1 | A1 | B53 | B53 | 230 | Se | A1 | A1 | B54 | B54 |
| 231 | Se | A1 | A1 | B55 | B55 | 232 | Se | A1 | A1 | B56 | B56 |
| 233 | Se | A1 | A1 | B57 | B57 | 234 | Se | A1 | A1 | B58 | B58 |
| 235 | Se | A1 | A1 | B59 | B59 | 236 | Se | A1 | A1 | B60 | B60 |
| 237 | Se | A1 | A1 | B61 | B61 | 238 | Se | A1 | A1 | B62 | B62 |
| 239 | Se | A1 | A1 | B63 | B63 | 240 | Se | A1 | A1 | B64 | B64 |
| 241 | Se | A1 | A1 | B65 | B65 | 242 | Se | A1 | A1 | B66 | B66 |
| 243 | Se | A1 | A1 | B67 | B67 | 244 | Se | A1 | A1 | B68 | B68 |
| 245 | Se | A1 | A1 | B69 | B69 | 246 | Se | A1 | A1 | B70 | B70 |
| 247 | Se | A1 | A1 | B71 | B71 | 248 | Se | A1 | A1 | B72 | B72 |
| 249 | Se | A1 | A1 | B73 | B73 | 250 | Se | A1 | A1 | B74 | B74 |
| 251 | Se | A1 | A1 | B75 | B75 | 252 | Se | A1 | A1 | B76 | B76 |
| 253 | Se | A1 | A1 | B77 | B77 | 254 | Se | A1 | A1 | B78 | B78 |
| 255 | Se | A1 | A1 | B79 | B79 | 256 | Se | A1 | A1 | B80 | B80 |
| 257 | Se | A1 | A1 | B81 | B81 | 258 | Se | A1 | A1 | B82 | B82 |
| 259 | Se | A1 | A1 | B83 | B83 | 260 | Se | A1 | A1 | B84 | B84 |
| 261 | Se | A1 | A1 | B85 | B85 | 262 | Se | A1 | A1 | B86 | B86 |
| 263 | Se | A1 | A1 | B87 | B87 | 264 | Se | A1 | A1 | B88 | B88 |
| 265 | O | A2 | A2 | B1 | B1 | 266 | O | A2 | A2 | B6 | B6 |
| 267 | O | A2 | A2 | B10 | B10 | 268 | O | A2 | A2 | B16 | B16 |
| 269 | O | A2 | A2 | B25 | B25 | 270 | O | A2 | A2 | B28 | B28 |
| 271 | O | A2 | A2 | B29 | B29 | 272 | O | A2 | A2 | B30 | B30 |
| 273 | O | A2 | A2 | B38 | B38 | 274 | O | A2 | A2 | B39 | B39 |
| 275 | O | A2 | A2 | B40 | B40 | 276 | O | A2 | A2 | B41 | B41 |
| 277 | O | A2 | A2 | B43 | B43 | 278 | O | A2 | A2 | B52 | B52 |
| 279 | O | A2 | A2 | B56 | B56 | 280 | O | A2 | A2 | B67 | B67 |
| 281 | O | A2 | A2 | B68 | B68 | 282 | O | A2 | A2 | B69 | B69 |
| 283 | O | A2 | A2 | B70 | B70 | 284 | O | A2 | A2 | B71 | B71 |
| 285 | O | A2 | A2 | B72 | B72 | 286 | O | A2 | A2 | B74 | B74 |
| 287 | O | A2 | A2 | B79 | B79 | 288 | O | A2 | A2 | B80 | B80 |
| 289 | O | A2 | A2 | B82 | B82 | 290 | O | A2 | A2 | B83 | B83 |
| 291 | O | A2 | A2 | B86 | B86 | 292 | O | A2 | A2 | B88 | B88 |
| 293 | S | A2 | A2 | B1 | B1 | 294 | S | A2 | A2 | B6 | B6 |
| 295 | S | A2 | A2 | B10 | B10 | 296 | S | A2 | A2 | B16 | B16 |
| 297 | S | A2 | A2 | B25 | B25 | 298 | S | A2 | A2 | B28 | B28 |
| 299 | S | A2 | A2 | B29 | B29 | 300 | S | A2 | A2 | B30 | B30 |
| 301 | S | A2 | A2 | B38 | B38 | 302 | S | A2 | A2 | B39 | B39 |
| 303 | S | A2 | A2 | B40 | B40 | 304 | S | A2 | A2 | B41 | B41 |
| 305 | S | A2 | A2 | B43 | B43 | 306 | S | A2 | A2 | B52 | B52 |
| 307 | S | A2 | A2 | B56 | B56 | 308 | S | A2 | A2 | B67 | B67 |
| 309 | S | A2 | A2 | B68 | B68 | 310 | S | A2 | A2 | B69 | B69 |
| 311 | S | A2 | A2 | B70 | B70 | 312 | S | A2 | A2 | B71 | B71 |
| 313 | S | A2 | A2 | B72 | B72 | 314 | S | A2 | A2 | B74 | B74 |
| 315 | S | A2 | A2 | B79 | B79 | 316 | S | A2 | A2 | B80 | B80 |
| 317 | S | A2 | A2 | B82 | B82 | 318 | S | A2 | A2 | B83 | B83 |
| 319 | S | A2 | A2 | B86 | B86 | 320 | S | A2 | A2 | B88 | B88 |
| 321 | Se | A2 | A2 | B1 | B1 | 322 | Se | A2 | A2 | B6 | B6 |
| 323 | Se | A2 | A2 | B10 | B10 | 324 | Se | A2 | A2 | B16 | B16 |
| 325 | Se | A2 | A2 | B25 | B25 | 326 | Se | A2 | A2 | B28 | B28 |
| 327 | Se | A2 | A2 | B29 | B29 | 328 | Se | A2 | A2 | B30 | B30 |
| 329 | Se | A2 | A2 | B38 | B38 | 330 | Se | A2 | A2 | B39 | B39 |
| 331 | Se | A2 | A2 | B40 | B40 | 332 | Se | A2 | A2 | B41 | B41 |
| 333 | Se | A2 | A2 | B43 | B43 | 334 | Se | A2 | A2 | B52 | B52 |
| 335 | Se | A2 | A2 | B56 | B56 | 336 | Se | A2 | A2 | B67 | B67 |
| 337 | Se | A2 | A2 | B68 | B68 | 338 | Se | A2 | A2 | B69 | B69 |
| 339 | Se | A2 | A2 | B70 | B70 | 340 | Se | A2 | A2 | B71 | B71 |
| 341 | Se | A2 | A2 | B72 | B72 | 342 | Se | A2 | A2 | B74 | B74 |
| 343 | Se | A2 | A2 | B79 | B79 | 344 | Se | A2 | A2 | B80 | B80 |
| 345 | Se | A2 | A2 | B82 | B82 | 346 | Se | A2 | A2 | B83 | B83 |
| 347 | Se | A2 | A2 | B86 | B86 | 348 | Se | A2 | A2 | B88 | B88 |
| 349 | O | A3 | A3 | B1 | B1 | 350 | O | A3 | A3 | B6 | B6 |
| 351 | O | A3 | A3 | B10 | B10 | 352 | O | A3 | A3 | B16 | B16 |
| 353 | O | A3 | A3 | B25 | B25 | 354 | O | A3 | A3 | B28 | B28 |
| 355 | O | A3 | A3 | B29 | B29 | 356 | O | A3 | A3 | B30 | B30 |
| 357 | O | A3 | A3 | B38 | B38 | 358 | O | A3 | A3 | B39 | B39 |
| 359 | O | A3 | A3 | B40 | B40 | 360 | O | A3 | A3 | B41 | B41 |
| 361 | O | A3 | A3 | B43 | B43 | 362 | O | A3 | A3 | B52 | B52 |
| 363 | O | A3 | A3 | B56 | B56 | 364 | O | A3 | A3 | B67 | B67 |
| 365 | O | A3 | A3 | B68 | B68 | 366 | O | A3 | A3 | B69 | B69 |
| 367 | O | A3 | A3 | B70 | B70 | 368 | O | A3 | A3 | B71 | B71 |
| 369 | O | A3 | A3 | B72 | B72 | 370 | O | A3 | A3 | B74 | B74 |
| 371 | O | A3 | A3 | B79 | B79 | 372 | O | A3 | A3 | B80 | B80 |
| 373 | O | A3 | A3 | B82 | B82 | 374 | O | A3 | A3 | B83 | B83 |
| 375 | O | A3 | A3 | B86 | B86 | 376 | O | A3 | A3 | B88 | B88 |
| 377 | S | A3 | A3 | B1 | B1 | 378 | S | A3 | A3 | B6 | B6 |
| 379 | S | A3 | A3 | B10 | B10 | 380 | S | A3 | A3 | B16 | B16 |
| 381 | S | A3 | A3 | B25 | B25 | 382 | S | A3 | A3 | B28 | B28 |
| 383 | S | A3 | A3 | B29 | B29 | 384 | S | A3 | A3 | B30 | B30 |
| 385 | S | A3 | A3 | B38 | B38 | 386 | S | A3 | A3 | B39 | B39 |
| 387 | S | A3 | A3 | B40 | B40 | 388 | S | A3 | A3 | B41 | B41 |
| 389 | S | A3 | A3 | B43 | B43 | 390 | S | A3 | A3 | B52 | B52 |
| 391 | S | A3 | A3 | B56 | B56 | 392 | S | A3 | A3 | B67 | B67 |
| 393 | S | A3 | A3 | B68 | B68 | 394 | S | A3 | A3 | B69 | B69 |
| 395 | S | A3 | A3 | B70 | B70 | 396 | S | A3 | A3 | B71 | B71 |
| 397 | S | A3 | A3 | B72 | B72 | 398 | S | A3 | A3 | B74 | B74 |
| 399 | S | A3 | A3 | B79 | B79 | 400 | S | A3 | A3 | B80 | B80 |
| 401 | S | A3 | A3 | B82 | B82 | 402 | S | A3 | A3 | B83 | B83 |
| 403 | S | A3 | A3 | B86 | B86 | 404 | S | A3 | A3 | B88 | B88 |
| 405 | Se | A3 | A3 | B1 | B1 | 406 | Se | A3 | A3 | B6 | B6 |
| 407 | Se | A3 | A3 | B10 | B10 | 408 | Se | A3 | A3 | B16 | B16 |
| 409 | Se | A3 | A3 | B25 | B25 | 410 | Se | A3 | A3 | B28 | B28 |
| 411 | Se | A3 | A3 | B29 | B29 | 412 | Se | A3 | A3 | B30 | B30 |
| 413 | Se | A3 | A3 | B38 | B38 | 414 | Se | A3 | A3 | B39 | B39 |
| 415 | Se | A3 | A3 | B40 | B40 | 416 | Se | A3 | A3 | B41 | B41 |
| 417 | Se | A3 | A3 | B43 | B43 | 418 | Se | A3 | A3 | B52 | B52 |
| 419 | Se | A3 | A3 | B56 | B56 | 420 | Se | A3 | A3 | B67 | B67 |
| 421 | Se | A3 | A3 | B68 | B68 | 422 | Se | A3 | A3 | B69 | B69 |
| 423 | Se | A3 | A3 | B70 | B70 | 424 | Se | A3 | A3 | B71 | B71 |
| 425 | Se | A3 | A3 | B72 | B72 | 426 | Se | A3 | A3 | B74 | B74 |
| 427 | Se | A3 | A3 | B79 | B79 | 428 | Se | A3 | A3 | B80 | B80 |
| 429 | Se | A3 | A3 | B82 | B82 | 430 | Se | A3 | A3 | B83 | B83 |
| 431 | Se | A3 | A3 | B86 | B86 | 432 | Se | A3 | A3 | B88 | B88 |
| 433 | O | A4 | A4 | B1 | B1 | 434 | O | A4 | A4 | B6 | B6 |
| 435 | O | A4 | A4 | B10 | B10 | 436 | O | A4 | A4 | B16 | B16 |
| 437 | O | A4 | A4 | B25 | B25 | 438 | O | A4 | A4 | B28 | B28 |
| 439 | O | A4 | A4 | B29 | B29 | 440 | O | A4 | A4 | B30 | B30 |
| 441 | O | A4 | A4 | B38 | B38 | 442 | O | A4 | A4 | B39 | B39 |
| 443 | O | A4 | A4 | B40 | B40 | 444 | O | A4 | A4 | B41 | B41 |
| 445 | O | A4 | A4 | B43 | B43 | 446 | O | A4 | A4 | B52 | B52 |
| 447 | O | A4 | A4 | B56 | B56 | 448 | O | A4 | A4 | B67 | B67 |
| 449 | O | A4 | A4 | B68 | B68 | 450 | O | A4 | A4 | B69 | B69 |
| 451 | O | A4 | A4 | B70 | B70 | 452 | O | A4 | A4 | B71 | B71 |
| 453 | O | A4 | A4 | B72 | B72 | 454 | O | A4 | A4 | B74 | B74 |
| 455 | O | A4 | A4 | B79 | B79 | 456 | O | A4 | A4 | B80 | B80 |
| 457 | O | A4 | A4 | B82 | B82 | 458 | O | A4 | A4 | B83 | B83 |
| 459 | O | A4 | A4 | B86 | B86 | 460 | O | A4 | A4 | B88 | B88 |
| 461 | S | A4 | A4 | B1 | B1 | 462 | S | A4 | A4 | B6 | B6 |
| 463 | S | A4 | A4 | B10 | B10 | 464 | S | A4 | A4 | B16 | B16 |
| 465 | S | A4 | A4 | B25 | B25 | 466 | S | A4 | A4 | B28 | B28 |
| 467 | S | A4 | A4 | B29 | B29 | 468 | S | A4 | A4 | B30 | B30 |
| 469 | S | A4 | A4 | B38 | B38 | 470 | S | A4 | A4 | B39 | B39 |
| 471 | S | A4 | A4 | B40 | B40 | 472 | S | A4 | A4 | B41 | B41 |
| 473 | S | A4 | A4 | B43 | B43 | 474 | S | A4 | A4 | B52 | B52 |
| 475 | S | A4 | A4 | B56 | B56 | 476 | S | A4 | A4 | B67 | B67 |
| 477 | S | A4 | A4 | B68 | B68 | 478 | S | A4 | A4 | B69 | B69 |
| 479 | S | A4 | A4 | B70 | B70 | 480 | S | A4 | A4 | B71 | B71 |
| 481 | S | A4 | A4 | B72 | B72 | 482 | S | A4 | A4 | B74 | B74 |
| 483 | S | A4 | A4 | B79 | B79 | 484 | S | A4 | A4 | B80 | B80 |
| 485 | S | A4 | A4 | B82 | B82 | 486 | S | A4 | A4 | B83 | B83 |
| 487 | S | A4 | A4 | B86 | B86 | 488 | S | A4 | A4 | B88 | B88 |
| 489 | Se | A4 | A4 | B1 | B1 | 490 | Se | A4 | A4 | B6 | B6 |
| 491 | Se | A4 | A4 | B10 | B10 | 492 | Se | A4 | A4 | B16 | B16 |
| 493 | Se | A4 | A4 | B25 | B25 | 494 | Se | A4 | A4 | B28 | B28 |
| 495 | Se | A4 | A4 | B29 | B29 | 496 | Se | A4 | A4 | B30 | B30 |
| 497 | Se | A4 | A4 | B38 | B38 | 498 | Se | A4 | A4 | B39 | B39 |
| 499 | Se | A4 | A4 | B40 | B40 | 500 | Se | A4 | A4 | B41 | B41 |
| 501 | Se | A4 | A4 | B43 | B43 | 502 | Se | A4 | A4 | B52 | B52 |
| 503 | Se | A4 | A4 | B56 | B56 | 504 | Se | A4 | A4 | B67 | B67 |
| 505 | Se | A4 | A4 | B68 | B68 | 506 | Se | A4 | A4 | B69 | B69 |
| 507 | Se | A4 | A4 | B70 | B70 | 508 | Se | A4 | A4 | B71 | B71 |
| 509 | Se | A4 | A4 | B72 | B72 | 510 | Se | A4 | A4 | B74 | B74 |
| 511 | Se | A4 | A4 | B79 | B79 | 512 | Se | A4 | A4 | B80 | B80 |
| 513 | Se | A4 | A4 | B82 | B82 | 514 | Se | A4 | A4 | B83 | B83 |
| 515 | Se | A4 | A4 | B86 | B86 | 516 | Se | A4 | A4 | B88 | B88 |
| 517 | O | A5 | A5 | B1 | B1 | 518 | O | A5 | A5 | B6 | B6 |
| 519 | O | A5 | A5 | B10 | B10 | 520 | O | A5 | A5 | B16 | B16 |
| 521 | O | A5 | A5 | B25 | B25 | 522 | O | A5 | A5 | B28 | B28 |
| 523 | O | A5 | A5 | B29 | B29 | 524 | O | A5 | A5 | B30 | B30 |
| 525 | O | A5 | A5 | B38 | B38 | 526 | O | A5 | A5 | B39 | B39 |
| 527 | O | A5 | A5 | B40 | B40 | 528 | O | A5 | A5 | B41 | B41 |
| 529 | O | A5 | A5 | B43 | B43 | 530 | O | A5 | A5 | B52 | B52 |
| 531 | O | A5 | A5 | B56 | B56 | 532 | O | A5 | A5 | B67 | B67 |
| 533 | O | A5 | A5 | B68 | B68 | 534 | O | A5 | A5 | B69 | B69 |
| 535 | O | A5 | A5 | B70 | B70 | 536 | O | A5 | A5 | B71 | B71 |
| 537 | O | A5 | A5 | B72 | B72 | 538 | O | A5 | A5 | B74 | B74 |
| 539 | O | A5 | A5 | B79 | B79 | 540 | O | A5 | A5 | B80 | B80 |
| 541 | O | A5 | A5 | B82 | B82 | 542 | O | A5 | A5 | B83 | B83 |
| 543 | O | A5 | A5 | B86 | B86 | 544 | O | A5 | A5 | B88 | B88 |
| 545 | S | A5 | A5 | B1 | B1 | 546 | S | A5 | A5 | B6 | B6 |
| 547 | S | A5 | A5 | B10 | B10 | 548 | S | A5 | A5 | B16 | B16 |
| 549 | S | A5 | A5 | B25 | B25 | 550 | S | A5 | A5 | B28 | B28 |
| 551 | S | A5 | A5 | B29 | B29 | 552 | S | A5 | A5 | B30 | B30 |
| 553 | S | A5 | A5 | B38 | B38 | 554 | S | A5 | A5 | B39 | B39 |
| 555 | S | A5 | A5 | B40 | B40 | 556 | S | A5 | A5 | B41 | B41 |
| 557 | S | A5 | A5 | B43 | B43 | 558 | S | A5 | A5 | B52 | B52 |
| 559 | S | A5 | A5 | B56 | B56 | 560 | S | A5 | A5 | B67 | B67 |
| 561 | S | A5 | A5 | B68 | B68 | 562 | S | A5 | A5 | B69 | B69 |
| 563 | S | A5 | A5 | B70 | B70 | 564 | S | A5 | A5 | B71 | B71 |
| 565 | S | A5 | A5 | B72 | B72 | 566 | S | A5 | A5 | B74 | B74 |
| 567 | S | A5 | A5 | B79 | B79 | 568 | S | A5 | A5 | B80 | B80 |
| 569 | S | A5 | A5 | B82 | B82 | 570 | S | A5 | A5 | B83 | B83 |
| 571 | S | A5 | A5 | B86 | B86 | 572 | S | A5 | A5 | B88 | B88 |
| 573 | Se | A5 | A5 | B1 | B1 | 574 | Se | A5 | A5 | B6 | B6 |
| 575 | Se | A5 | A5 | B10 | B10 | 576 | Se | A5 | A5 | B16 | B16 |
| 577 | Se | A5 | A5 | B25 | B25 | 578 | Se | A5 | A5 | B28 | B28 |
| 579 | Se | A5 | A5 | B29 | B29 | 580 | Se | A5 | A5 | B30 | B30 |
| 581 | Se | A5 | A5 | B38 | B38 | 582 | Se | A5 | A5 | B39 | B39 |
| 583 | Se | A5 | A5 | B40 | B40 | 584 | Se | A5 | A5 | B41 | B41 |
| 585 | Se | A5 | A5 | B43 | B43 | 586 | Se | A5 | A5 | B52 | B52 |
| 587 | Se | A5 | A5 | B56 | B56 | 588 | Se | A5 | A5 | B67 | B67 |
| 589 | Se | A5 | A5 | B68 | B68 | 590 | Se | A5 | A5 | B69 | B69 |
| 591 | Se | A5 | A5 | B70 | B70 | 592 | Se | A5 | A5 | B71 | B71 |
| 593 | Se | A5 | A5 | B72 | B72 | 594 | Se | A5 | A5 | B74 | B74 |
| 595 | Se | A5 | A5 | B79 | B79 | 596 | Se | A5 | A5 | B80 | B80 |
| 597 | Se | A5 | A5 | B82 | B82 | 598 | Se | A5 | A5 | B83 | B83 |
| 599 | Se | A5 | A5 | B86 | B86 | 600 | Se | A5 | A5 | B88 | B88 |
| 601 | O | A6 | A6 | B1 | B1 | 602 | O | A6 | A6 | B6 | B6 |
| 603 | O | A6 | A6 | B10 | B10 | 604 | O | A6 | A6 | B16 | B16 |
| 605 | O | A6 | A6 | B25 | B25 | 606 | O | A6 | A6 | B28 | B28 |
| 607 | O | A6 | A6 | B29 | B29 | 608 | O | A6 | A6 | B30 | B30 |
| 609 | O | A6 | A6 | B38 | B38 | 610 | O | A6 | A6 | B39 | B39 |
| 611 | O | A6 | A6 | B40 | B40 | 612 | O | A6 | A6 | B41 | B41 |
| 613 | O | A6 | A6 | B43 | B43 | 614 | O | A6 | A6 | B52 | B52 |
| 615 | O | A6 | A6 | B56 | B56 | 616 | O | A6 | A6 | B67 | B67 |
| 617 | O | A6 | A6 | B68 | B68 | 618 | O | A6 | A6 | B69 | B69 |
| 619 | O | A6 | A6 | B70 | B70 | 620 | O | A6 | A6 | B71 | B71 |
| 621 | O | A6 | A6 | B72 | B72 | 622 | O | A6 | A6 | B74 | B74 |
| 623 | O | A6 | A6 | B79 | B79 | 624 | O | A6 | A6 | B80 | B80 |
| 625 | O | A6 | A6 | B82 | B82 | 626 | O | A6 | A6 | B83 | B83 |
| 627 | O | A6 | A6 | B86 | B86 | 628 | O | A6 | A6 | B88 | B88 |
| 629 | S | A6 | A6 | B1 | B1 | 630 | S | A6 | A6 | B6 | B6 |
| 631 | S | A6 | A6 | B10 | B10 | 632 | S | A6 | A6 | B16 | B16 |
| 633 | S | A6 | A6 | B25 | B25 | 634 | S | A6 | A6 | B28 | B28 |
| 635 | S | A6 | A6 | B29 | B29 | 636 | S | A6 | A6 | B30 | B30 |
| 637 | S | A6 | A6 | B38 | B38 | 638 | S | A6 | A6 | B39 | B39 |
| 639 | S | A6 | A6 | B40 | B40 | 640 | S | A6 | A6 | B41 | B41 |
| 641 | S | A6 | A6 | B43 | B43 | 642 | S | A6 | A6 | B52 | B52 |
| 643 | S | A6 | A6 | B56 | B56 | 644 | S | A6 | A6 | B67 | B67 |
| 645 | S | A6 | A6 | B68 | B68 | 646 | S | A6 | A6 | B69 | B69 |
| 647 | S | A6 | A6 | B70 | B70 | 648 | S | A6 | A6 | B71 | B71 |
| 649 | S | A6 | A6 | B72 | B72 | 650 | S | A6 | A6 | B74 | B74 |
| 651 | S | A6 | A6 | B79 | B79 | 652 | S | A6 | A6 | B80 | B80 |
| 653 | S | A6 | A6 | B82 | B82 | 654 | S | A6 | A6 | B83 | B83 |
| 655 | S | A6 | A6 | B86 | B86 | 656 | S | A6 | A6 | B88 | B88 |
| 657 | Se | A6 | A6 | B1 | B1 | 658 | Se | A6 | A6 | B6 | B6 |
| 659 | Se | A6 | A6 | B10 | B10 | 660 | Se | A6 | A6 | B16 | B16 |
| 661 | Se | A6 | A6 | B25 | B25 | 662 | Se | A6 | A6 | B28 | B28 |
| 663 | Se | A6 | A6 | B29 | B29 | 664 | Se | A6 | A6 | B30 | B30 |
| 665 | Se | A6 | A6 | B38 | B38 | 666 | Se | A6 | A6 | B39 | B39 |
| 667 | Se | A6 | A6 | B40 | B40 | 668 | Se | A6 | A6 | B41 | B41 |
| 669 | Se | A6 | A6 | B43 | B43 | 670 | Se | A6 | A6 | B52 | B52 |
| 671 | Se | A6 | A6 | B56 | B56 | 672 | Se | A6 | A6 | B67 | B67 |
| 673 | Se | A6 | A6 | B68 | B68 | 674 | Se | A6 | A6 | B69 | B69 |
| 675 | Se | A6 | A6 | B70 | B70 | 676 | Se | A6 | A6 | B71 | B71 |
| 677 | Se | A6 | A6 | B72 | B72 | 678 | Se | A6 | A6 | B74 | B74 |
| 679 | Se | A6 | A6 | B79 | B79 | 680 | Se | A6 | A6 | B80 | B80 |
| 681 | Se | A6 | A6 | B82 | B82 | 682 | Se | A6 | A6 | B83 | B83 |
| 683 | Se | A6 | A6 | B86 | B86 | 684 | Se | A6 | A6 | B88 | B88 |
| 685 | O | A7 | A7 | B1 | B1 | 686 | O | A7 | A7 | B6 | B6 |
| 687 | O | A7 | A7 | B10 | B10 | 688 | O | A7 | A7 | B16 | B16 |
| 689 | O | A7 | A7 | B25 | B25 | 690 | O | A7 | A7 | B28 | B28 |
| 691 | O | A7 | A7 | B29 | B29 | 692 | O | A7 | A7 | B30 | B30 |
| 693 | O | A7 | A7 | B38 | B38 | 694 | O | A7 | A7 | B39 | B39 |
| 695 | O | A7 | A7 | B40 | B40 | 696 | O | A7 | A7 | B41 | B41 |
| 697 | O | A7 | A7 | B43 | B43 | 698 | O | A7 | A7 | B52 | B52 |
| 699 | O | A7 | A7 | B56 | B56 | 700 | O | A7 | A7 | B67 | B67 |
| 701 | O | A7 | A7 | B68 | B68 | 702 | O | A7 | A7 | B69 | B69 |
| 703 | O | A7 | A7 | B70 | B70 | 704 | O | A7 | A7 | B71 | B71 |
| 705 | O | A7 | A7 | B72 | B72 | 706 | O | A7 | A7 | B74 | B74 |
| 707 | O | A7 | A7 | B79 | B79 | 708 | O | A7 | A7 | B80 | B80 |
| 709 | O | A7 | A7 | B82 | B82 | 710 | O | A7 | A7 | B83 | B83 |
| 711 | O | A7 | A7 | B86 | B86 | 712 | O | A7 | A7 | B88 | B88 |
| 713 | S | A7 | A7 | B1 | B1 | 714 | S | A7 | A7 | B6 | B6 |
| 715 | S | A7 | A7 | B10 | B10 | 716 | S | A7 | A7 | B16 | B16 |
| 717 | S | A7 | A7 | B25 | B25 | 718 | S | A7 | A7 | B28 | B28 |
| 719 | S | A7 | A7 | B29 | B29 | 720 | S | A7 | A7 | B30 | B30 |
| 721 | S | A7 | A7 | B38 | B38 | 722 | S | A7 | A7 | B39 | B39 |
| 723 | S | A7 | A7 | B40 | B40 | 724 | S | A7 | A7 | B41 | B41 |
| 725 | S | A7 | A7 | B43 | B43 | 726 | S | A7 | A7 | B52 | B52 |
| 727 | S | A7 | A7 | B56 | B56 | 728 | S | A7 | A7 | B67 | B67 |
| 729 | S | A7 | A7 | B68 | B68 | 730 | S | A7 | A7 | B69 | B69 |
| 731 | S | A7 | A7 | B70 | B70 | 732 | S | A7 | A7 | B71 | B71 |
| 733 | S | A7 | A7 | B72 | B72 | 734 | S | A7 | A7 | B74 | B74 |
| 735 | S | A7 | A7 | B79 | B79 | 736 | S | A7 | A7 | B80 | B80 |
| 737 | S | A7 | A7 | B82 | B82 | 738 | S | A7 | A7 | B83 | B83 |
| 739 | S | A7 | A7 | B86 | B86 | 740 | S | A7 | A7 | B88 | B88 |
| 741 | Se | A7 | A7 | B1 | B1 | 742 | Se | A7 | A7 | B6 | B6 |
| 743 | Se | A7 | A7 | B10 | B10 | 744 | Se | A7 | A7 | B16 | B16 |
| 745 | Se | A7 | A7 | B25 | B25 | 746 | Se | A7 | A7 | B28 | B28 |
| 747 | Se | A7 | A7 | B29 | B29 | 748 | Se | A7 | A7 | B30 | B30 |
| 749 | Se | A7 | A7 | B38 | B38 | 750 | Se | A7 | A7 | B39 | B39 |
| 751 | Se | A7 | A7 | B40 | B40 | 752 | Se | A7 | A7 | B41 | B41 |
| 753 | Se | A7 | A7 | B43 | B43 | 754 | Se | A7 | A7 | B52 | B52 |
| 755 | Se | A7 | A7 | B56 | B56 | 756 | Se | A7 | A7 | B67 | B67 |
| 757 | Se | A7 | A7 | B68 | B68 | 758 | Se | A7 | A7 | B69 | B69 |
| 759 | Se | A7 | A7 | B70 | B70 | 760 | Se | A7 | A7 | B71 | B71 |
| 761 | Se | A7 | A7 | B72 | B72 | 762 | Se | A7 | A7 | B74 | B74 |
| 763 | Se | A7 | A7 | B79 | B79 | 764 | Se | A7 | A7 | B80 | B80 |
| 765 | Se | A7 | A7 | B82 | B82 | 766 | Se | A7 | A7 | B83 | B83 |
| 767 | Se | A7 | A7 | B86 | B86 | 768 | Se | A7 | A7 | B88 | B88 |
| 769 | O | O | O | B1 | B1 | 770 | O | O | O | B6 | B6 |
| 771 | O | O | O | B10 | B10 | 772 | O | O | O | B22 | B22 |
| 773 | O | O | O | B25 | B25 | 774 | O | O | O | B28 | B28 |
| 775 | O | O | O | B29 | B29 | 776 | O | O | O | B30 | B30 |
| 777 | O | O | O | B38 | B38 | 778 | O | O | O | B39 | B39 |
| 779 | O | O | O | B40 | B40 | 780 | O | O | O | B41 | B41 |
| 781 | O | O | O | B43 | B43 | 782 | O | O | O | B52 | B52 |
| 783 | O | O | O | B56 | B56 | 784 | O | O | O | B67 | B67 |
| 785 | O | O | O | B68 | B68 | 786 | O | O | O | B69 | B69 |
| 787 | O | O | O | B70 | B70 | 788 | O | O | O | B71 | B71 |
| 789 | O | O | O | B72 | B72 | 790 | O | O | O | B74 | B74 |
| 791 | O | O | O | B79 | B79 | 792 | O | O | O | B80 | B80 |
| 793 | O | O | O | B82 | B82 | 794 | O | O | O | B83 | B83 |
| 795 | O | O | O | B86 | B86 | 796 | O | O | O | B88 | B88 |
| 797 | S | O | O | B1 | B1 | 798 | S | O | O | B6 | B6 |
| 799 | S | O | O | B10 | B10 | 800 | S | O | O | B22 | B22 |
| 801 | S | O | O | B25 | B25 | 802 | S | O | O | B28 | B28 |
| 803 | S | O | O | B29 | B29 | 804 | S | O | O | B30 | B30 |
| 805 | S | O | O | B38 | B38 | 806 | S | O | O | B39 | B39 |
| 807 | S | O | O | B40 | B40 | 808 | S | O | O | B41 | B41 |
| 809 | S | O | O | B43 | B43 | 810 | S | O | O | B52 | B52 |
| 811 | S | O | O | B56 | B56 | 812 | S | O | O | B67 | B67 |
| 813 | S | O | O | B68 | B68 | 814 | S | O | O | B69 | B69 |
| 815 | S | O | O | B70 | B70 | 816 | S | O | O | B71 | B71 |
| 817 | S | O | O | B72 | B72 | 818 | S | O | O | B74 | B74 |
| 819 | S | O | O | B79 | B79 | 820 | S | O | O | B80 | B80 |
| 821 | S | O | O | B82 | B82 | 822 | S | O | O | B83 | B83 |
| 823 | S | O | O | B86 | B86 | 824 | S | O | O | B88 | B88 |
| 825 | Se | O | O | B1 | B1 | 826 | Se | O | O | B6 | B6 |
| 827 | Se | O | O | B10 | B10 | 828 | Se | O | O | B22 | B22 |
| 829 | Se | O | O | B25 | B25 | 830 | Se | O | O | B28 | B28 |
| 831 | Se | O | O | B29 | B29 | 832 | Se | O | O | B30 | B30 |
| 833 | Se | O | O | B38 | B38 | 834 | Se | O | O | B39 | B39 |
| 835 | Se | O | O | B40 | B40 | 836 | Se | O | O | B41 | B41 |
| 837 | Se | O | O | B43 | B43 | 838 | Se | O | O | B52 | B52 |
| 839 | Se | O | O | B56 | B56 | 840 | Se | O | O | B67 | B67 |
| 841 | Se | O | O | B68 | B68 | 842 | Se | O | O | B69 | B69 |
| 843 | Se | O | O | B70 | B70 | 844 | Se | O | O | B71 | B71 |
| 845 | Se | O | O | B72 | B72 | 846 | Se | O | O | B74 | B74 |
| 847 | Se | O | O | B79 | B79 | 848 | Se | O | O | B80 | B80 |
| 849 | Se | O | O | B82 | B82 | 850 | Se | O | O | B83 | B83 |
| 851 | Se | O | O | B86 | B86 | 852 | Se | O | O | B88 | B88 |
| 853 | O | S | S | B1 | B1 | 854 | O | O | O | B6 | B6 |
| 855 | O | S | S | B10 | B10 | 856 | O | S | S | B22 | B22 |
| 857 | O | S | S | B25 | B25 | 858 | O | S | S | B28 | B28 |
| 859 | O | S | S | B29 | B29 | 860 | O | S | S | B30 | B30 |
| 861 | O | S | S | B38 | B38 | 862 | O | S | S | B39 | B39 |
| 863 | O | S | S | B40 | B40 | 864 | O | S | S | B41 | B41 |
| 865 | O | S | S | B43 | B43 | 866 | O | S | S | B52 | B52 |
| 867 | O | S | S | B56 | B56 | 868 | O | S | S | B67 | B67 |
| 869 | O | S | S | B68 | B68 | 870 | O | S | S | B69 | B69 |
| 871 | O | S | S | B70 | B70 | 872 | O | S | S | B71 | B71 |
| 873 | O | S | S | B72 | B72 | 874 | O | S | S | B74 | B74 |
| 875 | O | S | S | B79 | B79 | 876 | O | S | S | B80 | B80 |
| 877 | O | S | S | B82 | B82 | 878 | O | S | S | B83 | B83 |
| 879 | O | S | S | B86 | B86 | 880 | O | S | S | B88 | B88 |
| 881 | S | S | S | B1 | B1 | 882 | S | S | S | B6 | B6 |
| 883 | S | S | S | B10 | B10 | 884 | S | S | S | B22 | B22 |
| 885 | S | S | S | B25 | B25 | 886 | S | S | S | B28 | B28 |
| 887 | S | S | S | B29 | B29 | 888 | S | S | S | B30 | B30 |
| 889 | S | S | S | B38 | B38 | 890 | S | S | S | B39 | B39 |
| 891 | S | S | S | B40 | B40 | 892 | S | S | S | B41 | B41 |
| 893 | S | S | S | B43 | B43 | 894 | S | S | S | B52 | B52 |
| 895 | S | S | S | B56 | B56 | 896 | S | S | S | B67 | B67 |
| 897 | S | S | S | B68 | B68 | 898 | S | S | S | B69 | B69 |
| 899 | S | S | S | B70 | B70 | 900 | S | S | S | B71 | B71 |
| 901 | S | S | S | B72 | B72 | 902 | S | S | S | B74 | B74 |
| 903 | S | S | S | B79 | B79 | 904 | S | S | S | B80 | B80 |
| 905 | S | S | S | B82 | B82 | 906 | S | S | S | B83 | B83 |
| 907 | S | S | S | B86 | B86 | 908 | S | S | S | B88 | B88 |
| 909 | Se | S | S | B1 | B1 | 910 | Se | S | S | B6 | B6 |
| 911 | Se | S | S | B10 | B10 | 912 | Se | S | S | B22 | B22 |
| 913 | Se | S | S | B25 | B25 | 914 | Se | S | S | B28 | B28 |
| 915 | Se | S | S | B29 | B29 | 916 | Se | S | S | B30 | B30 |
| 917 | Se | S | S | B38 | B38 | 918 | Se | S | S | B39 | B39 |
| 919 | Se | S | S | B40 | B40 | 920 | Se | S | S | B41 | B41 |
| 921 | Se | S | S | B43 | B43 | 922 | Se | S | S | B52 | B52 |
| 923 | Se | S | S | B56 | B56 | 924 | Se | S | S | B67 | B67 |
| 925 | Se | S | S | B68 | B68 | 926 | Se | S | S | B69 | B69 |
| 927 | Se | S | S | B70 | B70 | 928 | Se | S | S | B71 | B71 |
| 929 | Se | S | S | B72 | B72 | 930 | Se | S | S | B74 | B74 |
| 931 | Se | S | S | B79 | B79 | 932 | Se | S | S | B80 | B80 |
| 933 | Se | S | S | B82 | B82 | 934 | Se | S | S | B83 | B83 |
| 935 | Se | S | S | B86 | B86 | 936 | Se | S | S | B88 | B88 |
| 937 | O | Se | Se | B1 | B1 | 938 | O | Se | Se | B6 | B6 |
| 939 | O | Se | Se | B10 | B10 | 940 | O | Se | Se | B22 | B22 |
| 941 | O | Se | Se | B25 | B25 | 942 | O | Se | Se | B28 | B28 |
| 943 | O | Se | Se | B29 | B29 | 944 | O | Se | Se | B30 | B30 |
| 945 | O | Se | Se | B38 | B38 | 946 | O | Se | Se | B39 | B39 |
| 947 | O | Se | Se | B40 | B40 | 948 | O | Se | Se | B41 | B41 |
| 949 | O | Se | Se | B43 | B43 | 950 | O | Se | Se | B52 | B52 |
| 951 | O | Se | Se | B56 | B56 | 952 | O | Se | Se | B67 | B67 |
| 953 | O | Se | Se | B68 | B68 | 954 | O | Se | Se | B69 | B69 |
| 955 | O | Se | Se | B70 | B70 | 956 | O | Se | Se | B71 | B71 |
| 957 | O | Se | Se | B72 | B72 | 958 | O | Se | Se | B74 | B74 |
| 959 | O | Se | Se | B79 | B79 | 960 | O | Se | Se | B80 | B80 |
| 961 | O | Se | Se | B82 | B82 | 962 | O | Se | Se | B83 | B83 |
| 963 | O | Se | Se | B86 | B86 | 964 | O | Se | Se | B88 | B88 |
| 965 | S | Se | Se | B1 | B1 | 966 | S | Se | Se | B6 | B6 |
| 967 | S | Se | Se | B10 | B10 | 968 | S | Se | Se | B22 | B22 |
| 969 | S | Se | Se | B25 | B25 | 970 | S | Se | Se | B28 | B28 |
| 971 | S | Se | Se | B29 | B29 | 972 | S | Se | Se | B30 | B30 |
| 973 | S | Se | Se | B38 | B38 | 974 | S | Se | Se | B39 | B39 |
| 975 | S | Se | Se | B40 | B40 | 976 | S | Se | Se | B41 | B41 |
| 977 | S | Se | Se | B43 | B43 | 978 | S | Se | Se | B52 | B52 |
| 979 | S | Se | Se | B56 | B56 | 980 | S | Se | Se | B67 | B67 |
| 981 | S | Se | Se | B68 | B68 | 982 | S | Se | Se | B69 | B69 |
| 983 | S | Se | Se | B70 | B70 | 984 | S | Se | Se | B71 | B71 |
| 985 | S | Se | Se | B72 | B72 | 986 | S | Se | Se | B74 | B74 |
| 987 | S | Se | Se | B79 | B79 | 988 | S | Se | Se | B80 | B80 |
| 989 | S | Se | Se | B82 | B82 | 990 | S | Se | Se | B83 | B83 |
| 991 | S | Se | Se | B86 | B86 | 992 | S | Se | Se | B88 | B88 |
| 993 | Se | Se | Se | B1 | B1 | 994 | Se | Se | Se | B6 | B6 |
| 995 | Se | Se | Se | B10 | B10 | 996 | Se | Se | Se | B22 | B22 |
| 997 | Se | Se | Se | B25 | B25 | 998 | Se | Se | Se | B28 | B28 |
| 999 | Se | Se | Se | B29 | B29 | 1000 | Se | Se | Se | B30 | B30 |
| 1001 | Se | Se | Se | B38 | B38 | 1002 | Se | Se | Se | B39 | B39 |
| 1003 | Se | Se | Se | B40 | B40 | 1004 | Se | Se | Se | B41 | B41 |
| 1005 | Se | Se | Se | B43 | B43 | 1006 | Se | Se | Se | B52 | B52 |
| 1007 | Se | Se | Se | B56 | B56 | 1008 | Se | Se | Se | B67 | B67 |
| 1009 | Se | Se | Se | B68 | B68 | 1010 | Se | Se | Se | B69 | B69 |
| 1011 | Se | Se | Se | B70 | B70 | 1012 | Se | Se | Se | B71 | B71 |
| 1013 | Se | Se | Se | B72 | B72 | 1014 | Se | Se | Se | B74 | B74 |
| 1015 | Se | Se | Se | B79 | B79 | 1016 | Se | Se | Se | B80 | B80 |
| 1017 | Se | Se | Se | B82 | B82 | 1018 | Se | Se | Se | B83 | B83 |
| 1019 | Se | Se | Se | B86 | B86 | 1020 | Se | Se | Se | B88 | B88 |
| 1021 | O | A1 | A1 | B1 | B6 | 1022 | O | A1 | A1 | B2 | B6 |
| 1023 | O | A1 | A1 | B25 | B26 | 1024 | O | A1 | A1 | B27 | B28 |
| 1025 | O | A1 | A1 | B29 | B30 | 1026 | O | A1 | A1 | B39 | B40 |
| 1027 | O | A1 | A1 | B54 | B41 | 1028 | O | A1 | A1 | B54 | B52 |
| 1029 | O | A1 | A1 | B52 | B56 | 1030 | O | A1 | A1 | B55 | B56 |
| 1031 | O | A1 | A1 | B64 | B56 | 1032 | O | A1 | A1 | B68 | B69 |
| 1033 | O | A1 | A1 | B69 | B70 | 1034 | O | A1 | A1 | B71 | B72 |
| 1035 | O | A1 | A1 | B68 | B80 | 1036 | O | A1 | A1 | B68 | B83 |
| 1037 | S | A1 | A1 | B1 | B6 | 1038 | S | A1 | A1 | B2 | B6 |
| 1039 | S | A1 | A1 | B25 | B26 | 1040 | S | A1 | A1 | B27 | B28 |
| 1041 | S | A1 | A1 | B29 | B30 | 1042 | S | A1 | A1 | B39 | B40 |
| 1043 | S | A1 | A1 | B54 | B41 | 1044 | S | A1 | A1 | B54 | B52 |
| 1045 | S | A1 | A1 | B52 | B56 | 1046 | S | A1 | A1 | B55 | B56 |
| 1047 | S | A1 | A1 | B64 | B56 | 1048 | S | A1 | A1 | B68 | B69 |
| 1049 | S | A1 | A1 | B69 | B70 | 1050 | S | A1 | A1 | B71 | B72 |
| 1051 | S | A1 | A1 | B68 | B80 | 1052 | S | A1 | A1 | B68 | B83 |
| 1053 | Se | A1 | A1 | B1 | B6 | 1054 | Se | A1 | A1 | B2 | B6 |
| 1055 | Se | A1 | A1 | B25 | B26 | 1056 | Se | A1 | A1 | B27 | B28 |
| 1057 | Se | A1 | A1 | B29 | B30 | 1058 | Se | A1 | A1 | B39 | B40 |
| 1059 | Se | A1 | A1 | B54 | B41 | 1060 | Se | A1 | A1 | B54 | B52 |
| 1061 | Se | A1 | A1 | B52 | B56 | 1062 | Se | A1 | A1 | B55 | B56 |
| 1063 | Se | A1 | A1 | B64 | B56 | 1064 | Se | A1 | A1 | B68 | B69 |
| 1065 | Se | A1 | A1 | B69 | B70 | 1066 | Se | A1 | A1 | B71 | B72 |
| 1067 | Se | A1 | A1 | B68 | B80 | 1068 | Se | A1 | A1 | B68 | B83 |
| 1069 | O | A2 | A2 | B1 | B6 | 1070 | O | A2 | A2 | B2 | B6 |
| 1071 | O | A2 | A2 | B25 | B26 | 1072 | O | A2 | A2 | B27 | B28 |
| 1073 | O | A2 | A2 | B29 | B30 | 1074 | O | A2 | A2 | B39 | B40 |
| 1075 | O | A2 | A2 | B54 | B41 | 1076 | O | A2 | A2 | B54 | B52 |
| 1077 | O | A2 | A2 | B52 | B56 | 1078 | O | A2 | A2 | B55 | B56 |
| 1079 | O | A2 | A2 | B64 | B56 | 1080 | O | A2 | A2 | B68 | B69 |
| 1081 | O | A2 | A2 | B69 | B70 | 1082 | O | A2 | A2 | B71 | B72 |
| 1083 | O | A2 | A2 | B68 | B80 | 1084 | O | A2 | A2 | B68 | B83 |
| 1085 | S | A2 | A2 | B1 | B6 | 1086 | S | A2 | A2 | B2 | B6 |
| 1087 | S | A2 | A2 | B25 | B26 | 1088 | S | A2 | A2 | B27 | B28 |
| 1089 | S | A2 | A2 | B29 | B30 | 1090 | S | A2 | A2 | B39 | B40 |
| 1091 | S | A2 | A2 | B54 | B41 | 1092 | S | A2 | A2 | B54 | B52 |
| 1093 | S | A2 | A2 | B52 | B56 | 1094 | S | A2 | A2 | B55 | B56 |
| 1095 | S | A2 | A2 | B64 | B56 | 1096 | S | A2 | A2 | B68 | B69 |
| 1097 | S | A2 | A2 | B69 | B70 | 1098 | S | A2 | A2 | B71 | B72 |
| 1099 | S | A2 | A2 | B68 | B80 | 1100 | S | A2 | A2 | B68 | B83 |
| 1101 | Se | A2 | A2 | B1 | B6 | 1102 | Se | A2 | A2 | B2 | B6 |
| 1103 | Se | A2 | A2 | B25 | B26 | 1104 | Se | A2 | A2 | B27 | B28 |
| 1105 | Se | A2 | A2 | B29 | B30 | 1106 | Se | A2 | A2 | B39 | B40 |
| 1107 | Se | A2 | A2 | B54 | B41 | 1108 | Se | A2 | A2 | B54 | B52 |
| 1109 | Se | A2 | A2 | B52 | B56 | 1110 | Se | A2 | A2 | B55 | B56 |
| 1111 | Se | A2 | A2 | B64 | B56 | 1112 | Se | A2 | A2 | B68 | B69 |
| 1113 | Se | A2 | A2 | B69 | B70 | 1114 | Se | A2 | A2 | B71 | B72 |
| 1115 | Se | A2 | A2 | B68 | B80 | 1116 | Se | A2 | A2 | B68 | B83 |
| 1117 | O | A3 | A3 | B1 | B1 | 1118 | O | A3 | A3 | B6 | B6 |
| 1119 | O | A3 | A3 | B25 | B25 | 1120 | O | A3 | A3 | B28 | B28 |
| 1121 | O | A3 | A3 | B29 | B29 | 1122 | O | A3 | A3 | B30 | B30 |
| 1123 | O | A3 | A3 | B56 | B56 | 1124 | O | A3 | A3 | B67 | B67 |
| 1125 | O | A3 | A3 | B68 | B68 | 1126 | O | A3 | A3 | B69 | B69 |
| 1127 | O | A3 | A3 | B70 | B70 | 1128 | O | A3 | A3 | B71 | B71 |
| 1129 | O | A3 | A3 | B72 | B72 | 1130 | O | A3 | A3 | B74 | B74 |
| 1131 | O | A3 | A3 | B80 | B80 | 1132 | O | A3 | A3 | B83 | B83 |
| 1133 | S | A3 | A3 | B1 | B1 | 1134 | S | A3 | A3 | B6 | B6 |
| 1135 | S | A3 | A3 | B25 | B25 | 1136 | S | A3 | A3 | B28 | B28 |
| 1137 | S | A3 | A3 | B29 | B29 | 1138 | S | A3 | A3 | B30 | B30 |
| 1139 | S | A3 | A3 | B56 | B56 | 1140 | S | A3 | A3 | B67 | B67 |
| 1141 | S | A3 | A3 | B68 | B68 | 1142 | S | A3 | A3 | B69 | B69 |
| 1143 | S | A3 | A3 | B70 | B70 | 1144 | S | A3 | A3 | B71 | B71 |
| 1145 | S | A3 | A3 | B72 | B72 | 1146 | S | A3 | A3 | B74 | B74 |
| 1147 | S | A3 | A3 | B80 | B80 | 1148 | S | A3 | A3 | B83 | B83 |
| 1149 | Se | A3 | A3 | B1 | B1 | 1150 | Se | A3 | A3 | B6 | B6 |
| 1151 | Se | A3 | A3 | B25 | B25 | 1152 | Se | A3 | A3 | B28 | B28 |
| 1153 | Se | A3 | A3 | B29 | B29 | 1154 | Se | A3 | A3 | B30 | B30 |
| 1155 | Se | A3 | A3 | B56 | B56 | 1156 | Se | A3 | A3 | B67 | B67 |
| 1157 | Se | A3 | A3 | B68 | B68 | 1158 | Se | A3 | A3 | B69 | B69 |
| 1159 | Se | A3 | A3 | B70 | B70 | 1160 | Se | A3 | A3 | B71 | B71 |
| 1161 | Se | A3 | A3 | B72 | B72 | 1162 | Se | A3 | A3 | B74 | B74 |
| 1163 | Se | A3 | A3 | B80 | B80 | 1164 | Se | A3 | A3 | B83 | B83 |
| 1165 | O | O | A1 | B1 | B1 | 1166 | O | O | A1 | B6 | B6 |
| 1167 | O | O | A1 | B25 | B25 | 1168 | O | O | A1 | B28 | B28 |
| 1169 | O | O | A1 | B29 | B29 | 1170 | O | O | A1 | B30 | B30 |
| 1171 | O | O | A1 | B56 | B56 | 1172 | O | O | A1 | B67 | B67 |
| 1173 | O | O | A1 | B68 | B68 | 1174 | O | O | A1 | B69 | B69 |
| 1175 | O | O | A1 | B70 | B70 | 1176 | O | O | A1 | B71 | B71 |
| 1177 | O | O | A1 | B72 | B72 | 1178 | O | O | A1 | B74 | B74 |
| 1179 | O | O | A1 | B80 | B80 | 1180 | O | O | A1 | B83 | B83 |
| 1181 | S | O | A1 | B1 | B1 | 1182 | S | O | A1 | B6 | B6 |
| 1183 | S | O | A1 | B25 | B25 | 1184 | S | O | A1 | B28 | B28 |
| 1185 | S | O | A1 | B29 | B29 | 1186 | S | O | A1 | B30 | B30 |
| 1187 | S | O | A1 | B56 | B56 | 1188 | S | O | A1 | B67 | B67 |
| 1189 | S | O | A1 | B68 | B68 | 1190 | S | O | A1 | B69 | B69 |
| 1191 | S | O | A1 | B70 | B70 | 1192 | S | O | A1 | B71 | B71 |
| 1193 | S | O | A1 | B72 | B72 | 1194 | S | O | A1 | B74 | B74 |
| 1195 | S | O | A1 | B80 | B80 | 1196 | S | O | A1 | B83 | B83 |
| 1197 | Se | O | A1 | B1 | B1 | 1198 | Se | O | A1 | B6 | B6 |
| 1199 | Se | O | A1 | B25 | B25 | 1200 | Se | O | A1 | B28 | B28 |
| 1201 | Se | O | A1 | B29 | B29 | 1202 | Se | O | A1 | B30 | B30 |
| 1203 | Se | O | A1 | B56 | B56 | 1204 | Se | O | A1 | B67 | B67 |
| 1205 | Se | O | A1 | B68 | B68 | 1206 | Se | O | A1 | B69 | B69 |
| 1207 | Se | O | A1 | B70 | B70 | 1208 | Se | O | A1 | B71 | B71 |
| 1209 | Se | O | A1 | B72 | B72 | 1210 | Se | O | A1 | B74 | B74 |
| 1211 | Se | O | A1 | B80 | B80 | 1212 | Se | O | A1 | B83 | B83 |
| 1213 | O | A1 | A2 | B1 | B1 | 1214 | O | A1 | A2 | B6 | B6 |
| 1215 | O | A1 | A2 | B25 | B25 | 1216 | O | A1 | A2 | B28 | B28 |
| 1217 | O | A1 | A2 | B29 | B29 | 1218 | O | A1 | A2 | B30 | B30 |
| 1219 | O | A1 | A2 | B56 | B56 | 1220 | O | A1 | A2 | B67 | B67 |
| 1221 | O | A1 | A2 | B68 | B68 | 1222 | O | A1 | A2 | B69 | B69 |
| 1223 | O | A1 | A2 | B70 | B70 | 1224 | O | A1 | A2 | B71 | B71 |
| 1225 | O | A1 | A2 | B72 | B72 | 1226 | O | A1 | A2 | B74 | B74 |
| 1227 | O | A1 | A2 | B80 | B80 | 1228 | O | A1 | A2 | B83 | B83 |
| 1229 | S | A1 | A2 | B1 | B1 | 1230 | S | A1 | A2 | B6 | B6 |
| 1231 | S | A1 | A2 | B25 | B25 | 1232 | S | A1 | A2 | B28 | B28 |
| 1233 | S | A1 | A2 | B29 | B29 | 1234 | S | A1 | A2 | B30 | B30 |
| 1235 | S | A1 | A2 | B56 | B56 | 1236 | S | A1 | A2 | B67 | B67 |
| 1237 | S | A1 | A2 | B68 | B68 | 1238 | S | A1 | A2 | B69 | B69 |
| 1239 | S | A1 | A2 | B70 | B70 | 1240 | S | A1 | A2 | B71 | B71 |
| 1241 | S | A1 | A2 | B72 | B72 | 1242 | S | A1 | A2 | B74 | B74 |
| 1243 | S | A1 | A2 | B80 | B80 | 1244 | S | A1 | A2 | B83 | B83 |
| 1245 | Se | A1 | A2 | B1 | B1 | 1246 | Se | A1 | A2 | B6 | B6 |
| 1247 | Se | A1 | A2 | B25 | B25 | 1248 | Se | A1 | A2 | B28 | B28 |
| 1249 | Se | A1 | A2 | B29 | B29 | 1250 | Se | A1 | A2 | B30 | B30 |
| 1251 | Se | A1 | A2 | B56 | B56 | 1252 | Se | A1 | A2 | B67 | B67 |
| 1253 | Se | A1 | A2 | B68 | B68 | 1254 | Se | A1 | A2 | B69 | B69 |
| 1255 | Se | A1 | A2 | B70 | B70 | 1256 | Se | A1 | A2 | B71 | B71 |
| 1257 | Se | A1 | A2 | B72 | B72 | 1258 | Se | A1 | A2 | B74 | B74 |
| 1259 | Se | A1 | A2 | B80 | B80 | 1260 | Se | A1 | A2 | B83 | B83 |
| 1261 | O | A1 | A3 | B1 | B1 | 1262 | O | A1 | A3 | B6 | B6 |
| 1263 | O | A1 | A3 | B25 | B25 | 1264 | O | A1 | A3 | B28 | B28 |
| 1265 | O | A1 | A3 | B29 | B29 | 1266 | O | A1 | A3 | B30 | B30 |
| 1267 | O | A1 | A3 | B56 | B56 | 1268 | O | A1 | A3 | B67 | B67 |
| 1269 | O | A1 | A3 | B68 | B68 | 1270 | O | A1 | A3 | B69 | B69 |
| 1271 | O | A1 | A3 | B70 | B70 | 1272 | O | A1 | A3 | B71 | B71 |
| 1273 | O | A1 | A3 | B72 | B72 | 1274 | O | A1 | A3 | B74 | B74 |
| 1275 | O | A1 | A3 | B80 | B80 | 1276 | O | A1 | A3 | B83 | B83 |
| 1277 | S | A1 | A3 | B1 | B1 | 1278 | S | A1 | A3 | B6 | B6 |
| 1279 | S | A1 | A3 | B25 | B25 | 1280 | S | A1 | A3 | B28 | B28 |
| 1281 | S | A1 | A3 | B29 | B29 | 1282 | S | A1 | A3 | B30 | B30 |
| 1283 | S | A1 | A3 | B56 | B56 | 1284 | S | A1 | A3 | B67 | B67 |
| 1285 | S | A1 | A3 | B68 | B68 | 1286 | S | A1 | A3 | B69 | B69 |
| 1287 | S | A1 | A3 | B70 | B70 | 1288 | S | A1 | A3 | B71 | B71 |
| 1289 | S | A1 | A3 | B72 | B72 | 1290 | S | A1 | A3 | B74 | B74 |
| 1291 | S | A1 | A3 | B80 | B80 | 1292 | S | A1 | A3 | B83 | B83 |
| 1293 | Se | A1 | A3 | B1 | B1 | 1294 | Se | A1 | A3 | B6 | B6 |
| 1295 | Se | A1 | A3 | B25 | B25 | 1296 | Se | A1 | A3 | B28 | B28 |
| 1297 | Se | A1 | A3 | B29 | B29 | 1298 | Se | A1 | A3 | B30 | B30 |
| 1299 | Se | A1 | A3 | B56 | B56 | 1300 | Se | A1 | A3 | B67 | B67 |
| 1301 | Se | A1 | A3 | B68 | B68 | 1302 | Se | A1 | A3 | B69 | B69 |
| 1303 | Se | A1 | A3 | B70 | B70 | 1304 | Se | A1 | A3 | B71 | B71 |
| 1305 | Se | A1 | A3 | B72 | B72 | 1306 | Se | A1 | A3 | B74 | B74 |
| 1307 | Se | A1 | A3 | B80 | B80 | 1308 | Se | A1 | A3 | B83 | B83 |
| 1309 | O | A2 | A6 | B1 | B1 | 1310 | O | A2 | A6 | B6 | B6 |
| 1311 | O | A2 | A6 | B25 | B25 | 1312 | O | A2 | A6 | B28 | B28 |
| 1313 | O | A2 | A6 | B29 | B29 | 1314 | O | A2 | A6 | B30 | B30 |
| 1315 | O | A2 | A6 | B56 | B56 | 1316 | O | A2 | A6 | B67 | B67 |
| 1317 | O | A2 | A6 | B68 | B68 | 1318 | O | A2 | A6 | B69 | B69 |
| 1319 | O | A2 | A6 | B70 | B70 | 1320 | O | A2 | A6 | B71 | B71 |
| 1321 | O | A2 | A6 | B72 | B72 | 1322 | O | A2 | A6 | B74 | B74 |
| 1323 | O | A2 | A6 | B80 | B80 | 1324 | O | A2 | A6 | B83 | B83 |
| 1325 | S | A2 | A6 | B1 | B1 | 1326 | S | A2 | A6 | B6 | B6 |
| 1327 | S | A2 | A6 | B25 | B25 | 1328 | S | A2 | A6 | B28 | B28 |
| 1329 | S | A2 | A6 | B29 | B29 | 1330 | S | A2 | A6 | B30 | B30 |
| 1331 | S | A2 | A6 | B56 | B56 | 1332 | S | A2 | A6 | B67 | B67 |
| 1333 | S | A2 | A6 | B68 | B68 | 1334 | S | A2 | A6 | B69 | B69 |
| 1335 | S | A2 | A6 | B70 | B70 | 1336 | S | A2 | A6 | B71 | B71 |
| 1337 | S | A2 | A6 | B72 | B72 | 1338 | S | A2 | A6 | B74 | B74 |
| 1339 | S | A2 | A6 | B80 | B80 | 1340 | S | A2 | A6 | B83 | B83 |
| 1341 | Se | A2 | A6 | B1 | B1 | 1342 | Se | A2 | A6 | B6 | B6 |
| 1343 | Se | A2 | A6 | B25 | B25 | 1344 | Se | A2 | A6 | B28 | B28 |
| 1345 | Se | A2 | A6 | B29 | B29 | 1346 | Se | A2 | A6 | B30 | B30 |
| 1347 | Se | A2 | A6 | B56 | B56 | 1348 | Se | A2 | A6 | B67 | B67 |
| 1349 | Se | A2 | A6 | B68 | B68 | 1350 | Se | A2 | A6 | B69 | B69 |
| 1351 | Se | A2 | A6 | B70 | B70 | 1352 | Se | A2 | A6 | B71 | B71 |
| 1353 | Se | A2 | A6 | B72 | B72 | 1354 | Se | A2 | A6 | B74 | B74 |
| 1355 | Se | A2 | A6 | B80 | B80 | 1356 | Se | A2 | A6 | B83 | B83 |

11. The organic electroluminescent device of claim 1, wherein the second compound has a structure represented by any one of Formula 2-1 to Formula 2-12: 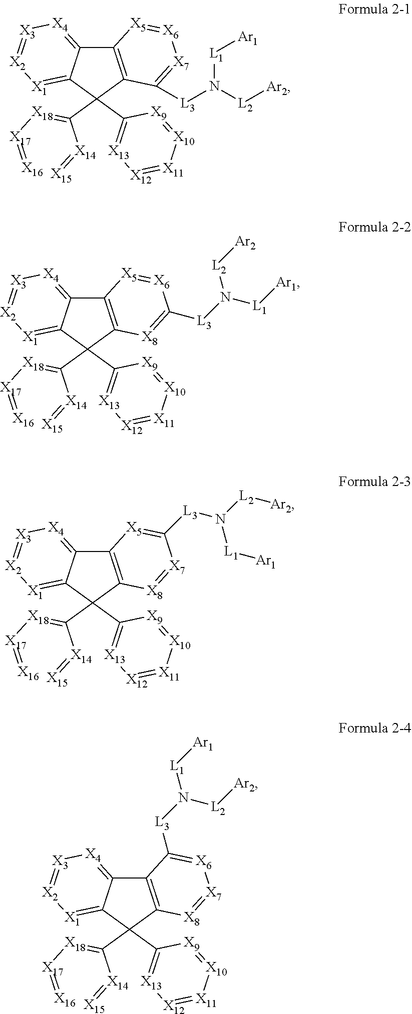 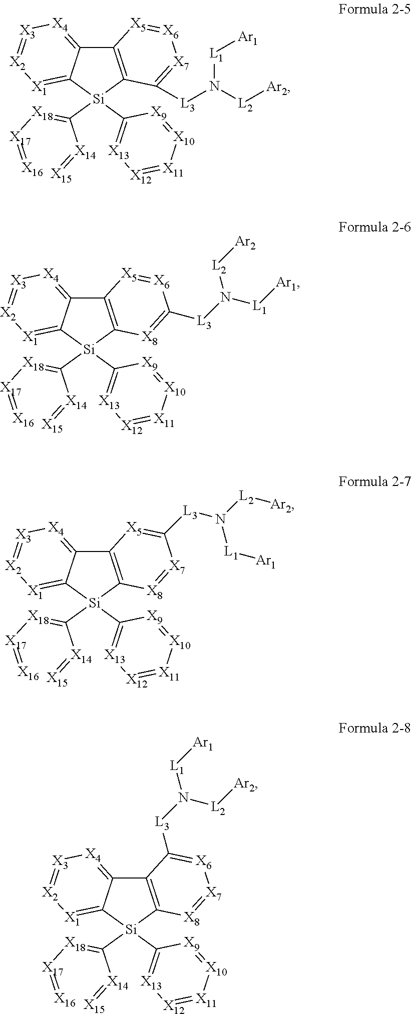 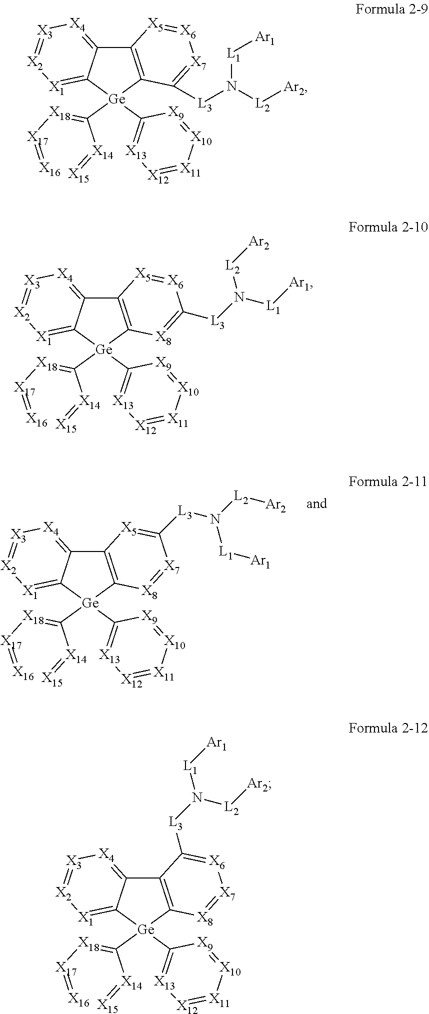
X₁ to X₁₈ are, at each occurrence identically or differently, selected from CR₁;
L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof,
Ar₁ and Ar₂ are, at each occurrence identically or differently, selected from substituted or unsubstituted aryl having 6 to 30 carbon atoms or substituted or unsubstituted heteroaryl having 3 to 30 carbon atoms;
R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents L₁, L₂, L₃, R₁, Ar₁ and Ar₂ can be optionally joined to form a ring;
preferably, the second compound has a structure represented by Formula 2-1, Formula 2-2, Formula 2-3, Formula 2-4, Formula 2-6 or Formula 2-10.

12. The organic electroluminescent device of claim 1, wherein in Formula 2, at least one of X₁ to X₁₈ is N.

13. The organic electroluminescent device of claim 1, wherein the second compound has a structure represented by any one of Formula 2-13 to Formula 2-24: 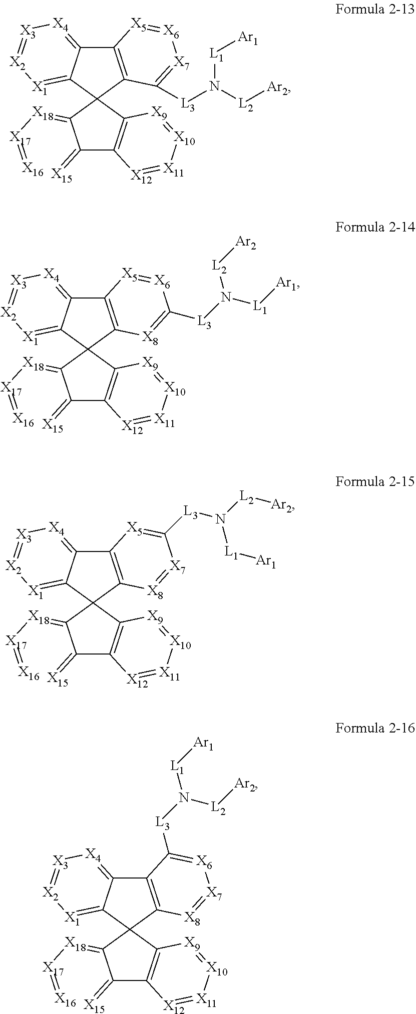 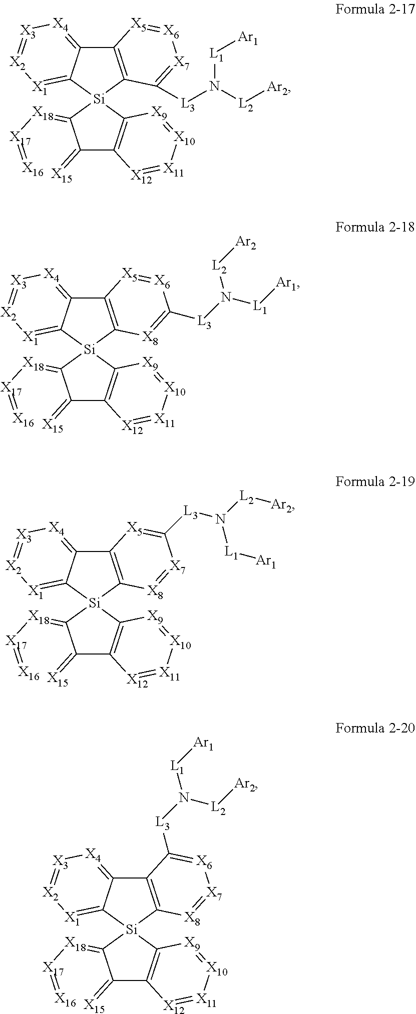 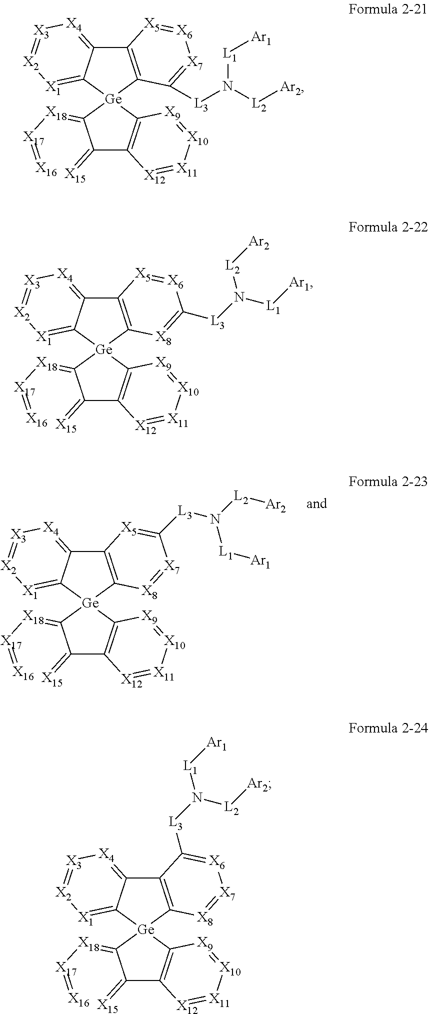
X₁ to X₁₈ are, at each occurrence identically or differently, selected from CR₁ or N;
L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof,
Ar₁ and Ar₂ are, at each occurrence identically or differently, selected from substituted or unsubstituted aryl having 6 to 30 carbon atoms or substituted or unsubstituted heteroaryl having 3 to 30 carbon atoms;
R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents L₁, L₂, L₃, R₁, Ar₁ and Ar₂ can be optionally joined to form a ring;
preferably, the second compound has a structure represented by Formula 2-13, Formula 2-14, Formula 2-15, Formula 2-16, Formula 2-18 or Formula 2-22.

14. The organic electroluminescent device of claim 1, wherein the L₁ to L₃ are, at each occurrence identically or differently, selected from a single bond, a substituted or unsubstituted arylene group having 6 to 24 carbon atoms, a substituted or unsubstituted heteroarylene group having 3 to 24 carbon atoms or a combination thereof,
preferably, L₁ to L₃ are, at each occurrence identically or differently, selected from a single bond, a substituted or unsubstituted phenylene group, a substituted or unsubstituted biphenylene group, a substituted or unsubstituted terphenylene group, a substituted or unsubstituted naphthylene group, a substituted or unsubstituted fluorenylidene group, a substituted or unsubstituted silafluorenylidene group, a substituted or unsubstituted carbazolylene group, a substituted or unsubstituted dibenzofuranylene group, a substituted or unsubstituted dibenzothienylene group, a substituted or unsubstituted dibenzoselenophenylene group, a substituted or unsubstituted phenanthrylene group, a substituted or unsubstituted triphenylenylene group, a substituted or unsubstituted pyridylene group, a substituted or unsubstituted spirobifluorenylidene group, a substituted or unsubstituted anthrylene group, a substituted or unsubstituted pyrenylene group or a combination thereof,
more preferably, L₁ to L₃ are, at each occurrence identically or differently, selected from the group consisting of the following:
a single bond 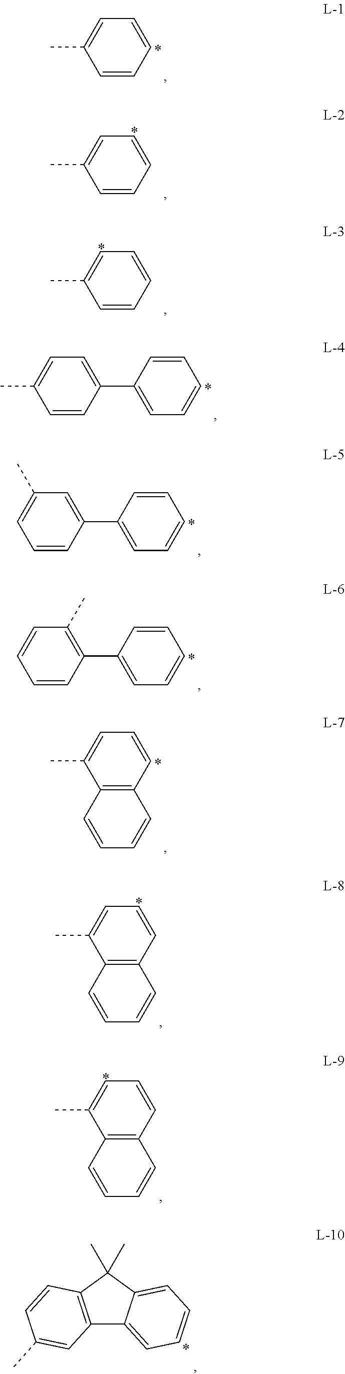 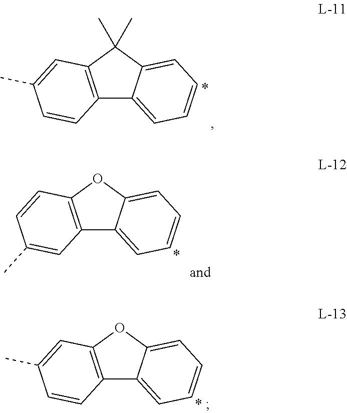
wherein “*” represents a position where nitrogen in Formula 2 is bonded in L-1 to L-13, and the dashed line represents a position where Ar₁, Ar₂ or any one of X₁ to X₈ in Formula 2 is bonded in L-1 to L-13.

15. The organic electroluminescent device of claim 1, wherein Ar₁ and Ar₂ have, at each occurrence identically or differently, a structure represented by any one of Formula 3-1 to Formula 3-4: 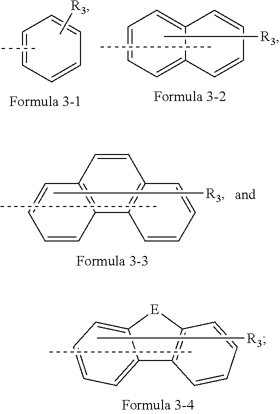
E is, at each occurrence identically or differently, selected from O, S, Se, C(R₄)₂, Si(R₄)₂ or Ge(R₄)₂;
R₃ represents, at each occurrence identically or differently, mono-substitution, multiple substitutions or non-substitution;
R₃ and R₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents R₃, R₄ can be optionally joined to form a ring;
preferably, R₃ and R₄ are, at each occurrence identically or differently, selected from hydrogen, deuterium, halogen, substituted or unsubstituted alkyl having 1 to 20 carbon atoms, substituted or unsubstituted aryl having 6 to 30 carbon atoms, substituted or unsubstituted heteroaryl having 3 to 30 carbon atoms or a combination thereof,
the dashed line represents a position where the L₁ is joined in the structure of Ar₁; and the dashed line also represents a position where the L₂ is joined in the structure of Ar₂.

16. The organic electroluminescent device of claim 15, wherein Ar₁ and Ar₂ are, at each occurrence identically or differently, selected from the group consisting of G1 to G37: 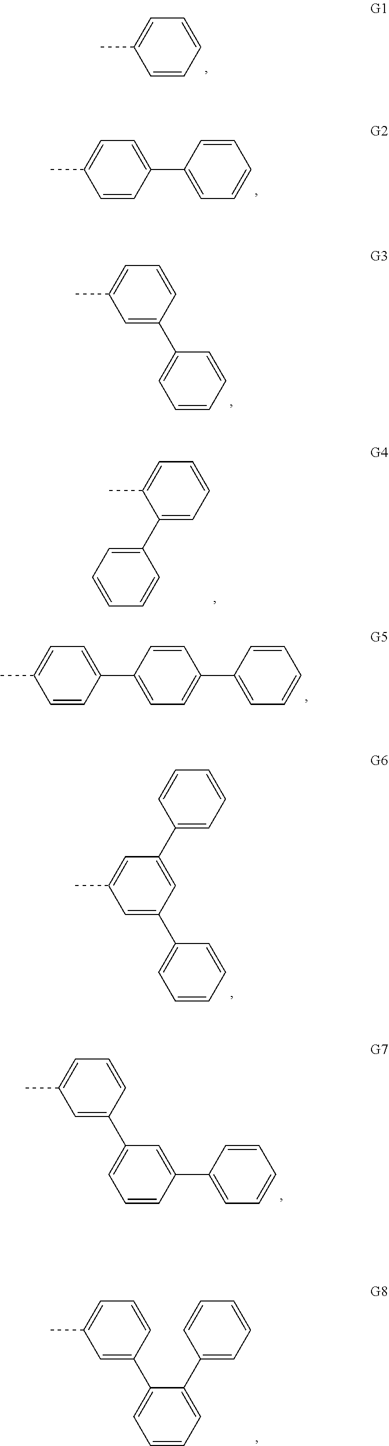 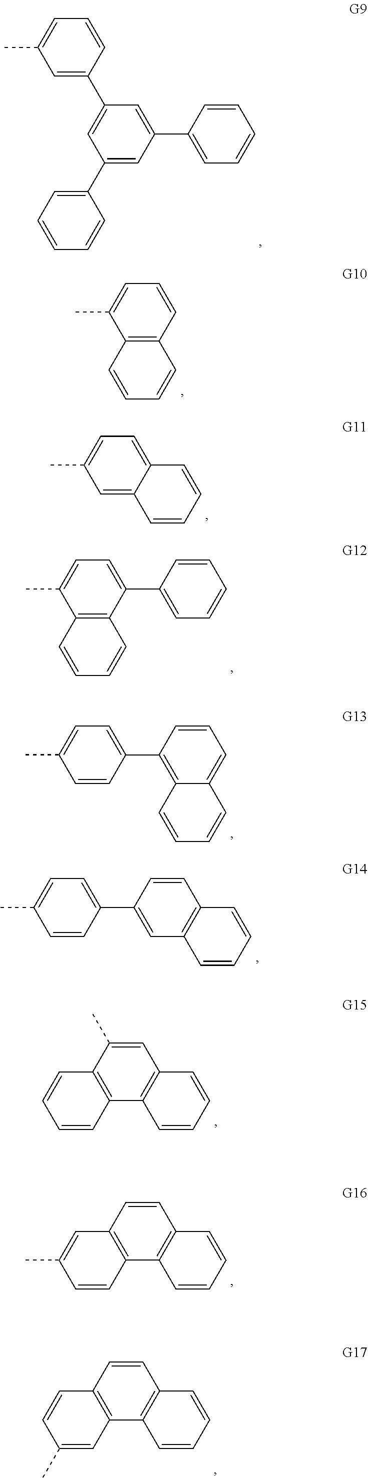 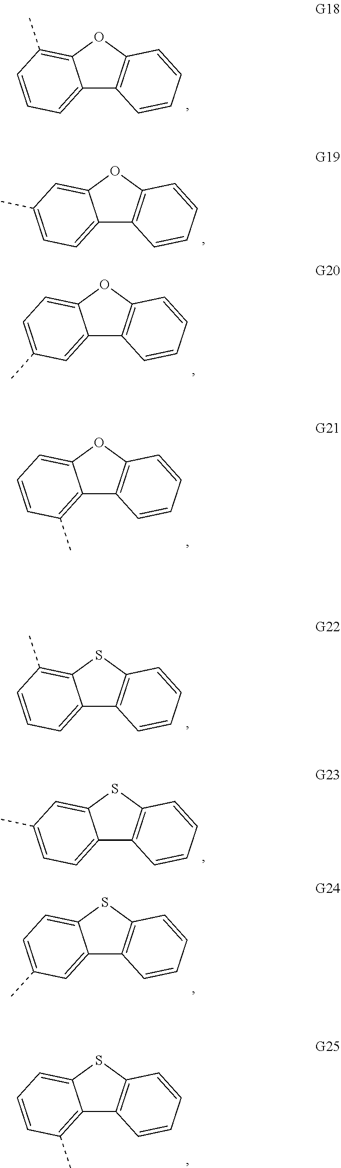 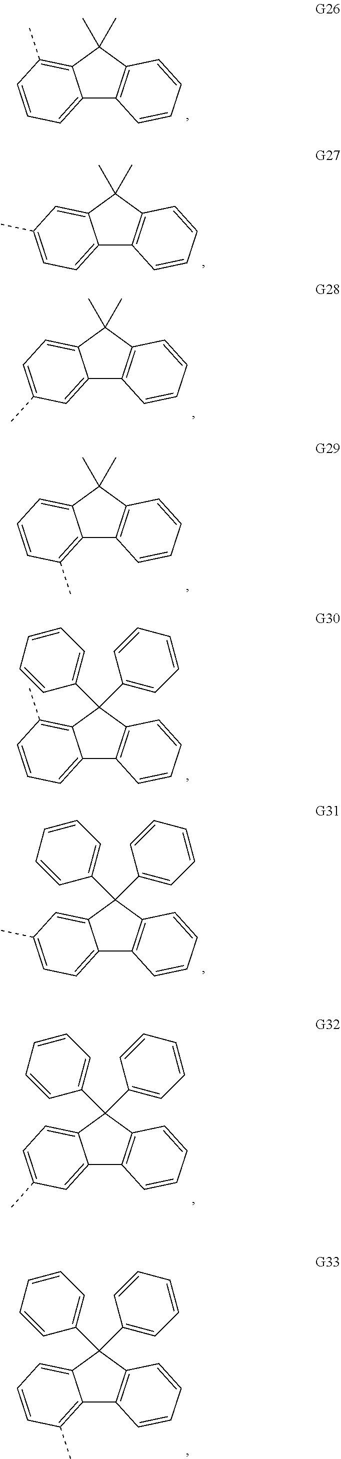 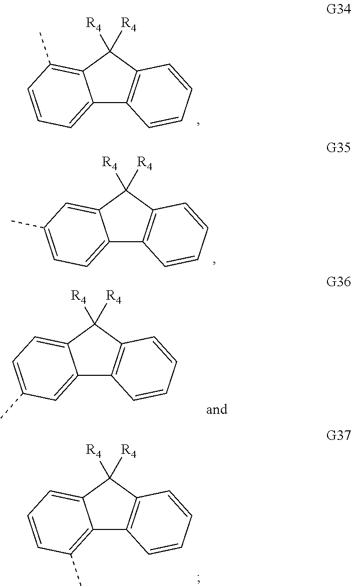
R₄ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents R₄ can be optionally joined to form a ring;
preferably, R₄ is, at each occurrence identically or differently, selected from hydrogen, deuterium, substituted or unsubstituted alkyl having 1 to 20 carbon atoms, substituted or unsubstituted aryl having 6 to 30 carbon atoms, substituted or unsubstituted heteroaryl having 3 to 30 carbon atoms or a combination thereof,
more preferably, R₄ is, at each occurrence identically or differently, selected from hydrogen, deuterium, methyl, ethyl, isopropyl, fluorenyl, phenyl, biphenyl, naphthyl or a combination thereof.

17. The organic electroluminescent device of claim 1, wherein R₁ is, at each occurrence identically or differently, selected from hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms or a combination thereof,
preferably, R₁ is, at each occurrence identically or differently, selected from hydrogen, deuterium, fluorine, methyl, n-propyl, isopropyl, n-butyl, isobutyl, sec-butyl, t-butyl, 2-methylbutyl, n-pentyl, sec-pentyl, neopentyl, cyclopentyl, n-hexyl, neohexyl, cyclohexyl, n-heptyl, phenyl, biphenyl, terphenyl, naphthyl, fluorenyl or a combination thereof.

18. The organic electroluminescent device of claim 1, wherein the Ar₁ and Ar₂ in the second compound are joined to form a ring;
preferably, L₁ and L₂ each are a single bond;
more preferably, the second compound has a structure represented by Formula 2-25: 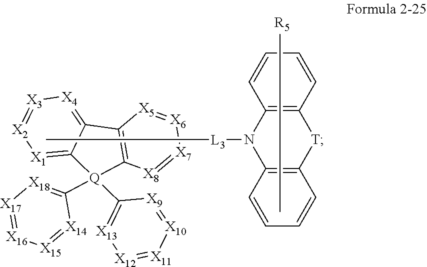
wherein
X₁ to X₈ are, at each occurrence identically or differently, selected from C, CR₁ or N;
X₉ to X₁₈ are, at each occurrence identically or differently, selected from CR₁ or N;
Q is, at each occurrence, selected from C, Si or Ge;
T is, at each occurrence identically or differently, selected from CR₅′R₅′, O, S or NR₅′;
R₅ represents, at each occurrence identically or differently, mono-substitution, multiple substitutions or non-substitution;
R₅ and R₅′ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof,
L₃ is, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof, and
adjacent substituents R₁, R₅ and R₅′ can be optionally joined to form a ring.

19. The organic electroluminescent device of claim 1, wherein the second compound is selected from Compound I-1 to Compound I-273, Compound II-1 to Compound II-332, Compound III-1 to Compound III-273, Compound IV-1 to Compound IV-273, Compound V-1 to Compound V-273, Compound VI-1 to Compound VI-273, Compound VII-1 to Compound VII-273, Compound VIII-1 to Compound VIII-273, Compound IX-1 to Compound IX-273 or Compound X-1 to Compound X273;
wherein Compound I-1 to Compound I-273 each have a structure represented by Formula 2-13: 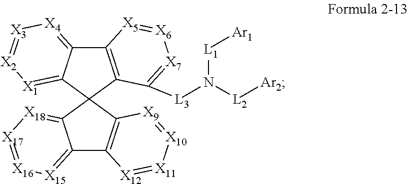
wherein in Formula 2-13, X₁ to X₇, X₉ to X₁₂ and X₁₅ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| I-1 | L-0 | G1 | G1 | I-2 | L-0 | G2 | G2 |
| I-3 | L-0 | G3 | G3 | I-4 | L-0 | G4 | G4 |
| I-5 | L-0 | G10 | G10 | I-6 | L-0 | G11 | G11 |
| I-7 | L-0 | G25 | G25 | | | | |
| | | | | I-12 | L-0 | G31 | G31 |
| I-13 | L-0 | G32 | G32 | I-14 | L-0 | G1 | G2 |
| I-15 | L-0 | G1 | G3 | I-16 | L-0 | G1 | G4 |
| I-17 | L-0 | G1 | G5 | I-18 | L-0 | G1 | G6 |
| I-19 | L-0 | G1 | G7 | I-20 | L-0 | G1 | G8 |
| I-21 | L-0 | G1 | G9 | I-22 | L-0 | G1 | G10 |
| I-23 | L-0 | G1 | G11 | I-24 | L-0 | G1 | G12 |
| I-25 | L-0 | G1 | G13 | I-26 | L-0 | G1 | G14 |
| I-27 | L-0 | G1 | G15 | I-28 | L-0 | G1 | G16 |
| I-29 | L-0 | G1 | G17 | I-30 | L-0 | G1 | G18 |
| I-31 | L-0 | G1 | G19 | I-32 | L-0 | G1 | G20 |
| I-33 | L-0 | G1 | G21 | I-34 | L-0 | G1 | G22 |
| I-35 | L-0 | G1 | G23 | I-36 | L-0 | G1 | G24 |
| I-37 | L-0 | G1 | G25 | I-38 | L-0 | G1 | G26 |
| I-39 | L-0 | G1 | G27 | I-40 | L-0 | G1 | G28 |
| I-41 | L-0 | G1 | G29 | I-42 | L-0 | G1 | G30 |
| I-43 | L-0 | G1 | G31 | I-44 | L-0 | G1 | G32 |
| I-45 | L-0 | G1 | G33 | I-46 | L-0 | G2 | G3 |
| I-47 | L-0 | G2 | G4 | I-48 | L-0 | G2 | G5 |
| I-49 | L-0 | G2 | G6 | I-50 | L-0 | G2 | G7 |
| I-51 | L-0 | G2 | G8 | I-52 | L-0 | G2 | G9 |
| I-53 | L-0 | G2 | G10 | I-54 | L-0 | G2 | G11 |
| I-55 | L-0 | G2 | G12 | I-56 | L-0 | G2 | G13 |
| I-57 | L-0 | G2 | G14 | I-58 | L-0 | G2 | G15 |
| I-59 | L-0 | G2 | G16 | I-60 | L-0 | G2 | G17 |
| I-61 | L-0 | G2 | G18 | I-62 | L-0 | G2 | G19 |
| I-63 | L-0 | G2 | G20 | I-64 | L-0 | G2 | G21 |
| I-65 | L-0 | G2 | G22 | I-66 | L-0 | G2 | G23 |
| I-67 | L-0 | G2 | G24 | I-68 | L-0 | G2 | G25 |
| I-69 | L-0 | G2 | G26 | I-70 | L-0 | G2 | G27 |
| I-71 | L-0 | G2 | G28 | I-72 | L-0 | G2 | G29 |
| I-73 | L-0 | G2 | G30 | I-74 | L-0 | G2 | G31 |
| I-75 | L-0 | G2 | G32 | I-76 | L-0 | G2 | G33 |
| I-77 | L-0 | G3 | G4 | I-78 | L-0 | G3 | G5 |
| I-79 | L-0 | G3 | G6 | I-80 | L-0 | G3 | G7 |
| I-81 | L-0 | G3 | G8 | I-82 | L-0 | G3 | G9 |
| I-83 | L-0 | G3 | G10 | I-84 | L-0 | G3 | G11 |
| I-85 | L-0 | G3 | G12 | I-86 | L-0 | G3 | G13 |
| I-87 | L-0 | G3 | G14 | I-88 | L-0 | G3 | G15 |
| I-89 | L-0 | G3 | G16 | I-90 | L-0 | G3 | G17 |
| I-91 | L-0 | G3 | G18 | I-92 | L-0 | G3 | G19 |
| I-93 | L-0 | G3 | G20 | I-94 | L-0 | G3 | G21 |
| I-95 | L-0 | G3 | G22 | I-96 | L-0 | G3 | G23 |
| I-97 | L-0 | G3 | G24 | I-98 | L-0 | G3 | G25 |
| I-99 | L-0 | G3 | G26 | I-100 | L-0 | G3 | G27 |
| I-101 | L-0 | G3 | G28 | I-102 | L-0 | G3 | G29 |
| I-103 | L-0 | G3 | G30 | I-104 | L-0 | G3 | G31 |
| I-105 | L-0 | G3 | G32 | I-106 | L-0 | G3 | G33 |
| I-107 | L-0 | G4 | G5 | I-108 | L-0 | G4 | G6 |
| I-109 | L-0 | G4 | G7 | I-110 | L-0 | G4 | G8 |
| I-111 | L-0 | G4 | G9 | I-112 | L-0 | G4 | G10 |
| I-113 | L-0 | G4 | G11 | I-114 | L-0 | G4 | G12 |
| I-115 | L-0 | G4 | G13 | I-116 | L-0 | G4 | G14 |
| I-117 | L-0 | G4 | G15 | I-118 | L-0 | G4 | G16 |
| I-119 | L-0 | G4 | G17 | I-120 | L-0 | G4 | G18 |
| I-121 | L-0 | G4 | G19 | I-122 | L-0 | G4 | G20 |
| I-123 | L-0 | G4 | G21 | I-124 | L-0 | G4 | G22 |
| I-125 | L-0 | G4 | G23 | I-126 | L-0 | G4 | G24 |
| I-127 | L-0 | G4 | G25 | I-128 | L-0 | G4 | G26 |
| I-129 | L-0 | G4 | G27 | I-130 | L-0 | G4 | G28 |
| I-131 | L-0 | G4 | G29 | I-132 | L-0 | G4 | G30 |
| I-133 | L-0 | G4 | G31 | I-134 | L-0 | G4 | G32 |
| I-135 | L-0 | G4 | G33 | I-136 | L-0 | G5 | G6 |
| I-137 | L-0 | G5 | G7 | I-138 | L-0 | G5 | G8 |
| I-139 | L-0 | G5 | G9 | I-140 | L-0 | G5 | G10 |
| I-141 | L-0 | G5 | G11 | I-142 | L-0 | G5 | G12 |
| I-143 | L-0 | G5 | G13 | I-144 | L-0 | G5 | G14 |
| I-145 | L-0 | G5 | G15 | I-146 | L-0 | G5 | G16 |
| I-147 | L-0 | G5 | G17 | I-148 | L-0 | G5 | G18 |
| I-149 | L-0 | G5 | G19 | I-150 | L-0 | G5 | G20 |
| I-151 | L-0 | G5 | G21 | I-152 | L-0 | G5 | G22 |
| I-153 | L-0 | G5 | G23 | I-154 | L-0 | G5 | G24 |
| I-155 | L-0 | G5 | G25 | I-156 | L-0 | G5 | G26 |
| I-157 | L-0 | G5 | G27 | I-158 | L-0 | G5 | G28 |
| I-159 | L-0 | G5 | G29 | I-160 | L-0 | G5 | G30 |
| I-161 | L-0 | G5 | G31 | I-162 | L-0 | G5 | G32 |
| I-163 | L-0 | G5 | G33 | I-164 | L-0 | G10 | G26 |
| I-165 | L-0 | G10 | G27 | I-166 | L-0 | G10 | G28 |
| I-167 | L-0 | G10 | G29 | I-168 | L-0 | G10 | G30 |
| I-169 | L-0 | G10 | G31 | I-170 | L-0 | G10 | G32 |
| I-171 | L-0 | G10 | G33 | I-172 | L-0 | G11 | G26 |
| I-173 | L-0 | G11 | G27 | I-174 | L-0 | G11 | G28 |
| I-175 | L-0 | G11 | G29 | I-176 | L-0 | G11 | G30 |
| I-177 | L-0 | G11 | G31 | I-178 | L-0 | G11 | G32 |
| I-179 | L-0 | G11 | G33 | I-180 | L-1 | G1 | G1 |
| I-181 | L-1 | G2 | G2 | I-182 | L-1 | G5 | G5 |
| I-185 | L-1 | G31 | G31 | I-186 | L-1 | G32 | G32 |
| I-187 | L-1 | G1 | G2 | I-188 | L-1 | G1 | G3 |
| I-189 | L-1 | G1 | G4 | I-190 | L-1 | G1 | G5 |
| I-191 | L-1 | G1 | G10 | I-192 | L-1 | G1 | G11 |
| I-193 | L-1 | G1 | G19 | I-194 | L-1 | G1 | G20 |
| I-195 | L-1 | G1 | G27 | I-196 | L-1 | G1 | G28 |
| I-197 | L-1 | G1 | G31 | I-198 | L-1 | G1 | G32 |
| I-199 | L-1 | G2 | G3 | I-200 | L-1 | G2 | G4 |
| I-201 | L-1 | G2 | G5 | I-202 | L-1 | G2 | G10 |
| I-203 | L-1 | G2 | G11 | I-204 | L-1 | G2 | G19 |
| I-205 | L-1 | G2 | G20 | I-206 | L-1 | G2 | G27 |
| I-207 | L-1 | G2 | G28 | I-208 | L-1 | G2 | G31 |
| I-209 | L-1 | G2 | G32 | I-210 | L-1 | G4 | G5 |
| I-211 | L-1 | G4 | G10 | I-212 | L-1 | G4 | G11 |
| I-213 | L-1 | G4 | G19 | I-214 | L-1 | G4 | G20 |
| I-215 | L-1 | G4 | G27 | I-216 | L-1 | G4 | G28 |
| I-217 | L-1 | G4 | G31 | I-218 | L-1 | G4 | G32 |
| I-219 | L-1 | G5 | G10 | I-220 | L-1 | G5 | G11 |
| I-221 | L-1 | G5 | G19 | I-222 | L-1 | G5 | G20 |
| I-223 | L-1 | G5 | G27 | I-224 | L-1 | G5 | G28 |
| I-225 | L-1 | G5 | G31 | I-226 | L-1 | G5 | G32 |
| I-227 | L-4 | G1 | G1 | I-228 | L-4 | G2 | G2 |
| I-229 | L-4 | G5 | G5 | | | | |
| | | | | I-232 | L-4 | G31 | G31 |
| I-233 | L-4 | G32 | G32 | I-234 | L-4 | G1 | G2 |
| I-235 | L-4 | G1 | G3 | I-236 | L-4 | G1 | G4 |
| I-237 | L-4 | G1 | G5 | I-238 | L-4 | G1 | G10 |
| I-239 | L-4 | G1 | G11 | I-240 | L-4 | G1 | G19 |
| I-241 | L-4 | G1 | G20 | I-242 | L-4 | G1 | G27 |
| I-243 | L-4 | G1 | G28 | I-244 | L-4 | G1 | G31 |
| I-245 | L-4 | G1 | G32 | I-246 | L-4 | G2 | G3 |
| I-247 | L-4 | G2 | G4 | I-248 | L-4 | G2 | G5 |
| I-249 | L-4 | G2 | G10 | I-250 | L-4 | G2 | G11 |
| I-251 | L-4 | G2 | G19 | I-252 | L-4 | G2 | G20 |
| I-253 | L-4 | G2 | G27 | I-254 | L-4 | G2 | G28 |
| I-255 | L-4 | G2 | G31 | I-256 | L-4 | G2 | G32 |
| I-257 | L-4 | G4 | G5 | I-258 | L-4 | G4 | G10 |
| I-259 | L-4 | G4 | G11 | I-260 | L-4 | G4 | G19 |
| I-261 | L-4 | G4 | G20 | I-262 | L-4 | G4 | G27 |
| I-263 | L-4 | G4 | G28 | I-264 | L-4 | G4 | G31 |
| I-265 | L-4 | G4 | G32 | I-266 | L-4 | G5 | G10 |
| I-267 | L-4 | G5 | G11 | I-268 | L-4 | G5 | G19 |
| I-269 | L-4 | G5 | G20 | I-270 | L-4 | G5 | G27 |
| I-271 | L-4 | G5 | G28 | I-272 | L-4 | G5 | G31 |
| I-273 | L-4 | G5 | G32 | | | | |

wherein Compound II-1 to Compound II-332 are shown as follows: 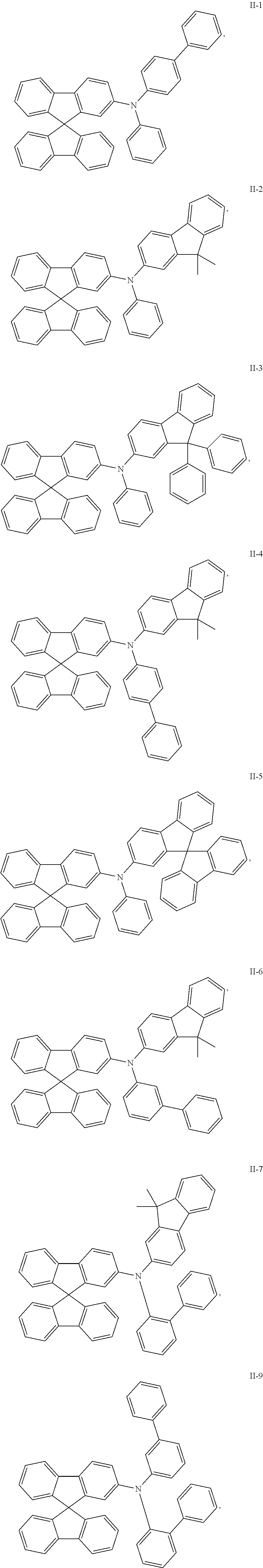 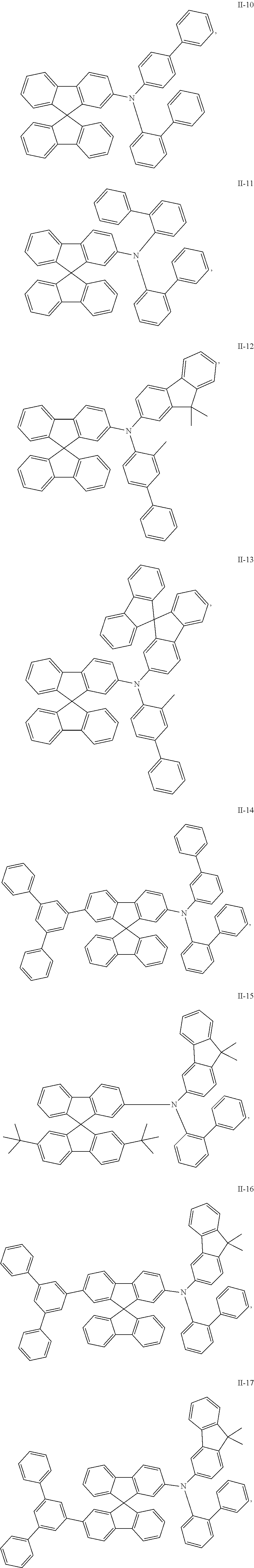 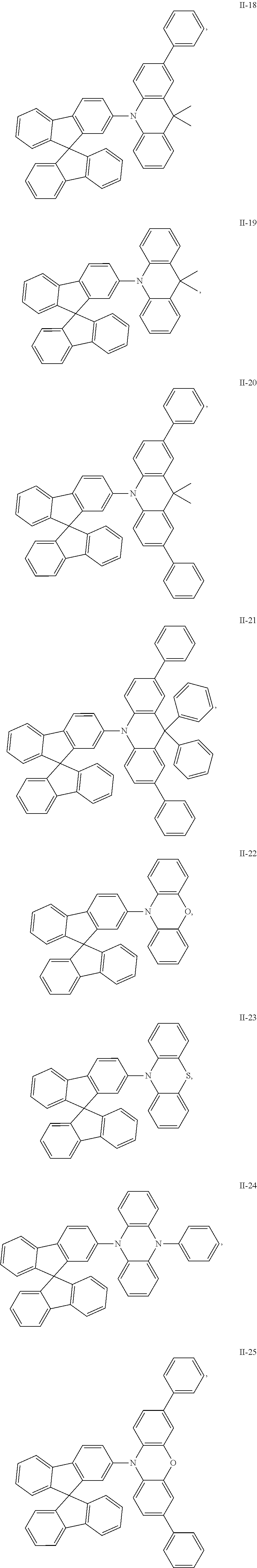 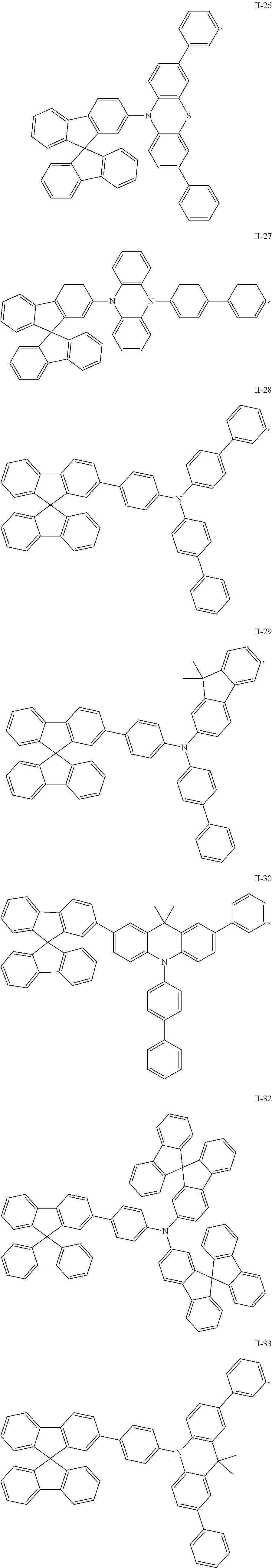 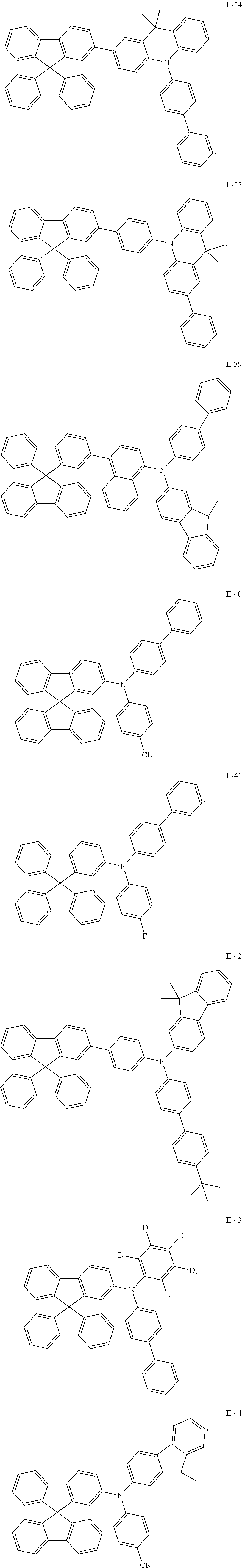 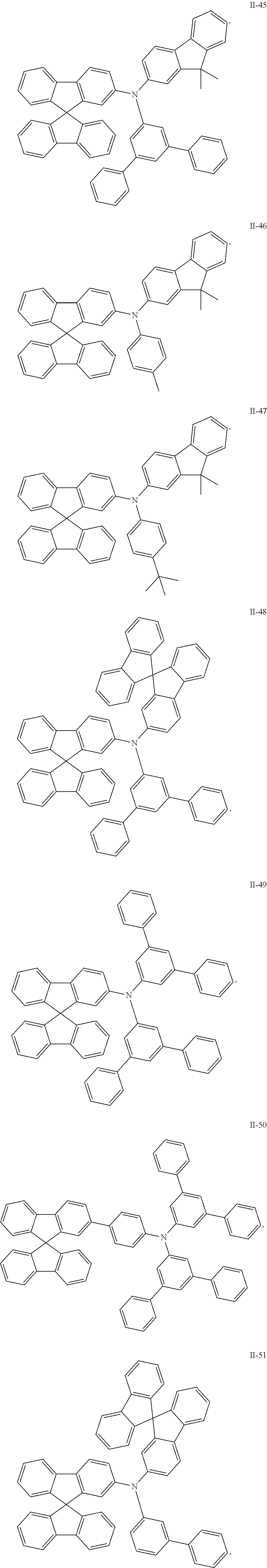 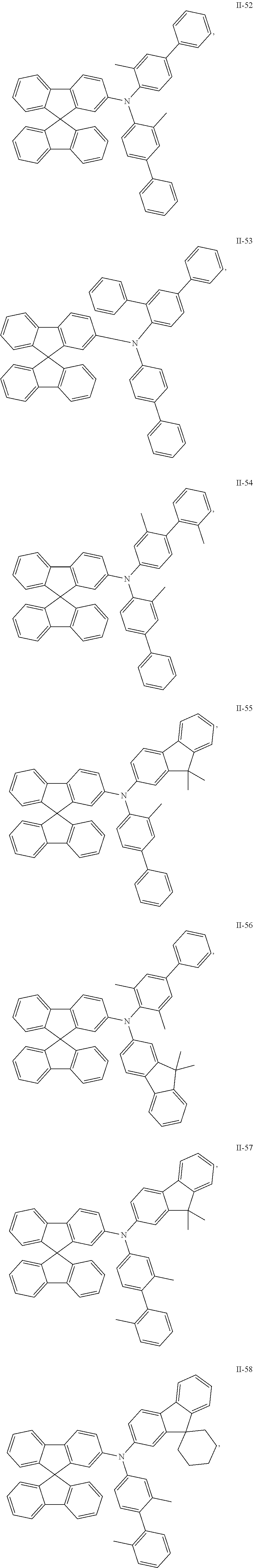 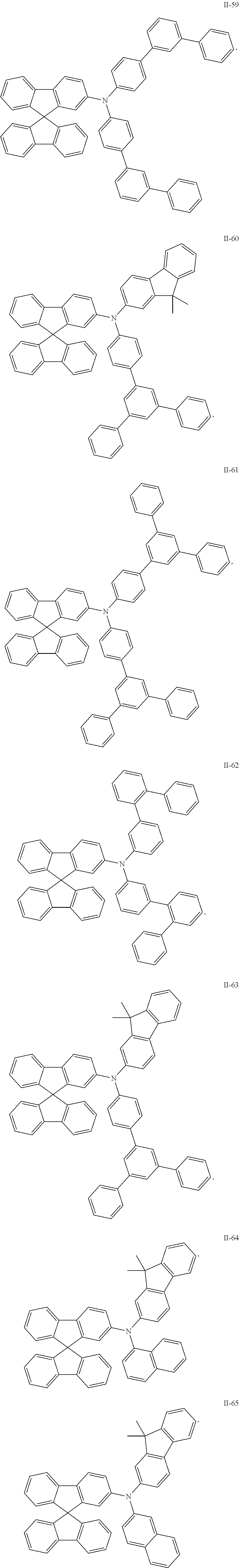 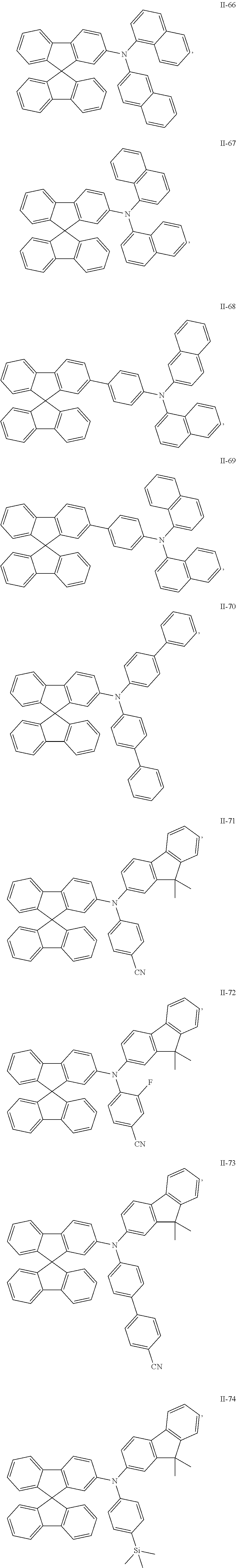 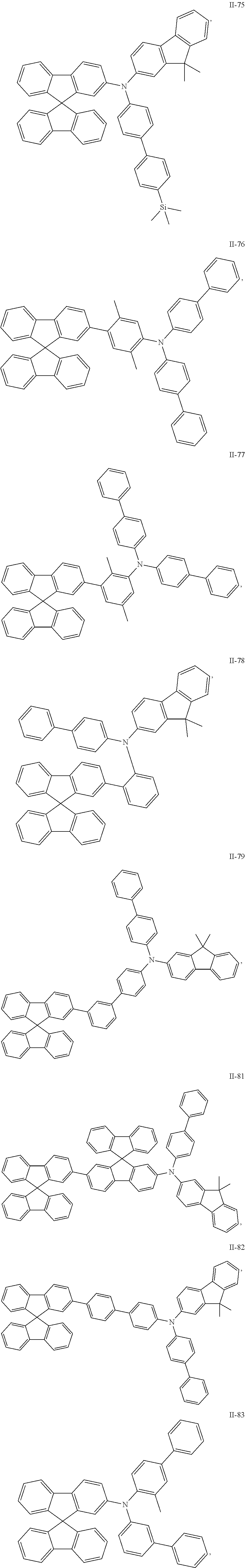 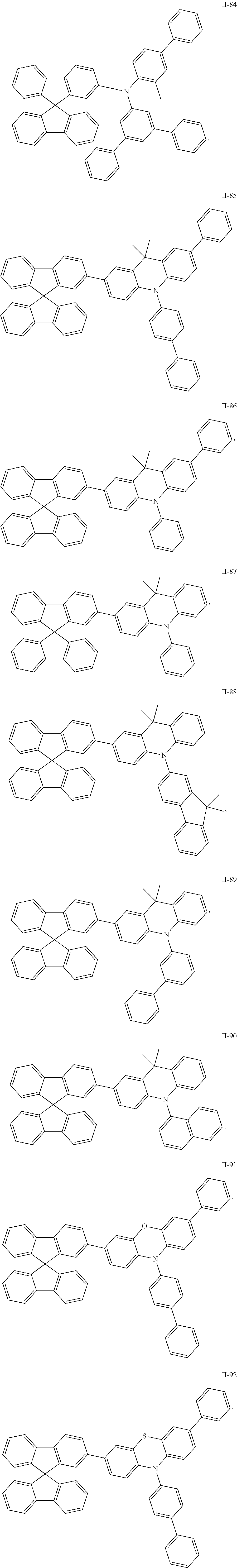 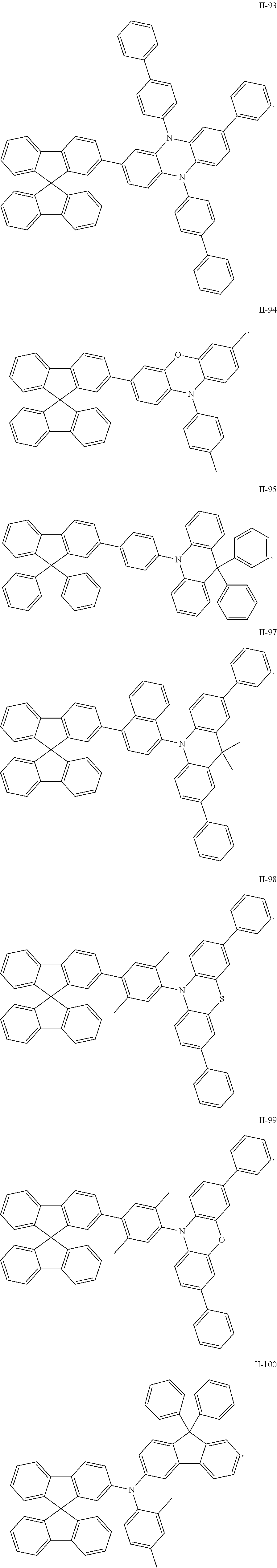 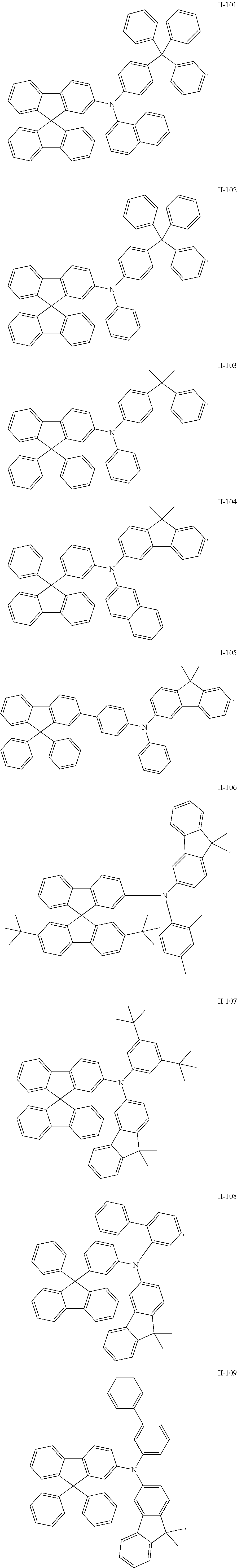 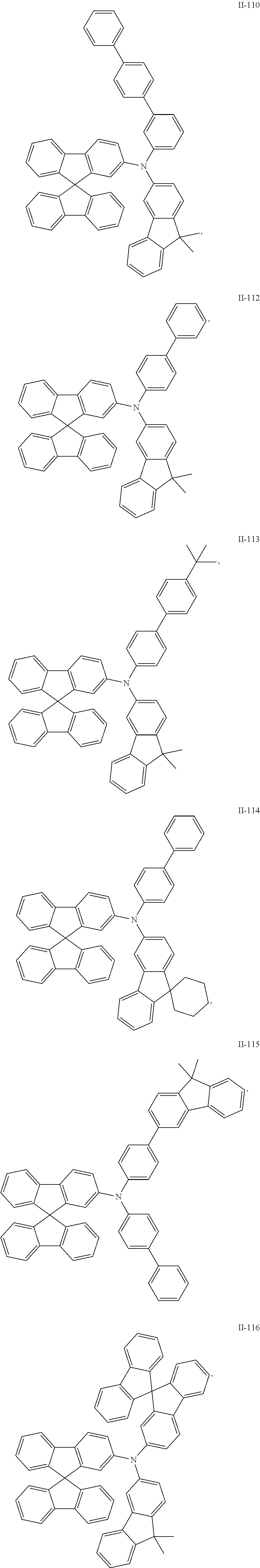 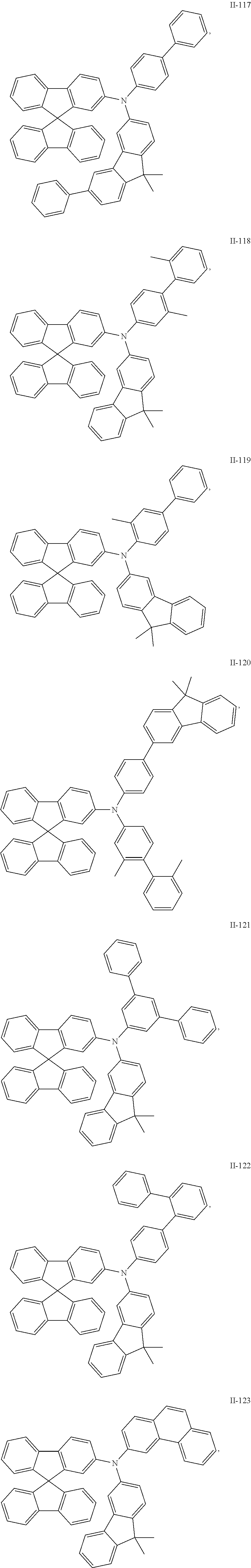 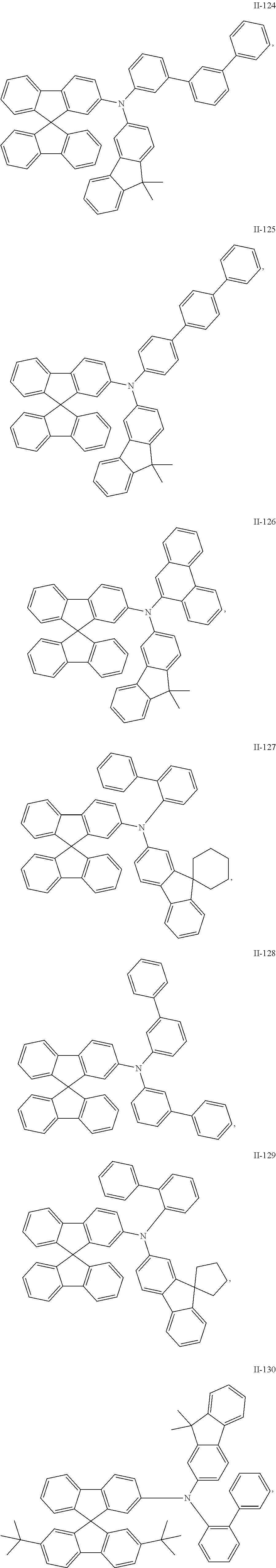 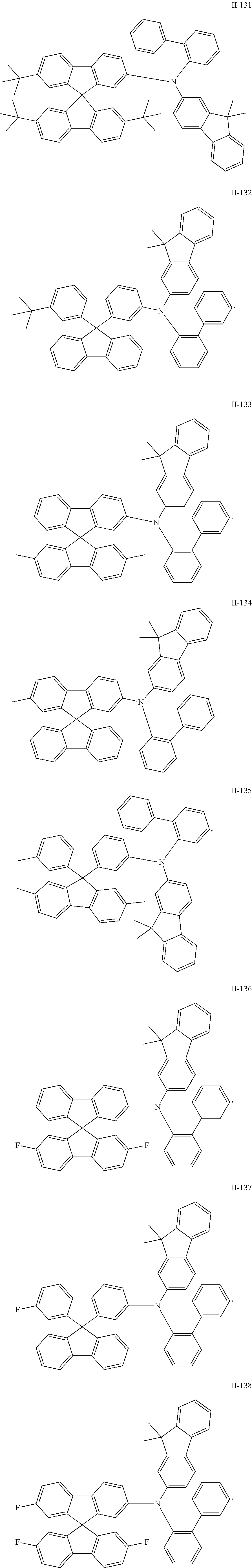 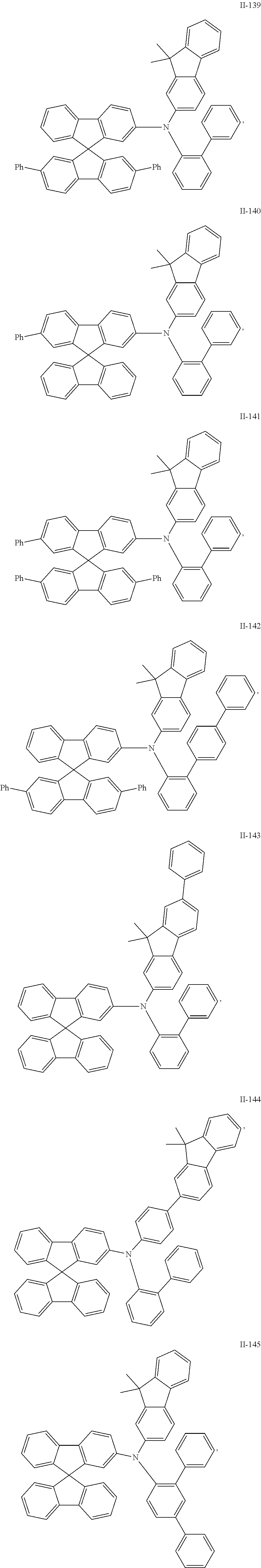 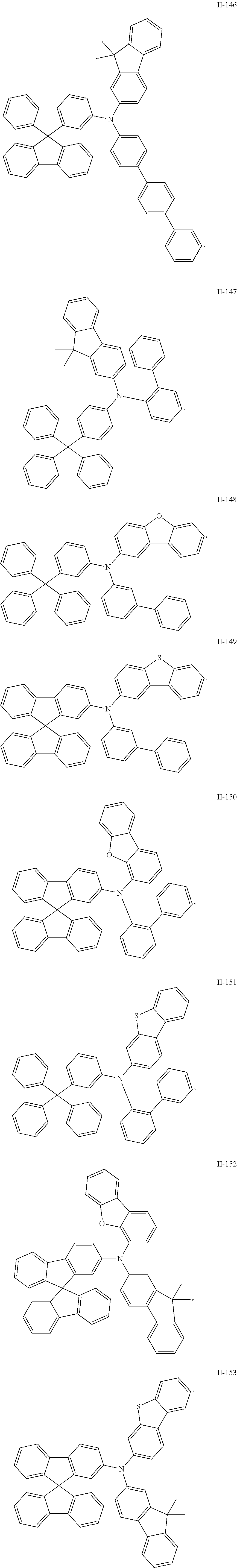 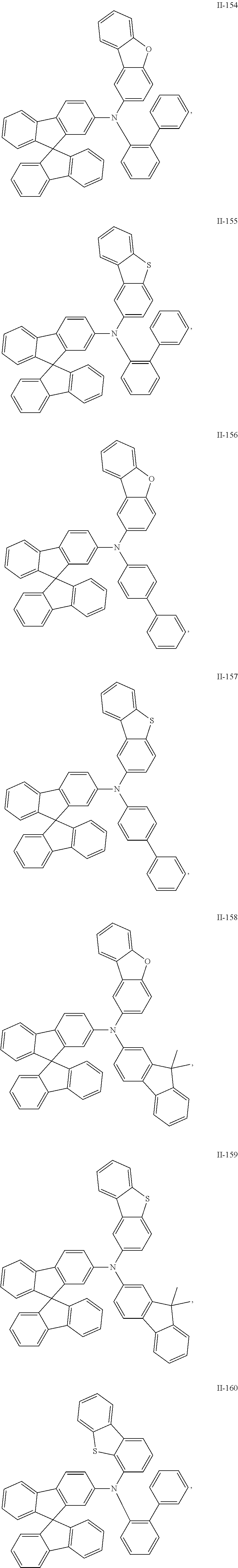 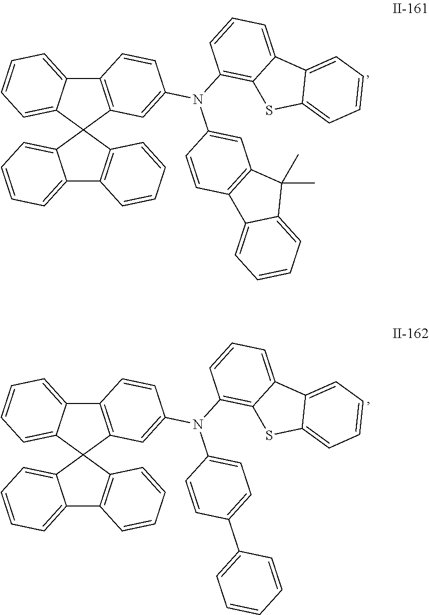 [SMILES: CC1(C)c2ccccc2-c2ccc(N(c3ccc4c(c3)C3(c5ccccc5-c5ccccc53)c3ccccc3-4)c3cccc4c3sc3ccccc34)cc21 ; c1ccc(-c2ccc(N(c3ccc4c(c3)C3(c5ccccc5-c5ccccc53)c3ccccc3-4)c3cccc4c3sc3ccccc34)cc2)cc1] 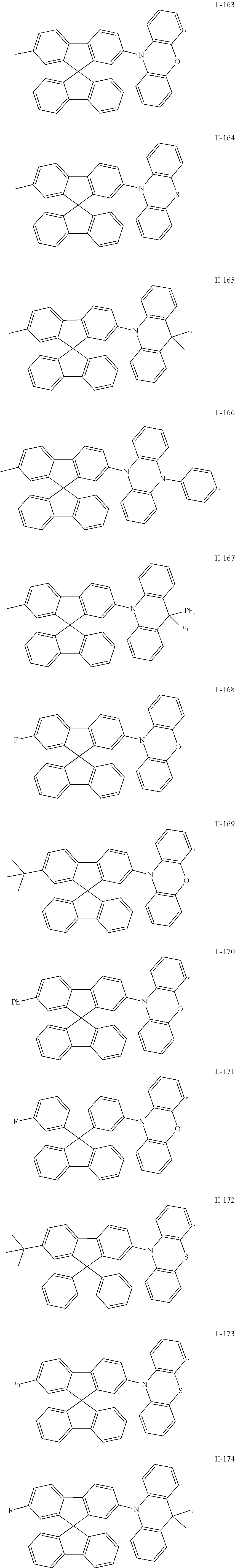 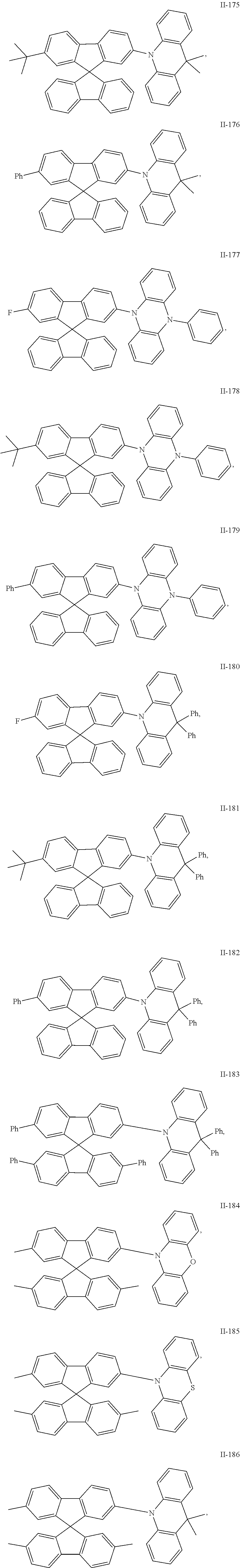 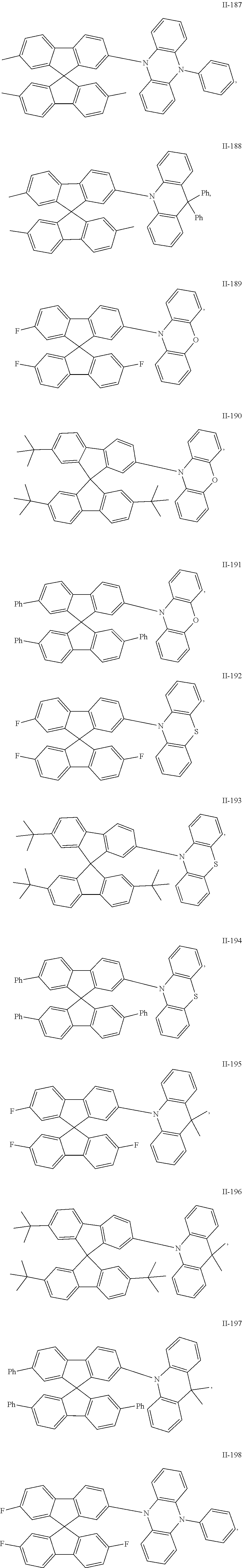 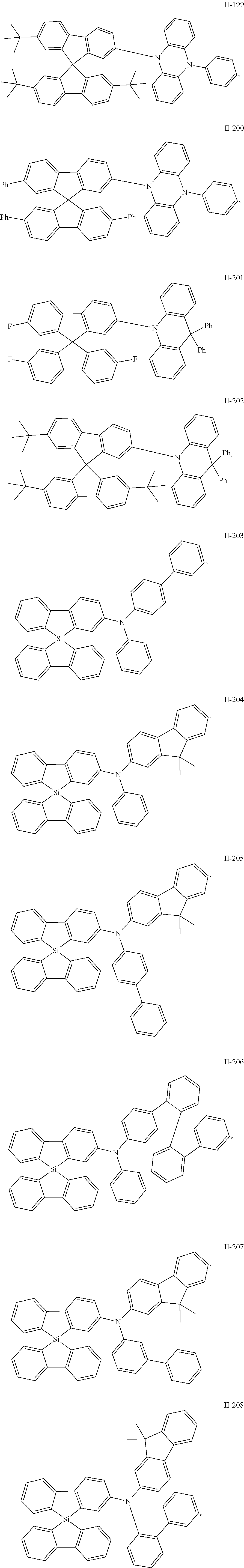 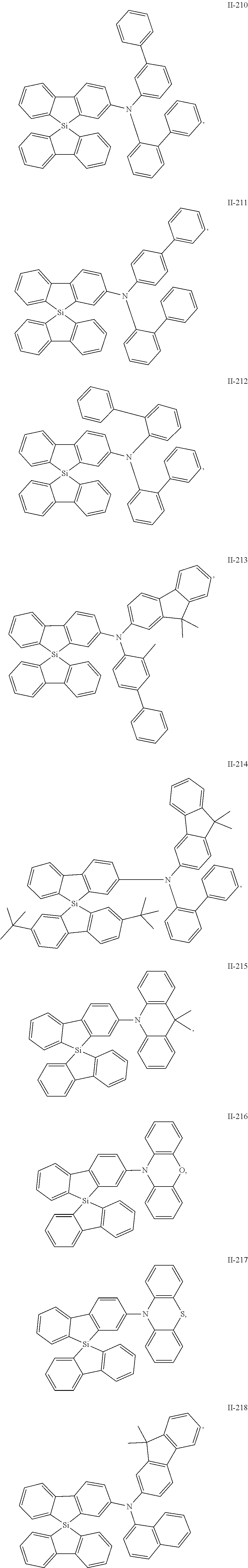 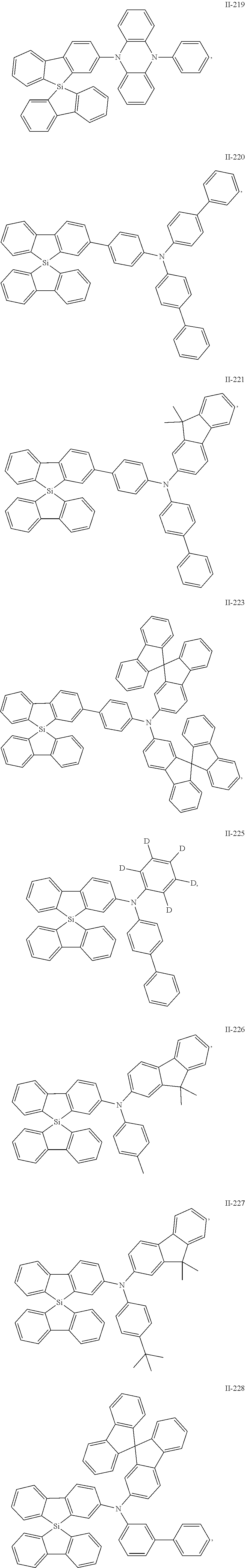 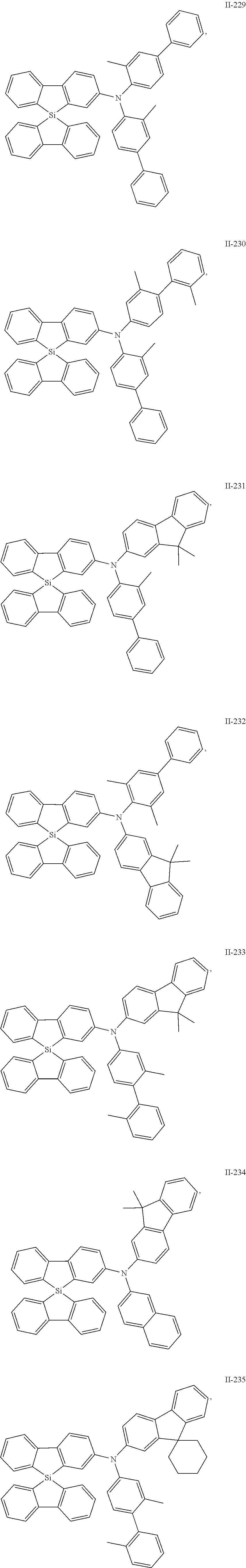 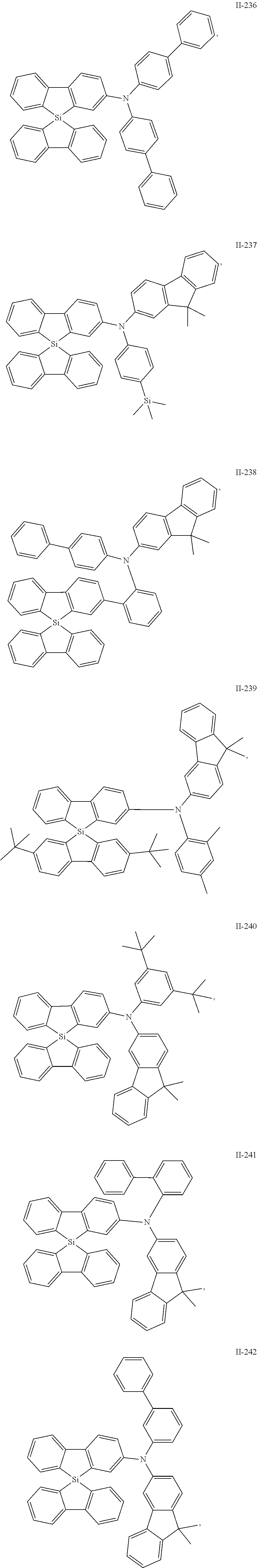 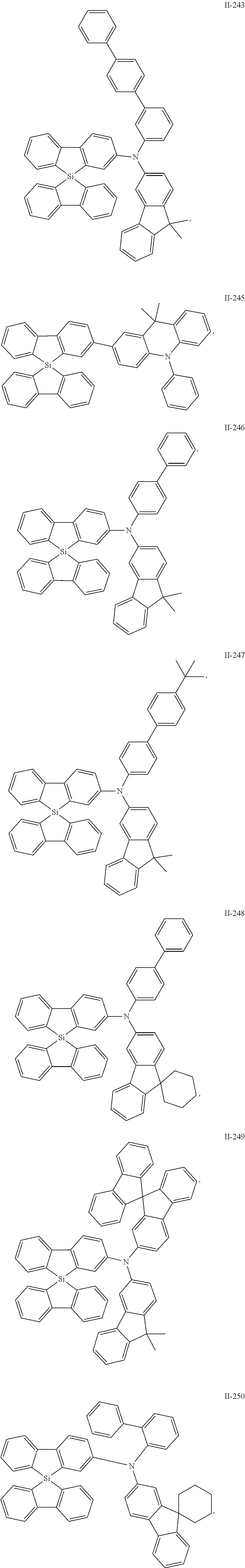 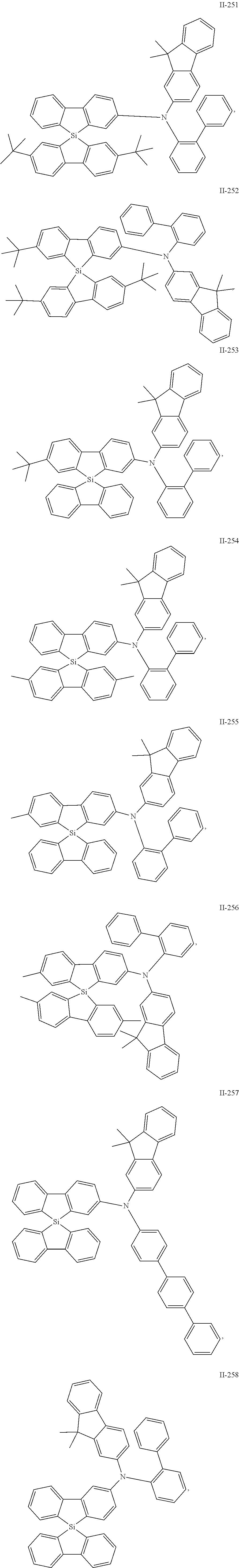 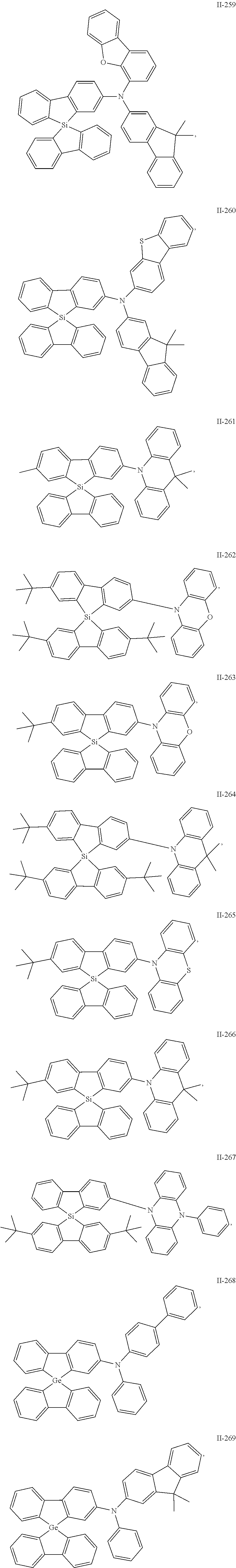 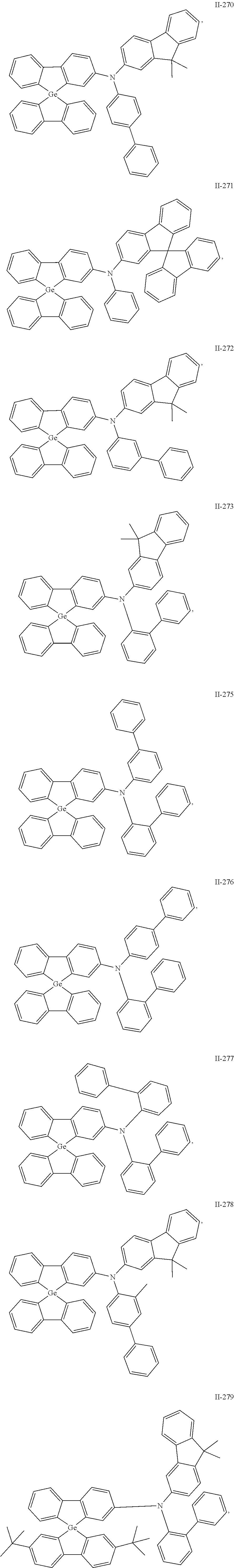 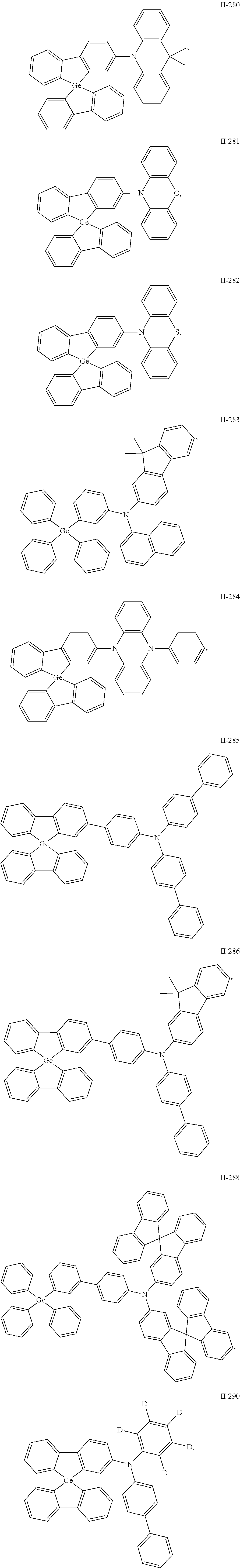 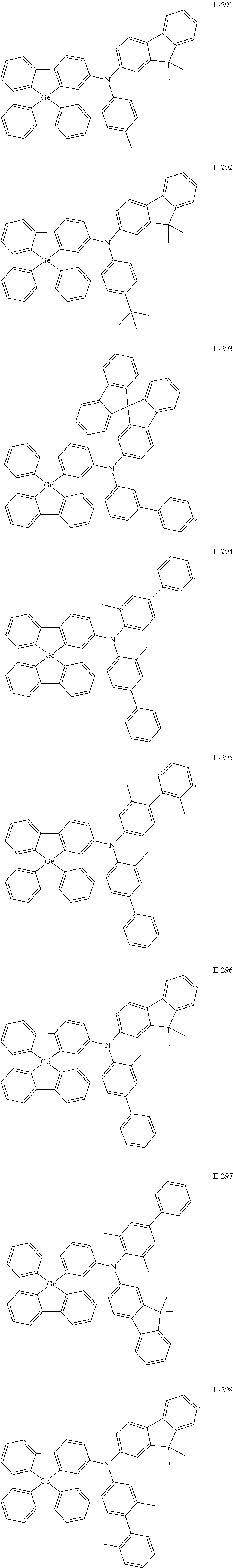 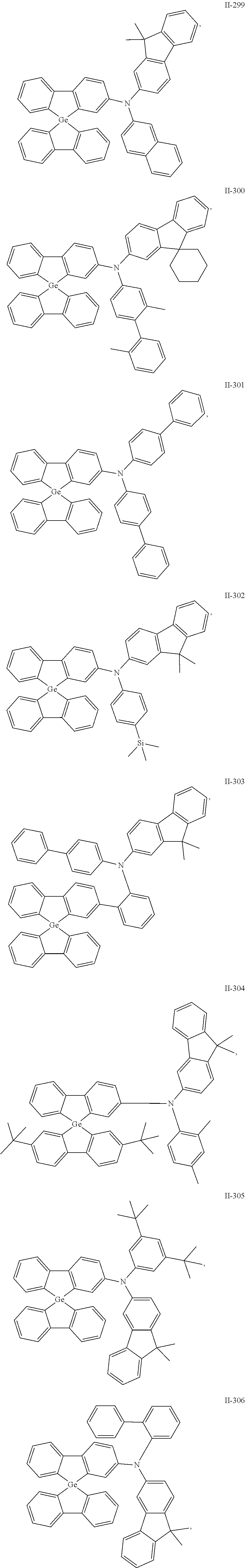 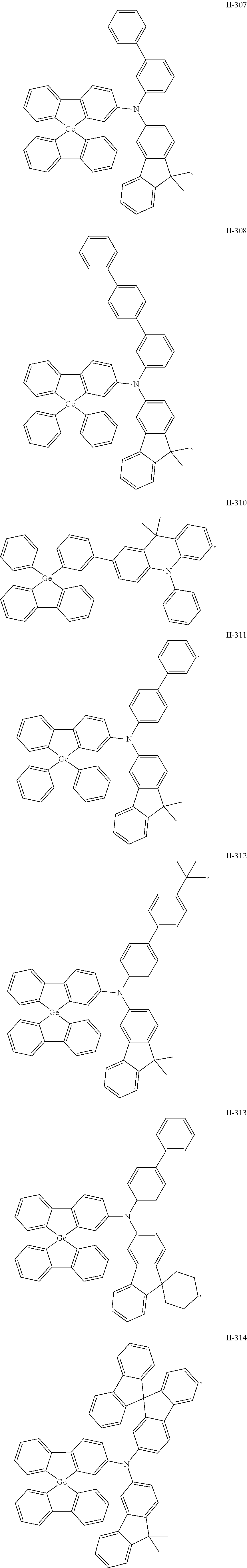 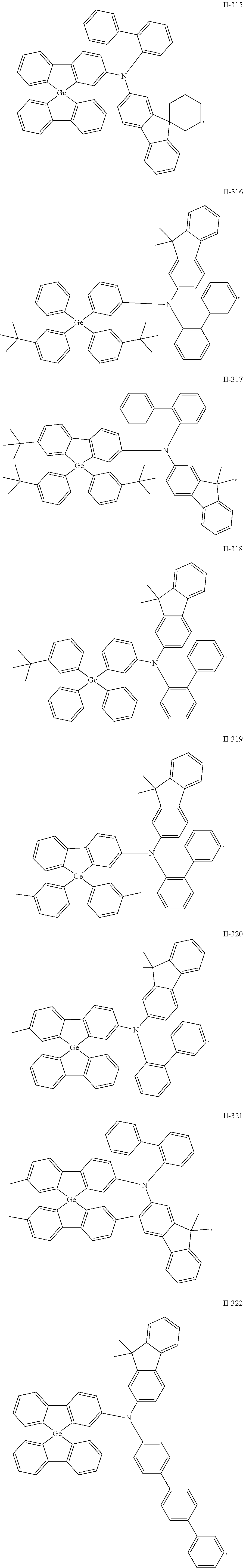 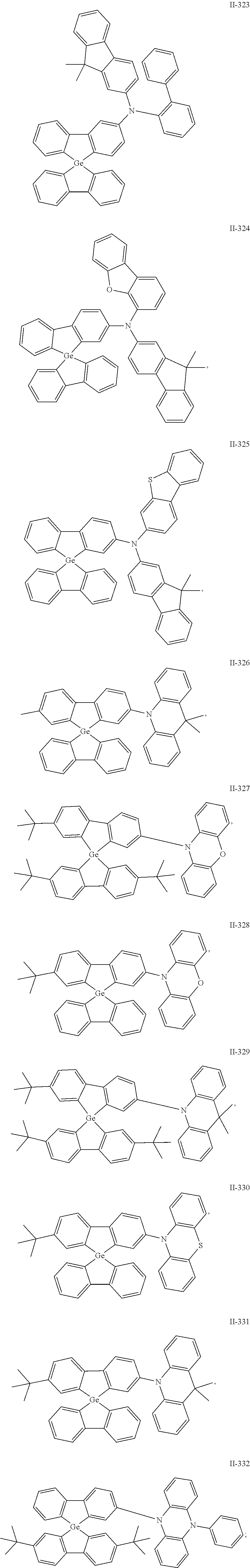
wherein in the structures of Compound II-1 to Compound II-332, Ph represents phenyl;
wherein Compound III-1 to Compound III-273 each have a structure represented by Formula 2-15: 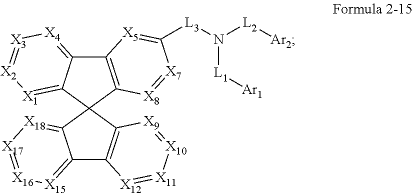
wherein in Formula 2-15, X₁ to X₅, X₇ to X₁₂ and X₁₅ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| III-1 | L-0 | G1 | G1 | III-2 | L-0 | G2 | G2 |
| III-3 | L-0 | G3 | G3 | III-4 | L-0 | G4 | G4 |
| III-5 | L-0 | G10 | G10 | III-6 | L-0 | G11 | G11 |
| III-7 | L-0 | G25 | G25 | | | | |
| | | | | III-12 | L-0 | G31 | G31 |
| III-13 | L-0 | G32 | G32 | III-14 | L-0 | G1 | G2 |
| III-15 | L-0 | G1 | G3 | III-16 | L-0 | G1 | G4 |
| III-17 | L-0 | G1 | G5 | III-18 | L-0 | G1 | G6 |
| III-19 | L-0 | G1 | G7 | III-20 | L-0 | G1 | G8 |
| III-21 | L-0 | G1 | G9 | III-22 | L-0 | G1 | G10 |
| III-23 | L-0 | G1 | G11 | III-24 | L-0 | G1 | G12 |
| III-25 | L-0 | G1 | G13 | III-26 | L-0 | G1 | G14 |
| III-27 | L-0 | G1 | G15 | III-28 | L-0 | G1 | G16 |
| III-29 | L-0 | G1 | G17 | III-30 | L-0 | G1 | G18 |
| III-31 | L-0 | G1 | G19 | III-32 | L-0 | G1 | G20 |
| III-33 | L-0 | G1 | G21 | III-34 | L-0 | G1 | G22 |
| III-35 | L-0 | G1 | G23 | III-36 | L-0 | G1 | G24 |
| III-37 | L-0 | G1 | G25 | III-38 | L-0 | G1 | G26 |
| III-39 | L-0 | G1 | G27 | III-40 | L-0 | G1 | G28 |
| III-41 | L-0 | G1 | G29 | III-42 | L-0 | G1 | G30 |
| III-43 | L-0 | G1 | G31 | III-44 | L-0 | G1 | G32 |
| III-45 | L-0 | G1 | G33 | III-46 | L-0 | G2 | G3 |
| III-47 | L-0 | G2 | G4 | III-48 | L-0 | G2 | G5 |
| III-49 | L-0 | G2 | G6 | III-50 | L-0 | G2 | G7 |
| III-51 | L-0 | G2 | G8 | III-52 | L-0 | G2 | G9 |
| III-53 | L-0 | G2 | G10 | III-54 | L-0 | G2 | G11 |
| III-55 | L-0 | G2 | G12 | III-56 | L-0 | G2 | G13 |
| III-57 | L-0 | G2 | G14 | III-58 | L-0 | G2 | G15 |
| III-59 | L-0 | G2 | G16 | III-60 | L-0 | G2 | G17 |
| III-61 | L-0 | G2 | G18 | III-62 | L-0 | G2 | G19 |
| III-63 | L-0 | G2 | G20 | III-64 | L-0 | G2 | G21 |
| III-65 | L-0 | G2 | G22 | III-66 | L-0 | G2 | G23 |
| III-67 | L-0 | G2 | G24 | III-68 | L-0 | G2 | G25 |
| III-69 | L-0 | G2 | G26 | III-70 | L-0 | G2 | G27 |
| III-71 | L-0 | G2 | G28 | III-72 | L-0 | G2 | G29 |
| III-73 | L-0 | G2 | G30 | III-74 | L-0 | G2 | G31 |
| III-75 | L-0 | G2 | G32 | III-76 | L-0 | G2 | G33 |
| III-77 | L-0 | G3 | G4 | III-78 | L-0 | G3 | G5 |
| III-79 | L-0 | G3 | G6 | III-80 | L-0 | G3 | G7 |
| III-81 | L-0 | G3 | G8 | III-82 | L-0 | G3 | G9 |
| III-83 | L-0 | G3 | G10 | III-84 | L-0 | G3 | G11 |
| III-85 | L-0 | G3 | G12 | III-86 | L-0 | G3 | G13 |
| III-87 | L-0 | G3 | G14 | III-88 | L-0 | G3 | G15 |
| III-89 | L-0 | G3 | G16 | III-90 | L-0 | G3 | G17 |
| III-91 | L-0 | G3 | G18 | III-92 | L-0 | G3 | G19 |
| III-93 | L-0 | G3 | G20 | III-94 | L-0 | G3 | G21 |
| III-95 | L-0 | G3 | G22 | III-96 | L-0 | G3 | G23 |
| III-97 | L-0 | G3 | G24 | III-98 | L-0 | G3 | G25 |
| III-99 | L-0 | G3 | G26 | III-100 | L-0 | G3 | G27 |
| III-101 | L-0 | G3 | G28 | III-102 | L-0 | G3 | G29 |
| III-103 | L-0 | G3 | G30 | III-104 | L-0 | G3 | G31 |
| III-105 | L-0 | G3 | G32 | III-106 | L-0 | G3 | G33 |
| III-107 | L-0 | G4 | G5 | III-108 | L-0 | G4 | G6 |
| III-109 | L-0 | G4 | G7 | III-110 | L-0 | G4 | G8 |
| III-111 | L-0 | G4 | G9 | III-112 | L-0 | G4 | G10 |
| III-113 | L-0 | G4 | G11 | III-114 | L-0 | G4 | G12 |
| III-115 | L-0 | G4 | G13 | III-116 | L-0 | G4 | G14 |
| III-117 | L-0 | G4 | G15 | III-118 | L-0 | G4 | G16 |
| III-119 | L-0 | G4 | G17 | III-120 | L-0 | G4 | G18 |
| III-121 | L-0 | G4 | G19 | III-122 | L-0 | G4 | G20 |
| III-123 | L-0 | G4 | G21 | III-124 | L-0 | G4 | G22 |
| III-125 | L-0 | G4 | G23 | III-126 | L-0 | G4 | G24 |
| III-127 | L-0 | G4 | G25 | III-128 | L-0 | G4 | G26 |
| III-129 | L-0 | G4 | G27 | III-130 | L-0 | G4 | G28 |
| III-131 | L-0 | G4 | G29 | III-132 | L-0 | G4 | G30 |
| III-133 | L-0 | G4 | G31 | III-134 | L-0 | G4 | G32 |
| III-135 | L-0 | G4 | G33 | III-136 | L-0 | G5 | G6 |
| III-137 | L-0 | G5 | G7 | III-138 | L-0 | G5 | G8 |
| III-139 | L-0 | G5 | G9 | III-140 | L-0 | G5 | G10 |
| III-141 | L-0 | G5 | G11 | III-142 | L-0 | G5 | G12 |
| III-143 | L-0 | G5 | G13 | III-144 | L-0 | G5 | G14 |
| III-145 | L-0 | G5 | G15 | III-146 | L-0 | G5 | G16 |
| III-147 | L-0 | G5 | G17 | III-148 | L-0 | G5 | G18 |
| III-149 | L-0 | G5 | G19 | III-150 | L-0 | G5 | G20 |
| III-151 | L-0 | G5 | G21 | III-152 | L-0 | G5 | G22 |
| III-153 | L-0 | G5 | G23 | III-154 | L-0 | G5 | G24 |
| III-155 | L-0 | G5 | G25 | III-156 | L-0 | G5 | G26 |
| III-157 | L-0 | G5 | G27 | III-158 | L-0 | G5 | G28 |
| III-159 | L-0 | G5 | G29 | III-160 | L-0 | G5 | G30 |
| III-161 | L-0 | G5 | G31 | III-162 | L-0 | G5 | G32 |
| III-163 | L-0 | G5 | G33 | III-164 | L-0 | G10 | G26 |
| III-165 | L-0 | G10 | G27 | III-166 | L-0 | G10 | G28 |
| III-167 | L-0 | G10 | G29 | III-168 | L-0 | G10 | G30 |
| III-169 | L-0 | G10 | G31 | III-170 | L-0 | G10 | G32 |
| III-171 | L-0 | G10 | G33 | III-172 | L-0 | G11 | G26 |
| III-173 | L-0 | G11 | G27 | III-174 | L-0 | G11 | G28 |
| III-175 | L-0 | G11 | G29 | III-176 | L-0 | G11 | G30 |
| III-177 | L-0 | G11 | G31 | III-178 | L-0 | G11 | G32 |
| III-179 | L-0 | G11 | G33 | III-180 | L-1 | G1 | G1 |
| III-181 | L-1 | G2 | G2 | III-182 | L-1 | G5 | G5 |
| III-185 | L-1 | G31 | G31 | III-186 | L-1 | G32 | G32 |
| III-187 | L-1 | G1 | G2 | III-188 | L-1 | G1 | G3 |
| III-189 | L-1 | G1 | G4 | III-190 | L-1 | G1 | G5 |
| III-191 | L-1 | G1 | G10 | III-192 | L-1 | G1 | G11 |
| III-193 | L-1 | G1 | G19 | III-194 | L-1 | G1 | G20 |
| III-195 | L-1 | G1 | G27 | III-196 | L-1 | G1 | G28 |
| III-197 | L-1 | G1 | G31 | III-198 | L-1 | G1 | G32 |
| III-199 | L-1 | G2 | G3 | III-200 | L-1 | G2 | G4 |
| III-201 | L-1 | G2 | G5 | III-202 | L-1 | G2 | G10 |
| III-203 | L-1 | G2 | G11 | III-204 | L-1 | G2 | G19 |
| III-205 | L-1 | G2 | G20 | III-206 | L-1 | G2 | G27 |
| III-207 | L-1 | G2 | G28 | III-208 | L-1 | G2 | G31 |
| III-209 | L-1 | G2 | G32 | III-210 | L-1 | G4 | G5 |
| III-211 | L-1 | G4 | G10 | III-212 | L-1 | G4 | G11 |
| III-213 | L-1 | G4 | G19 | III-214 | L-1 | G4 | G20 |
| III-215 | L-1 | G4 | G27 | III-216 | L-1 | G4 | G28 |
| III-217 | L-1 | G4 | G31 | III-218 | L-1 | G4 | G32 |
| III-219 | L-1 | G5 | G10 | III-220 | L-1 | G5 | G11 |
| III-221 | L-1 | G5 | G19 | III-222 | L-1 | G5 | G20 |
| III-223 | L-1 | G5 | G27 | III-224 | L-1 | G5 | G28 |
| III-225 | L-1 | G5 | G31 | III-226 | L-1 | G5 | G32 |
| III-227 | L-4 | G1 | G1 | III-228 | L-4 | G2 | G2 |
| III-229 | L-4 | G5 | G5 | | | | |
| | | | | III-232 | L-4 | G31 | G31 |
| III-233 | L-4 | G32 | G32 | III-234 | L-4 | G1 | G2 |
| III-235 | L-4 | G1 | G3 | III-236 | L-4 | G1 | G4 |
| III-237 | L-4 | G1 | G5 | III-238 | L-4 | G1 | G10 |
| III-239 | L-4 | G1 | G11 | III-240 | L-4 | G1 | G19 |
| III-241 | L-4 | G1 | G20 | III-242 | L-4 | G1 | G27 |
| III-243 | L-4 | G1 | G28 | III-244 | L-4 | G1 | G31 |
| III-245 | L-4 | G1 | G32 | III-246 | L-4 | G2 | G3 |
| III-247 | L-4 | G2 | G4 | III-248 | L-4 | G2 | G5 |
| III-249 | L-4 | G2 | G10 | III-250 | L-4 | G2 | G11 |
| III-251 | L-4 | G2 | G19 | III-252 | L-4 | G2 | G20 |
| III-253 | L-4 | G2 | G27 | III-254 | L-4 | G2 | G28 |
| III-255 | L-4 | G2 | G31 | III-256 | L-4 | G2 | G32 |
| III-257 | L-4 | G4 | G5 | III-258 | L-4 | G4 | G10 |
| III-259 | L-4 | G4 | G11 | III-260 | L-4 | G4 | G19 |
| III-261 | L-4 | G4 | G20 | III-262 | L-4 | G4 | G27 |
| III-263 | L-4 | G4 | G28 | III-264 | L-4 | G4 | G31 |
| III-265 | L-4 | G4 | G32 | III-266 | L-4 | G5 | G10 |
| III-267 | L-4 | G5 | G11 | III-268 | L-4 | G5 | G19 |
| III-269 | L-4 | G5 | G20 | III-270 | L-4 | G5 | G27 |
| III-271 | L-4 | G5 | G28 | III-272 | L-4 | G5 | G31 |
| III-273 | L-4 | G5 | G32 | | | | |

wherein Compound IV-1 to Compound IV-273 each have a structure represented by Formula 2-16: 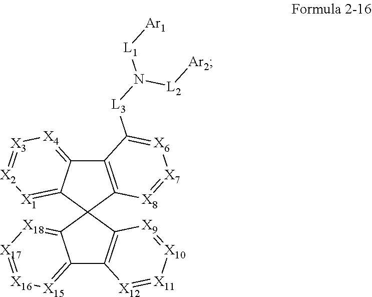
wherein in Formula 2-16, X₁ to X₄, X₆ to X₁₂ and X₁₅ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| IV-1 | L-0 | G1 | G1 | IV-2 | L-0 | G2 | G2 |
| IV-3 | L-0 | G3 | G3 | IV-4 | L-0 | G4 | G4 |
| IV-5 | L-0 | G10 | G10 | IV-6 | L-0 | G11 | G11 |
| IV-7 | L-0 | G25 | G25 | | | | |
| | | | | IV-12 | L-0 | G31 | G31 |
| IV-13 | L-0 | G32 | G32 | IV-14 | L-0 | G1 | G2 |
| IV-15 | L-0 | G1 | G3 | IV-16 | L-0 | G1 | G4 |
| IV-17 | L-0 | G1 | G5 | IV-18 | L-0 | G1 | G6 |
| IV-19 | L-0 | G1 | G7 | IV-20 | L-0 | G1 | G8 |
| IV-21 | L-0 | G1 | G9 | IV-22 | L-0 | G1 | G10 |
| IV-23 | L-0 | G1 | G11 | IV-24 | L-0 | G1 | G12 |
| IV-25 | L-0 | G1 | G13 | IV-26 | L-0 | G1 | G14 |
| IV-27 | L-0 | G1 | G15 | IV-28 | L-0 | G1 | G16 |
| IV-29 | L-0 | G1 | G17 | IV-30 | L-0 | G1 | G18 |
| IV-31 | L-0 | G1 | G19 | IV-32 | L-0 | G1 | G20 |
| IV-33 | L-0 | G1 | G21 | IV-34 | L-0 | G1 | G22 |
| IV-35 | L-0 | G1 | G23 | IV-36 | L-0 | G1 | G24 |
| IV-37 | L-0 | G1 | G25 | IV-38 | L-0 | G1 | G26 |
| IV-39 | L-0 | G1 | G27 | IV-40 | L-0 | G1 | G28 |
| IV-41 | L-0 | G1 | G29 | IV-42 | L-0 | G1 | G30 |
| IV-43 | L-0 | G1 | G31 | IV-44 | L-0 | G1 | G32 |
| IV-45 | L-0 | G1 | G33 | IV-46 | L-0 | G2 | G3 |
| IV-47 | L-0 | G2 | G4 | IV-48 | L-0 | G2 | G5 |
| IV-49 | L-0 | G2 | G6 | IV-50 | L-0 | G2 | G7 |
| IV-51 | L-0 | G2 | G8 | IV-52 | L-0 | G2 | G9 |
| IV-53 | L-0 | G2 | G10 | IV-54 | L-0 | G2 | G11 |
| IV-55 | L-0 | G2 | G12 | IV-56 | L-0 | G2 | G13 |
| IV-57 | L-0 | G2 | G14 | IV-58 | L-0 | G2 | G15 |
| IV-59 | L-0 | G2 | G16 | IV-60 | L-0 | G2 | G17 |
| IV-61 | L-0 | G2 | G18 | IV-62 | L-0 | G2 | G19 |
| IV-63 | L-0 | G2 | G20 | IV-64 | L-0 | G2 | G21 |
| IV-65 | L-0 | G2 | G22 | IV-66 | L-0 | G2 | G23 |
| IV-67 | L-0 | G2 | G24 | IV-68 | L-0 | G2 | G25 |
| IV-69 | L-0 | G2 | G26 | IV-70 | L-0 | G2 | G27 |
| IV-71 | L-0 | G2 | G28 | IV-72 | L-0 | G2 | G29 |
| IV-73 | L-0 | G2 | G30 | IV-74 | L-0 | G2 | G31 |
| IV-75 | L-0 | G2 | G32 | IV-76 | L-0 | G2 | G33 |
| IV-77 | L-0 | G3 | G4 | IV-78 | L-0 | G3 | G5 |
| IV-79 | L-0 | G3 | G6 | IV-80 | L-0 | G3 | G7 |
| IV-81 | L-0 | G3 | G8 | IV-82 | L-0 | G3 | G9 |
| IV-83 | L-0 | G3 | G10 | IV-84 | L-0 | G3 | G11 |
| IV-85 | L-0 | G3 | G12 | IV-86 | L-0 | G3 | G13 |
| IV-87 | L-0 | G3 | G14 | IV-88 | L-0 | G3 | G15 |
| IV-89 | L-0 | G3 | G16 | IV-90 | L-0 | G3 | G17 |
| IV-91 | L-0 | G3 | G18 | IV-92 | L-0 | G3 | G19 |
| IV-93 | L-0 | G3 | G20 | IV-94 | L-0 | G3 | G21 |
| IV-95 | L-0 | G3 | G22 | IV-96 | L-0 | G3 | G23 |
| IV-97 | L-0 | G3 | G24 | IV-98 | L-0 | G3 | G25 |
| IV-99 | L-0 | G3 | G26 | IV-100 | L-0 | G3 | G27 |
| IV-101 | L-0 | G3 | G28 | IV-102 | L-0 | G3 | G29 |
| IV-103 | L-0 | G3 | G30 | IV-104 | L-0 | G3 | G31 |
| IV-105 | L-0 | G3 | G32 | IV-106 | L-0 | G3 | G33 |
| IV-107 | L-0 | G4 | G5 | IV-108 | L-0 | G4 | G6 |
| IV-109 | L-0 | G4 | G7 | IV-110 | L-0 | G4 | G8 |
| IV-111 | L-0 | G4 | G9 | IV-112 | L-0 | G4 | G10 |
| IV-113 | L-0 | G4 | G11 | IV-114 | L-0 | G4 | G12 |
| IV-115 | L-0 | G4 | G13 | IV-116 | L-0 | G4 | G14 |
| IV-117 | L-0 | G4 | G15 | IV-118 | L-0 | G4 | G16 |
| IV-119 | L-0 | G4 | G17 | IV-120 | L-0 | G4 | G18 |
| IV-121 | L-0 | G4 | G19 | IV-122 | L-0 | G4 | G20 |
| IV-123 | L-0 | G4 | G21 | IV-124 | L-0 | G4 | G22 |
| IV-125 | L-0 | G4 | G23 | IV-126 | L-0 | G4 | G24 |
| IV-127 | L-0 | G4 | G25 | IV-128 | L-0 | G4 | G26 |
| IV-129 | L-0 | G4 | G27 | IV-130 | L-0 | G4 | G28 |
| IV-131 | L-0 | G4 | G29 | IV-132 | L-0 | G4 | G30 |
| IV-133 | L-0 | G4 | G31 | IV-134 | L-0 | G4 | G32 |
| IV-135 | L-0 | G4 | G33 | IV-136 | L-0 | G5 | G6 |
| IV-137 | L-0 | G5 | G7 | IV-138 | L-0 | G5 | G8 |
| IV-139 | L-0 | G5 | G9 | IV-140 | L-0 | G5 | G10 |
| IV-141 | L-0 | G5 | G11 | IV-142 | L-0 | G5 | G12 |
| IV-143 | L-0 | G5 | G13 | IV-144 | L-0 | G5 | G14 |
| IV-145 | L-0 | G5 | G15 | IV-146 | L-0 | G5 | G16 |
| IV-147 | L-0 | G5 | G17 | IV-148 | L-0 | G5 | G18 |
| IV-149 | L-0 | G5 | G19 | IV-150 | L-0 | G5 | G20 |
| IV-151 | L-0 | G5 | G21 | IV-152 | L-0 | G5 | G22 |
| IV-153 | L-0 | G5 | G23 | IV-154 | L-0 | G5 | G24 |
| IV-155 | L-0 | G5 | G25 | IV-156 | L-0 | G5 | G26 |
| IV-157 | L-0 | G5 | G27 | IV-158 | L-0 | G5 | G28 |
| IV-159 | L-0 | G5 | G29 | IV-160 | L-0 | G5 | G30 |
| IV-161 | L-0 | G5 | G31 | IV-162 | L-0 | G5 | G32 |
| IV-163 | L-0 | G5 | G33 | IV-164 | L-0 | G10 | G26 |
| IV-165 | L-0 | G10 | G27 | IV-166 | L-0 | G10 | G28 |
| IV-167 | L-0 | G10 | G29 | IV-168 | L-0 | G10 | G30 |
| IV-169 | L-0 | G10 | G31 | IV-170 | L-0 | G10 | G32 |
| IV-171 | L-0 | G10 | G33 | IV-172 | L-0 | G11 | G26 |
| IV-173 | L-0 | G11 | G27 | IV-174 | L-0 | G11 | G28 |
| IV-175 | L-0 | G11 | G29 | IV-176 | L-0 | G11 | G30 |
| IV-177 | L-0 | G11 | G31 | IV-178 | L-0 | G11 | G32 |
| IV-179 | L-0 | G11 | G33 | IV-180 | L-1 | G1 | G1 |
| IV-181 | L-1 | G2 | G2 | IV-182 | L-1 | G5 | G5 |
| IV-185 | L-1 | G31 | G31 | IV-186 | L-1 | G32 | G32 |
| IV-187 | L-1 | G1 | G2 | IV-188 | L-1 | G1 | G3 |
| IV-189 | L-1 | G1 | G4 | IV-190 | L-1 | G1 | G5 |
| IV-191 | L-1 | G1 | G10 | IV-192 | L-1 | G1 | G11 |
| IV-193 | L-1 | G1 | G19 | IV-194 | L-1 | G1 | G20 |
| IV-195 | L-1 | G1 | G27 | IV-196 | L-1 | G1 | G28 |
| IV-197 | L-1 | G1 | G31 | IV-198 | L-1 | G1 | G32 |
| IV-199 | L-1 | G2 | G3 | IV-200 | L-1 | G2 | G4 |
| IV-201 | L-1 | G2 | G5 | IV-202 | L-1 | G2 | G10 |
| IV-203 | L-1 | G2 | G11 | IV-204 | L-1 | G2 | G19 |
| IV-205 | L-1 | G2 | G20 | IV-206 | L-1 | G2 | G27 |
| IV-207 | L-1 | G2 | G28 | IV-208 | L-1 | G2 | G31 |
| IV-209 | L-1 | G2 | G32 | IV-210 | L-1 | G4 | G5 |
| IV-211 | L-1 | G4 | G10 | IV-212 | L-1 | G4 | G11 |
| IV-213 | L-1 | G4 | G19 | IV-214 | L-1 | G4 | G20 |
| IV-215 | L-1 | G4 | G27 | IV-216 | L-1 | G4 | G28 |
| IV-217 | L-1 | G4 | G31 | IV-218 | L-1 | G4 | G32 |
| IV-219 | L-1 | G5 | G10 | IV-220 | L-1 | G5 | G11 |
| IV-221 | L-1 | G5 | G19 | IV-222 | L-1 | G5 | G20 |
| IV-223 | L-1 | G5 | G27 | IV-224 | L-1 | G5 | G28 |
| IV-225 | L-1 | G5 | G31 | IV-226 | L-1 | G5 | G32 |
| IV-227 | L-4 | G1 | G1 | IV-228 | L-4 | G2 | G2 |
| IV-229 | L-4 | G5 | G5 | | | | |
| | | | | IV-232 | L-4 | G31 | G31 |
| IV-233 | L-4 | G32 | G32 | IV-234 | L-4 | G1 | G2 |
| IV-235 | L-4 | G1 | G3 | IV-236 | L-4 | G1 | G4 |
| IV-237 | L-4 | G1 | G5 | IV-238 | L-4 | G1 | G10 |
| IV-239 | L-4 | G1 | G11 | IV-240 | L-4 | G1 | G19 |
| IV-241 | L-4 | G1 | G20 | IV-242 | L-4 | G1 | G27 |
| IV-243 | L-4 | G1 | G28 | IV-244 | L-4 | G1 | G31 |
| IV-245 | L-4 | G1 | G32 | IV-246 | L-4 | G2 | G3 |
| IV-247 | L-4 | G2 | G4 | IV-248 | L-4 | G2 | G5 |
| IV-249 | L-4 | G2 | G10 | IV-250 | L-4 | G2 | G11 |
| IV-251 | L-4 | G2 | G19 | IV-252 | L-4 | G2 | G20 |
| IV-253 | L-4 | G2 | G27 | IV-254 | L-4 | G2 | G28 |
| IV-255 | L-4 | G2 | G31 | IV-256 | L-4 | G2 | G32 |
| IV-257 | L-4 | G4 | G5 | IV-258 | L-4 | G4 | G10 |
| IV-259 | L-4 | G4 | G11 | IV-260 | L-4 | G4 | G19 |
| IV-261 | L-4 | G4 | G20 | IV-262 | L-4 | G4 | G27 |
| IV-263 | L-4 | G4 | G28 | IV-264 | L-4 | G4 | G31 |
| IV-265 | L-4 | G4 | G32 | IV-266 | L-4 | G5 | G10 |
| IV-267 | L-4 | G5 | G11 | IV-268 | L-4 | G5 | G19 |
| IV-269 | L-4 | G5 | G20 | IV-270 | L-4 | G5 | G27 |
| IV-271 | L-4 | G5 | G28 | IV-272 | L-4 | G5 | G31 |
| IV-273 | L-4 | G5 | G32 | | | | |

wherein Compound V-1 to Compound V-273 each have a structure represented by Formula 2-1: 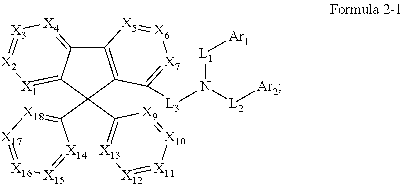
wherein in Formula 2-1, X₁ to X₇ and X₉ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| V-1 | L-0 | G1 | G1 | V-2 | L-0 | G2 | G2 |
| V-3 | L-0 | G3 | G3 | V-4 | L-0 | G4 | G4 |
| V-5 | L-0 | G10 | G10 | V-6 | L-0 | G11 | G11 |
| V-7 | L-0 | G25 | G25 | | | | |
| | | | | V-12 | L-0 | G31 | G31 |
| V-13 | L-0 | G32 | G32 | V-14 | L-0 | G1 | G2 |
| V-15 | L-0 | G1 | G3 | V-16 | L-0 | G1 | G4 |
| V-17 | L-0 | G1 | G5 | V-18 | L-0 | G1 | G6 |
| V-19 | L-0 | G1 | G7 | V-20 | L-0 | G1 | G8 |
| V-21 | L-0 | G1 | G9 | V-22 | L-0 | G1 | G10 |
| V-23 | L-0 | G1 | G11 | V-24 | L-0 | G1 | G12 |
| V-25 | L-0 | G1 | G13 | V-26 | L-0 | G1 | G14 |
| V-27 | L-0 | G1 | G15 | V-28 | L-0 | G1 | G16 |
| V-29 | L-0 | G1 | G17 | V-30 | L-0 | G1 | G18 |
| V-31 | L-0 | G1 | G19 | V-32 | L-0 | G1 | G20 |
| V-33 | L-0 | G1 | G21 | V-34 | L-0 | G1 | G22 |
| V-35 | L-0 | G1 | G23 | V-36 | L-0 | G1 | G24 |
| V-37 | L-0 | G1 | G25 | V-38 | L-0 | G1 | G26 |
| V-39 | L-0 | G1 | G27 | V-40 | L-0 | G1 | G28 |
| V-41 | L-0 | G1 | G29 | V-42 | L-0 | G1 | G30 |
| V-43 | L-0 | G1 | G31 | V-44 | L-0 | G1 | G32 |
| V-45 | L-0 | G1 | G33 | V-46 | L-0 | G2 | G3 |
| V-47 | L-0 | G2 | G4 | V-48 | L-0 | G2 | G5 |
| V-49 | L-0 | G2 | G6 | V-50 | L-0 | G2 | G7 |
| V-51 | L-0 | G2 | G8 | V-52 | L-0 | G2 | G9 |
| V-53 | L-0 | G2 | G10 | V-54 | L-0 | G2 | G11 |
| V-55 | L-0 | G2 | G12 | V-56 | L-0 | G2 | G13 |
| V-57 | L-0 | G2 | G14 | V-58 | L-0 | G2 | G15 |
| V-59 | L-0 | G2 | G16 | V-60 | L-0 | G2 | G17 |
| V-61 | L-0 | G2 | G18 | V-62 | L-0 | G2 | G19 |
| V-63 | L-0 | G2 | G20 | V-64 | L-0 | G2 | G21 |
| V-65 | L-0 | G2 | G22 | V-66 | L-0 | G2 | G23 |
| V-67 | L-0 | G2 | G24 | V-68 | L-0 | G2 | G25 |
| V-69 | L-0 | G2 | G26 | V-70 | L-0 | G2 | G27 |
| V-71 | L-0 | G2 | G28 | V-72 | L-0 | G2 | G29 |
| V-73 | L-0 | G2 | G30 | V-74 | L-0 | G2 | G31 |
| V-75 | L-0 | G2 | G32 | V-76 | L-0 | G2 | G33 |
| V-77 | L-0 | G3 | G4 | V-78 | L-0 | G3 | G5 |
| V-79 | L-0 | G3 | G6 | V-80 | L-0 | G3 | G7 |
| V-81 | L-0 | G3 | G8 | V-82 | L-0 | G3 | G9 |
| V-83 | L-0 | G3 | G10 | V-84 | L-0 | G3 | G11 |
| V-85 | L-0 | G3 | G12 | V-86 | L-0 | G3 | G13 |
| V-87 | L-0 | G3 | G14 | V-88 | L-0 | G3 | G15 |
| V-89 | L-0 | G3 | G16 | V-90 | L-0 | G3 | G17 |
| V-91 | L-0 | G3 | G18 | V-92 | L-0 | G3 | G19 |
| V-93 | L-0 | G3 | G20 | V-94 | L-0 | G3 | G21 |
| V-95 | L-0 | G3 | G22 | V-96 | L-0 | G3 | G23 |
| V-97 | L-0 | G3 | G24 | V-98 | L-0 | G3 | G25 |
| V-99 | L-0 | G3 | G26 | V-100 | L-0 | G3 | G27 |
| V-101 | L-0 | G3 | G28 | V-102 | L-0 | G3 | G29 |
| V-103 | L-0 | G3 | G30 | V-104 | L-0 | G3 | G31 |
| V-105 | L-0 | G3 | G32 | V-106 | L-0 | G3 | G33 |
| V-107 | L-0 | G4 | G5 | V-108 | L-0 | G4 | G6 |
| V-109 | L-0 | G4 | G7 | V-110 | L-0 | G4 | G8 |
| V-111 | L-0 | G4 | G9 | V-112 | L-0 | G4 | G10 |
| V-113 | L-0 | G4 | G11 | V-114 | L-0 | G4 | G12 |
| V-115 | L-0 | G4 | G13 | V-116 | L-0 | G4 | G14 |
| V-117 | L-0 | G4 | G15 | V-118 | L-0 | G4 | G16 |
| V-119 | L-0 | G4 | G17 | V-120 | L-0 | G4 | G18 |
| V-121 | L-0 | G4 | G19 | V-122 | L-0 | G4 | G20 |
| V-123 | L-0 | G4 | G21 | V-124 | L-0 | G4 | G22 |
| V-125 | L-0 | G4 | G23 | V-126 | L-0 | G4 | G24 |
| V-127 | L-0 | G4 | G25 | V-128 | L-0 | G4 | G26 |
| V-129 | L-0 | G4 | G27 | V-130 | L-0 | G4 | G28 |
| V-131 | L-0 | G4 | G29 | V-132 | L-0 | G4 | G30 |
| V-133 | L-0 | G4 | G31 | V-134 | L-0 | G4 | G32 |
| V-135 | L-0 | G4 | G33 | V-136 | L-0 | G5 | G6 |
| V-137 | L-0 | G5 | G7 | V-138 | L-0 | G5 | G8 |
| V-139 | L-0 | G5 | G9 | V-140 | L-0 | G5 | G10 |
| V-141 | L-0 | G5 | G11 | V-142 | L-0 | G5 | G12 |
| V-143 | L-0 | G5 | G13 | V-144 | L-0 | G5 | G14 |
| V-145 | L-0 | G5 | G15 | V-146 | L-0 | G5 | G16 |
| V-147 | L-0 | G5 | G17 | V-148 | L-0 | G5 | G18 |
| V-149 | L-0 | G5 | G19 | V-150 | L-0 | G5 | G20 |
| V-151 | L-0 | G5 | G21 | V-152 | L-0 | G5 | G22 |
| V-153 | L-0 | G5 | G23 | V-154 | L-0 | G5 | G24 |
| V-155 | L-0 | G5 | G25 | V-156 | L-0 | G5 | G26 |
| V-157 | L-0 | G5 | G27 | V-158 | L-0 | G5 | G28 |
| V-159 | L-0 | G5 | G29 | V-160 | L-0 | G5 | G30 |
| V-161 | L-0 | G5 | G31 | V-162 | L-0 | G5 | G32 |
| V-163 | L-0 | G5 | G33 | V-164 | L-0 | G10 | G26 |
| V-165 | L-0 | G10 | G27 | V-166 | L-0 | G10 | G28 |
| V-167 | L-0 | G10 | G29 | V-168 | L-0 | G10 | G30 |
| V-169 | L-0 | G10 | G31 | V-170 | L-0 | G10 | G32 |
| V-171 | L-0 | G10 | G33 | V-172 | L-0 | G11 | G26 |
| V-173 | L-0 | G11 | G27 | V-174 | L-0 | G11 | G28 |
| V-175 | L-0 | G11 | G29 | V-176 | L-0 | G11 | G30 |
| V-177 | L-0 | G11 | G31 | V-178 | L-0 | G11 | G32 |
| V-179 | L-0 | G11 | G33 | V-180 | L-1 | G1 | G1 |
| V-181 | L-1 | G2 | G2 | V-182 | L-1 | G5 | G5 |
| V-185 | L-1 | G31 | G31 | V-186 | L-1 | G32 | G32 |
| V-187 | L-1 | G1 | G2 | V-188 | L-1 | G1 | G3 |
| V-189 | L-1 | G1 | G4 | V-190 | L-1 | G1 | G5 |
| V-191 | L-1 | G1 | G10 | V-192 | L-1 | G1 | G11 |
| V-193 | L-1 | G1 | G19 | V-194 | L-1 | G1 | G20 |
| V-195 | L-1 | G1 | G27 | V-196 | L-1 | G1 | G28 |
| V-197 | L-1 | G1 | G31 | V-198 | L-1 | G1 | G32 |
| V-199 | L-1 | G2 | G3 | V-200 | L-1 | G2 | G4 |
| V-201 | L-1 | G2 | G5 | V-202 | L-1 | G2 | G10 |
| V-203 | L-1 | G2 | G11 | V-204 | L-1 | G2 | G19 |
| V-205 | L-1 | G2 | G20 | V-206 | L-1 | G2 | G27 |
| V-207 | L-1 | G2 | G28 | V-208 | L-1 | G2 | G31 |
| V-209 | L-1 | G2 | G32 | V-210 | L-1 | G4 | G5 |
| V-211 | L-1 | G4 | G10 | V-212 | L-1 | G4 | G11 |
| V-213 | L-1 | G4 | G19 | V-214 | L-1 | G4 | G20 |
| V-215 | L-1 | G4 | G27 | V-216 | L-1 | G4 | G28 |
| V-217 | L-1 | G4 | G31 | V-218 | L-1 | G4 | G32 |
| V-219 | L-1 | G5 | G10 | V-220 | L-1 | G5 | G11 |
| V-221 | L-1 | G5 | G19 | V-222 | L-1 | G5 | G20 |
| V-223 | L-1 | G5 | G27 | V-224 | L-1 | G5 | G28 |
| V-225 | L-1 | G5 | G31 | V-226 | L-1 | G5 | G32 |
| V-227 | L-4 | G1 | G1 | V-228 | L-4 | G2 | G2 |
| V-229 | L-4 | G5 | G5 | | | | |
| | | | | V-232 | L-4 | G31 | G31 |
| V-233 | L-4 | G32 | G32 | V-234 | L-4 | G1 | G2 |
| V-235 | L-4 | G1 | G3 | V-236 | L-4 | G1 | G4 |
| V-237 | L-4 | G1 | G5 | V-238 | L-4 | G1 | G10 |
| V-239 | L-4 | G1 | G11 | V-240 | L-4 | G1 | G19 |
| V-241 | L-4 | G1 | G20 | V-242 | L-4 | G1 | G27 |
| V-243 | L-4 | G1 | G28 | V-244 | L-4 | G1 | G31 |
| V-245 | L-4 | G1 | G32 | V-246 | L-4 | G2 | G3 |
| V-247 | L-4 | G2 | G4 | V-248 | L-4 | G2 | G5 |
| V-249 | L-4 | G2 | G10 | V-250 | L-4 | G2 | G11 |
| V-251 | L-4 | G2 | G19 | V-252 | L-4 | G2 | G20 |
| V-253 | L-4 | G2 | G27 | V-254 | L-4 | G2 | G28 |
| V-255 | L-4 | G2 | G31 | V-256 | L-4 | G2 | G32 |
| V-257 | L-4 | G4 | G5 | V-258 | L-4 | G4 | G10 |
| V-259 | L-4 | G4 | G11 | V-260 | L-4 | G4 | G19 |
| V-261 | L-4 | G4 | G20 | V-262 | L-4 | G4 | G27 |
| V-263 | L-4 | G4 | G28 | V-264 | L-4 | G4 | G31 |
| V-265 | L-4 | G4 | G32 | V-266 | L-4 | G5 | G10 |
| V-267 | L-4 | G5 | G11 | V-268 | L-4 | G5 | G19 |
| V-269 | L-4 | G5 | G20 | V-270 | L-4 | G5 | G27 |
| V-271 | L-4 | G5 | G28 | V-272 | L-4 | G5 | G31 |
| V-273 | L-4 | G5 | G32 | | | | |

wherein Compound VI-1 to Compound VI-273 each have a structure represented by Formula 2-2: 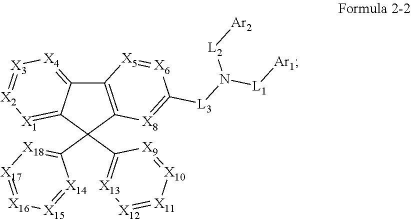
wherein in Formula 2-2, X₁ to X₆ and X₈ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| VI-1 | L-0 | G1 | G1 | VI-2 | L-0 | G2 | G2 |
| VI-3 | L-0 | G3 | G3 | VI-4 | L-0 | G4 | G4 |
| VI-5 | L-0 | G10 | G10 | VI-6 | L-0 | G11 | G11 |
| VI-7 | L-0 | G25 | G25 | | | | |
| | | | | VI-12 | L-0 | G31 | G31 |
| VI-13 | L-0 | G32 | G32 | VI-14 | L-0 | G1 | G2 |
| VI-15 | L-0 | G1 | G3 | VI-16 | L-0 | G1 | G4 |
| VI-17 | L-0 | G1 | G5 | VI-18 | L-0 | G1 | G6 |
| VI-19 | L-0 | G1 | G7 | VI-20 | L-0 | G1 | G8 |
| VI-21 | L-0 | G1 | G9 | VI-22 | L-0 | G1 | G10 |
| VI-23 | L-0 | G1 | G11 | VI-24 | L-0 | G1 | G12 |
| VI-25 | L-0 | G1 | G13 | VI-26 | L-0 | G1 | G14 |
| VI-27 | L-0 | G1 | G15 | VI-28 | L-0 | G1 | G16 |
| VI-29 | L-0 | G1 | G17 | VI-30 | L-0 | G1 | G18 |
| VI-31 | L-0 | G1 | G19 | VI-32 | L-0 | G1 | G20 |
| VI-33 | L-0 | G1 | G21 | VI-34 | L-0 | G1 | G22 |
| VI-35 | L-0 | G1 | G23 | VI-36 | L-0 | G1 | G24 |
| VI-37 | L-0 | G1 | G25 | VI-38 | L-0 | G1 | G26 |
| VI-39 | L-0 | G1 | G27 | VI-40 | L-0 | G1 | G28 |
| VI-41 | L-0 | G1 | G29 | VI-42 | L-0 | G1 | G30 |
| VI-43 | L-0 | G1 | G31 | VI-44 | L-0 | G1 | G32 |
| VI-45 | L-0 | G1 | G33 | VI-46 | L-0 | G2 | G3 |
| VI-47 | L-0 | G2 | G4 | VI-48 | L-0 | G2 | G5 |
| VI-49 | L-0 | G2 | G6 | VI-50 | L-0 | G2 | G7 |
| VI-51 | L-0 | G2 | G8 | VI-52 | L-0 | G2 | G9 |
| VI-53 | L-0 | G2 | G10 | VI-54 | L-0 | G2 | G11 |
| VI-55 | L-0 | G2 | G12 | VI-56 | L-0 | G2 | G13 |
| VI-57 | L-0 | G2 | G14 | VI-58 | L-0 | G2 | G15 |
| VI-59 | L-0 | G2 | G16 | VI-60 | L-0 | G2 | G17 |
| VI-61 | L-0 | G2 | G18 | VI-62 | L-0 | G2 | G19 |
| VI-63 | L-0 | G2 | G20 | VI-64 | L-0 | G2 | G21 |
| VI-65 | L-0 | G2 | G22 | VI-66 | L-0 | G2 | G23 |
| VI-67 | L-0 | G2 | G24 | VI-68 | L-0 | G2 | G25 |
| VI-69 | L-0 | G2 | G26 | VI-70 | L-0 | G2 | G27 |
| VI-71 | L-0 | G2 | G28 | VI-72 | L-0 | G2 | G29 |
| VI-73 | L-0 | G2 | G30 | VI-74 | L-0 | G2 | G31 |
| VI-75 | L-0 | G2 | G32 | VI-76 | L-0 | G2 | G33 |
| VI-77 | L-0 | G3 | G4 | VI-78 | L-0 | G3 | G5 |
| VI-79 | L-0 | G3 | G6 | VI-80 | L-0 | G3 | G7 |
| VI-81 | L-0 | G3 | G8 | VI-82 | L-0 | G3 | G9 |
| VI-83 | L-0 | G3 | G10 | VI-84 | L-0 | G3 | G11 |
| VI-85 | L-0 | G3 | G12 | VI-86 | L-0 | G3 | G13 |
| VI-87 | L-0 | G3 | G14 | VI-88 | L-0 | G3 | G15 |
| VI-89 | L-0 | G3 | G16 | VI-90 | L-0 | G3 | G17 |
| VI-91 | L-0 | G3 | G18 | VI-92 | L-0 | G3 | G19 |
| VI-93 | L-0 | G3 | G20 | VI-94 | L-0 | G3 | G21 |
| VI-95 | L-0 | G3 | G22 | VI-96 | L-0 | G3 | G23 |
| VI-97 | L-0 | G3 | G24 | VI-98 | L-0 | G3 | G25 |
| VI-99 | L-0 | G3 | G26 | VI-100 | L-0 | G3 | G27 |
| VI-101 | L-0 | G3 | G28 | VI-102 | L-0 | G3 | G29 |
| VI-103 | L-0 | G3 | G30 | VI-104 | L-0 | G3 | G31 |
| VI-105 | L-0 | G3 | G32 | VI-106 | L-0 | G3 | G33 |
| VI-107 | L-0 | G4 | G5 | VI-108 | L-0 | G4 | G6 |
| VI-109 | L-0 | G4 | G7 | VI-110 | L-0 | G4 | G8 |
| VI-111 | L-0 | G4 | G9 | VI-112 | L-0 | G4 | G10 |
| VI-113 | L-0 | G4 | G11 | VI-114 | L-0 | G4 | G12 |
| VI-115 | L-0 | G4 | G13 | VI-116 | L-0 | G4 | G14 |
| VI-117 | L-0 | G4 | G15 | VI-118 | L-0 | G4 | G16 |
| VI-119 | L-0 | G4 | G17 | VI-120 | L-0 | G4 | G18 |
| VI-121 | L-0 | G4 | G19 | VI-122 | L-0 | G4 | G20 |
| VI-123 | L-0 | G4 | G21 | VI-124 | L-0 | G4 | G22 |
| VI-125 | L-0 | G4 | G23 | VI-126 | L-0 | G4 | G24 |
| VI-127 | L-0 | G4 | G25 | VI-128 | L-0 | G4 | G26 |
| VI-129 | L-0 | G4 | G27 | VI-130 | L-0 | G4 | G28 |
| VI-131 | L-0 | G4 | G29 | VI-132 | L-0 | G4 | G30 |
| VI-133 | L-0 | G4 | G31 | VI-134 | L-0 | G4 | G32 |
| VI-135 | L-0 | G4 | G33 | VI-136 | L-0 | G5 | G6 |
| VI-137 | L-0 | G5 | G7 | VI-138 | L-0 | G5 | G8 |
| VI-139 | L-0 | G5 | G9 | VI-140 | L-0 | G5 | G10 |
| VI-141 | L-0 | G5 | G11 | VI-142 | L-0 | G5 | G12 |
| VI-143 | L-0 | G5 | G13 | VI-144 | L-0 | G5 | G14 |
| VI-145 | L-0 | G5 | G15 | VI-146 | L-0 | G5 | G16 |
| VI-147 | L-0 | G5 | G17 | VI-148 | L-0 | G5 | G18 |
| VI-149 | L-0 | G5 | G19 | VI-150 | L-0 | G5 | G20 |
| VI-151 | L-0 | G5 | G21 | VI-152 | L-0 | G5 | G22 |
| VI-153 | L-0 | G5 | G23 | VI-154 | L-0 | G5 | G24 |
| VI-155 | L-0 | G5 | G25 | VI-156 | L-0 | G5 | G26 |
| VI-157 | L-0 | G5 | G27 | VI-158 | L-0 | G5 | G28 |
| VI-159 | L-0 | G5 | G29 | VI-160 | L-0 | G5 | G30 |
| VI-161 | L-0 | G5 | G31 | VI-162 | L-0 | G5 | G32 |
| VI-163 | L-0 | G5 | G33 | VI-164 | L-0 | G10 | G26 |
| VI-165 | L-0 | G10 | G27 | VI-166 | L-0 | G10 | G28 |
| VI-167 | L-0 | G10 | G29 | VI-168 | L-0 | G10 | G30 |
| VI-169 | L-0 | G10 | G31 | VI-170 | L-0 | G10 | G32 |
| VI-171 | L-0 | G10 | G33 | VI-172 | L-0 | G11 | G26 |
| VI-173 | L-0 | G11 | G27 | VI-174 | L-0 | G11 | G28 |
| VI-175 | L-0 | G11 | G29 | VI-176 | L-0 | G11 | G30 |
| VI-177 | L-0 | G11 | G31 | VI-178 | L-0 | G11 | G32 |
| VI-179 | L-0 | G11 | G33 | VI-180 | L-l | G1 | G1 |
| VI-181 | L-1 | G2 | G2 | VI-182 | L-1 | G5 | G5 |
| VI-185 | L-1 | G31 | G31 | VI-186 | L-1 | G32 | G32 |
| VI-187 | L-1 | G1 | G2 | VI-188 | L-1 | G1 | G3 |
| VI-189 | L-1 | G1 | G4 | VI-190 | L-1 | G1 | G5 |
| VI-191 | L-1 | G1 | G10 | VI-192 | L-1 | G1 | G11 |
| VI-193 | L-1 | G1 | G19 | VI-194 | L-1 | G1 | G20 |
| VI-195 | L-1 | G1 | G27 | VI-196 | L-1 | G1 | G28 |
| VI-197 | L-1 | G1 | G31 | VI-198 | L-1 | G1 | G32 |
| VI-199 | L-1 | G2 | G3 | VI-200 | L-1 | G2 | G4 |
| VI-201 | L-1 | G2 | G5 | VI-202 | L-1 | G2 | G10 |
| VI-203 | L-1 | G2 | G11 | VI-204 | L-1 | G2 | G19 |
| VI-205 | L-1 | G2 | G20 | VI-206 | L-1 | G2 | G27 |
| VI-207 | L-1 | G2 | G28 | VI-208 | L-1 | G2 | G31 |
| VI-209 | L-1 | G2 | G32 | VI-210 | L-1 | G4 | G5 |
| VI-211 | L-1 | G4 | G10 | VI-212 | L-1 | G4 | G11 |
| VI-213 | L-1 | G4 | G19 | VI-214 | L-1 | G4 | G20 |
| VI-215 | L-1 | G4 | G27 | VI-216 | L-1 | G4 | G28 |
| VI-217 | L-1 | G4 | G31 | VI-218 | L-1 | G4 | G32 |
| VI-219 | L-1 | G5 | G10 | VI-220 | L-1 | G5 | G11 |
| VI-221 | L-1 | G5 | G19 | VI-222 | L-1 | G5 | G20 |
| VI-223 | L-1 | G5 | G27 | VI-224 | L-1 | G5 | G28 |
| VI-225 | L-1 | G5 | G31 | VI-226 | L-1 | G5 | G32 |
| VI-227 | L-4 | G1 | G1 | VI-228 | L-4 | G2 | G2 |
| VI-229 | L-4 | G5 | G5 | | | | |
| | | | | VI-232 | L-4 | G31 | G31 |
| VI-233 | L-4 | G32 | G32 | VI-234 | L-4 | G1 | G2 |
| VI-235 | L-4 | G1 | G3 | VI-236 | L-4 | G1 | G4 |
| VI-237 | L-4 | G1 | G5 | VI-238 | L-4 | G1 | G10 |
| VI-239 | L-4 | G1 | G11 | VI-240 | L-4 | G1 | G19 |
| VI-241 | L-4 | G1 | G20 | VI-242 | L-4 | G1 | G27 |
| VI-243 | L-4 | G1 | G28 | VI-244 | L-4 | G1 | G31 |
| VI-245 | L-4 | G1 | G32 | VI-246 | L-4 | G2 | G3 |
| VI-247 | L-4 | G2 | G4 | VI-248 | L-4 | G2 | G5 |
| VI-249 | L-4 | G2 | G10 | VI-250 | L-4 | G2 | G11 |
| VI-251 | L-4 | G2 | G19 | VI-252 | L-4 | G2 | G20 |
| VI-253 | L-4 | G2 | G27 | VI-254 | L-4 | G2 | G28 |
| VI-255 | L-4 | G2 | G31 | VI-256 | L-4 | G2 | G32 |
| VI-257 | L-4 | G4 | G5 | VI-258 | L-4 | G4 | G10 |
| VI-259 | L-4 | G4 | G11 | VI-260 | L-4 | G4 | G19 |
| VI-261 | L-4 | G4 | G20 | VI-262 | L-4 | G4 | G27 |
| VI-263 | L-4 | G4 | G28 | VI-264 | L-4 | G4 | G31 |
| VI-265 | L-4 | G4 | G32 | VI-266 | L-4 | G5 | G10 |
| VI-267 | L-4 | G5 | G11 | VI-268 | L-4 | G5 | G19 |
| VI-269 | L-4 | G5 | G20 | VI-270 | L-4 | G5 | G27 |
| VI-271 | L-4 | G5 | G28 | VI-272 | L-4 | G5 | G31 |
| VI-273 | L-4 | G5 | G32 | | | | |

wherein Compound VII-1 to Compound VII-273 each have a structure represented by Formula 2-3: 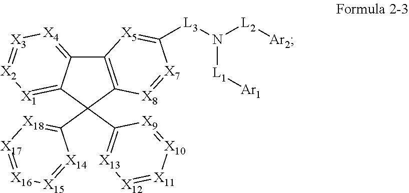
wherein in Formula 2-3, X₁ to X₅ and X₇ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| VII-1 | L-0 | G1 | G1 | VII-2 | L-0 | G2 | G2 |
| VII-3 | L-0 | G3 | G3 | VII-4 | L-0 | G4 | G4 |
| VII-5 | L-0 | G10 | G10 | VII-6 | L-0 | G11 | G11 |
| VII-7 | L-0 | G25 | G25 | | | | |
| | | | | VII-12 | L-0 | G31 | G31 |
| VII-13 | L-0 | G32 | G32 | VII-14 | L-0 | G1 | G2 |
| VII-15 | L-0 | G1 | G3 | VII-16 | L-0 | G1 | G4 |
| VII-17 | L-0 | G1 | G5 | VII-18 | L-0 | G1 | G6 |
| VII-19 | L-0 | G1 | G7 | VII-20 | L-0 | G1 | G8 |
| VII-21 | L-0 | G1 | G9 | VII-22 | L-0 | G1 | G10 |
| VII-23 | L-0 | G1 | G11 | VII-24 | L-0 | G1 | G12 |
| VII-25 | L-0 | G1 | G13 | VII-26 | L-0 | G1 | G14 |
| VII-27 | L-0 | G1 | G15 | VII-28 | L-0 | G1 | G16 |
| VII-29 | L-0 | G1 | G17 | VII-30 | L-0 | G1 | G18 |
| VII-31 | L-0 | G1 | G19 | VII-32 | L-0 | G1 | G20 |
| VII-33 | L-0 | G1 | G21 | VII-34 | L-0 | G1 | G22 |
| VII-35 | L-0 | G1 | G23 | VII-36 | L-0 | G1 | G24 |
| VII-37 | L-0 | G1 | G25 | VII-38 | L-0 | G1 | G26 |
| VII-39 | L-0 | G1 | G27 | VII-40 | L-0 | G1 | G28 |
| VII-41 | L-0 | G1 | G29 | VII-42 | L-0 | G1 | G30 |
| VII-43 | L-0 | G1 | G31 | VII-44 | L-0 | G1 | G32 |
| VII-45 | L-0 | G1 | G33 | VII-46 | L-0 | G2 | G3 |
| VII-47 | L-0 | G2 | G4 | VII-48 | L-0 | G2 | G5 |
| VII-49 | L-0 | G2 | G6 | VII-50 | L-0 | G2 | G7 |
| VII-51 | L-0 | G2 | G8 | VII-52 | L-0 | G2 | G9 |
| VII-53 | L-0 | G2 | G10 | VII-54 | L-0 | G2 | G11 |
| VII-55 | L-0 | G2 | G12 | VII-56 | L-0 | G2 | G13 |
| VII-57 | L-0 | G2 | G14 | VII-58 | L-0 | G2 | G15 |
| VII-59 | L-0 | G2 | G16 | VII-60 | L-0 | G2 | G17 |
| VII-61 | L-0 | G2 | G18 | VII-62 | L-0 | G2 | G19 |
| VII-63 | L-0 | G2 | G20 | VII-64 | L-0 | G2 | G21 |
| VII-65 | L-0 | G2 | G22 | VII-66 | L-0 | G2 | G23 |
| VII-67 | L-0 | G2 | G24 | VII-68 | L-0 | G2 | G25 |
| VII-69 | L-0 | G2 | G26 | VII-70 | L-0 | G2 | G27 |
| VII-71 | L-0 | G2 | G28 | VII-72 | L-0 | G2 | G29 |
| VII-73 | L-0 | G2 | G30 | VII-74 | L-0 | G2 | G31 |
| VII-75 | L-0 | G2 | G32 | VII-76 | L-0 | G2 | G33 |
| VII-77 | L-0 | G3 | G4 | VII-78 | L-0 | G3 | G5 |
| VII-79 | L-0 | G3 | G6 | VII-80 | L-0 | G3 | G7 |
| VII-81 | L-0 | G3 | G8 | VII-82 | L-0 | G3 | G9 |
| VII-83 | L-0 | G3 | G10 | VII-84 | L-0 | G3 | G11 |
| VII-85 | L-0 | G3 | G12 | VII-86 | L-0 | G3 | G13 |
| VII-87 | L-0 | G3 | G14 | VII-88 | L-0 | G3 | G15 |
| VII-89 | L-0 | G3 | G16 | VII-90 | L-0 | G3 | G17 |
| VII-91 | L-0 | G3 | G18 | VII-92 | L-0 | G3 | G19 |
| VII-93 | L-0 | G3 | G20 | VII-94 | L-0 | G3 | G21 |
| VII-95 | L-0 | G3 | G22 | VII-96 | L-0 | G3 | G23 |
| VII-97 | L-0 | G3 | G24 | VII-98 | L-0 | G3 | G25 |
| VII-99 | L-0 | G3 | G26 | VII-100 | L-0 | G3 | G27 |
| VII-101 | L-0 | G3 | G28 | VII-102 | L-0 | G3 | G29 |
| VII-103 | L-0 | G3 | G30 | VII-104 | L-0 | G3 | G31 |
| VII-105 | L-0 | G3 | G32 | VII-106 | L-0 | G3 | G33 |
| VII-107 | L-0 | G4 | G5 | VII-108 | L-0 | G4 | G6 |
| VII-109 | L-0 | G4 | G7 | VII-110 | L-0 | G4 | G8 |
| VII-111 | L-0 | G4 | G9 | VII-112 | L-0 | G4 | G10 |
| VII-113 | L-0 | G4 | G11 | VII-114 | L-0 | G4 | G12 |
| VII-115 | L-0 | G4 | G13 | VII-116 | L-0 | G4 | G14 |
| VII-117 | L-0 | G4 | G15 | VII-118 | L-0 | G4 | G16 |
| VII-119 | L-0 | G4 | G17 | VII-120 | L-0 | G4 | G18 |
| VII-121 | L-0 | G4 | G19 | VII-122 | L-0 | G4 | G20 |
| VII-123 | L-0 | G4 | G21 | VII-124 | L-0 | G4 | G22 |
| VII-125 | L-0 | G4 | G23 | VII-126 | L-0 | G4 | G24 |
| VII-127 | L-0 | G4 | G25 | VII-128 | L-0 | G4 | G26 |
| VII-129 | L-0 | G4 | G27 | VII-130 | L-0 | G4 | G28 |
| VII-131 | L-0 | G4 | G29 | VII-132 | L-0 | G4 | G30 |
| VII-133 | L-0 | G4 | G31 | VII-134 | L-0 | G4 | G32 |
| VII-135 | L-0 | G4 | G33 | VII-136 | L-0 | G5 | G6 |
| VII-137 | L-0 | G5 | G7 | VII-138 | L-0 | G5 | G8 |
| VII-139 | L-0 | G5 | G9 | VII-140 | L-0 | G5 | G10 |
| VII-141 | L-0 | G5 | G11 | VII-142 | L-0 | G5 | G12 |
| VII-143 | L-0 | G5 | G13 | VII-144 | L-0 | G5 | G14 |
| VII-145 | L-0 | G5 | G15 | VII-146 | L-0 | G5 | G16 |
| VII-147 | L-0 | G5 | G17 | VII-148 | L-0 | G5 | G18 |
| VII-149 | L-0 | G5 | G19 | VII-150 | L-0 | G5 | G20 |
| VII-151 | L-0 | G5 | G21 | VII-152 | L-0 | G5 | G22 |
| VII-153 | L-0 | G5 | G23 | VII-154 | L-0 | G5 | G24 |
| VII-155 | L-0 | G5 | G25 | VII-156 | L-0 | G5 | G26 |
| VII-157 | L-0 | G5 | G27 | VII-158 | L-0 | G5 | G28 |
| VII-159 | L-0 | G5 | G29 | VII-160 | L-0 | G5 | G30 |
| VII-161 | L-0 | G5 | G31 | VII-162 | L-0 | G5 | G32 |
| VII-163 | L-0 | G5 | G33 | VII-164 | L-0 | G10 | G26 |
| VII-165 | L-0 | G10 | G27 | VII-166 | L-0 | G10 | G28 |
| VII-167 | L-0 | G10 | G29 | VII-168 | L-0 | G10 | G30 |
| VII-169 | L-0 | G10 | G31 | VII-170 | L-0 | G10 | G32 |
| VII-171 | L-0 | G10 | G33 | VII-172 | L-0 | G11 | G26 |
| VII-173 | L-0 | G11 | G27 | VII-174 | L-0 | G11 | G28 |
| VII-175 | L-0 | G11 | G29 | VII-176 | L-0 | G11 | G30 |
| VII-177 | L-0 | G11 | G31 | VII-178 | L-0 | G11 | G32 |
| VII-179 | L-0 | G11 | G33 | VII-180 | L-1 | G1 | G1 |
| VII-181 | L-1 | G2 | G2 | VII-182 | L-1 | G5 | G5 |
| VII-185 | L-1 | G31 | G31 | VII-186 | L-1 | G32 | G32 |
| VII-187 | L-1 | G1 | G2 | VII-188 | L-1 | G1 | G3 |
| VII-189 | L-1 | G1 | G4 | VII-190 | L-1 | G1 | G5 |
| VII-191 | L-1 | G1 | G10 | VII-192 | L-1 | G1 | G11 |
| VII-193 | L-1 | G1 | G19 | VII-194 | L-1 | G1 | G20 |
| VII-195 | L-1 | G1 | G27 | VII-196 | L-1 | G1 | G28 |
| VII-197 | L-1 | G1 | G31 | VII-198 | L-1 | G1 | G32 |
| VII-199 | L-1 | G2 | G3 | VII-200 | L-1 | G2 | G4 |
| VII-201 | L-1 | G2 | G5 | VII-202 | L-1 | G2 | G10 |
| VII-203 | L-1 | G2 | G11 | VII-204 | L-1 | G2 | G19 |
| VII-205 | L-1 | G2 | G20 | VII-206 | L-1 | G2 | G27 |
| VII-207 | L-1 | G2 | G28 | VII-208 | L-1 | G2 | G31 |
| VII-209 | L-1 | G2 | G32 | VII-210 | L-1 | G4 | G5 |
| VII-211 | L-1 | G4 | G10 | VII-212 | L-1 | G4 | G11 |
| VII-213 | L-1 | G4 | G19 | VII-214 | L-1 | G4 | G20 |
| VII-215 | L-1 | G4 | G27 | VII-216 | L-1 | G4 | G28 |
| VII-217 | L-1 | G4 | G31 | VII-218 | L-1 | G4 | G32 |
| VII-219 | L-1 | G5 | G10 | VII-220 | L-1 | G5 | G11 |
| VII-221 | L-1 | G5 | G19 | VII-222 | L-1 | G5 | G20 |
| VII-223 | L-1 | G5 | G27 | VII-224 | L-1 | G5 | G28 |
| VII-225 | L-1 | G5 | G31 | VII-226 | L-1 | G5 | G32 |
| VII-227 | L-4 | G1 | G1 | VII-228 | L-4 | G2 | G2 |
| VII-229 | L-4 | G5 | G5 | | | | |
| | | | | VII-232 | L-4 | G31 | G31 |
| VII-233 | L-4 | G32 | G32 | VII-234 | L-4 | G1 | G2 |
| VII-235 | L-4 | G1 | G3 | VII-236 | L-4 | G1 | G4 |
| VII-237 | L-4 | G1 | G5 | VII-238 | L-4 | G1 | G10 |
| VII-239 | L-4 | G1 | G11 | VII-240 | L-4 | G1 | G19 |
| VII-241 | L-4 | G1 | G20 | VII-242 | L-4 | G1 | G27 |
| VII-243 | L-4 | G1 | G28 | VII-244 | L-4 | G1 | G31 |
| VII-245 | L-4 | G1 | G32 | VII-246 | L-4 | G2 | G3 |
| VII-247 | L-4 | G2 | G4 | VII-248 | L-4 | G2 | G5 |
| VII-249 | L-4 | G2 | G10 | VII-250 | L-4 | G2 | G11 |
| VII-251 | L-4 | G2 | G19 | VII-252 | L-4 | G2 | G20 |
| VII-253 | L-4 | G2 | G27 | VII-254 | L-4 | G2 | G28 |
| VII-255 | L-4 | G2 | G31 | VII-256 | L-4 | G2 | G32 |
| VII-257 | L-4 | G4 | G5 | VII-258 | L-4 | G4 | G10 |
| VII-259 | L-4 | G4 | G11 | VII-260 | L-4 | G4 | G19 |
| VII-261 | L-4 | G4 | G20 | VII-262 | L-4 | G4 | G27 |
| VII-263 | L-4 | G4 | G28 | VII-264 | L-4 | G4 | G31 |
| VII-265 | L-4 | G4 | G32 | VII-266 | L-4 | G5 | G10 |
| VII-267 | L-4 | G5 | G11 | VII-268 | L-4 | G5 | G19 |
| VII-269 | L-4 | G5 | G20 | VII-270 | L-4 | G5 | G27 |
| VII-271 | L-4 | G5 | G28 | VII-272 | L-4 | G5 | G31 |
| VII-273 | L-4 | G5 | G32 | | | | |

wherein Compound VIII-1 to Compound VIII-273 each have a structure represented by Formula 2-4: 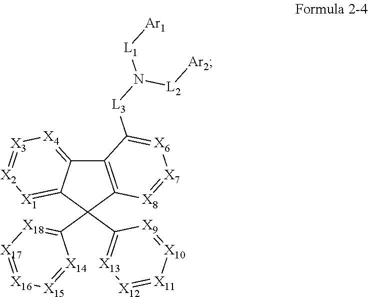
wherein in Formula 2-4, X₁ to X₄ and X₆ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| VIII-1 | L-0 | G1 | G1 | VIII-2 | L-0 | G2 | G2 |
| VIII-3 | L-0 | G3 | G3 | VIII-4 | L-0 | G4 | G4 |
| VIII-5 | L-0 | G10 | G10 | VIII-6 | L-0 | G11 | G11 |
| VIII-7 | L-0 | G25 | G25 | | | | |
| | | | | VIII-12 | L-0 | G31 | G31 |
| VIII-13 | L-0 | G32 | G32 | VIII-14 | L-0 | G1 | G2 |
| VIII-15 | L-0 | G1 | G3 | VIII-16 | L-0 | G1 | G4 |
| VIII-17 | L-0 | G1 | G5 | VIII-18 | L-0 | G1 | G6 |
| VIII-19 | L-0 | G1 | G7 | VIII-20 | L-0 | G1 | G8 |
| VIII-21 | L-0 | G1 | G9 | VIII-22 | L-0 | G1 | G10 |
| VIII-23 | L-0 | G1 | G11 | VIII-24 | L-0 | G1 | G12 |
| VIII-25 | L-0 | G1 | G13 | VIII-26 | L-0 | G1 | G14 |
| VIII-27 | L-0 | G1 | G15 | VIII-28 | L-0 | G1 | G16 |
| VIII-29 | L-0 | G1 | G17 | VIII-30 | L-0 | G1 | G18 |
| VIII-31 | L-0 | G1 | G19 | VIII-32 | L-0 | G1 | G20 |
| VIII-33 | L-0 | G1 | G21 | VIII-34 | L-0 | G1 | G22 |
| VIII-35 | L-0 | G1 | G23 | VIII-36 | L-0 | G1 | G24 |
| VIII-37 | L-0 | G1 | G25 | VIII-38 | L-0 | G1 | G26 |
| VIII-39 | L-0 | G1 | G27 | VIII-40 | L-0 | G1 | G28 |
| VIII-41 | L-0 | G1 | G29 | VIII-42 | L-0 | G1 | G30 |
| VIII-43 | L-0 | G1 | G31 | VIII-44 | L-0 | G1 | G32 |
| VIII-45 | L-0 | G1 | G33 | VIII-46 | L-0 | G2 | G3 |
| VIII-47 | L-0 | G2 | G4 | VIII-48 | L-0 | G2 | G5 |
| VIII-49 | L-0 | G2 | G6 | VIII-50 | L-0 | G2 | G7 |
| VIII-51 | L-0 | G2 | G8 | VIII-52 | L-0 | G2 | G9 |
| VIII-53 | L-0 | G2 | G10 | VIII-54 | L-0 | G2 | G11 |
| VIII-55 | L-0 | G2 | G12 | VIII-56 | L-0 | G2 | G13 |
| VIII-57 | L-0 | G2 | G14 | VIII-58 | L-0 | G2 | G15 |
| VIII-59 | L-0 | G2 | G16 | VIII-60 | L-0 | G2 | G17 |
| VIII-61 | L-0 | G2 | G18 | VIII-62 | L-0 | G2 | G19 |
| VIII-63 | L-0 | G2 | G20 | VIII-64 | L-0 | G2 | G21 |
| VIII-65 | L-0 | G2 | G22 | VIII-66 | L-0 | G2 | G23 |
| VIII-67 | L-0 | G2 | G24 | VIII-68 | L-0 | G2 | G25 |
| VIII-69 | L-0 | G2 | G26 | VIII-70 | L-0 | G2 | G27 |
| VIII-71 | L-0 | G2 | G28 | VIII-72 | L-0 | G2 | G29 |
| VIII-73 | L-0 | G2 | G30 | VIII-74 | L-0 | G2 | G31 |
| VIII-75 | L-0 | G2 | G32 | VIII-76 | L-0 | G2 | G33 |
| VIII-77 | L-0 | G3 | G4 | VIII-78 | L-0 | G3 | G5 |
| VIII-79 | L-0 | G3 | G6 | VIII-80 | L-0 | G3 | G7 |
| VIII-81 | L-0 | G3 | G8 | VIII-82 | L-0 | G3 | G9 |
| VIII-83 | L-0 | G3 | G10 | VIII-84 | L-0 | G3 | G11 |
| VIII-85 | L-0 | G3 | G12 | VIII-86 | L-0 | G3 | G13 |
| VIII-87 | L-0 | G3 | G14 | VIII-88 | L-0 | G3 | G15 |
| VIII-89 | L-0 | G3 | G16 | VIII-90 | L-0 | G3 | G17 |
| VIII-91 | L-0 | G3 | G18 | VIII-92 | L-0 | G3 | G19 |
| VIII-93 | L-0 | G3 | G20 | VIII-94 | L-0 | G3 | G21 |
| VIII-95 | L-0 | G3 | G22 | VIII-96 | L-0 | G3 | G23 |
| VIII-97 | L-0 | G3 | G24 | VIII-98 | L-0 | G3 | G25 |
| VIII-99 | L-0 | G3 | G26 | VIII-100 | L-0 | G3 | G27 |
| VIII-101 | L-0 | G3 | G28 | VIII-102 | L-0 | G3 | G29 |
| VIII-103 | L-0 | G3 | G30 | VIII-104 | L-0 | G3 | G31 |
| VIII-105 | L-0 | G3 | G32 | VIII-106 | L-0 | G3 | G33 |
| VIII-107 | L-0 | G4 | G5 | VIII-108 | L-0 | G4 | G6 |
| VIII-109 | L-0 | G4 | G7 | VIII-110 | L-0 | G4 | G8 |
| VIII-111 | L-0 | G4 | G9 | VIII-112 | L-0 | G4 | G10 |
| VIII-113 | L-0 | G4 | G11 | VIII-114 | L-0 | G4 | G12 |
| VIII-115 | L-0 | G4 | G13 | VIII-116 | L-0 | G4 | G14 |
| VIII-117 | L-0 | G4 | G15 | VIII-118 | L-0 | G4 | G16 |
| VIII-119 | L-0 | G4 | G17 | VIII-120 | L-0 | G4 | G18 |
| VIII-121 | L-0 | G4 | G19 | VIII-122 | L-0 | G4 | G20 |
| VIII-123 | L-0 | G4 | G21 | VIII-124 | L-0 | G4 | G22 |
| VIII-125 | L-0 | G4 | G23 | VIII-126 | L-0 | G4 | G24 |
| VIII-127 | L-0 | G4 | G25 | VIII-128 | L-0 | G4 | G26 |
| VIII-129 | L-0 | G4 | G27 | VIII-130 | L-0 | G4 | G28 |
| VIII-131 | L-0 | G4 | G29 | VIII-132 | L-0 | G4 | G30 |
| VIII-133 | L-0 | G4 | G31 | VIII-134 | L-0 | G4 | G32 |
| VIII-135 | L-0 | G4 | G33 | VIII-136 | L-0 | G5 | G6 |
| VIII-137 | L-0 | G5 | G7 | VIII-138 | L-0 | G5 | G8 |
| VIII-139 | L-0 | G5 | G9 | VIII-140 | L-0 | G5 | G10 |
| VIII-141 | L-0 | G5 | G11 | VIII-142 | L-0 | G5 | G12 |
| VIII-143 | L-0 | G5 | G13 | VIII-144 | L-0 | G5 | G14 |
| VIII-145 | L-0 | G5 | G15 | VIII-146 | L-0 | G5 | G16 |
| VIII-147 | L-0 | G5 | G17 | VIII-148 | L-0 | G5 | G18 |
| VIII-149 | L-0 | G5 | G19 | VIII-150 | L-0 | G5 | G20 |
| VIII-151 | L-0 | G5 | G21 | VIII-152 | L-0 | G5 | G22 |
| VIII-153 | L-0 | G5 | G23 | VIII-154 | L-0 | G5 | G24 |
| VIII-155 | L-0 | G5 | G25 | VIII-156 | L-0 | G5 | G26 |
| VIII-157 | L-0 | G5 | G27 | VIII-158 | L-0 | G5 | G28 |
| VIII-159 | L-0 | G5 | G29 | VIII-160 | L-0 | G5 | G30 |
| VIII-161 | L-0 | G5 | G31 | VIII-162 | L-0 | G5 | G32 |
| VIII-163 | L-0 | G5 | G33 | VIII-164 | L-0 | G10 | G26 |
| VIII-165 | L-0 | G10 | G27 | VIII-166 | L-0 | G10 | G28 |
| VIII-167 | L-0 | G10 | G29 | VIII-168 | L-0 | G10 | G30 |
| VIII-169 | L-0 | G10 | G31 | VIII-170 | L-0 | G10 | G32 |
| VIII-171 | L-0 | G10 | G33 | VIII-172 | L-0 | G11 | G26 |
| VIII-173 | L-0 | G11 | G27 | VIII-174 | L-0 | G11 | G28 |
| VIII-175 | L-0 | G11 | G29 | VIII-176 | L-0 | G11 | G30 |
| VIII-177 | L-0 | G11 | G31 | VIII-178 | L-0 | G11 | G32 |
| VIII-179 | L-0 | G11 | G33 | VIII-180 | L-1 | G1 | G1 |
| VIII-181 | L-1 | G2 | G2 | VIII-182 | L-1 | G5 | G5 |
| VIII-185 | L-1 | G31 | G31 | VIII-186 | L-1 | G32 | G32 |
| VIII-187 | L-1 | G1 | G2 | VIII-188 | L-1 | G1 | G3 |
| VIII-189 | L-1 | G1 | G4 | VIII-190 | L-1 | G1 | G5 |
| VIII-191 | L-1 | G1 | G10 | VIII-192 | L-1 | G1 | G11 |
| VIII-193 | L-1 | G1 | G19 | VIII-194 | L-1 | G1 | G20 |
| VIII-195 | L-1 | G1 | G27 | VIII-196 | L-1 | G1 | G28 |
| VIII-197 | L-1 | G1 | G31 | VIII-198 | L-1 | G1 | G32 |
| VIII-199 | L-1 | G2 | G3 | VIII-200 | L-1 | G2 | G4 |
| VIII-201 | L-1 | G2 | G5 | VIII-202 | L-1 | G2 | G10 |
| VIII-203 | L-1 | G2 | G11 | VIII-204 | L-1 | G2 | G19 |
| VIII-205 | L-1 | G2 | G20 | VIII-206 | L-1 | G2 | G27 |
| VIII-207 | L-1 | G2 | G28 | VIII-208 | L-1 | G2 | G31 |
| VIII-209 | L-1 | G2 | G32 | VIII-210 | L-1 | G4 | G5 |
| VIII-211 | L-1 | G4 | G10 | VIII-212 | L-1 | G4 | G11 |
| VIII-213 | L-1 | G4 | G19 | VIII-214 | L-1 | G4 | G20 |
| VIII-215 | L-1 | G4 | G27 | VIII-216 | L-1 | G4 | G28 |
| VIII-217 | L-1 | G4 | G31 | VIII-218 | L-1 | G4 | G32 |
| VIII-219 | L-1 | G5 | G10 | VIII-220 | L-1 | G5 | G11 |
| VIII-221 | L-1 | G5 | G19 | VIII-222 | L-1 | G5 | G20 |
| VIII-223 | L-1 | G5 | G27 | VIII-224 | L-1 | G5 | G28 |
| VIII-225 | L-1 | G5 | G31 | VIII-226 | L-1 | G5 | G32 |
| VIII-227 | L-4 | G1 | G1 | VIII-228 | L-4 | G2 | G2 |
| VIII-229 | L-4 | G5 | G5 | | | | |
| | | | | VIII-232 | L-4 | G31 | G31 |
| VIII-233 | L-4 | G32 | G32 | VIII-234 | L-4 | G1 | G2 |
| VIII-235 | L-4 | G1 | G3 | VIII-236 | L-4 | G1 | G4 |
| VIII-237 | L-4 | G1 | G5 | VIII-238 | L-4 | G1 | G10 |
| VIII-239 | L-4 | G1 | G11 | VIII-240 | L-4 | G1 | G19 |
| VIII-241 | L-4 | G1 | G20 | VIII-242 | L-4 | G1 | G27 |
| VIII-243 | L-4 | G1 | G28 | VIII-244 | L-4 | G1 | G31 |
| VIII-245 | L-4 | G1 | G32 | VIII-246 | L-4 | G2 | G3 |
| VIII-247 | L-4 | G2 | G4 | VIII-248 | L-4 | G2 | G5 |
| VIII-249 | L-4 | G2 | G10 | VIII-250 | L-4 | G2 | G11 |
| VIII-251 | L-4 | G2 | G19 | VIII-252 | L-4 | G2 | G20 |
| VIII-253 | L-4 | G2 | G27 | VIII-254 | L-4 | G2 | G28 |
| VIII-255 | L-4 | G2 | G31 | VIII-256 | L-4 | G2 | G32 |
| VIII-257 | L-4 | G4 | G5 | VIII-258 | L-4 | G4 | G10 |
| VIII-259 | L-4 | G4 | G11 | VIII-260 | L-4 | G4 | G19 |
| VIII-261 | L-4 | G4 | G20 | VIII-262 | L-4 | G4 | G27 |
| VIII-263 | L-4 | G4 | G28 | VIII-264 | L-4 | G4 | G31 |
| VIII-265 | L-4 | G4 | G32 | VIII-266 | L-4 | G5 | G10 |
| VIII-267 | L-4 | G5 | G11 | VIII-268 | L-4 | G5 | G19 |
| VIII-269 | L-4 | G5 | G20 | VIII-270 | L-4 | G5 | G27 |
| VIII-271 | L-4 | G5 | G28 | VIII-272 | L-4 | G5 | G31 |
| VIII-273 | L-4 | G5 | G32 | | | | |

wherein Compound IX-1 to Compound NX-273 each have a structure represented by Formula 2-6: 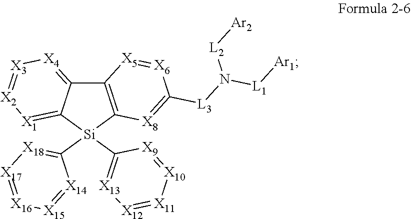
wherein in Formula 2-6, X₁ to X₆ and X₁ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| IX-1 | L-0 | G1 | G1 | IX-2 | L-0 | G2 | G2 |
| IX-3 | L-0 | G3 | G3 | IX-4 | L-0 | G4 | G4 |
| IX-5 | L-0 | G10 | G10 | IX-6 | L-0 | G11 | G11 |
| IX-7 | L-0 | G25 | G25 | | | | |
| | | | | IX-12 | L-0 | G31 | G31 |
| IX-13 | L-0 | G32 | G32 | IX-14 | L-0 | G1 | G2 |
| IX-15 | L-0 | G1 | G3 | IX-16 | L-0 | G1 | G4 |
| IX-17 | L-0 | G1 | G5 | IX-18 | L-0 | G1 | G6 |
| IX-19 | L-0 | G1 | G7 | IX-20 | L-0 | G1 | G8 |
| IX-21 | L-0 | G1 | G9 | IX-22 | L-0 | G1 | G10 |
| IX-23 | L-0 | G1 | G11 | IX-24 | L-0 | G1 | G12 |
| IX-25 | L-0 | G1 | G13 | IX-26 | L-0 | G1 | G14 |
| IX-27 | L-0 | G1 | G15 | IX-28 | L-0 | G1 | G16 |
| IX-29 | L-0 | G1 | G17 | IX-30 | L-0 | G1 | G18 |
| IX-31 | L-0 | G1 | G19 | IX-32 | L-0 | G1 | G20 |
| IX-33 | L-0 | G1 | G21 | IX-34 | L-0 | G1 | G22 |
| IX-35 | L-0 | G1 | G23 | IX-36 | L-0 | G1 | G24 |
| IX-37 | L-0 | G1 | G25 | IX-38 | L-0 | G1 | G26 |
| IX-39 | L-0 | G1 | G27 | IX-40 | L-0 | G1 | G28 |
| IX-41 | L-0 | G1 | G29 | IX-42 | L-0 | G1 | G30 |
| IX-43 | L-0 | G1 | G31 | IX-44 | L-0 | G1 | G32 |
| IX-45 | L-0 | G1 | G33 | IX-46 | L-0 | G2 | G3 |
| IX-47 | L-0 | G2 | G4 | IX-48 | L-0 | G2 | G5 |
| IX-49 | L-0 | G2 | G6 | IX-50 | L-0 | G2 | G7 |
| IX-51 | L-0 | G2 | G8 | IX-52 | L-0 | G2 | G9 |
| IX-53 | L-0 | G2 | G10 | IX-54 | L-0 | G2 | G11 |
| IX-55 | L-0 | G2 | G12 | IX-56 | L-0 | G2 | G13 |
| IX-57 | L-0 | G2 | G14 | IX-58 | L-0 | G2 | G15 |
| IX-59 | L-0 | G2 | G16 | IX-60 | L-0 | G2 | G17 |
| IX-61 | L-0 | G2 | G18 | IX-62 | L-0 | G2 | G19 |
| IX-63 | L-0 | G2 | G20 | IX-64 | L-0 | G2 | G21 |
| IX-65 | L-0 | G2 | G22 | IX-66 | L-0 | G2 | G23 |
| IX-67 | L-0 | G2 | G24 | IX-68 | L-0 | G2 | G25 |
| IX-69 | L-0 | G2 | G26 | IX-70 | L-0 | G2 | G27 |
| IX-71 | L-0 | G2 | G28 | IX-72 | L-0 | G2 | G29 |
| IX-73 | L-0 | G2 | G30 | IX-74 | L-0 | G2 | G31 |
| IX-75 | L-0 | G2 | G32 | IX-76 | L-0 | G2 | G33 |
| IX-77 | L-0 | G3 | G4 | IX-78 | L-0 | G3 | G5 |
| IX-79 | L-0 | G3 | G6 | IX-80 | L-0 | G3 | G7 |
| IX-81 | L-0 | G3 | G8 | IX-82 | L-0 | G3 | G9 |
| IX-83 | L-0 | G3 | G10 | IX-84 | L-0 | G3 | G11 |
| IX-85 | L-0 | G3 | G12 | IX-86 | L-0 | G3 | G13 |
| IX-87 | L-0 | G3 | G14 | IX-88 | L-0 | G3 | G15 |
| IX-89 | L-0 | G3 | G16 | IX-90 | L-0 | G3 | G17 |
| IX-91 | L-0 | G3 | G18 | IX-92 | L-0 | G3 | G19 |
| IX-93 | L-0 | G3 | G20 | IX-94 | L-0 | G3 | G21 |
| IX-95 | L-0 | G3 | G22 | IX-96 | L-0 | G3 | G23 |
| IX-97 | L-0 | G3 | G24 | IX-98 | L-0 | G3 | G25 |
| IX-99 | L-0 | G3 | G26 | IX-100 | L-0 | G3 | G27 |
| IX-101 | L-0 | G3 | G28 | IX-102 | L-0 | G3 | G29 |
| IX-103 | L-0 | G3 | G30 | IX-104 | L-0 | G3 | G31 |
| IX-105 | L-0 | G3 | G32 | IX-106 | L-0 | G3 | G33 |
| IX-107 | L-0 | G4 | G5 | IX-108 | L-0 | G4 | G6 |
| IX-109 | L-0 | G4 | G7 | IX-110 | L-0 | G4 | G8 |
| IX-111 | L-0 | G4 | G9 | IX-112 | L-0 | G4 | G10 |
| IX-113 | L-0 | G4 | G11 | IX-114 | L-0 | G4 | G12 |
| IX-115 | L-0 | G4 | G13 | IX-116 | L-0 | G4 | G14 |
| IX-117 | L-0 | G4 | G15 | IX-118 | L-0 | G4 | G16 |
| IX-119 | L-0 | G4 | G17 | IX-120 | L-0 | G4 | G18 |
| IX-121 | L-0 | G4 | G19 | IX-122 | L-0 | G4 | G20 |
| IX-123 | L-0 | G4 | G21 | IX-124 | L-0 | G4 | G22 |
| IX-125 | L-0 | G4 | G23 | IX-126 | L-0 | G4 | G24 |
| IX-127 | L-0 | G4 | G25 | IX-128 | L-0 | G4 | G26 |
| IX-129 | L-0 | G4 | G27 | IX-130 | L-0 | G4 | G28 |
| IX-131 | L-0 | G4 | G29 | IX-132 | L-0 | G4 | G30 |
| IX-133 | L-0 | G4 | G31 | IX-134 | L-0 | G4 | G32 |
| IX-135 | L-0 | G4 | G33 | IX-136 | L-0 | G5 | G6 |
| IX-137 | L-0 | G5 | G7 | IX-138 | L-0 | G5 | G8 |
| IX-139 | L-0 | G5 | G9 | IX-140 | L-0 | G5 | G10 |
| IX-141 | L-0 | G5 | G11 | IX-142 | L-0 | G5 | G12 |
| IX-143 | L-0 | G5 | G13 | IX-144 | L-0 | G5 | G14 |
| IX-145 | L-0 | G5 | G15 | IX-146 | L-0 | G5 | G16 |
| IX-147 | L-0 | G5 | G17 | IX-148 | L-0 | G5 | G18 |
| IX-149 | L-0 | G5 | G19 | IX-150 | L-0 | G5 | G20 |
| IX-151 | L-0 | G5 | G21 | IX-152 | L-0 | G5 | G22 |
| IX-153 | L-0 | G5 | G23 | IX-154 | L-0 | G5 | G24 |
| IX-155 | L-0 | G5 | G25 | IX-156 | L-0 | G5 | G26 |
| IX-157 | L-0 | G5 | G27 | IX-158 | L-0 | G5 | G28 |
| IX-159 | L-0 | G5 | G29 | IX-160 | L-0 | G5 | G30 |
| IX-161 | L-0 | G5 | G31 | IX-162 | L-0 | G5 | G32 |
| IX-163 | L-0 | G5 | G33 | IX-164 | L-0 | G10 | G26 |
| IX-165 | L-0 | G10 | G27 | IX-166 | L-0 | G10 | G28 |
| IX-167 | L-0 | G10 | G29 | IX-168 | L-0 | G10 | G30 |
| IX-169 | L-0 | G10 | G31 | IX-170 | L-0 | G10 | G32 |
| IX-171 | L-0 | G10 | G33 | IX-172 | L-0 | G11 | G26 |
| IX-173 | L-0 | G11 | G27 | IX-174 | L-0 | G11 | G28 |
| IX-175 | L-0 | G11 | G29 | IX-176 | L-0 | G11 | G30 |
| IX-177 | L-0 | G11 | G31 | IX-178 | L-0 | G11 | G32 |
| IX-179 | L-0 | G11 | G33 | IX-180 | L-1 | G1 | G1 |
| IX-181 | L-1 | G2 | G2 | IX-182 | L-1 | G5 | G5 |
| IX-185 | L-1 | G31 | G31 | IX-186 | L-1 | G32 | G32 |
| IX-187 | L-1 | G1 | G2 | IX-188 | L-1 | G1 | G3 |
| IX-189 | L-1 | G1 | G4 | IX-190 | L-1 | G1 | G5 |
| IX-191 | L-1 | G1 | G10 | IX-192 | L-1 | G1 | G11 |
| IX-193 | L-1 | G1 | G19 | IX-194 | L-1 | G1 | G20 |
| IX-195 | L-1 | G1 | G27 | IX-196 | L-1 | G1 | G28 |
| IX-197 | L-1 | G1 | G31 | IX-198 | L-1 | G1 | G32 |
| IX-199 | L-1 | G2 | G3 | IX-200 | L-1 | G2 | G4 |
| IX-201 | L-1 | G2 | G5 | IX-202 | L-1 | G2 | G10 |
| IX-203 | L-1 | G2 | G11 | IX-204 | L-1 | G2 | G19 |
| IX-205 | L-1 | G2 | G20 | IX-206 | L-1 | G2 | G27 |
| IX-207 | L-1 | G2 | G28 | IX-208 | L-1 | G2 | G31 |
| IX-209 | L-1 | G2 | G32 | IX-210 | L-1 | G4 | G5 |
| IX-211 | L-1 | G4 | G10 | IX-212 | L-1 | G4 | G11 |
| IX-213 | L-1 | G4 | G19 | IX-214 | L-1 | G4 | G20 |
| IX-215 | L-1 | G4 | G27 | IX-216 | L-1 | G4 | G28 |
| IX-217 | L-1 | G4 | G31 | IX-218 | L-1 | G4 | G32 |
| IX-219 | L-1 | G5 | G10 | IX-220 | L-1 | G5 | G11 |
| IX-221 | L-1 | G5 | G19 | IX-222 | L-1 | G5 | G20 |
| IX-223 | L-1 | G5 | G27 | IX-224 | L-1 | G5 | G28 |
| IX-225 | L-1 | G5 | G31 | IX-226 | L-1 | G5 | G32 |
| IX-227 | L-4 | G1 | G1 | IX-228 | L-4 | G2 | G2 |
| IX-229 | L-4 | G5 | G5 | | | | |
| | | | | IX-232 | L-4 | G31 | G31 |
| IX-233 | L-4 | G32 | G32 | IX-234 | L-4 | G1 | G2 |
| IX-235 | L-4 | G1 | G3 | IX-236 | L-4 | G1 | G4 |
| IX-237 | L-4 | G1 | G5 | IX-238 | L-4 | G1 | G10 |
| IX-239 | L-4 | G1 | G11 | IX-240 | L-4 | G1 | G19 |
| IX-241 | L-4 | G1 | G20 | IX-242 | L-4 | G1 | G27 |
| IX-243 | L-4 | G1 | G28 | IX-244 | L-4 | G1 | G31 |
| IX-245 | L-4 | G1 | G32 | IX-246 | L-4 | G2 | G3 |
| IX-247 | L-4 | G2 | G4 | IX-248 | L-4 | G2 | G5 |
| IX-249 | L-4 | G2 | G10 | IX-250 | L-4 | G2 | G11 |
| IX-251 | L-4 | G2 | G19 | IX-252 | L-4 | G2 | G20 |
| IX-253 | L-4 | G2 | G27 | IX-254 | L-4 | G2 | G28 |
| IX-255 | L-4 | G2 | G31 | IX-256 | L-4 | G2 | G32 |
| IX-257 | L-4 | G4 | G5 | IX-258 | L-4 | G4 | G10 |
| IX-259 | L-4 | G4 | G11 | IX-260 | L-4 | G4 | G19 |
| IX-261 | L-4 | G4 | G20 | IX-262 | L-4 | G4 | G27 |
| IX-263 | L-4 | G4 | G28 | IX-264 | L-4 | G4 | G31 |
| IX-265 | L-4 | G4 | G32 | IX-266 | L-4 | G5 | G10 |
| IX-267 | L-4 | G5 | G11 | IX-268 | L-4 | G5 | G19 |
| IX-269 | L-4 | G5 | G20 | IX-270 | L-4 | G5 | G27 |
| IX-271 | L-4 | G5 | G28 | IX-272 | L-4 | G5 | G31 |
| IX-273 | L-4 | G5 | G32 | | | | |

wherein Compound X-1 to Compound X-273 each have a structure represented by Formula 2-10: 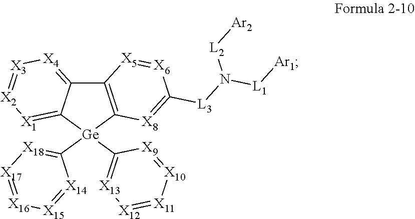
wherein in Formula 2-10, X₁ to X₆ and X₈ to X₁₈ are CH, L₁ and L₂ are single bonds, and L₃, Ar₁ and Ar₂ correspond to atoms or groups listed in the following table, respectively:

| Compound | | | | Compound | | | |
|---|---|---|---|---|---|---|---|
| No. | L₃ | Ar₁ | Ar₂ | No. | L₃ | Ar₁ | Ar₂ |
| X-1 | L-0 | G1 | G1 | X-2 | L-0 | G2 | G2 |
| X-3 | L-0 | G3 | G3 | X-4 | L-0 | G4 | G4 |
| X-5 | L-0 | G10 | G10 | X-6 | L-0 | G11 | G11 |
| X-7 | L-0 | G25 | G25 | | | | |
| | | | | X-12 | L-0 | G31 | G31 |
| X-13 | L-0 | G32 | G32 | X-14 | L-0 | G1 | G2 |
| X-15 | L-0 | G1 | G3 | X-16 | L-0 | G1 | G4 |
| X-17 | L-0 | G1 | G5 | X-18 | L-0 | G1 | G6 |
| X-19 | L-0 | G1 | G7 | X-20 | L-0 | G1 | G8 |
| X-21 | L-0 | G1 | G9 | X-22 | L-0 | G1 | G10 |
| X-23 | L-0 | G1 | G11 | X-24 | L-0 | G1 | G12 |
| X-25 | L-0 | G1 | G13 | X-26 | L-0 | G1 | G14 |
| X-27 | L-0 | G1 | G15 | X-28 | L-0 | G1 | G16 |
| X-29 | L-0 | G1 | G17 | X-30 | L-0 | G1 | G18 |
| X-31 | L-0 | G1 | G19 | X-32 | L-0 | G1 | G20 |
| X-33 | L-0 | G1 | G21 | X-34 | L-0 | G1 | G22 |
| X-35 | L-0 | G1 | G23 | X-36 | L-0 | G1 | G24 |
| X-37 | L-0 | G1 | G25 | X-38 | L-0 | G1 | G26 |
| X-39 | L-0 | G1 | G27 | X-40 | L-0 | G1 | G28 |
| X-41 | L-0 | G1 | G29 | X-42 | L-0 | G1 | G30 |
| X-43 | L-0 | G1 | G31 | X-44 | L-0 | G1 | G32 |
| X-45 | L-0 | G1 | G33 | X-46 | L-0 | G2 | G3 |
| X-47 | L-0 | G2 | G4 | X-48 | L-0 | G2 | G5 |
| X-49 | L-0 | G2 | G6 | X-50 | L-0 | G2 | G7 |
| X-51 | L-0 | G2 | G8 | X-52 | L-0 | G2 | G9 |
| X-53 | L-0 | G2 | G10 | X-54 | L-0 | G2 | G11 |
| X-55 | L-0 | G2 | G12 | X-56 | L-0 | G2 | G13 |
| X-57 | L-0 | G2 | G14 | X-58 | L-0 | G2 | G15 |
| X-59 | L-0 | G2 | G16 | X-60 | L-0 | G2 | G17 |
| X-61 | L-0 | G2 | G18 | X-62 | L-0 | G2 | G19 |
| X-63 | L-0 | G2 | G20 | X-64 | L-0 | G2 | G21 |
| X-65 | L-0 | G2 | G22 | X-66 | L-0 | G2 | G23 |
| X-67 | L-0 | G2 | G24 | X-68 | L-0 | G2 | G25 |
| X-69 | L-0 | G2 | G26 | X-70 | L-0 | G2 | G27 |
| X-71 | L-0 | G2 | G28 | X-72 | L-0 | G2 | G29 |
| X-73 | L-0 | G2 | G30 | X-74 | L-0 | G2 | G31 |
| X-75 | L-0 | G2 | G32 | X-76 | L-0 | G2 | G33 |
| X-77 | L-0 | G3 | G4 | X-78 | L-0 | G3 | G5 |
| X-79 | L-0 | G3 | G6 | X-80 | L-0 | G3 | G7 |
| X-81 | L-0 | G3 | G8 | X-82 | L-0 | G3 | G9 |
| X-83 | L-0 | G3 | G10 | X-84 | L-0 | G3 | G11 |
| X-85 | L-0 | G3 | G12 | X-86 | L-0 | G3 | G13 |
| X-87 | L-0 | G3 | G14 | X-88 | L-0 | G3 | G15 |
| X-89 | L-0 | G3 | G16 | X-90 | L-0 | G3 | G17 |
| X-91 | L-0 | G3 | G18 | X-92 | L-0 | G3 | G19 |
| X-93 | L-0 | G3 | G20 | X-94 | L-0 | G3 | G21 |
| X-95 | L-0 | G3 | G22 | X-96 | L-0 | G3 | G23 |
| X-97 | L-0 | G3 | G24 | X-98 | L-0 | G3 | G25 |
| X-99 | L-0 | G3 | G26 | X-100 | L-0 | G3 | G27 |
| X-101 | L-0 | G3 | G28 | X-102 | L-0 | G3 | G29 |
| X-103 | L-0 | G3 | G30 | X-104 | L-0 | G3 | G31 |
| X-105 | L-0 | G3 | G32 | X-106 | L-0 | G3 | G33 |
| X-107 | L-0 | G4 | G5 | X-108 | L-0 | G4 | G6 |
| X-109 | L-0 | G4 | G7 | X-110 | L-0 | G4 | G8 |
| X-111 | L-0 | G4 | G9 | X-112 | L-0 | G4 | G10 |
| X-113 | L-0 | G4 | G11 | X-114 | L-0 | G4 | G12 |
| X-115 | L-0 | G4 | G13 | X-116 | L-0 | G4 | G14 |
| X-117 | L-0 | G4 | G15 | X-118 | L-0 | G4 | G16 |
| X-119 | L-0 | G4 | G17 | X-120 | L-0 | G4 | G18 |
| X-121 | L-0 | G4 | G19 | X-122 | L-0 | G4 | G20 |
| X-123 | L-0 | G4 | G21 | X-124 | L-0 | G4 | G22 |
| X-125 | L-0 | G4 | G23 | X-126 | L-0 | G4 | G24 |
| X-127 | L-0 | G4 | G25 | X-128 | L-0 | G4 | G26 |
| X-129 | L-0 | G4 | G27 | X-130 | L-0 | G4 | G28 |
| X-131 | L-0 | G4 | G29 | X-132 | L-0 | G4 | G30 |
| X-133 | L-0 | G4 | G31 | X-134 | L-0 | G4 | G32 |
| X-135 | L-0 | G4 | G33 | X-136 | L-0 | G5 | G6 |
| X-137 | L-0 | G5 | G7 | X-138 | L-0 | G5 | G8 |
| X-139 | L-0 | G5 | G9 | X-140 | L-0 | G5 | G10 |
| X-141 | L-0 | G5 | G11 | X-142 | L-0 | G5 | G12 |
| X-143 | L-0 | G5 | G13 | X-144 | L-0 | G5 | G14 |
| X-145 | L-0 | G5 | G15 | X-146 | L-0 | G5 | G16 |
| X-147 | L-0 | G5 | G17 | X-148 | L-0 | G5 | G18 |
| X-149 | L-0 | G5 | G19 | X-150 | L-0 | G5 | G20 |
| X-151 | L-0 | G5 | G21 | X-152 | L-0 | G5 | G22 |
| X-153 | L-0 | G5 | G23 | X-154 | L-0 | G5 | G24 |
| X-155 | L-0 | G5 | G25 | X-156 | L-0 | G5 | G26 |
| X-157 | L-0 | G5 | G27 | X-158 | L-0 | G5 | G28 |
| X-159 | L-0 | G5 | G29 | X-160 | L-0 | G5 | G30 |
| X-161 | L-0 | G5 | G31 | X-162 | L-0 | G5 | G32 |
| X-163 | L-0 | G5 | G33 | X-164 | L-0 | G10 | G26 |
| X-165 | L-0 | G10 | G27 | X-166 | L-0 | G10 | G28 |
| X-167 | L-0 | G10 | G29 | X-168 | L-0 | G10 | G30 |
| X-169 | L-0 | G10 | G31 | X-170 | L-0 | G10 | G32 |
| X-171 | L-0 | G10 | G33 | X-172 | L-0 | G11 | G26 |
| X-173 | L-0 | G11 | G27 | X-174 | L-0 | G11 | G28 |
| X-175 | L-0 | G11 | G29 | X-176 | L-0 | G11 | G30 |
| X-177 | L-0 | G11 | G31 | X-178 | L-0 | G11 | G32 |
| X-179 | L-0 | G11 | G33 | X-180 | L-1 | G1 | G1 |
| X-181 | L-1 | G2 | G2 | X-182 | L-1 | G5 | G5 |
| X-185 | L-1 | G31 | G31 | X-186 | L-1 | G32 | G32 |
| X-187 | L-1 | G1 | G2 | X-188 | L-1 | G1 | G3 |
| X-189 | L-1 | G1 | G4 | X-190 | L-1 | G1 | G5 |
| X-191 | L-1 | G1 | G10 | X-192 | L-1 | G1 | G11 |
| X-193 | L-1 | G1 | G19 | X-194 | L-1 | G1 | G20 |
| X-195 | L-1 | G1 | G27 | X-196 | L-1 | G1 | G28 |
| X-197 | L-1 | G1 | G31 | X-198 | L-1 | G1 | G32 |
| X-199 | L-1 | G2 | G3 | X-200 | L-1 | G2 | G4 |
| X-201 | L-1 | G2 | G5 | X-202 | L-1 | G2 | G10 |
| X-203 | L-1 | G2 | G11 | X-204 | L-1 | G2 | G19 |
| X-205 | L-1 | G2 | G20 | X-206 | L-1 | G2 | G27 |
| X-207 | L-1 | G2 | G28 | X-208 | L-1 | G2 | G31 |
| X-209 | L-1 | G2 | G32 | X-210 | L-1 | G4 | G5 |
| X-211 | L-1 | G4 | G10 | X-212 | L-1 | G4 | G11 |
| X-213 | L-1 | G4 | G19 | X-214 | L-1 | G4 | G20 |
| X-215 | L-1 | G4 | G27 | X-216 | L-1 | G4 | G28 |
| X-217 | L-1 | G4 | G31 | X-218 | L-1 | G4 | G32 |
| X-219 | L-1 | G5 | G10 | X-220 | L-1 | G5 | G11 |
| X-221 | L-1 | G5 | G19 | X-222 | L-1 | G5 | G20 |
| X-223 | L-1 | G5 | G27 | X-224 | L-1 | G5 | G28 |
| X-225 | L-1 | G5 | G31 | X-226 | L-1 | G5 | G32 |
| X-227 | L-4 | G1 | G1 | X-228 | L-4 | G2 | G2 |
| X-229 | L-4 | G5 | G5 | | | | |
| | | | | X-232 | L-4 | G31 | G31 |
| X-233 | L-4 | G32 | G32 | X-234 | L-4 | G1 | G2 |
| X-235 | L-4 | G1 | G3 | X-236 | L-4 | G1 | G4 |
| X-237 | L-4 | G1 | G5 | X-238 | L-4 | G1 | G10 |
| X-239 | L-4 | G1 | G11 | X-240 | L-4 | G1 | G19 |
| X-241 | L-4 | G1 | G20 | X-242 | L-4 | G1 | G27 |
| X-243 | L-4 | G1 | G28 | X-244 | L-4 | G1 | G31 |
| X-245 | L-4 | G1 | G32 | X-246 | L-4 | G2 | G3 |
| X-247 | L-4 | G2 | G4 | X-248 | L-4 | G2 | G5 |
| X-249 | L-4 | G2 | G10 | X-250 | L-4 | G2 | G11 |
| X-251 | L-4 | G2 | G19 | X-252 | L-4 | G2 | G20 |
| X-253 | L-4 | G2 | G27 | X-254 | L-4 | G2 | G28 |
| X-255 | L-4 | G2 | G31 | X-256 | L-4 | G2 | G32 |
| X-257 | L-4 | G4 | G5 | X-258 | L-4 | G4 | G10 |
| X-259 | L-4 | G4 | G11 | X-260 | L-4 | G4 | G19 |
| X-261 | L-4 | G4 | G20 | X-262 | L-4 | G4 | G27 |
| X-263 | L-4 | G4 | G28 | X-264 | L-4 | G4 | G31 |
| X-265 | L-4 | G4 | G32 | X-266 | L-4 | G5 | G10 |
| X-267 | L-4 | G5 | G11 | X-268 | L-4 | G5 | G19 |
| X-269 | L-4 | G5 | G20 | X-270 | L-4 | G5 | G27 |
| X-271 | L-4 | G5 | G28 | X-272 | L-4 | G5 | G31 |
| X-273 | L-4 | G5 | G32. | | | | |

20. The organic electroluminescent device of claim 1, wherein the first organic layer is a hole injection layer, and the hole injection layer is in contact with the anode.

21. The organic electroluminescent device of claim 1, wherein the organic electroluminescent device further comprises at least one light-emitting layer; preferably, the at least one light-emitting layer contains at least one host material and at least one doping material;
more preferably, the organic electroluminescent device has a maximum emission wavelength between 300 nm and 1200 nm.

22. The organic electroluminescent device of claim 21, wherein the organic electroluminescent device further comprises a second organic layer, and the second organic layer is disposed between the first organic layer and the at least one light-emitting layer;
preferably, the second organic layer contains one compound containing any one or more chemical structural units selected from the following group: triarylamine, carbazole, fluorene, spirobifluorene, thiophene, furan, phenyl, oligophenylene ethylene, oligofluorene and combinations thereof,
more preferably, the one compound in the second organic layer is the second compound.

23. The organic electroluminescent device of claim 22, wherein the organic electroluminescent device further comprises a third organic layer, and the third organic layer is disposed between the second organic layer and the light-emitting layer;
preferably, the third organic layer contains another compound containing any one or more chemical structural units selected from the following group: triarylamine, carbazole, fluorene, spirobifluorene, thiophene, furan, phenyl, oligophenylene ethylene, oligofluorene and combinations thereof,
more preferably, the another compound in the third organic layer is the second compound.

24. The organic electroluminescent device of claim 22, wherein the first organic layer has a thickness ranging from 0.1 nm to 40 nm, and the second organic layer has a thickness ranging from 0.1 nm to 300 nm.

25. A display assembly, comprising the organic electroluminescent device of claim 1.

26. A compound combination containing a first compound and a second compound, wherein the first compound has a structure represented by Formula 1: 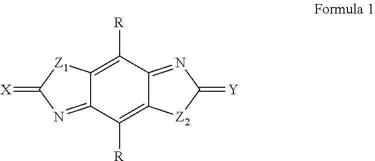
wherein in Formula 1,
X and Y are, at each occurrence identically or differently, selected from NR′, CR″R′″, O, S or Se;
Z₁ and Z₂ are, at each occurrence identically or differently, selected from O, S or Se;
R, R′, R″ and R′″ are, at each occurrence identically or differently, selected from the group consisting of: hydrogen, deuterium, halogen, a nitroso group, a nitro group, an acyl group, a carbonyl group, a carboxylic acid group, an ester group, a cyano group, an isocyano group, SCN, OCN, SF₅, a boryl group, a sulfinyl group, a sulfonyl group, a phosphoroso group, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,
each R may be the same or different, and at least one of R, R′, R″ and R′″ is a group having at least one electron withdrawing group;
in Formula 1, adjacent substituents can be optionally joined to form a ring;
wherein the second compound has a structure represented by Formula 2: 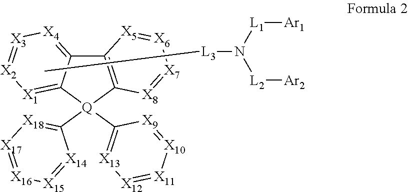
wherein in Formula 2,
X₁ to X₈ are, at each occurrence identically or differently, selected from C, CR₁ or N; and X₉ to X₁₈ are, at each occurrence identically or differently, selected from CR₁ or N;
Q is selected from C, Si or Ge;
L₁ to L₃ are, at each occurrence identically or differently, selected from a single bond, a substituted or unsubstituted arylene group having 6 to 30 carbon atoms, a substituted or unsubstituted heteroarylene group having 3 to 30 carbon atoms or a combination thereof,
Ar₁ and Ar₂ are, at each occurrence identically or differently, selected from substituted or unsubstituted aryl having 6 to 30 carbon atoms or substituted or unsubstituted heteroaryl having 3 to 30 carbon atoms;
R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents L₁, L₂, L₃, R₁, Ar₁ and Ar₂ can be optionally joined to form a ring.
